(19) United States
(12) Patent Application Publication
(10) Pub. No.: US 2012/0058989 A1
(43) Pub. Date: Mar. 8, 2012

(54) ANTIBACTERIAL FLUOROQUINOLONE ANALOGS

(75) Inventors: Allan S. Wagman, Belmont, CA (US); Heinz E. Moser, San Mateo, CA (US); Glenn A. McEnroe, San Mateo, CA (US); James B. Aggen, Burlingame, CA (US); Martin S. Linsell, San Mateo, CA (US); Adam A. Goldblum, Berkeley, CA (US); John H. Griffin, Atherton, CA (US); Lloyd J. Simons, Mattawan, MI (US); Thomas R. Belliotti, Portage, MI (US); Christina R. Harris, Weimar, CA (US); Toni-Jo Poel, Plainwell, MI (US); Michael J. Melnick, Portage, MI (US); Ricky D. Gaston, Kalamazoo, MI (US)

(73) Assignee: Achaogen, Inc., South San Francisco, CA (US)

(21) Appl. No.: 13/082,269

(22) Filed: Apr. 7, 2011

Related U.S. Application Data
(63) Continuation of application No. 12/853,051, filed on Aug. 9, 2010, now abandoned.
Application No. 12/853,051 is a continuation of application No. PCT/US2009/033946, filed on Feb. 12, 2009.
(60) Provisional application No. 61/027,952, filed on Feb. 12, 2008.

(51) Int. Cl. A61K 31/553; A61P 31/04; C07D 498/14; C07D 471/14; A61K 31/55; A61K 31/5517

(52) U.S. Cl. 514/211.1; 514/214.02; 514/219; 540/546; 540/555; 540/579

(57) ABSTRACT

Compounds having antibacterial activity are disclosed. The compounds have the following structure (I):

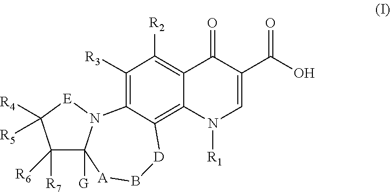

including stereoisomers, pharmaceutically acceptable salts and prodrugs thereof, wherein A, B, D, E, G, R₁, R₂, R₃, R₄, R₅, R₆ and R₇ are as defined herein. Methods associated with preparation and use of such compounds, as well as pharmaceutical compositions comprising such compounds, are also disclosed.

### CROSS-REFERENCE TO RELATED APPLICATIONS

This application is a continuation of U.S. patent application Ser. No. 12/853,051, filed Aug. 9, 2010, now pending; which is a continuation of International PCT Application No. PCT/US2009/033946, filed Feb. 12, 2009, now pending; which claims the benefit under 35 U.S.C. §119(e) of U.S. Provisional Patent Application No. 61/027,952, filed Feb. 12, 2008. The foregoing applications are incorporated herein by reference in their entireties.

### STATEMENT OF GOVERNMENT INTEREST

This invention was made with government support under Contract No. HDTRA1-07-C-0005, awarded by the Defense Threat Reduction Agency, an agency of the United States Department of Defense. The government has certain rights in this invention.

### BACKGROUND OF THE INVENTION

1. Field of the Invention

The present invention is directed to novel fluoroquinolone compounds, and methods for their preparation and use as therapeutic or prophylactic agents.

2. Description of the Related Art

Antibiotics are chemical substances produced by various species of microorganisms (bacteria, fungi, actinomycetes) that suppress the growth of other microorganisms and may eventually destroy them. However, common usage often extends the term antibiotics to include synthetic antibacterial agents, such as the sulfonamides, oxazolidinones, or quinolones, that are not products of microbes. The number of antibiotics that have been identified now extends into the hundreds, and many of these have been developed to the stage where they are of value in the therapy of infectious diseases. Antibiotics differ markedly in physical, chemical, and pharmacological properties, antibacterial spectra, and mechanisms of action. In recent years, knowledge of molecular mechanisms of bacterial, fungal, and viral replication has greatly facilitated rational development of compounds that can interfere with the life cycles of these microorganisms.

At least 30% of all hospitalized patients now receive one or more courses of therapy with antibiotics, and millions of potentially fatal infections have been cured. At the same time, these pharmaceutical agents have become among the most misused of those available to the practicing physician. One result of widespread use of antimicrobial agents has been the emergence of resistance, which in turn has increasingly rendered existing antibiotics inactive against multi-drug resistant pathogens and has created an ever-increasing need for new drugs.

The fluoroquinolone class of antibiotics are a powerful tool in combating bacterial infections. Fluoroquinolones have been used extensively to treat respiratory tract infections (including for example, bronchitis, pneumonia, tuberculosis), urinary tract infections, diarrhea, postoperative-wound infections, bone and joint infections, skin infections, inflammation of the prostate, ear infections, various sexually transmitted diseases, various infections that affect people with AIDS, and other conditions, in animals and humans. Fluoroquinolones are active against a wide spectrum of Gram-positive and Gram-negative bacteria. For example, various fluoroquinolones have been found to be effective against Staphylococcus aureus, Streptococcus pneumoniae, coagulase-negative staphylococci, Streptococcus pyogenes, Staphylococcus epidermis, Escherichia coli, Klebsiella pneumoniae, Enterobacter cloacae, Pseudomonas aeruginosa, Proteus mirabilis, Proteus vulgaris, Providencia stuartii, Morganella morganii, Citrobacter diversus, Citrobacter freundii, Haemophilus influenzae, and Neisseria gonorrhea, and other organisms. Indeed, the mounting resistance of Staphylococcus aureus to both penicillin and erythromycin has made the fluoroquinolone antibiotics a viable alternative for the treatment of skin diseases and pneumoniae.

Fluoroquinolones were first developed in the early 1960s. The first precursor of fluoroquinolones, nalidixic acid, was approved by the FDA in 1963 for the treatment of urinary tract infections. Nalidixic acid is rapidly absorbed after oral administration and is excreted into the urine in bactericidal concentrations. Nalidixic acid, however, has several limitations that has prevented its use in other types of infections. Specifically, nalidixic acid has a narrow spectrum of activity and microorganisms easily developed resistance to the drug. The development of other fluoroquinolones by chemically altering the basic structure of nalidixic acid, however, has led to improved fluoroquinolones that are more effective against resistant bacteria and effective against a broader range of bacteria.

Ciprofloxacin was approved by the FDA in 1986 for the oral treatment of bacterial infections and set a benchmark especially for Gram-negative organisms. More compounds from the fluoroquinolone class were approved in the following years: levofloxacin (1993, initially approved as the racemate ofloxacin in 1985), gatifloxacin (1999), moxifloxacin (1999), and gemifloxacin (2003), to just name a few. The latter compounds were greatly improved for their potency against Gram-positive organisms including S. aureus and S. pneumoniae such that they even cover multi-drug resistant organisms (gemifloxacin for S. pneumoniae). However, the level of resistance has constantly be on the rise especially in Gram-negative organisms and reached an extent that many clinical isolates can not be treated any longer with the currently approved fluoroquinolones. The two major reasons for this observation are mutations of the target proteins (mutations in the “quinolone-resistance determining region” or QRDR in the genes encoding gyrase and topoisomerase IV) and an increased level of efflux (more important in Gram-negative organisms). See, e.g., Bryskier, A., “Fluororquinolones” in Antimicrobial Agents: Antibacterials and Antifungals, Bryskier, A. ed., ASM Press, Washington, D.C., 2005, pp 668-788; Domagala, J. M. and Hagen, S. E., “Structure-Activity Relationships of the Quinolone Antibacterials in the New Millennium: Some Things Change and Some Do Not” in Quinolone Antimicrobial Agents, 3^{rd} ed., Hooper, D. C. and Rubinstein, E. eds., ASM Press, Washington, D.C., 2003, pp 3-18; Gootz, T. D. and Brighty, K. E., “Fluoroquinolone Antibacterials: SAR, Mechanism of Action, Resistance, and Clinical Aspects” Medicinal Research Reviews 16(5): 433-486 (1996); and Zhanel, G. G, et al., “A Critical Review of the Fluoroquinolones: Focus on Respiratory Tract Infections” Drugs 62(1): 13-59 (2002).

Accordingly, while progress has been made in this field, there remains a need in the art for new chemical entities that possess antibacterial activity against fluoroquinolone-resistant clinical isolates. The present invention fulfills this need and provides further related advantages.

### BRIEF SUMMARY OF THE INVENTION

In brief, the present invention is directed to novel fluoroquinolone compounds having antibacterial activity, including stereoisomers, pharmaceutically acceptable salts and prodrugs thereof, and the use of such compounds in the treatment of bacterial infections.

In one embodiment, compounds having the following structure (I) are provided:

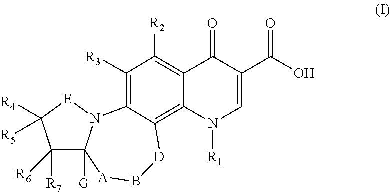

or a stereoisomer, pharmaceutically acceptable salt or prodrug thereof,

wherein:

A, B and D are as follows:
A is —C(R_{8b})₂—, —C(═O)—, —C(R_{8b})(OR₈ₐ)—, —C(R₈ₐ)(N(R₈ₐ)₂)—, —C(═NOR₈ₐ)—, —S(═O)— or —SO₂—;
B is —C(R_{8b})₂—, —C(═O)—, —C(R_{8b})(OR₈ₐ)—, —C(R_{8b})(N(R₈ₐ)₂)—, —C(═NOR₈ₐ)—, —O—, —S—, —S(═O)—, —SO₂— or —N(R₈ₐ)—; and
D is —C(R_{8b})₂—, —C(═O)—, —O—, —S—, —SO₂—, —N(R₈ₐ)—, —C(R_{8b})(OR₈ₐ)— or —C(R_{8b})(N(R₈ₐ)₂)—;
or A-B, taken together, are —C(R_{8b})═C(R_{8b})—, —C(R_{8b})═N— or

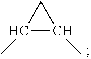

or
or B-D, taken together, are —C(R_{8b})═C(R_{8b})—, —N═C(R_{8b})— or

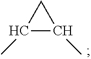

E is —C(R_{8c})₂— or —C(═O)—;

G is hydrogen or methyl;

R₁ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl or optionally substituted heteroarylalkyl;

R₂ is hydrogen, methyl or amino;

R₃ is hydrogen, fluorine or chlorine;

R₄, R₅, R₆, R₇ are, independently, hydrogen, halogen, amino, hydroxyl, optionally substituted alkyl, optionally substituted cycloalkyl, optionally substituted alkoxy, optionally substituted alkylamino or —N(R₈ₐ)₂, or R₄ and R₅, taken together, are ═CHR_{8b}, ═NOR₈ₐ, ═NNR₈ₐ or ═O or, together with the atom to which they are attached, form an optionally substituted heterocyclic ring having from 3 to 6 ring atoms, or R₆ and R₇, taken together, are ═CHR_{8b}, ═NOR₈ₐ, ═NNR₈ₐ or ═O, or, together with the atom to which they are attached, form an optionally substituted heterocyclic ring having from 3 to 6 ring atoms, or R₅ and R₆, R₅ and R₇, R₄ and R₆, or R₄ and R₇, taken together with the atoms to which they are attached, form a heterocyclic ring having from 3 to 6 ring atoms;

each R₈ₐ is, independently, hydrogen, C₁-C₆ alkyl, C₁-C₆ cycloalkyl, C₁-C₆ cycloalkylalkyl or —C(═O)R_{8c};

each R_{8b} is, independently, hydrogen, halogen, C₁-C₆ alkyl, C₁-C₆ cycloalkyl, C₁-C₆ haloalkyl or C₁-C₆ cycloalkylalkyl; and

each R_{8c} is, independently, hydrogen or C₁-C₆ alkyl.

In another embodiment, a pharmaceutical composition is provided comprising a compound having structure (I), or a stereoisomer, pharmaceutically acceptable salt or prodrug thereof, and a pharmaceutically acceptable carrier, diluent or excipient.

In another embodiment, a method of using a compound having structure (I) in therapy is provided. In particular, the present invention provides a method of treating a bacterial infection in a mammal, comprising administering to the mammal an effective amount of a compound having structure (I), or a stereoisomer, pharmaceutically acceptable salt or prodrug thereof.

In other embodiments, methods for the preparation of a compound having structure (I) is provided. Intermediate compounds intermediates useful in such methods are also provided.

These and other aspects of the invention will be apparent upon reference to the following detailed description.

### DETAILED DESCRIPTION OF THE INVENTION

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ radical.

“Cyano” refers to the —CN radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” refers to a straight or branched hydrocarbon chain radical consisting solely of carbon and hydrogen atoms, which is saturated or unsaturated (i.e., contains one or more double and/or triple bonds), having from one to twelve carbon atoms (C₁-C₁₂ alkyl), preferably one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group may be optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Alkoxy” refers to a radical of the formula —ORₐ where Rₐ is an alkyl radical as defined above containing one to twelve carbon atoms. Unless stated otherwise specifically in the specification, an alkoxy group may be optionally substituted.

“Alkylamino” refers to a radical of the formula —NHRₐ or —NRₐRₐ where each Rₐ is, independently, an alkyl radical as defined above containing one to twelve carbon atoms. Unless stated otherwise specifically in the specification, an alkylamino group may be optionally substituted.

“Thioalkyl” refers to a radical of the formula —SRₐ where Rₐ is an alkyl radical as defined above containing one to twelve carbon atoms. Unless stated otherwise specifically in the specification, a thioalkyl group may be optionally substituted.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. Unless stated otherwise specifically in the specification, an aralkyl group may be optionally substituted.

“Cycloalkyl” or “carbocyclic ring” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted.

“Cycloalkylalkyl” refers to a radical of the formula —R_{b}R_{d} where R_{d} is an alkylene chain as defined above and R_{g} is a cycloalkyl radical as defined above. C₁-C₆ cycloalkylalkyl refers to a radical wherein the alkylene chain has from one to six carbon atoms. Unless stated otherwise specifically in the specification, a cycloalkylalkyl group may be optionally substituted.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, Unless stated otherwise specifically in the specification, a heterocyclyl group may be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group may be optionally substituted.

“Heterocyclylalkyl” refers to a radical of the formula —R_{b}Rₑ where R_{b} is an alkylene chain as defined above and Rₑ is a heterocyclyl radical as defined above, and if the heterocyclyl is a nitrogen-containing heterocyclyl, the heterocyclyl may be attached to the alkyl radical at the nitrogen atom. Unless stated otherwise specifically in the specification, a heterocyclylalkyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group may be optionally substituted.

“Heteroarylalkyl” refers to a radical of the formula —R_{b}R_{f} where R_{b} is an alkylene chain as defined above and R_{f} is a heteroaryl radical as defined above. Unless stated otherwise specifically in the specification, a heteroarylalkyl group may be optionally substituted.

The term “substituted” used herein means any of the above groups (i.e., alkyl, alkylene, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{B}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

“Prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of structure (I) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action, or binding affinity to pharmacologically important site of action. Certain isotopically-labelled compounds of structure (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of structure (I) can generally be prepared by conventional techniques known to those skilled in the art or by processes analogous to those described in the Preparations and Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound of the invention which, when administered to a mammal, preferably a human, is sufficient to effect treatment, as defined below, of a bacterial infection in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

“Bacterial infection” refers to the establishment of a sufficient population of a pathogenic bacteria in a patient to have a deleterious effect on the health and well-being of the patient and/or to give rise to discernable symptoms associated with the particular bacteria.

“Fluoroquinolone antibiotic resistant bacterium” or “fluoroquinolone-resistant bacterium” refers to bacterium against which at least one of the following known fluoroquinolone antibiotics, namely, ciprofloxacin, levofloxacin, moxifloxacin and gemifloxacin, has a minimum inhibitory concentration (MIC) greater than or equal to 4 μg/mL.

As noted above, in one embodiment of the present invention, compounds having antibacterial activity are provided, the compounds having the following structure (I):

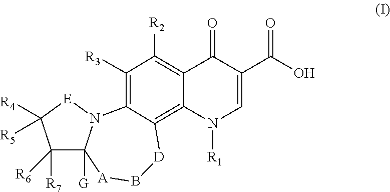

or a stereoisomer, pharmaceutically acceptable salt or prodrug thereof,

wherein:

A, B and D are as follows:
A is —C(R_{8b})₂—, —C(═O)—, —C(R_{8b})(OR₈ₐ)—, —C(R_{8b})(N(R₈ₐ)₂)—, —C(═NOR₈ₐ)—, —S(═O)— or —SO₂—;
B is —C(R_{8b})₂—, —C(═O)—, —C(R_{8b})(OR₈ₐ)—, —C(R_{8b})(N(R₈ₐ)₂)—, —C(═NOR₈ₐ)—, —O—, —S—, —S(═O)—, —SO₂— or —N(R₈ₐ)—; and
D is —C(R_{8b})₂—, —C(═O)—, —O—, —S—, —S(═O)—, —SO₂—, —N(R₈ₐ)—, —C(R_{8b})(OR₈ₐ)— or —C(R_{8b})(N(R₈ₐ)₂)—;
or A-B, taken together, are —C(R_{8b})═C(R_{8b})—, —C(R_{8b})═N— or

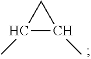

or
or B-D, taken together, are —C(R_{8b})═C(R_{8b})—, —N═C(R_{8b})— or

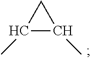

E is —C(R_{8c})₂— or —C(═O)—;

G is hydrogen or methyl;

R₁ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl or optionally substituted heteroarylalkyl;

R₂ is hydrogen, methyl or amino;

R₃ is hydrogen, fluorine or chlorine;

R₄, R₅, R₆, R₇ are, independently, hydrogen, halogen, amino, hydroxyl, optionally substituted alkyl, optionally substituted cycloalkyl, optionally substituted alkoxy, optionally substituted alkylamino or —N(R₈ₐ)₂, or R₄ and R₅, taken together, are ═CHR_{8b}, ═NOR₈ₐ, ═NNR₈ₐ or ═O or, together with the atom to which they are attached, form an optionally substituted heterocyclic ring having from 3 to 6 ring atoms, or R₆ and R₇, taken together, are ═CHR_{8b}, ═NOR₈ₐ, ═NNR₈ₐ or ═O, or, together with the atom to which they are attached, form an optionally substituted heterocyclic ring having from 3 to 6 ring atoms, or R₅ and R₆, R₅ and R₇, R₄ and R₆, or R₄ and R₇, taken together with the atoms to which they are attached, form a heterocyclic ring having from 3 to 6 ring atoms;

each R₈ₐ is, independently, hydrogen, C₁-C₆ alkyl, C₁-C₆ cycloalkyl, C₁-C₆ cycloalkylalkyl or —C(═O)R_{8c};

each R_{8b} is, independently, hydrogen, halogen, C₁-C₆ alkyl, C₁-C₆ cycloalkyl, C₁-C₆ haloalkyl or C₁-C₆ cycloalkylalkyl; and

each R_{8c} is, independently, hydrogen or C₁-C₆ alkyl.

In certain embodiments, the compounds have the following structure (I):

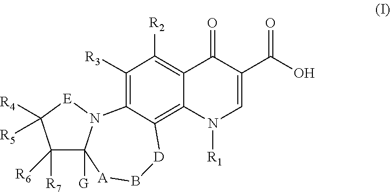

or a stereoisomer, pharmaceutically acceptable salt or prodrug thereof,

wherein:

A, B and D are as follows:
A is —C(R_{8b})₂—, —C(═O)—, —CR_{8b}OR₈ₐ—, —CR_{8b}N(R₈ₐ)₂—, —C(═NOR₈ₐ)—, —S(═O)— or —SO₂—;
B is —C(R_{8b})₂—, —C(═O)—, —CR_{8b}OR₈ₐ—, —CR_{8b}N(R₈ₐ)₂—, —C(═NOR₈ₐ)—, —O—, —S—, —S(═O)—, —SO₂— or —NR₈ₐ—; and
D is —C(R_{8b})₂—, —C(═O)—, —O—, —S—, —S(═O)—, —SO₂—, —NR₈ₐ—, —CR_{8b}OR₈ₐ— or —CR_{8b}N(R₈ₐ)₂—;
or A-B, taken together, are —CR_{8b}═CR_{8b}— or

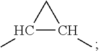

or

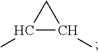

or B-D, taken together, are —CR_{8b}═CR_{8b}

E is —C(R_{8c})₂— or —C(═O)—;

G is hydrogen or methyl;

R₁ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl or optionally substituted heteroarylalkyl;

R₂ is hydrogen, methyl or amino;

R₃ is hydrogen, fluorine or chlorine;

R₄, R₅, R₆, R₇ are, independently, hydrogen, amino, optionally substituted alkyl, optionally substituted alkoxy or optionally substituted alkylamino, or R₄ and R₅, taken together, are ═CHR_{8b}, ═NOR₈ₐ or ═O or, together with the atom to which they are attached, form an optionally substituted heterocyclic ring having from 3 to 6 ring atoms, or R₆ and R₇, taken together, are ═CHR_{8b}, ═NOR₈ₐ or ═O, or, together with the atom to which they are attached, form an optionally substituted heterocyclic ring having from 3 to 6 ring atoms, or R₅ and R₆, R₅ and R₇, R₄ and R₆, or R₄ and R₇, taken together with the atoms to which they are attached, form a heterocyclic ring having from 3 to 6 ring atoms;

each R₈ₐ is, independently, hydrogen, C₁-C₆ alkyl, C₁-C₆ cycloalkyl or C₁-C₆ cycloalkylalkyl;

each R_{8b} is, independently, hydrogen, halogen, C₁-C₆ alkyl, C₁-C₆ cycloalkyl, C₁-C₆ haloalkyl or C₁-C₆ cycloalkylalkyl; and

each R_{8c} is, independently, hydrogen or C₁-C₆ alkyl.

In certain embodiments, A-B-D, taken together, are —CH₂CH₂CH₂—, —CH(CH₃)CH₂CH₂—, —C(CH₃)₂CH₂CH₂—, —CH₂CH═CH—, —CH═CHCH₂—, —CH(OR₈ₐ)CH₂CH₂—, —C(CH₃)(OR₈ₐ)CH₂CH₂—, —CH(N(R₈ₐ)₂)CH₂CH₂—, —C(CH₃)(N(R₈ₐ)₂)CH₂CH₂—, —C(═NOR₈ₐ)CH₂CH₂—, —C(═O)N(R₈ₐ)CH₂—, —CH₂CH(OR₈ₐ)CH₂—, —CH₂C(CH₃)(OR₈ₐ)CH₂—, —CH₂CH(N(R₈ₐ)₂)CH₂—, CH₂C(═NOR₈ₐ)CH₂—, —CH₂C(═O)CH₂—, —CH₂C(CH₃)₂CH₂—, —CH₂OCH₂—, —CH₂SCH₂—, —CH₂S(═O)CH₂—, —CH₂SO₂CH₂—, —CH₂N(R₈ₐ)CH₂—, —CH₂CH₂CH(CH₃)—, —CH₂CH₂C(CH₃)₂—, —CH₂C(═O)N(R₈ₐ)—, —CH₂N(R₈ₐ)C(═O)—, —CH₂SO₂N(R₈ₐ)—, —CH₂N(R₈ₐ)SO₂—, —CH₂CH₂O—, —CH₂CH₂N(R₈ₐ)—, —CH₂CH₂S—, —CH₂CH₂S(═O)— or —CH₂CH₂SO₂—.

In certain embodiments, A-B-D, taken together, are —C(R_{8b})₂C(R_{8b})₂C(R_{8b})₂—, —C(R_{8b})₂C(R_{8b})—C(R_{8b})— or —C(R_{8b})═C(R_{8b})C(R_{8b})₂—. In such embodiments, each R_{8b} may be hydrogen.

In certain embodiments, A is —CH₂—. In such embodiments, B-D, taken together, may be —C(R_{8b})₂O—, —OC(R_{8b})₂—, —C(R_{8b})₂S—, —SC(R_{8b})₂—, —C(R_{8b})₂N(R₈ₐ)—, —N(R₈ₐ)C(R_{8b})₂—, —C(R_{8b})₂C(R_{8b})₂—, —C(R_{8b})═C(R_{8b})— or —N═C(R_{8b})—. In addition, R₈ₐ and R_{8b} may be hydrogen, C₁-C₆ alkyl or C₁-C₆ cycloalkyl.

In certain embodiments, B is —CH₂—. In such embodiments, A may be —C(R_{8b})₂— and D may be —C(R_{8b})₂— or —O—. In addition, R_{8b} may be hydrogen, C₁-C₆ alkyl or C₁-C₆ cycloalkyl.

In certain embodiments, B is —O—. In such embodiments, A may be —C(R_{8b})₂— and D may be —C(R_{8b})₂—. In addition, R_{8b} may be hydrogen, C₁-C₆ alkyl or C₁-C₆ cycloalkyl.

In certain embodiments, B is —S—. In such embodiments, A may be —C(R_{8b})₂— and D may be —C(R_{8b})₂—. In addition, R_{8b} may be hydrogen, C₁-C₆ alkyl or C₁-C₆ cycloalkyl.

In certain embodiments, D is —CH₂—. In such embodiments, A-B, taken together, may be —CH₂CH(R_{8b})—, —CH(R_{8b})CH₂—, —C(CH₃)₂CH₂—, —CH₂C(CH₃)₂—,

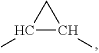

—C(R_{8b})═C(R_{8b})—, —CH₂C(═NOR₈ₐ)—, —C(═NOR₈ₐ)CH₂—, —CH₂O—, —CH₂S—, —CH₂S(═O)—, —CH₂SO₂—, —CH₂N(R₈ₐ)—, —N(R₈ₐ)CH₂—, —CH(OR₈ₐ)CH₂—, —CH(N(R₈ₐ)₂)CH₂—, —CH₂CH(N(R₈ₐ)₂)—, —C(═O)N(R₈ₐ)—, —CH₂C(═O)—, or —CH₂CH(OR_{8b})— or —C(R_{8b})═N—. In addition, R₈ₐ and R_{8b} may be hydrogen, C₁-C₆ alkyl or C₁-C₆ cycloalkyl.

In certain embodiments, D is —O—. In such embodiments, A-B taken together, may be —CH₂CH₂—, —CH═CH—, —CH(OR₈ₐ)CH₂—,

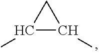

—CH(N(R₈ₐ)₂)CH₂—, —C(CH₃)₂CH₂—, —CH₂C(CH₃)₂—, —CH₂CH(R_{8b})— or —CH(R_{8b})CH₂—. In addition, R₈ₐ and R_{8b} may be hydrogen, C₁-C₆ alkyl or C₁-C₆ cycloalkyl.

In certain embodiments, D is —N(R₈ₐ)—. In such embodiments, R₈ₐ may be hydrogen or methyl. In addition, A-B, taken together, may be —CH₂CH(R_{8b})—, —CH(R_{8b})CH₂—, —C(CH₃)₂CH₂—, —CH₂C(CH₃)₂—, —CH(OR₈ₐ)CH₂—, —CH(N(R₈ₐ)₂)CH₂—, —CH₂C(═O)— or —CH₂SO₂—. In addition, R₈ₐ and R_{8b} may be hydrogen, C₁-C₆ alkyl or C₁-C₆ cycloalkyl.

In certain embodiments, B-D, taken together, are —C(R_{8b})═C(R_{8b})—. In such embodiments, A may be —C(R_{8b})₂—. In addition, R_{8b} may be hydrogen, C₁-C₆ alkyl or C₁-C₆ cycloalkyl.

In certain embodiments, R₄, R₅, R₆ and R₇ are, independently, hydrogen, amino, optionally substituted alkyl, optionally substituted cycloalkyl, optionally substituted alkylamino or —N(R₈ₐ)₂. In further embodiments, R₄, R₅, R₆ and R₇ may each be hydrogen. In other further embodiments, R₄, R₅ and R₆ may each be hydrogen and R₇ may be amino, substituted alkyl, substituted cycloalkyl, alkylamino, or —N(R₈ₐ)₂, wherein substituted alkyl is —(C₁-C₆ alkyl)N(R₈ₐ)₂ and substituted cycloalkyl is —(C₃-C₆ cycloalkyl)N(R₈ₐ)₂. In such embodiments, each R₈ₐ may be hydrogen and R₇ may be —NH₂, —CH₂NH₂, —CH(CH₃)NH₂, —C(CH₃)₂NH₂, or 1-amino-cycloprop-1-yl. In other further embodiments, R₄, R₆ and R₇ may each be hydrogen and R₅ may be amino, substituted alkyl, substituted cycloalkyl, alkylamino, or —N(R₈ₐ)₂, wherein substituted alkyl is —(C₁-C₆ alkyl)N(R₈ₐ)₂ and substituted cycloalkyl is —(C₃-C₆ cycloalkyl)N(R₈ₐ)₂. In such embodiments, each R₈ₐ may be hydrogen and R₅ may be —NH₂, —CH₂NH₂, —CH(CH₃)NH₂, —C(CH₃)₂NH₂, or 1-amino-cycloprop-1-yl.

In certain embodiments, R₄ and R₅, taken together, are ═N(OR₈ₐ) or ═O. In such embodiments, R₆ may be hydrogen and R₇ may be amino, substituted alkyl, substituted cycloalkyl, alkylamino, or —N(R₈ₐ)₂, wherein substituted alkyl is —(C₁-C₆ alkyl)N(R₈ₐ)₂ and substituted cycloalkyl is —(C₃-C₆cycloalkyl)N(R₈ₐ)₂.

In certain embodiments, R₆ and R₇, taken together, are ═N(OR₈ₐ) or ═O. In such embodiments, R₄ may be hydrogen and R₅ may be amino, substituted alkyl, substituted cycloalkyl, alkylamino, or —N(R₈ₐ)₂, wherein substituted alkyl is —(C₁-C₆alkyl)N(R₈ₐ)₂ and substituted cycloalkyl is —(C₃-C₆cycloalkyl)N(R₈ₐ)₂.

In certain embodiments, R₄ and R₅, taken together with the atom to which they are attached, form a heterocyclic ring having from 3 to 6 ring atoms and the compound has the following structure:

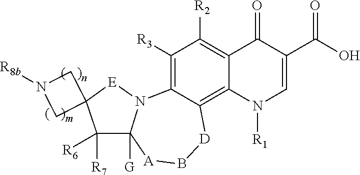

wherein n and m are, independently, 0, 1 or 2, provided that n and m are not both 0.

In certain embodiments, R₆ and R₇, taken together with the atom to which they are attached, form a heterocyclic ring having from 3 to 6 ring atoms and the compound has the following structure:

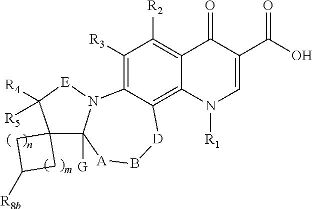

wherein n and m are, independently, 0, 1 or 2, provided that n and m are not both 0.

In certain embodiments, R₁ is optionally substituted alkyl. For example, R₁ may be C₁-C₆ alkyl.

In certain embodiments, R₁ is optionally substituted cycloalkyl. For example, R₁ may be cyclopropyl.

In certain embodiments, R₂ is hydrogen.

In certain embodiments, R₃ is fluorine.

In certain embodiments, R₃ is hydrogen.

In certain embodiments, E is —CH₂—.

In certain embodiments, E is —C(═O)—.

In certain embodiments, E is —CH(CH₃)— or —C(CH₃)₂—.

In certain embodiments, G is hydrogen.

In certain embodiments, the compound has the following structure:

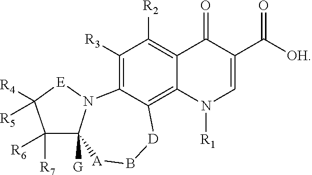

It is understood that any embodiment of the compounds of structure (I), as set forth above, and any specific substituent set forth herein for a A, B, D, E, G, R₁, R₂, R₃, R₄, R₅, R₆ and R₇ group in the compounds of structure (I), as set forth above, may be independently combined with other embodiments and/or substituents of compounds of structure (I) to form embodiments of the inventions not specifically set forth above. In addition, in the event that a list of substitutents is listed for any particular R group in a particular embodiment and/or claim, it is understood that each individual substituent may be deleted from the particular embodiment and/or claim and that the remaining list of substituents will be considered to be within the scope of the invention.

It is further understood that in the present description, any specific combination set forth herein for the A, B and D groups in the compounds of structure (I) is specific with respect to the position of such groups. For example, it is understood that the terminology “A-B-D, taken together, are —CH₂N(R₈ₐ)SO₂-” indicates that A is —CH₂—, B is —N(R₈ₐ)— and D is —SO₂—.

It is further understood that in the present description, combinations of substituents and/or variables of the depicted formulae are permissible only if such contributions result in stable compounds. For example, in the above embodiments of the compounds of structure (I), it is understood that:

(i) A and B are not both —S(═O)—, —SO₂— or —C(═O)—;

(ii) B and D are not both —O—, —S—, —S(═O)—, —SO₂— or —C(═O)—;

(iii) B and D are not both —O—, —S—, —S(═O)— or —NR₈ₐ—;

(iv) A and D are not both —S(═O)—, —SO₂— or —C(═O)—;

(v) if B is —C(R₈ₐ)₂, then A and D are not both —O—, —S—, —S(═O)— or —NR₈ₐ—;

(vi) if B is —CR_{8b}OR₈ₐ— or —CR_{8b}N(R₈ₐ)₂—, then D is not —NR₈ₐ—, —O— or —S—;

(vii) if B is —NR₈ₐ—, then A is not —S(═O)— or —SO₂— and D is not —O—, —S—, or —NR₈ₐ—;

(viii) if A is —CR_{8b}OR₈ₐ— or —CR_{8b}N(R₈ₐ)₂—, then B is not —NR₈ₐ—, —O— or —S—; and

(ix) if D is —CR_{8b}OR₈ₐ— or —CR_{8b}N(R₈ₐ)₂—, then B is not —NR₈ₐ—, —O— or —S—.

For the purposes of administration, the compounds of the present invention may be administered as a raw chemical or may be formulated as pharmaceutical compositions. Pharmaceutical compositions of the present invention comprise a compound of structure (I) and a pharmaceutically acceptable carrier, diluent or excipient. The compound of structure (I) is present in the composition in an amount which is effective to treat a particular disease or condition of interest—that is, in an amount sufficient to treat a bacterial infection, and preferably with acceptable toxicity to the patient. The antibacterial activity of compounds of structure (I) can be determined by one skilled in the art, for example, as described in the Examples below. Appropriate concentrations and dosages can be readily determined by one skilled in the art.

The compounds of the present invention possess antibacterial activity against a wide spectrum of gram positive and gram negative bacteria, as well as enterobacteria and anaerobes. Representative susceptible organisms generally include those Gram-positive and Gram-negative, aerobic and anaerobic organisms whose growth can be inhibited by the compounds of the invention, such as species of Staphylococcus, Enterococcus, Streptococcus, Sarcina, Escherichia, Enterobacter, Klebsiella, Pseudomonas, Burkholderia, Acinetobacter, Aeromonas, Proteus, Campylobacter, Pasteurella, Citrobacter, Legionella, Neisseria, Bordetella, Baccillus, Bacteroides, Moraxella, Morganella, Edwardsiella, Peptococcus, Clostridium, Providencia, Salmonella, Stenotrophomonas, Shigella, Serratia, Haemophilus, Vibrio and Yersinia, and other similar organisms, as well as Mycobacterium organisms, such as Mycobacterium tuberculosis, Mycobacterium avium, and the like.

For example, the compounds possess antibacterial activity against the following bacteria: Enterococcus faecium, Staphylococcus epidermidis, Staphylococcus haemolyticus, Staphylococcus hominis, Staphylococcus saprophyticus, Streptococcus agalactiae, Streptococcus (Group C/F), Streptococcus (Group G), Viridans group streptococci, Acinetobacter baumannii, Acinetobacter calcoaceticus, Acinetobacter lwoffii, Aeromonas hydrophila, Bordetella pertussis, Burkholderia cepacia, Campylobacter jejuni, Citrobacter diversus, Citrobacter freundii, Enterobaeter aerogenes, Enterobacter agglomerans, Enterobacter sakazaki, Edwardsiella tarda, Haemophilus influenzae, Haemophilus parainfluenzae, Klebsiella oxytoca, Klebsiella pneumoniae, Legionella pneumophila, Moraxella catarrhalis, Morganella morganii, Neisseria gonorrhoeae, Pasteurella multocida, Proteus mirabilis, Proteus vulgaris, Providencia rettgeri, Providencia stuartii, Pseudomonas aeruginosa, Pseudomonas fluorescens, Salmonella enteritidis, Salmonella typhi, Serratia liquefaciens, Serratia marcescens, Shigella boydii, Shigella dysenteriae, Shigella flexneri, Shigella sonnei, Stenotrophomonas maltophilia, Vibrio cholerae, Vibrio parahaemolyticus, Vibrio vulnificus, Yersinia enterocolitica, Clostridium difficile and Clostridium perfringens.

In addition, the compounds of the present invention have MIC ≦2 μg/mL for each of (i) one or more Gram-negative bacteria selected from the group consisting of Acinetobacter anitratus, Acinetobacter baumannii, Acinetobacter calcoaceticus, Acinetobacter lwoffii, Aeromonas hydrophila, Bordetella pertussis, Burkholderia cepacia, Campylobacter jejuni, Citrobacter diversus, Citrobacter freundii, Enterobaeter aerogenes, Enterobacter agglomerans, Enterobacter cloacae, Enterobacter sakazaki, Escherichia coli, Edwardsiella tarda, Haemophilus influenzae, Haemophilus parainfluenzae, Klebsiella oxytoca, Klebsiella pneumoniae, Legionella pneumophila, Moraxella catarrhalis, Morganella morganii, Neisseria gonorrhoeae, Pasteurella multocida, Proteus mirabilis, Proteus vulgaris, Providencia rettgeri, Providencia stuartii, Pseudomonas aeruginosa, Pseudomonas fluorescens, Salmonella enteritidis, Salmonella typhi, Serratia liquefaciens, Serratia marcescens, Shigella boydii, Shigella dysenteriae, Shigella flexneri, Shigella sonnei, Stenotrophomonas maltophilia, Vibrio cholerae, Vibrio parahaemolyticus, Vibrio vulnificus and Yersinia enterocolitica; and (ii) one or more Gram-positive bacteria selected from the group consisting of Enterococcus faecalis, Enterococcus faecium, Staphylococcus aureus, Staphylococcus epidermidis, Staphylococcus haemolyticus, Staphylococcus hominis, Staphylococcus saprophyticus, Streptococcus agalactiae, Streptococcus (Group C/F), Streptococcus (Group G), Streptococcus pneumoniae, Streptococcus pyogenes, Viridans group streptococci, Clostridium difficile and Clostridium perfringens. In particular, the compounds of the present invention have MIC ≦2 μg/mL for each of (i) one or more Gram-negative bacteria selected from the group consisting of Acinetobacter baumannii, Acinetobacter calcoaceticus, Burkholderia cepacia, Citrobacter freundii, Enterobaeter aerogenes, Enterobacter cloacae, Escherichia coli, Haemophilus influenzae, Klebsiella oxytoca, Klebsiella pneumoniae, Morganella morganii, Proteus mirabilis, Proteus vulgaris, Providencia stuartii, Pseudomonas aeruginosa, Salmonella enteritidis, Serratia liquefaciens, Serratia marcescens, Shigella dysenteriae, Shigella flexneri and Yersinia enterocolitica, and (ii) one or more Gram-positive bacteria selected from the group consisting of Staphylococcus aureus, Staphylococcus epidermidis and Streptococcus pneumoniae.

In addition, it has been discovered that the compounds of the present invention possess antibacterial activity against bacterial species resistant to conventional fluoroquinolone antibiotics, such as fluoroquinolone resistant Acinetobacter baumannii, Pseudomonas aeruginosa, Escherichia coli, Staphylococcus aureus, Streptococcus pneumoniae, Klebsiella pneumoniae, Morganella morganii, Proteus mirabilis, Enterobaeter aerogenes, Enterobacter cloacae, Providencia stuartii or Serratia marcescens bacterium. In particular, fluoroquinolone resistant Acinetobacter baumannii, Pseudomonas aeruginosa, Escherichia coli, Staphylococcus aureus or Streptococcus pneumoniae bacterium.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of the invention, or pharmaceutically acceptable derivatives thereof, may also be administered simultaneously with, prior to, or after administration of one or more other therapeutic agents. Such combination therapy includes administration of a single pharmaceutical dosage formulation which contains a compound of the invention and one or more additional active agents, as well as administration of the compound of the invention and each active agent in its own separate pharmaceutical dosage formulation. For example, a compound of the invention and the other active agent can be administered to the patient together in a single oral dosage composition such as a tablet or capsule, or each agent administered in separate oral dosage formulations. Where separate dosage formulations are used, the compounds of the invention and one or more additional active agents can be administered at essentially the same time, i.e., concurrently, or at separately staggered times, i.e., sequentially; combination therapy is understood to include all these regimens.

The following Examples illustrate various methods of making compounds of this invention, i.e., compound of structure (I):

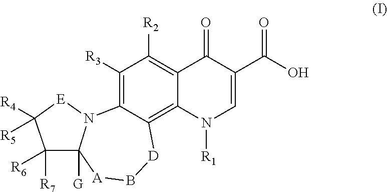

wherein A, B, D, E, G, R₁, R₂, R₃, R₄, R₅, R₆ and R₇ are as defined above. It is understood that one skilled in the art may be able to make these compounds by similar methods or by combining other methods known to one skilled in the art. It is also understood that one skilled in the art would be able to make, in a similar manner as described below, other compounds of structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described in this invention.

It will be appreciated by those skilled in the art that in the methods described herein the functional groups of intermediate compounds may need to be protected by suitable “protecting groups”. Such functional groups include hydroxy, amino, mercapto and carboxylic acid. Suitable protecting groups for hydroxy include, for example, trialkylsilyl or diarylalkylsilyl (for example, triethylsilyl (TES), triisopropylsilyl (TIPS), t-butyldimethylsilyl (TBS), t-butyldiphenylsilyl (TBDPS) or trimethylsilyl (TMS)), tert-butoxycarbonyl (Boc), allyloxycarbonyl (Alloc), carboxybenzyl (Cbz), fluorenylmethoxycarbonyl (Fmoc), trichloroethoxycarbonyl (Troc), trityl (Trt), benzyl, methoxybenzyl, dimethoxybenzyl, chlorobenzyl, dichlorobenzyl, trifluoroacetic acid amide (TFA), phenacyl amide and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”, as defined herein.

Furthermore, all compounds of the invention which exist in free base or acid form can be converted to their pharmaceutically acceptable salts by treatment with the appropriate inorganic or organic base or acid by methods known to one skilled in the art. Salts of the compounds of the invention can be converted to their free base or acid form by standard techniques.

For example, in one embodiment, a compound having the following structure (INT-I) is provided:

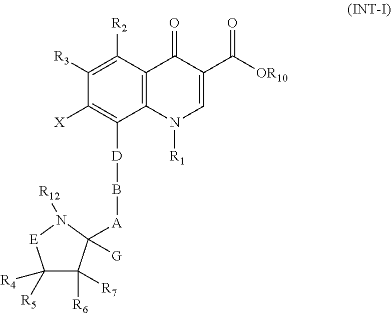

or a stereoisomer or salt thereof,

wherein,
A-B-D taken together is selected from —C(R_{8b})₂C≡C— and —C(R_{8b})₂C(R_{8b})═C(R_{8b})—;
R₁ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl or optionally substituted heteroarylalkyl;
R₂ is hydrogen, methyl or amino;
R₃ is hydrogen, fluorine or chlorine;
R₄, R₅, R₆, R₇ are, independently, hydrogen, halogen, amino, hydroxyl, optionally substituted alkyl, optionally substituted cycloalkyl, optionally substituted alkoxy, optionally substituted alkylamino or —N(R₈ₐ)₂, or R₄ and R₅, taken together, are ═CHR_{8b}, ═NOR₈ₐ, ═NNR₈ₐ or ═O or, together with the atom to which they are attached, form an optionally substituted heterocyclic ring having from 3 to 6 ring atoms, or R₆ and R₇, taken together, are ═CHR_{8b}, ═NOR₈ₐ, ═NNR₈ₐ or ═O, or, together with the atom to which they are attached, form an optionally substituted heterocyclic ring having from 3 to 6 ring atoms, or R₅ and R₆, R₅ and R₇, R₄ and R₆, or R₄ and R₇, taken together with the atoms to which they are attached, form a heterocyclic ring having from 3 to 6 ring atoms;
each R₈ₐ is, independently, hydrogen, C₁-C₆ alkyl, C₁-C₆ cycloalkyl, C₁-C₆ cycloalkylalkyl or —C(═O)R_{8c};
each R_{8b} is, independently, hydrogen, halogen, C₁-C₆ alkyl, C₁-C₆ cycloalkyl, C₁-C₆ haloalkyl or C₁-C₆ cycloalkylalkyl;
each R_{8c} is, independently, hydrogen or C₁-C₆ alkyl;
R₁₀ is hydrogen, optionally substituted C₁-C₆ alkyl, optionally substituted aryl or optionally substituted aralkyl
R₁₂ is hydrogen or a protecting group;
X is halogen or triflate;
E is —C(R_{8c})₂— or —C(═O)—; and
G is hydrogen or methyl.

In further embodiments, the compound has the following structure (INT-IA1):

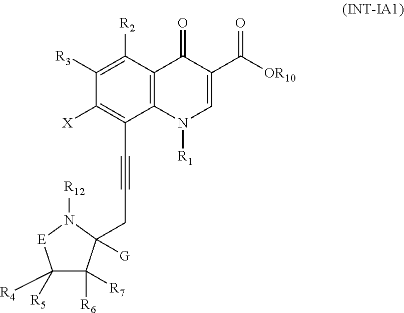

or a stereoisomer or salt thereof.

In other further embodiments, the compound has the following structure (INT-IA2):

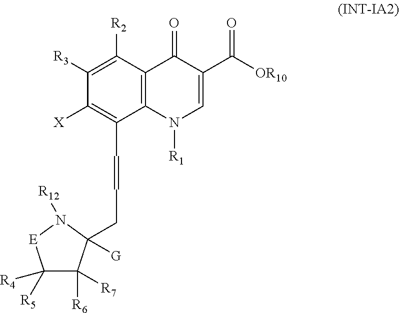

or a stereoisomer or salt thereof.

In more specific embodiments, X is fluoro and/or R₁₂ is hydrogen or a protecting group selected from the group consisting of tert-butoxycarbonyl (Boc), allyloxycarbonyl (Alloc), carboxybenzyl (Cbz), fluorenylmethoxycarbonyl (Fmoc), trichloroethoxycarbonyl (Troc), trityl (Trt), benzyl, methoxybenzyl, dimethoxybenzyl, chlorobenzyl, dichlorobenzyl, trifluoroacetic acid amide (TFA) and phenacyl amide. For example, in certain embodiments, X is fluoro and R₁₂ is tert-butoxycarbonyl (Boc), carboxybenzyl (Cbz) or trifluoroacetic acid amide (TFA).

In other more specific embodiments, R₁₀ is selected from the group consisting of methyl, ethyl, propyl, isopropyl, butyl, tert-butyl, allyl, phenyl, benzyl, methoxybenzyl, dimethoxybenzyl, chlorobenzyl and dichlorobenzyl. For example, in certain embodiments, R₁₀ is selected from the group consisting of methyl, ethyl, tert-butyl and benzyl.

In other more specific embodiments, the compound has the following structure (INT-IB1):

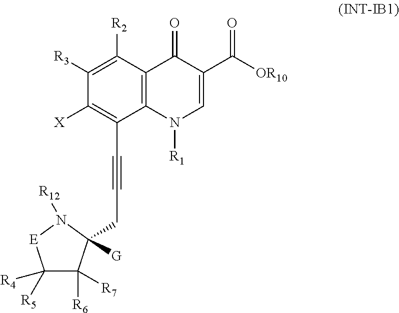

or a stereoisomer or salt thereof.

In other more specific embodiments, the compound has the following structure (INT-IB2):

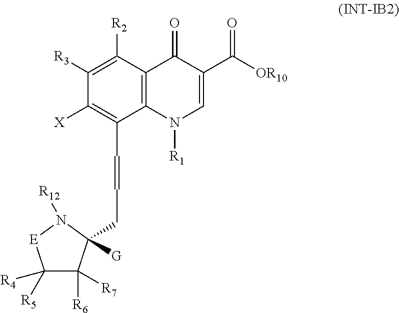

or a stereoisomer or salt thereof.

In another embodiment, a method for preparing a compound of structure (I) is provided, the method comprising:

(i) providing a compound having the following structure (INT-I):

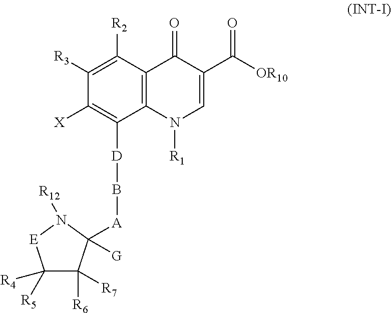

or a stereoisomer or salt thereof,

wherein:

R₁₀ is optionally substituted. C₁-C₆ alkyl, optionally substituted aryl or optionally substituted aralkyl;

R₁₂ is hydrogen or a protecting group;

X is halogen or triflate;

(ii) cyclizing the pyrrolidine nitrogen with the quinolone moiety under reaction conditions to form a seven-membered heterocyclic moiety comprising A-B-D; and

(iii) optionally hydrolyzing the —OR₁₀ ester moiety, to form a compound of structure (I).

In more specific embodiments, the compound of structure (INT-I) is a compound having the following structure (INT-IA2):

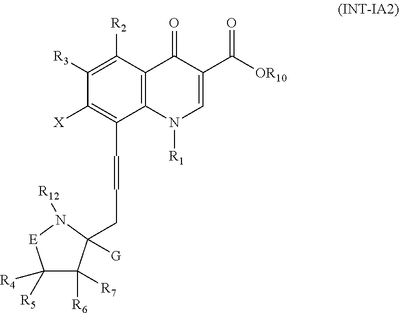

or a stereoisomer or salt thereof.

In a more specific embodiment, the compound of structure (INT-IA2) is prepared by reducing a compound having the following structure (INT-IA1):

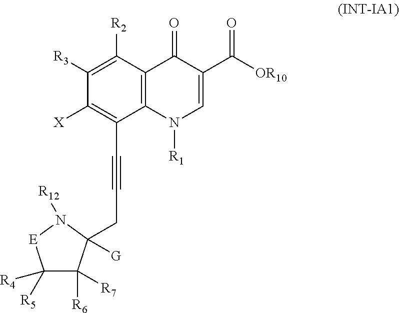

or a stereoisomer or salt thereof.

In another embodiment, a method for preparing a compound of structure (I) is provided, the method comprising:

(i) providing a compound having the following structure (INT-II):

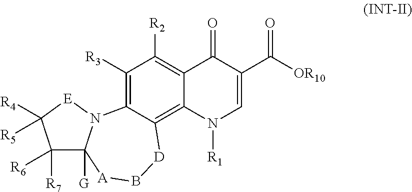

or a stereoisomer or salt thereof,

wherein:

A-B-D taken together is an unsaturated moiety —C(R_{8b})₂C(R_{8b})═C(R_{8b})—; and

R₁₀ is optionally substituted C₁-C₆ alkyl, optionally substituted aryl or optionally substituted aralkyl;

(ii) reducing the unsaturated A-B-D moiety to form the saturated A-B-D moiety, and

(iii) optionally hydrolyzing the —OR₁₀ ester moiety, to form a compound of structure (I).

In a more specific embodiment, the compound of structure (INT-II) is prepared by a method comprising:

(i) providing a compound having the following structure (INT-I):

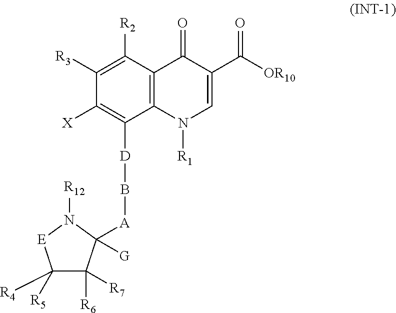

or a stereoisomer or salt thereof,

wherein:

A-B-D taken together is an unsaturated moiety —C(R_{8b})₂C(R_{8b})═C(R_{8b})—;

R₁₂ is hydrogen or a protecting group; and

X is halogen or triflate;

(ii) cyclizing the pyrrolidine nitrogen with the quinolone moiety under reaction conditions to form a seven-membered heterocyclic moiety comprising the unsaturated A-B-D moiety.

In another more specific embodiment, the compound of structure (INT-I) is a compound having the following structure (INT-IA2):

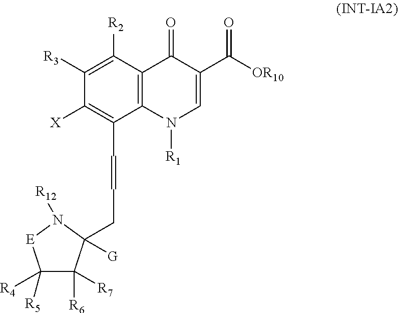

or a stereoisomer or salt thereof.

In another more specific embodiment, the compound of structure (INT-IA2) is prepared by reducing a compound having the following structure (INT-IA1):

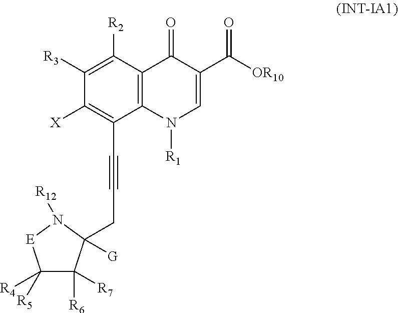

or a stereoisomer or salt thereof.

In another embodiment, a compound having the following structure (INT-III) is provided:

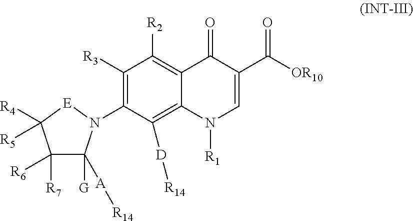

or a stereoisomer or salt thereof,

wherein,

A and D are, independently, —C(R_{8b})₂—;

R₁ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl or optionally substituted heteroarylalkyl;

R₂ is hydrogen, methyl or amino;

R₃ is hydrogen, fluorine or chlorine;

R₄, R₅, R₆, R₇ are, independently, hydrogen, halogen, amino, hydroxyl, optionally substituted alkyl, optionally substituted cycloalkyl, optionally substituted alkoxy, optionally substituted alkylamino or —N(R₈ₐ)₂, or R₄ and R₅, taken together, are ═CHR_{8b}, ═NOR₈ₐ, ═NNR₈ₐ or ═O or, together with the atom to which they are attached, form an optionally substituted heterocyclic ring having from 3 to 6 ring atoms, or R₆ and R₇, taken together, are ═CHR_{8b}, ═NOR₈ₐ, ═NNR₈ₐ or ═O, or, together with the atom to which they are attached, form an optionally substituted heterocyclic ring having from 3 to 6 ring atoms, or R₅ and R₆, R₅ and R₇, R₄ and R₆, or R₄ and R₇, taken together with the atoms to which they are attached, form a heterocyclic ring having from 3 to 6 ring atoms;

each R₈ₐ is, independently, hydrogen, C₁-C₆ alkyl, C₁-C₆ cycloalkyl, C₆ cycloalkylalkyl or —C(═O)R_{8c};

each R_{8b} is, independently, hydrogen, halogen, C₁-C₆ alkyl, C₁-C₆ cycloalkyl, C₁-C₆ haloalkyl or C₁-C₆ cycloalkylalkyl;

each R_{8c} is, independently, hydrogen or C₁-C₆ alkyl;

R₁₀ is hydrogen, optionally substituted C₁-C₆ alkyl, optionally substituted aryl or optionally substituted aralkyl;

each R₁₄ is, independently, —OR₁₄ₐ or halogen, wherein each R₁₄ₐ is, independently, hydrogen, trifluoromethanesulfonate, mesylate, tosylate, tert-butyl, allyl, trimethylsilyl (TMS), triethylsilyl (TES), tert-butyldimethylsilyl (TBS), triisopropylsilyl (TIPS), tert-butyldiphenylsilyl (TBDPS), tert-butyldimethyl silyl (TBDMS), acetyl, benzyl, methoxybenzyl, dimethoxybenzyl, chlorobenzyl or dichlorobenzyl;

E is —C(R_{8c})₂— or —C(═O)—; and

G is hydrogen or methyl.

In a more specific embodiment, the compound has the following structure (INT-IIIA):

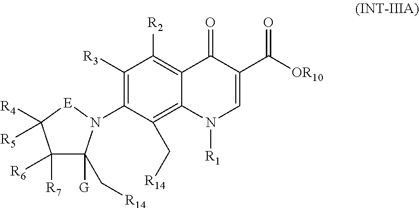

or a stereoisomer or salt thereof.

In other more specific embodiments, R₁₀ is selected from the group consisting of methyl, ethyl, propyl, isopropyl, butyl, tert-butyl, allyl, phenyl, benzyl, methoxybenzyl, dimethoxybenzyl, chlorobenzyl and dichlorobenzyl.

In other more specific embodiments, R₁₄ is —OR₁₄ₐ and R₁₄ₐ is hydrogen.

In other more specific embodiments, the compound has the following structure (INT-IIIB):

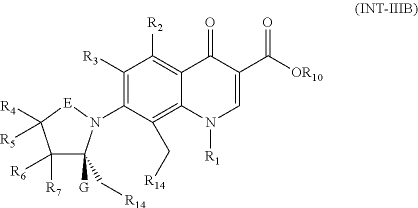

or a stereoisomer or salt thereof.

In another embodiment, a method for preparing a compound of structure (I) is provided, the method comprising:

(i) providing a compound having the following structure (INT-III):

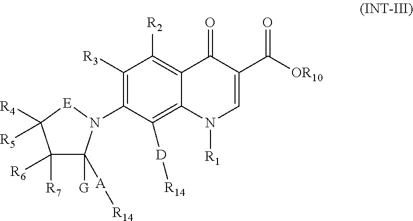

or a stereoisomer or salt thereof,

wherein:

A and D are, independently, —C(R_{8b})₂—; and

R₁₀ is optionally substituted C₁-C₆ alkyl, optionally substituted aryl or optionally substituted aralkyl;

each R₁₄ is, independently, —OR₁₄ₐ or halogen, wherein each R₁₄ₐ is, independently, hydrogen, trifluoromethanesulfonate, mesylate, tosylate, tert-butyl, allyl, trimethylsilyl (TMS), triethylsilyl (TES), tert-butyldimethylsilyl (TBS), triisopropylsilyl (TIPS), tert-butyldiphenylsilyl (TBDPS), tert-butyldimethyl silyl (TBDMS), acetyl, benzyl, methoxybenzyl, dimethoxybenzyl, chlorobenzyl or dichlorobenzyl;

(ii) condensing the —R₁₄ pendant from the pyrrolidine moiety with the —R₁₄ pendant from the quinolone moiety under reaction conditions to form a seven-membered heterocyclic moiety comprising the ether-containing moiety —C(R_{8b})₂OC(R_{8b})₂—, and

(iii) optionally, hydrolyzing the —OR₁₀ ester moiety to form a compound of structure (I).

In more specific embodiments, the compound of structure (INT-III) has the following structure (INT-IIIA):

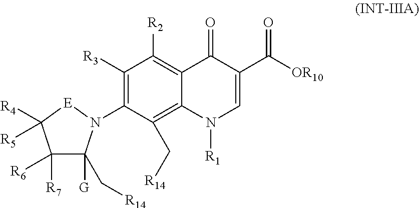

or a stereoisomer or salt thereof.

The following examples are provided for purposes of illustration, not limitation.

### EXAMPLES

### Example 1

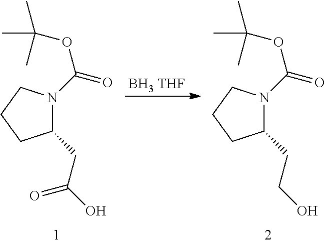

(S)-2-(1-(tert-butoxycarbonyl)pyrrolidin-2-yl)acetic acid (1) (5 g, 21.8 mmol) was dissolved in 100 mL anhydrous tetrahydrofuran, cooled to 0° C. and treated with borane-tetrahydrofuran (1 M THF; 26.2 mL; 26.2 mmol; 1.2 equiv.). The reaction mixture was stirred and temperature allowed to warm to room temp for 2 hours. The reaction mixture was cooled to 0° C. and 100 mL cold water was added carefully. The mixture was extracted with ethyl acetate (3×80 mL), the combined extracts washed with saturated sodium chloride solution, dried over anhydrous sodium sulfate and the solvent evaporated under reduced pressure to give 5.56 g (S)-tert-butyl-2-(2-hydroxyethyl)pyrrolidine-1-carboxylate (2) that was used without further purification in next step. MH⁺215.8 ¹H NMR (DMSO-d₆): δ ppm 4.40 (bs 1H); 3.73 (m 2H); 3.37 (m 2H); 3.20 (m 2H); 1.78 (m 6H); 1.35 (s 9H).

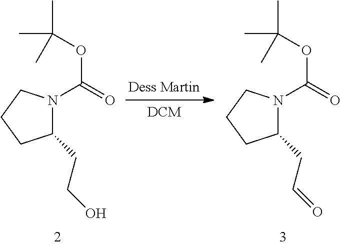

(S)-tert-butyl-2-(2-hydroxyethyl)pyrrolidine-1-carboxylate (2) (5.56 g; 25.8 mmol) was dissolved in dichloromethane. Dess-Martin periodinane (16.41 g; 38.7 mmol; 1.5 equiv.) was added and the reaction mixture was stirred at room temperature for 3 days. The reaction mixture was diluted with 150 mL diethyl ether and treated with 100 mL saturated sodium bicarbonate and 25 mL 1 M sodium thiosulfate, the mixture stirred for 10 min. The resulting mixture was filtered to remove precipitate. The filtrate was allowed to separate and the aqueous phase was extracted with diethyl ether (3×75 mL), the combined organic extracts were dried over anhydrous sodium sulfate and evaporated under vacuum. The residue was chromatographed on silica gel eluted with a gradient of 10% to 100% ethyl acetate/hexanes. Fractions containing desired product were combined and evaporated under vacuum to give the product (S)-tert-butyl-2-(2-oxoethyl)pyrrolidine-1-carboxylate (3) as an oil. 3.16 g were obtained. 1H NMR (DMSO-d₆): δ ppm 9.68 (s 1H); 4.14 (m 1H); 2.27 (m 2H); 2.60 (m 2H); 2.05 (m 1H); 1.80 (m 2H); 1.62 (m 1H); 1.41 (s 9H).

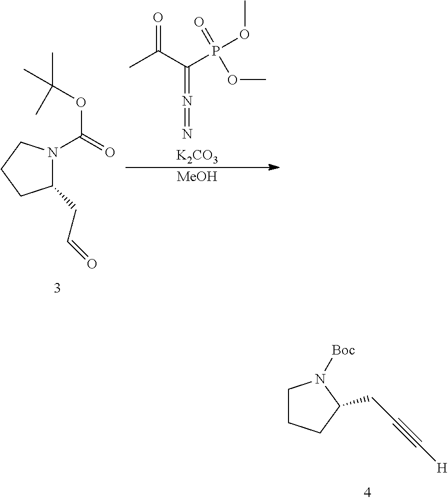

(S)-tert-butyl-2-(2-oxoethyl)pyrrolidine-1-carboxylate (3) (1.59 g, 7.46 mmol) was dissolved in 40 mL anhydrous methanol and cooled to 0° C. Potassium carbonate (1.10 g; 14.92 mol; 2 equiv.) Freshly prepared dimethyl-1-diazo-2-oxopropylphosphonate (1.52 g; 7.96 mmol) was dissolved in 5 mL methanol and added to the reaction mixture drop wise. The mixture was stirred at room temperature overnight. The solvent was removed under reduced pressure and the residue was suspended in diethyl ether (100 mL), washed with 5% sodium bicarbonate solution. The aqueous wash was extracted with diethyl ether (2×50 mL), the combined organic extracts were dried over anhydrous sodium sulfate and the solvent was removed under vacuum to give 690 mg (S)-tert-butyl-2-(prop-2-ynyl)pyrrolidine-1-carboxylate (4). ¹H NMR (CDCl₃): δ ppm 3.83 (d 1H); 2.06 (m 2H); 2.60 (m 1H); 3.34 (m 1H); 1.92 (m 4H); 1.34 (m 1H); 1.44 (s 9H).

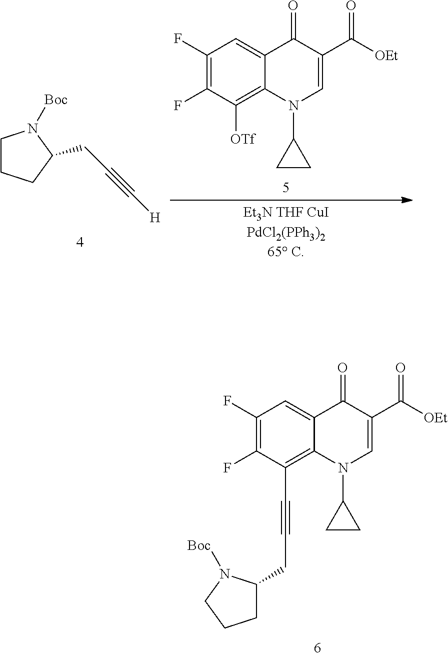

(S)-tert-butyl-2-(prop-2-ynyl)pyrrolidine-1-carboxylate (4) (114 mg; 0.544 mmol; 1.2 equiv.), ethyl-1-cyclopropyl-6,7-difluoro-4-oxo-8-(trifluoromethylsulfonyloxy)-1,4-dihydroquinoline-3-carboxylate (5) (200 mg; 0.454 mmol); dichlorobis(triphenylphosphine)Palladium(II) (32 mg; 0.045 mmol), triphenylphosphine (6 mg; 0.023 mmol) and triethylamine (91 mg; 0.908 mmol) were dissolved in anhydrous tetrahydrofuran at room temperature for 20 minutes under nitrogen. Copper iodide (9 mg; 0.0454 mmol) was added in one portion under nitrogen and the reaction mixture was heated to 60° C. for 5 hours. Reaction mixture was evaporated and residue dissolved in N-methylpyrrolidinone and purified on reversed phase HPLC. Fractions contained desired product were combined evaporated, dissolved in absolute ethanol, evaporated, dissolved in dichloromethane basified solution with triethylamine and evaporated to dryness to give 183 mg (S)-ethyl-8-(3-(1-(tert-butoxycarbonyl)pyrrolidin-2-yl)prop-1-ynyl)-1-cyclopropyl-6,7-difluoro-4-oxo-1,4-dihydroquinoline-3-carboxylate (6) as a white solid. MH⁺501.2.

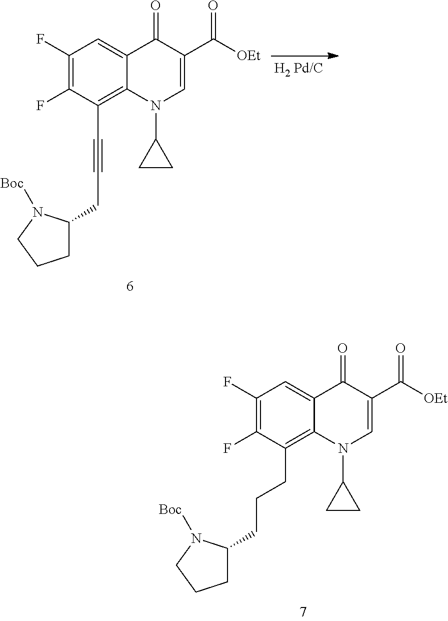

(S)-Ethyl-8-(3-(1-(tert-butoxycarbonyl)pyrrolidin-2-yl)prop-1-ynyl)-1-cyclopropyl-6,7-difluoro-4-oxo-1,4-dihydroquinoline-3-carboxylate (6) (183 mg; 0.366 mmol) was dissolved in absolute ethanol and 10% Pd/C (30 mg) was added to the mixture at room temperature. Reaction mixture was subjected to hydrogenation under balloon pressure for 16 hours. The reaction mixture was diluted with ethanol and filtered. The filtrate was evaporated to give 215 mg (R)-ethyl-8-(3-(1-(tert-butoxycarbonyl)pyrrolidin-2-yl)propyl)-1-cyclopropyl-6,7-difluoro-4-oxo-1,4-dihydroquinoline-3-carboxylate (7). MH⁺505.3.

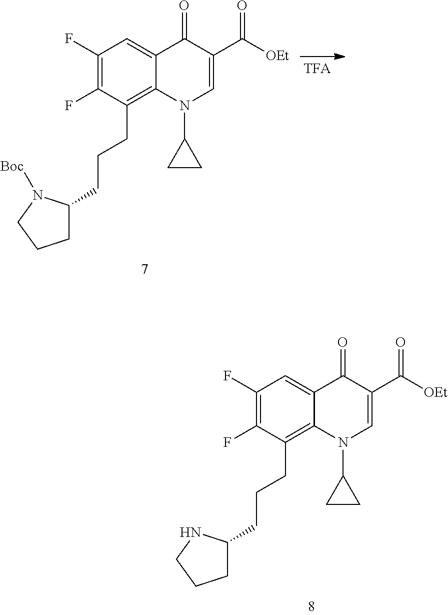

(R)-ethyl-8-(3-(1-(tert-butoxycarbonyl)pyrrolidin-2-yl)propyl)-1-cyclopropyl-6,7-difluoro-4-oxo-1,4-dihydroquinoline-3-carboxylate (7) (183 mg; 0.366 mmol) was dissolved in 5 mL dichloromethane and treated with trifluoroacetic acid (0.5 mL). The mixture was stirred at room temperature for 2 hours. The mixture was evaporated and dissolved in dichloromethane and evaporated again to give 200 mg (R)-ethyl-1-cyclopropyl-6,7-difluoro-4-oxo-8-(3-(pyrrolidin-2-yl)propyl)-1,4-dihydroquinoline-3-carboxylate (8). MH⁺405.2.

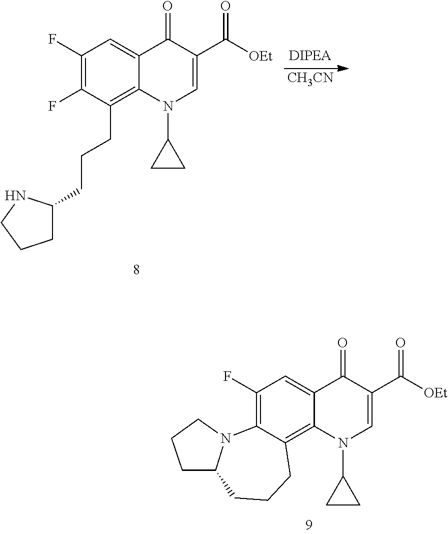

(R)-ethyl-1-cyclopropyl-6,7-difluoro-4-oxo-8-(3-(pyrrolidin-2-yl)propyl)-1,4-dihydroquinoline-3-carboxylate (8) (148 mg; 0.366 mmol) was dissolved in 5 mL anhydrous acetonitrile. diisopropylethylamine (189 mg; 0.255 mL; 4 equiv.) was added to the mixture in a sealed vial. The reaction mixture was heated to 70° C. for 2 days, then 80° C. for 8 hours followed by 85° C. for 2 days. Evaporated mixture and dissolved in N-methylpyrrolidinone and purified by reversed phase HPLC to give 31 mg product (9). MH⁺: 385.2.

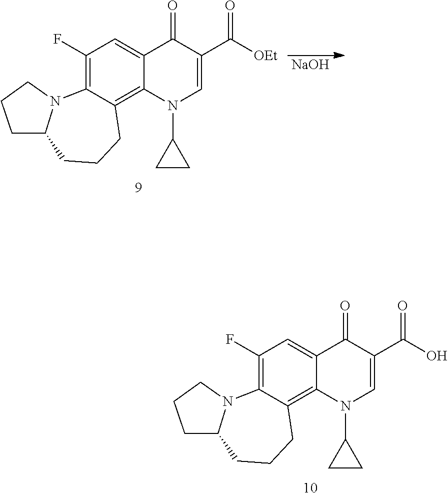

Compound (9) (31 mg; 0.081 mmol) was added to acetonitrile (3 mL) and water (2 mL). Enough 1N sodium hydroxide solution was added to make the solution slightly basic. The mixture was heated to 60° C. for 12 hours. Additional 1N sodium hydroxide solution was added (0.010 mL) and the mixture was heated to 70° C. for 3 hours. The reaction mixture was acidified by the addition of 2 drops glacial acetic acid to pH ˜5. The mixture was evaporated to dryness, dissolved in N-methylpyrrolidinone and subjected to reversed phase HPLC purification to give 14 mg product (10) as a yellow solid. MH⁺357.1; ¹H NMR (CDCl₃): δ ppm 8.819 (s 1H); 7.779 (d, 14 Hz 1H); 4.007 (m 1H); 3.749 (m 1H); 3.54 (q 1H); 2.01 (m 4H); 1.76 (m 4H); 2.38 (m 5H); 0.997 (m 1H); 0.819 (m 2H).

### Example 2

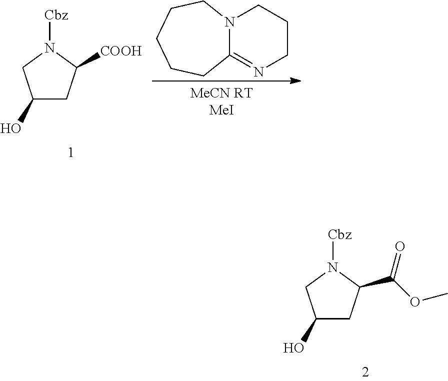

(2R,4R)-1-(benzyloxycarbonyl)-4-hydroxypyrrolidine-2-carboxylic acid (1) (25 g; 94.25 mmol) was suspended in 125 mL acetonitrile. 1,8-diazabicyclo[5.4.0]undec-7ene (14.34 g; 94.25 mmol; 1 equiv.) was added to the solution with stirring and after 15 min at room temperature methyl iodide (13.37 g; 94.25 mmol; 1 equiv.) was added and the reaction was stirred for 3 days at room temperature. The solvent was removed under reduced pressure and residue suspended in ethyl acetate (200 mL). A 0.1 M potassium hydrogen sulfate (200 mL) was added and the product extracted into the organic phase. Extracted aqueous wash with 200 mL ethyl acetate and combined extracts were washed with saturated sodium chloride solution, the organic phase was dried over anhydrous sodium sulfate and evaporated to dryness under vacuum, to give product as a yellow oil. Obtained 25.24 g (2R,4R)-1-benzyl-2-methyl-4-hydroxypyrrolidine-1,2-dicarboxylate (2). ¹H NMR (DMSO-d₆): δ ppm 7.34 (m 5H); 5.01 (m 3H); 4.39 (m 0.5H rotamers); 4.38 (m 0.5H rotamers); 4.24 (m 1H); 3.59 (m 4H); 3.22 (m 1H); 2.31 (m 1H); 1.94 (m 1H).

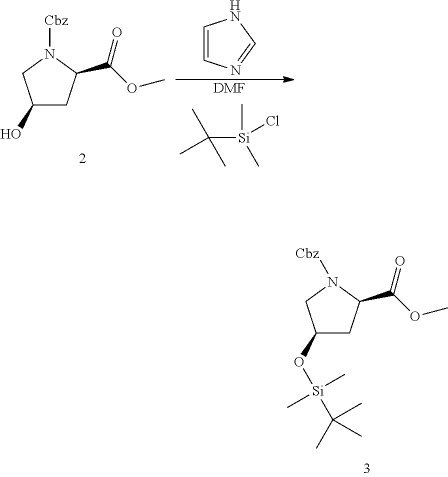

(2R,4R)-1-benzyl-2-methyl-4-hydroxypyrrolidine-1,2-dicarboxylate (2) (25.2 g; 90.23 mmol) was dissolved in 80 mL anhydrous dimethylformamide. Imidazole (14.74 g; 216.55 mmol; 2.4 equiv) was added to the reaction mixture followed by drop wise addition of tert-butyldimethylsilyl chloride (16.32 g; 108.27 mmol; 1.2 equiv) dissolved in 30 mL dimethylformamide with stirring at room temperature. Reaction mixture was stirred overnight. Ethyl acetate was added to the reaction mixture and the mixture was washed with 0.1 M potassium hydrogen sulfate solution. Organic extract was washed with saturated sodium chloride, dried over anhydrous sodium sulfate and evaporated under reduced pressure to give 35.45 g (2R,4R)-1-benzyl-2-methyl-4-(tert-butyldimethylsilyloxy)pyrrolidine-1,2-dicarboxylate (3). ¹H NMR (DMSO-d₆): δ ppm 7.34 (m 5H); 5.11 (m 2H); 4.41 (m 2H); 3.63 (m 0.5H rotamers); 3.60 (2 s 3H rotamers); 3.43 (m 0.5H); 3.17 (m 1H) 2.20 (m 1H); 1.96 (m 1H); 0.84 (s 9H); 0.01 (s 6H).

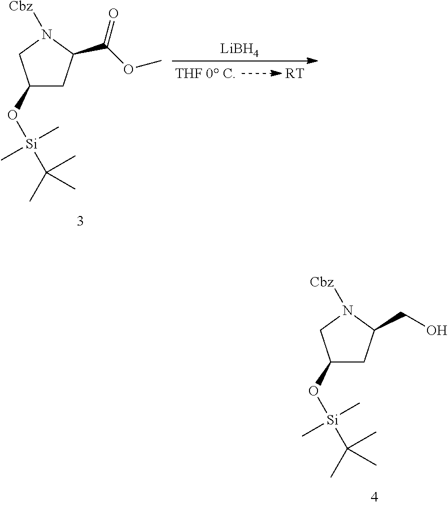

(2R,4R)-1-benzyl-2-methyl-4-(tert-butyldimethylsilyloxy)pyrrolidine-1,2-dicarboxylate (3) (9.75 g; 24.77 mmol) was dissolved in 100 mL anhydrous tetrahydrofuran and cooled to 0° C. Lithium borohydride (0.81 g; 37.05 mmol; 1.5 equiv.) was added portion wise over 30 min while stirring the cooled reaction mixture. The reaction mixture was allowed to warm to room temperature and stirred for 3 hours. The mixture was cooled to 0° C. and treated with 30 mL cold water followed by careful acidification with ice cold 1 M hydrochloric acid (50 mL). The mixture was extracted with ethyl acetate. The combined extracts were washed with saturated sodium chloride, dried over sodium sulfate and solvent removed under reduced pressure to give crude product. Crude product was purified by silica gel chromatography eluting with gradient of 0% EtOAc/hexanes to 100% ethyl acetate to give 9.82 g (2R,4R)-benzyl-4-(tert-butyldimethylsilyloxy)-2-(hydroxymethyl)pyrrolidine-1-carboxylate (4) as a clear oil. ¹H NMR (DMSO-d₆): δ ppm 7.30 (m 5H); 5.05 (m 2H); 4.65 (m 1H); 4.38 (bs 1H); 3.78 (m 1H); 3.61 (m 2H); 3.51 (m 1H); 3.11 (q 1H); 2.03 (m 1H); 1.91 (m 1H); 0.85 (s 9H); 0.05 (s 6H).

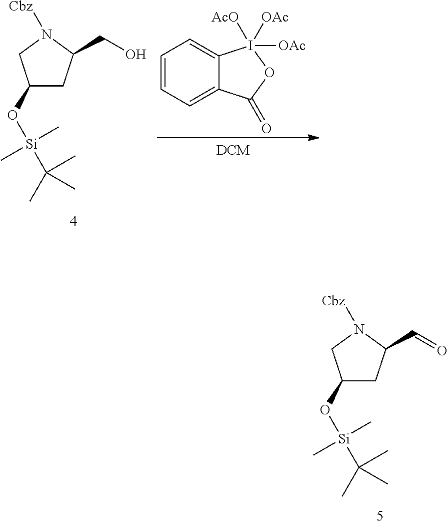

(2R,4R)-benzyl-4-(tert-butyldimethylsilyloxy)-2-(hydroxymethyl)pyrrolidine-1-carboxylate (4) (9.82 g; 26.89 mmol) was dissolved in 100 mL dichloromethane. Dess Martin periodinane (12.54 g; 29.57 mmol; 1.10 equiv.) was added and the reaction mixture was stirred overnight at room temperature. The reaction mixture was diluted with diethyl ether (100 mL) and the mixture was filtered. The filtrate was washed with a solution of 5% sodium bicarbonate (50 mL) containing 5 mL 1 M sodium thiosulfate. The organic phase was evaporated and residue subjected to column chromatography on silica gel eluting with gradient of 0% ethyl acetate/hexanes to 100% ethyl acetate to give 5.36 g (2R,4R)-benzyl-4-(tert-butyldimethylsilyloxy)-2-formylpyrrolidine-1-carboxylate (5) as a clear oil. ¹H NMR (DMSO-d₆): δ ppm 9.50 (s 1H); 7.33 (m 5H); 5.12 (m 2H); 4.44 (bs 1H); 4.25 (q 1H); 4.05 (m 1H); 3.52 (m 1H); 3.33 (m 1H); 2.29 (m 1H); 1.90 (m 1H); 0.85 (s 9H); 0.05 (s 6H).

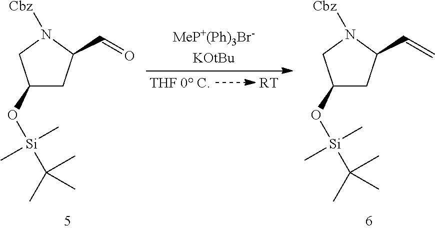

Potassium t-butoxide (0.561 g; 5 mmol) was dissolved in anhydrous tetrahydrofuran (50 mL) and cooled to 0° C. under nitrogen. Methyl triphenylphosphonium bromide (1.786 g; 5 mmol) was added to the solution to produce a bright yellow color. The mixture was stirred for 2 hours at 0° C. (2R,4R)-benzyl-4-(tert-butyldimethylsilyloxy)-2-formylpyrrolidine-1-carboxylate (5) (1.82 g; 5 mmol) was dissolved in 5 mL anhydrous tetrahydrofuran and added drop wise to the reaction mixture with stirring. After 2 hours the reaction mixture was allowed to warm to room temperature with stirring overnight. Reaction mixture was treated with saturated ammonium chloride solution (30 mL) then extracted with diethyl ether (3×50 mL), combined extracts were washed with saturated sodium chloride, dried over anhydrous sodium sulfate and evaporated under reduced pressure to give crude product. Product was purified by column chromatography on silica gel with gradient elution 20% to 100% ethyl acetate/hexanes. Obtained 750 mg (2R,4R)-benzyl-4-(tert-butyldimethylsilyloxy)-2-vinylpyrrolidine-1-carboxylate (6). ¹H NMR (DMSO-d₆): δ ppm 7.34 (m 5H); 5.92 (m 1H); 5.03 (m 4H); 4.41 (bs 1H); 4.31 (m 1H); 3.60 (m 1H); 3.12 (m 1H); 2.26 (m 1H); 1.64 (m 1H); 0.85 (s 9H); 0.05 (s 6H).

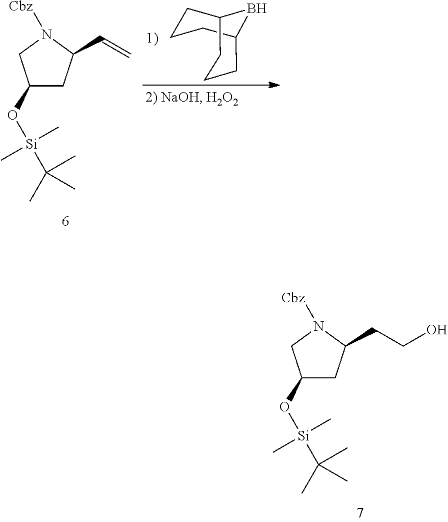

(2R,4R)-benzyl-4-(tert-butyldimethylsilyloxy)-2-vinylpyrrolidine-1-carboxylate (6) (1.80 g; 4.97 mmol) was dissolved in 80 mL anhydrous tetrahydrofuran and 9-BBN solution (0.5 M THF, 20 mL; 10 mmol) was added and the mixture was stirred at room temperature overnight. The reaction mixture was cooled to 0° C. and 30 mL water added with stirring followed by 10 mL 3M sodium hydroxide solution. A 30% hydrogen peroxide solution (11 mL) was added slowly and the mixture was stirred at room temperature for 4 hours. Reaction mixture was extracted with diethyl ether (3×50 mL), the combined extracts were washed with saturated sodium chloride, dried over anhydrous sodium sulfate and solvent removed under reduce pressure to give crude product. Product was purified by column chromatography on silica gel eluting with gradient of 0% ethyl acetate/hexanes to 100% ethyl acetate/hexanes to give 1.66 g (2S,4R)-benzyl-4-(tert-butyldimethylsilyloxy)-2-(2-hydroxyethyl)pyrrolidine-1-carboxylate (7). ¹H NMR (DMSO-d₆): δ ppm 5.32 (m 5H), 5.05 (m 2H); 4.38 (m 2H); 3.89 (m 1H); 3.58 (m 1H); 3.36 (m 3H); 3.13 (m 1H); 2.08 (m 2H); 1.66 (m 2H); 0.84 (s 9H); 0.04 (s 6H).

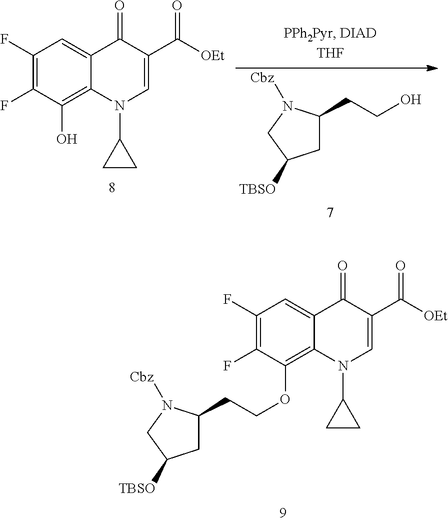

(2S,4R)-benzyl-4-(tert-butyldimethylsilyloxy)-2-(2-hydroxyethyl)pyrrolidine-1-carboxylate (7) (759 mg; 2.0 mmol) was dissolved in 10 mL anhydrous tetrahydrofuran. Diphenyl-2-pyridylphosphine (526 mg; 2.0 mmol) and ethyl 1-cyclopropyl-6,7-difluoro-8-hydroxy-4-oxo-1,4-dihydroquinoline-3-carboxylate (8) (618 mg; 2.0 mmol) were added in succession. Diisopropyl azodicarboxylate (404 mg; 393 μL; 2.0 mmol) was added in 4 portions in 5 minutes to the stirred reaction mixture. The reaction mixture was stirred under nitrogen atmosphere at room temperature for 2.5 hours. The solvent was removed under reduced pressure, the residue was dissolved in ethyl acetate, washed with saturated sodium chloride, and the organic phase dried over anhydrous sodium sulfate. Evaporated the solvent and dissolved the residue in dichloromethane. Product was purified on silica gel TLC plates (20 cm×20 cm, 1500 microns) eluting with 5% methanol/dichloromethane, to give 910 mg ethyl-8-(2-((2S,4R)-1-(benzyloxycarbonyl)-4-(tert-butyldimethylsilyloxy)pyrrolidin-2-yl)ethoxy)-1-cyclopropyl-6,7-difluoro-4-oxo-1,4-dihydroquinoline-3-carboxylate (9). MH⁺: 671.1, MNa⁺: 694.1, M₂Na⁺: 1363.2.

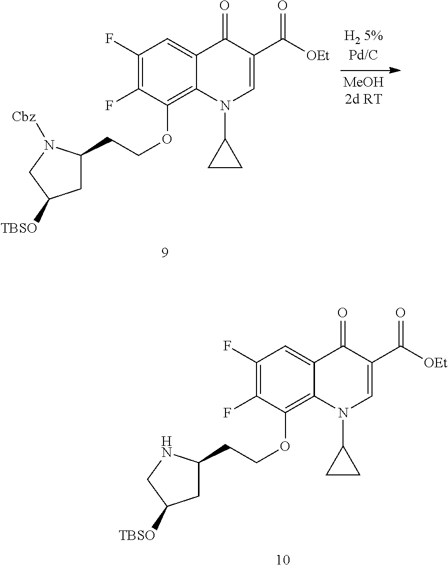

Ether (9) (840 mg) was dissolved in 50 mL methanol. 80 mg 5% Pd/C catalyst was added and hydrogenation performed under balloon pressure for 3 days. The reaction mixture was filtered over celite to remove catalyst and evaporated under reduced pressure. Residue was dissolved in ethyl acetate and washed with saturated sodium chloride, the organic phase was dried over anhydrous sodium sulfate and solvent removed to give ethyl 8-(2-((2S,4R)-4-(tert-butyldimethylsilyloxy)pyrrolidin-2-yl)ethoxy)-1-cyclopropyl-6,7-difluoro-4-oxo-1,4-dihydroquinoline-3-carboxylate (10) as a yellow oil (490 mg). MH⁺: 537.1, MNa⁺: 559.1, M₂Na⁺: 1095.1.

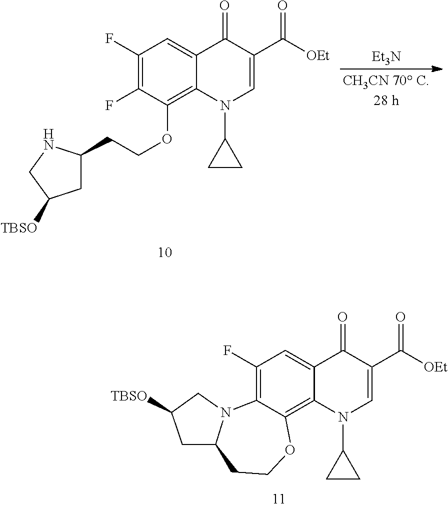

Ethyl-8-(2-((2S,4R)-4-(tert-butyldimethylsilyloxy)pyrrolidin-2-yl)ethoxy)-1-cyclopropyl-6,7-difluoro-4-oxo-1,4-dihydroquinoline-3-carboxylate (10) (490 mg) was dissolved in 5 mL anhydrous acetonitrile. Added triethylamine (0.106 mL; 1 equiv). The sealed vial was heated to 70° C. under nitrogen for 27 hours. Evaporated solvent under reduced pressure dissolved residue in dichloromethane and purified by preparative TLC (silica gel 20 cm×20 cm×1500 micron plates) eluting with 5% methanol/dichloromethane to give 250 mg product (11). MH⁺: 517.2, MNa⁺: 519.2, M₂Na⁺: 1055.2.

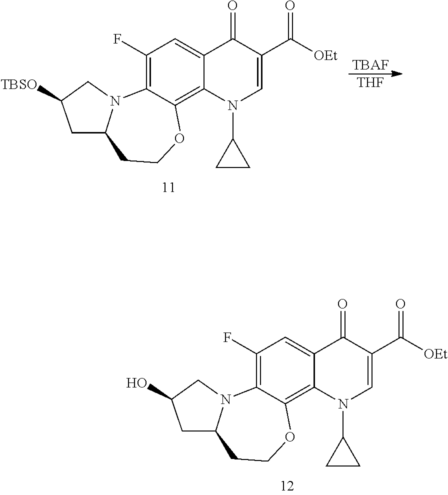

Compound (11) (250 mg; 0.484 mmol) was dissolved in 5 mL anhydrous tetrahydrofuran. A solution of tetrabutyl ammonium fluoride (1M TBAF in THF; 0.629 mL) was added to the mixture and it was stirred for 1 hour. Evaporated mixture under reduced pressure, dissolved residue in ethyl acetate and washed with 1M sodium citrate (1 mL), extracted aqueous wash with ethyl acetate and combine extracts were dried over anhydrous sodium sulfate. Removed the solvent under reduced pressure to give ˜284 mg product (12) containing some residual impurity. MH⁺403.1, MNa⁺: 425.2, M₂Na⁺: 827.0.

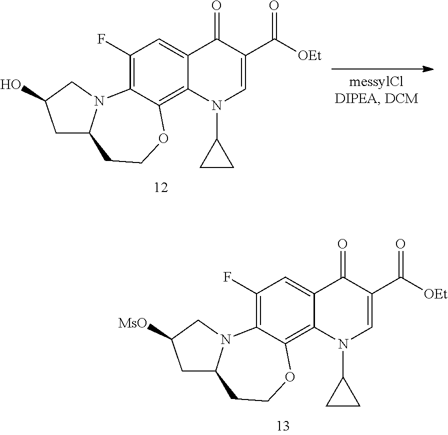

Alcohol (12) (284 mg; 0.706 mmol) was dissolved in 4 mL anhydrous dichloromethane. Diisopropylethylamine (0.369 mL; 2.12 mmol; 3 equiv.) was added followed by drop wise addition of methanesulfonyl chloride (0.071 mL; 0.907 mmol; 1.3 equiv.) dissolved in 1 mL dichloromethane. After 1 hour added 0.020 mL methanesulfonyl chloride and stirred for another hour. Concentrated reaction mixture and purified product by preparative TLC on silica gel (20 cm×20 cm×1500 micron plate) eluting with 5% methanol/dichloromethane to give 102 mg purified product (13). MH⁺: 481.1

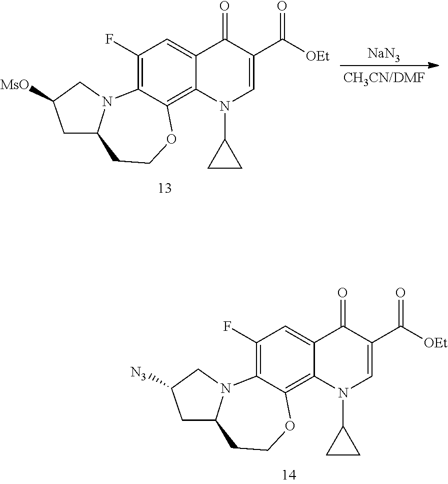

Mesylate (13) (51 mg; 0.106 mmol) wad dissolved in mixture of acetonitrile/dimethylformamide (1.5 mL/0.3 mL). Sodium azide (68 mg; 1.06 mmol; 10 equiv.) was added and the mixture was heated to 85° C. overnight under nitrogen. Evaporated solvent and dissolved residue in ethyl acetate, washed with saturated sodium chloride, dried ethyl acetate with anhydrous sodium sulfate and evaporated under reduced pressure to give crude product (14). (˜50 mg). MH⁺: 428.1

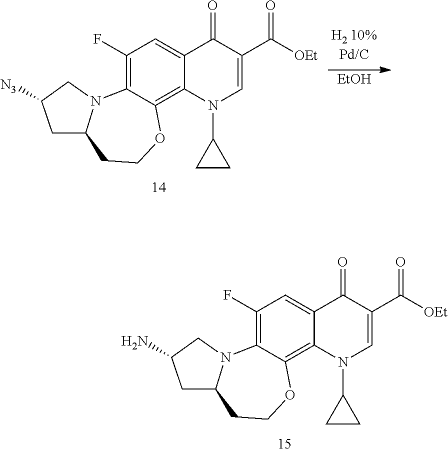

Azide (14) (50 mg) was dissolved in ethanol. Added 10% Pd/C (5 mg) and placed under hydrogen balloon at room temperature overnight. Filtered reaction mixture over celite and evaporated under reduced pressure. Product was purified by preparative reversed phase HPLC eluting with 20-40% acetonitrile/0.1% TFA/water gradient. Acetonitrile was under reduced pressure to give the desired purified product (15). MH⁺: 402.1, MNa⁺: 424.2, M₂Na⁺: 825.0.

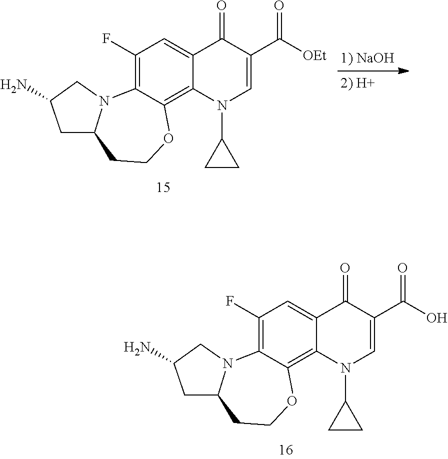

Ester (15) (20 mg was dissolved in 0.2 mL water, added 15 microliters 3 M sodium hydroxide and heated to 70° C. for 10 min. Acidified mixture with 15 microliters acetic acid and subjected mixture to reversed phase HPLC eluting with acetonitril/water/0.1% TFA gradient (5%-45% acetonitrile). Fractions containing desired product (16) were lyophilized to give 0.5 mg. MH⁺: 374.1.

### Example 3

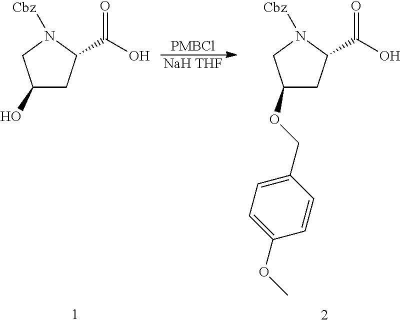

(2S,4R)-1-(benzyloxycarbonyl)-4-hydroxypyrrolidine-2-carboxylic acid (1) (75 g; 283 mmol) in 200 mL anhydrous tetrahydrofuran was added to a stirred suspension of sodium hydride (24.9 g 60% in oil; 14.95 g NaH; 623 mmol) in 750 mL tetrahydrofuran. After stirred at room temperature for 1 hour 4-methoxybenzyl chloride (66.6 g; 425 mmol) was added drop wise to the reaction mixture. After addition was complete (20 min) the reaction mixture was heated to 65° C. overnight. The reaction mixture was concentrated under reduced pressure. Diethyl ether (600 mL) was added to the residue followed by 500 mL water. The mixture was stirred and layers were separated. The aqueous phase was washed with diethyl ether (2×200 mL). The aqueous phase was acidified to pH 2-3 with 1M potassium hydrogen sulfate solution. Diethyl ether (300 mL) was added and layers were separated. The aqueous layer was extracted with diethyl ether (300 mL+200 mL). The combined diethyl ether extracts were washed with saturated sodium chloride, dried over anhydrous sodium sulfate, and evaporated. The residue was chromatographed on silica gel column eluted with 5% methanol/dichloromethane to give 69 g (2S,4R)-1-(benzyloxycarbonyl)-4-(4-methoxybenzyloxy)pyrrolidine-2-carboxylic acid (2). LC/MS MH⁺: 386.1 MNa⁺: 408.1; M₂Na⁺: 793.3.

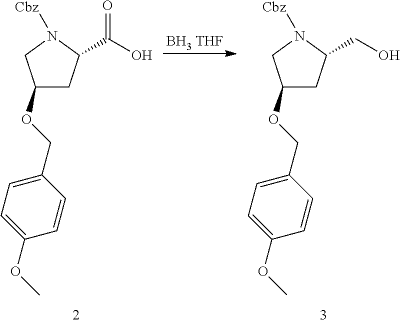

(2S,4R)-1-(benzyloxycarbonyl)-4-(4-methoxybenzyloxy)pyrrolidine-2-carboxylic acid (2) (32 g; 83 mmol) was dissolved in anhydrous tetrahydrofuran (400 mL) and cooled to 0° C. Borane-tetrahydrofuran (1 M borane tetrahydrofuran, 91.3 mL; 91.3 mmol; 1.1 equiv.) was added to the mixture under nitrogen atmosphere. Additional borane tetrahydrofuran was added to drive the reaction to completion, stirring the mixture overnight at room temperature. Methanol was added to the mixture followed by water. Evaporated mixture and the residue was dissolved in ethyl acetate (1 liter), washed with 50% sodium bicarbonate (200 mL), washed with water, washed with saturated sodium chloride, dried over anhydrous sodium sulfate and filtrate evaporated. The residue was dissolved in dichloromethane and dried overnight by stirring with anhydrous sodium sulfate. After filtration and evaporated of the filtrate under reduced pressure obtained 31.35 g of (2S,4R)-benzyl-2-(hydroxymethyl)-4-(4-methoxybenzyloxy)pyrrolidine-1-carboxylate (3). MH⁺372.1.

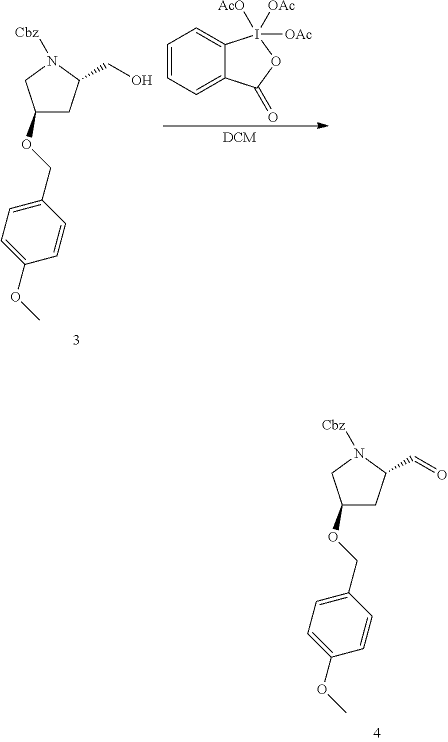

(2S,4R)-benzyl-2-(hydroxymethyl)-4-(4-methoxybenzyloxy)pyrrolidine-1-carboxylate (3) (30.8 g; 83 mmol) was dissolved in dichloromethane and cooled to 0° C. Dess martin periodinane (42.25 g; 99.6 mmol; 1.2 equiv.) was added to the mixture and the mixture was stirred while warming to room temperature overnight. An addition 0.1 equiv Dess Martin periodinane was added (3.52 g; 8.30 mmol). Added 40 mL water saturated dichloromethane to the reaction mixture and stirred overnight. Diluted reaction mixture with diethyl ether (800 mL) and treated with saturated sodium bicarbonate (200 mL) and 1 M sodium thiosulfate (100 mL). The layers were separated, washed organic phase with sodium bicarbonate/sodium thiosulfate solution. Washed with saturated sodium bicarbonate (200 mL), saturated sodium chloride (100 mL) and dried over anhydrous sodium sulfate. Filtered solution and evaporated under reduced pressure. Evaporated solution and chromatographed on silica gel eluting with a gradient of 0% methanol/dichloromethane to 5% methanol/dichloromethane to give 26 g (2S,4R)-benzyl-2-formyl-4-(4-methoxybenzyloxy)pyrrolidine-1-carboxylate (4). MH⁺ 370.1.

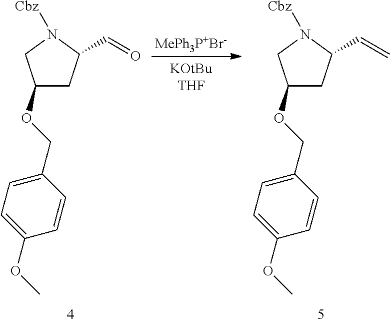

Potassium t-butoxide (5.69 g; 50.7 mmol; 1.10 equiv.) was dissolved in 400 mL anhydrous tetrahydrofuran and cooled to 0° C., methyl triphenylphosphonium bromide (18.11 g; 50.7 mmol; 1.10 equiv.) was added and the mixture was stirred for 1.5 hours at 0° C. Aldehyde (4) (17.0 g; 46.1 mmol; 1 equiv.) was dissolved in 100 mL anhydrous tetrahydrofuran and added drop wise with stirring. The mixture was stirred overnight while allowing the temperature to warm to room temperature. Treated reaction mixture with saturated ammonium chloride (300 mL), separated layers and extracted aqueous layer with diethyl ether (3×100 mL), combined organic extracts were washed with saturated sodium chloride, dried over anhydrous sodium sulfate and evaporated. Product was purified by silica gel chromatography eluting with gradient 10% ethyl acetate/hexanes to 100% ethyl acetate. Obtained 7.32 g (2S,4R)-benzyl-4-(4-methoxybenzyloxy)-2-vinylpyrrolidine-1-carboxylate (5). MH⁺ 368.0; ¹H NMR (DMSO-d₆): δ ppm 7.33 (m 5H); 7.23 (d 2H); 6.87 (d 2H); 5.78 (m 1H); 4.19 (m 4H); 4.38 (m 3H); 3.02 (m 3H); 4.07 (bs 1H); 3.73 (s 3H); 3.55 (m 1H); 3.43 (m 1H); 2.16 (m 1H); 1.84 (m 1H).

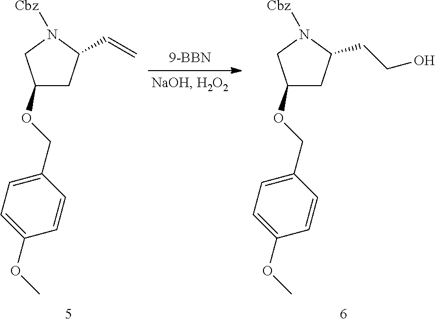

(2S,4R)-benzyl-4-(4-methoxybenzyloxy)-2-vinylpyrrolidine-1-carboxylate (5) (7.32 g; 19.92 mmol; 1 equiv.) was dissolved in 250 mL anhydrous tetrahydrofuran and treated with 9-BBN (0.5 M THF; 80 mL; 2 equiv.) in two portions over four hours. A duplicate prep was run using alkene (3.71 g; 10.1 mol) under identical conditions and reactions combine for workup. Cooled mixture to 0° C. and added 100 mL water, 60 mL 3 M sodium hydroxide solution followed by 66 mL 30% hydrogen peroxide solution and stirred for 30 min at room temperature. Added 30 mL saturated sodium chloride and separated layers. Extracted aqueous phase with ethyl acetate (3×100 mL), combined organic extracts were washed with saturated sodium chloride, dried over anhydrous sodium sulfate and evaporated. Product was purified by silica gel chromatography on silica gel eluting with gradient of 30-100% ethyl acetate/hexane to give 11.4 g (2R,4R)-benzyl-2-(2-hydroxyethyl)-4-(4-methoxybenzyloxy)pyrrolidine-1-carboxylate (6) as a clear oil. MH⁺385.9; ¹H NMR (DMSO-d₆): δ ppm 7.33 (m 5H); 7.21 (d 2H); 6.84 (d 2H); 5.06 (m 2H); 4.44 (m 3H); 4.06 (m 1H); 3.90 (m 1H); 3.73 (s 3H); 3.56 (m 1H); 3.41 (m 3H); 2.14 (m 1H); 1.89 (m 1H); 1.81 (m 1H); 1.44 (m 1H).

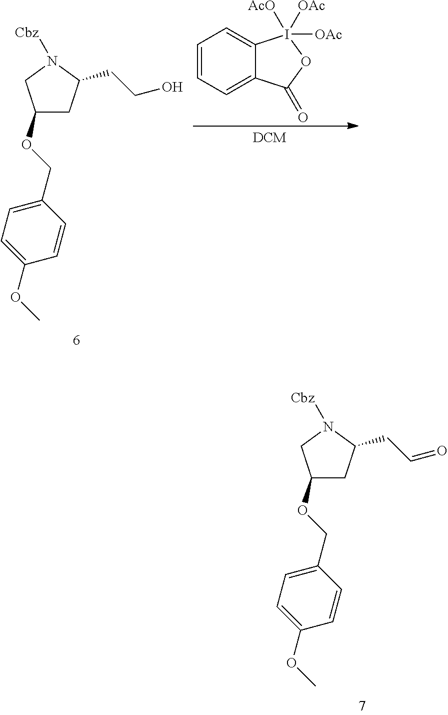

(2R,4R)-benzyl-2-(2-hydroxyethyl)-4-(4-methoxybenzyloxy)pyrrolidine-1-carboxylate (6) (11.40 g; 29.6 mmol) was dissolved in dichloromethane and treated with Dess Martin periodinane (15.05 g; 35.5 mmol; 1.2 equiv.) and the mixture was stirred overnight at room temperature. Diethyl ether (180 mL) was added, 150 mL saturated sodium bicarbonate and 25 mL 1 M sodium thiosulfate solution was added mixture allowed to separate into layers. Aqueous layer was extracted with diethyl ether, combined organic extracts were washed with saturated sodium chloride, dried over anhydrous sodium sulfate and solvent removed under reduced pressure. Product was purified on silica gel eluting with gradient 25% ethyl acetate/hexanes to 100% ethyl acetate to give 4.77 g (2S,4R)-benzyl-4-(4-methoxybenzyloxy)-2-(2-oxoethyl)pyrrolidine-1-carboxylate (7) as a clear oil. MH⁺383.9. ¹H NMR (DMSO-d₆): δ ppm 9.66 (bs 1H); 7.32 (m 5H); 7.22 (d 2H 9 Hz); 6.90 (d 2H 8 Hz); 5.05 (m 2H); 4.37 (m 2H); 4.19 (m 1H); 4.08 (bs 1H); 3.73 (s 3H); 3.56 (m 1H); 3.97 (m 1H); 2.81 (m 1H); 2.62 (m 1H); 2.25 (m 1H); 1.78 (m 1H).

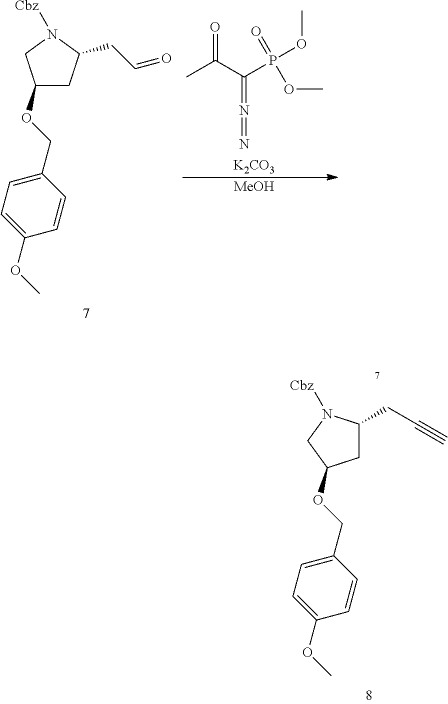

(2S,4R)-benzyl-4-(4-methoxybenzyloxy)-2-(2-oxoethyl)pyrrolidine-1-carboxylate (7) (4.77 g; 12.4 mmol; 1 equiv) was dissolved in 90 mL methanol and cooled to 0° C. Added potassium carbonate (3.43 g; 24.9 mmol; 2 equiv.) followed by freshly prepared dimethyl-1-diazo-2-oxopropylphosphonate (2.52 g; 13.14 mmol; 1.06 equiv.) dissolved in 15 mL methanol added drop wise. The reaction was stirred overnight at room temperature. Removed solvent under reduced pressure and dissolved residue in diethyl ether (100 mL) and washed with 5% sodium bicarbonate, saturated sodium chloride and dried over anhydrous sodium sulfate. Evaporated solvent to give product that was purified by silica gel chromatography eluting with gradient of 10% ethyl acetate/hexanes to 100% ethyl acetate to give 2.51 g (2R,4R)-benzyl-4-(4-methoxybenzyloxy)-2-(prop-2-ynyl)pyrrolidine-1-carboxylate (8). ¹H NMR (DMSO-d₆): 4.91 (5H m); 7.22 (2H d 8 Hz); 6.89 (2H d 8 Hz); 5.07 (2H m); 4.37 (m 2H); 4.14 (bs 1H); 3.92 (m 1H); 3.73 (s 3H); 3.62 (m 1H); 3.39 (m 1H); 3.33 (s 1H); 2.83 (bs 1H); 2.58 (m 1H); 2.19 (m 1H); 1.99 (m 1H).

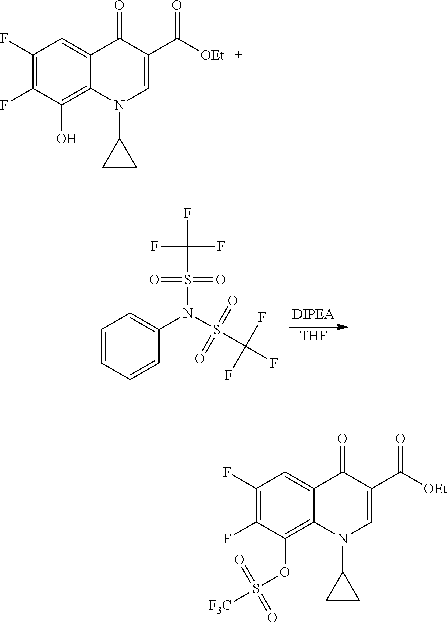

Ethyl-1-cyclopropyl-6,7-difluoro-8-hydroxy-4-oxo-1,4-dihydroquinoline-3-carboxylate (3.1 g; 10 mmol) was dissolved in tetrahydrofuran and diisopropylethylamine (2.6 g; 3.5 mL; 20 mmol) was added to the mixture. N-Phenyl-bis(trifluoromethanesulfonimide (3.76 g; 10.53 mmol) was added to the mixture at room temperature with stirring. The mixture was stirred overnight at room temperature. The mixture was evaporated under reduced pressure and residue dissolved in ethyl acetate (600 mL), washed with 1N citric acid (200 mL); water followed by saturated sodium bicarbonate (200 mL), water, and saturated sodium chloride. The ethyl acetate extract was dried over anhydrous sodium sulfate, filtered and filtrate evaporated. The crude product was purified by chromatography on silica gel eluting with gradient of 0% Ethyl acetate/hexane to 50% ethyl acetate/hexane to give 3.67 g ethyl-1-cyclopropyl-6,7-difluoro-4-oxo-8-(trifluoromethylsulfonyloxy)-1,4-dihydroquinoline-3-carboxylate.

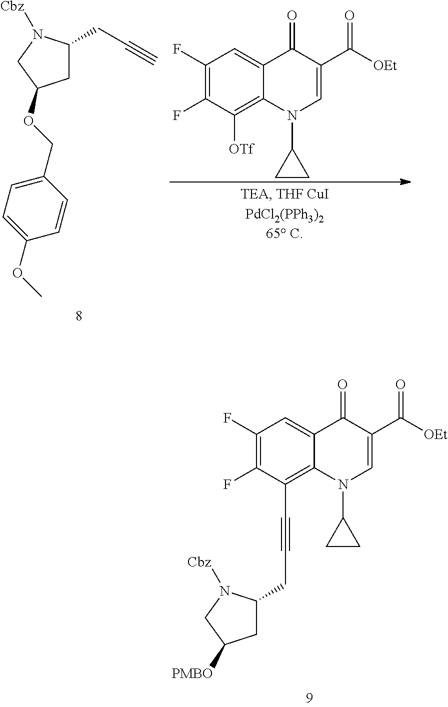

Ethyl-cyclopropyl-6,7-difluoro-4-oxo-8-(trifluoromethylsulfonyloxy)-1,4-dihydroquinoline-3-carboxylate (1.72 g; 3.90 mmol; 1 equiv.) was combined with (2R,4R)-benzyl-4-(4-methoxybenzyloxy)-2-(prop-2-ynyl)pyrrolidine-1-carboxylate (8) (1.63 g; 4.30 mmol; 1.1 equiv) in 9 mL anhydrous tetrahydrofuran. Added triphenyl phosphine (51 mg; 0.195 mmol; 0.05 equiv), triethylamine (1.09 mL; 7.8 mmol; 2.0 equiv); dichlorobis(triphenylphosphine)Palladium(II) (273 mg; 0.39 mmol; 0.1 equiv.) under an inert atmosphere. Stirred mixture for 20 min at room temperature then added copper iodide (74 mg; 0.390 mmol; 0.1 equiv). Heated the mixture to 65° C. for 30 min then to 70° C. for 8 hours. Partially purified product by silica gel chromatography 0% methanol/dichloromethane to 3% methanol/dichloromethane. Further purification was performed by reversed phase HPLC to give 1.0 g ethyl-8-(3-((2R,4R)-1-(benzyloxycarbonyl)-4-(4-methoxybenzyloxy)pyrrolidin-2-yl)prop-1-ynyl)-1-cyclopropyl-6,7-difluoro-4-oxo-1,4-dihydroquinoline-3-carboxylate (9). MH⁺: 671.2.

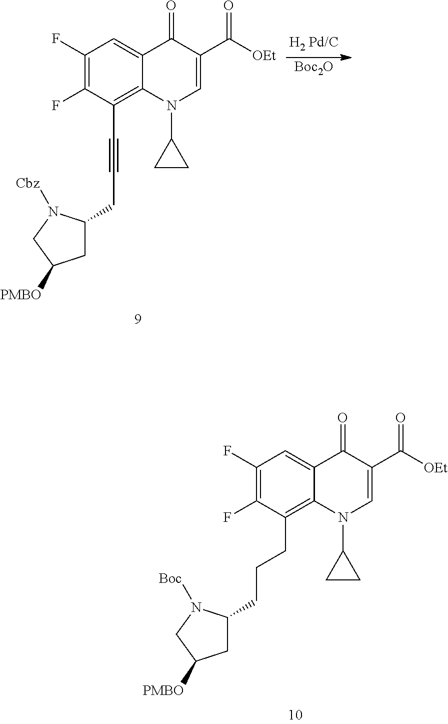

Ethyl-8-(3-((2R,4R)-1-(benzyloxycarbonyl)-4-(4-methoxybenzyloxy)pyrrolidin-2-yl)prop-1-ynyl)-1-cyclopropyl-6,7-difluoro-4-oxo-1,4-dihydroquinoline-3-carboxylate (9) (1.0 g; 1.49 mmol) was dissolved in absolute ethanol (15 mL) and pH adjusted to 9 with triethylamine (0.250 mL). 10% Pd/C (150 mg) was added to the mixture and the mixture was subjected to balloon hydrogenation. Di-t-butyldicarbonate (357 mg 1.64 mmol; 1.1 eq) was added to the reaction mixture and the mixture was stirred for 4 hours. After completion of reaction, the mixture was diluted with ethanol, filtered over celite to remove catalyst and evaporated under reduced pressure to give 953 mg ethyl-1-cyclopropyl-6,7-difluoro-8-(3-((2R,4R)-4-(4-methoxybenzyloxy)pyrrolidin-2-yl)propyl)-4-oxo-1,4-dihydroquinoline-3-carboxylate (10). MH⁺: 641.2.

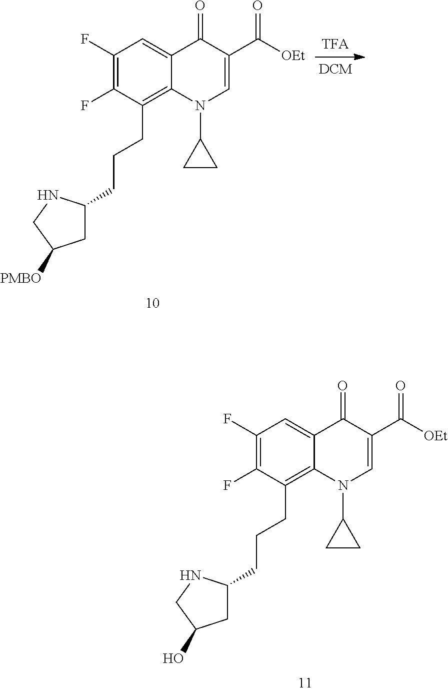

Ethyl-1-cyclopropyl-6,7-difluoro-8-(3-((2R,4R)-4-(4-methoxybenzyloxy)pyrrolidin-2-yl)propyl)-4-oxo-1,4-dihydroquinoline-3-carboxylate (10) was dissolved in dichloromethane (15 mL) and treated with 1.5 mL trifluoroacetic acid at room temperature for several hours. Evaporated to dryness, residue was dissolved in dichloromethane and evaporated. Repeated dissolution and evaporation gave 626 mg ethyl 1-cyclopropyl-6,7-difluoro-8-(3-((2R,4R)-4-hydroxypyrrolidin-2-yl)propyl)-4-oxo-1,4-dihydroquinoline-3-carboxylate (11). MH⁺: 421.2.

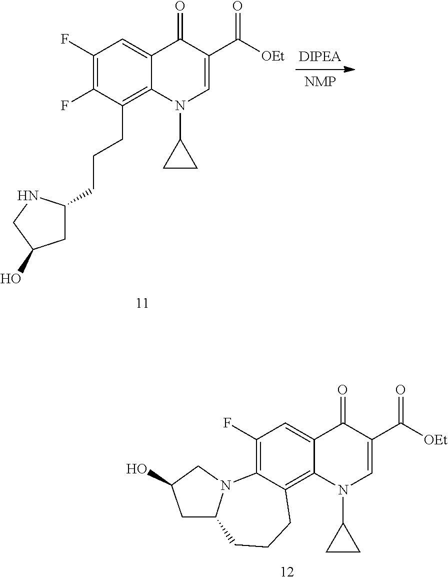

Ethyl-1-cyclopropyl-6,7-difluoro-8-(3-((2R,4R)-4-hydroxypyrrolidin-2-yl)propyl)-4-oxo-1,4-dihydroquinoline-3-carboxylate (11) (626 mg; 1.49 mmol) was dissolved in N-methylpyrrolidinone (10 mL) and diisopropylethylamine (1.04 mL; 6 mmol) was added to the mixture. After sparging with nitrogen, the mixture was heated to 70° C. in a sealed vial overnight, increased temperature to 130° C. and stirred overnight. Diluted reaction mixture with ethyl acetate and washed with 1 M citric acid (2×80 mL), followed by wash with saturated sodium bicarbonate (2×80 mL), saturated sodium chloride wash, dried over anhydrous sodium sulfate, filtered and evaporated under reduced pressure to give 521 mg product (12). MH⁺: 401.2.

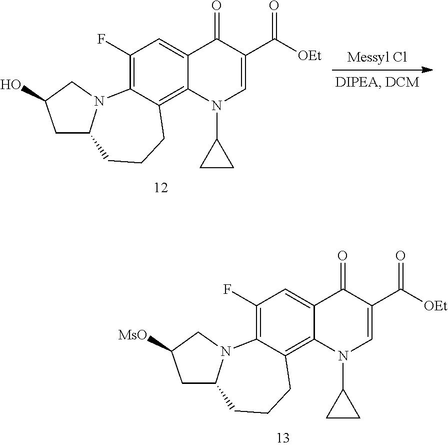

Compound (12) (521 mg; 1.3 mmol) was dissolved in anhydrous dichloromethane (15 mL). Added diisopropylethylamine (0.906 mL; 5.2 mmol) followed by methane sulfonyl chloride (194 mg; 1.7 mmol) and the mixture was stirred at room temperature for 30 min. Evaporated reaction mixture under reduced pressure to give 382 mg product (13). MH⁺: 479.1.

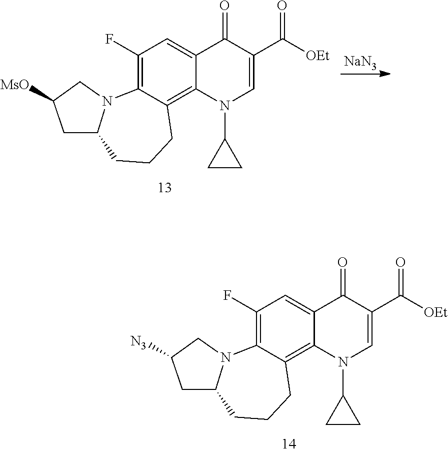

Compound (13) (382 mg; 0.8 mmol) was dissolved in acetonitrile and sodium azide (520 mg; 8 mmol) was added to the solution at room temperature. The reaction mixture was heated in a sealed vial to 80° C., overnight, then temperature increased to 85° C. for 8 hours. The mixture was filtered and evaporated under reduced pressure to give 0.45 g product (14) as a dark colored oil. MH⁺: 426.2.

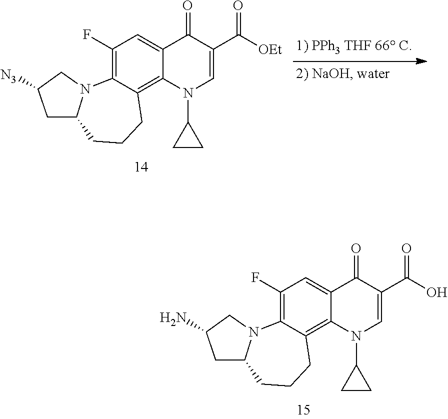

Compound (14) (340 mg, 0.8 mmol) was dissolved in anhydrous tetrahydrofuran (6 mL) and treated with triphenyl phosphine (315 mg; 0.96 mmol). The mixture was heated to reflux to 20 min. Added 1M sodium hydroxide (0.600 mL) to the reaction mixture and heated to 70° C. for 10 min. Added a mixture of acetonitrile (4 mL) and 1M sodium hydroxide (2 mL) and stirred the reaction mixture at 60° C. for 12 hours. The mixture was neutralized by addition of 1 M hydrochloric acid to pH 2-3. Evaporated to dryness with ethanol and dissolved residue in N-methylpyrrolidinone and purified by reversed phase HPLC. Combined fractions were evaporated and lyophilized to give product as a semisolid. Sonicated mixture with diethyl ether removed ether to give the product (15) as a solid (8.5 mg). MH⁺: 372.2

### Example 4

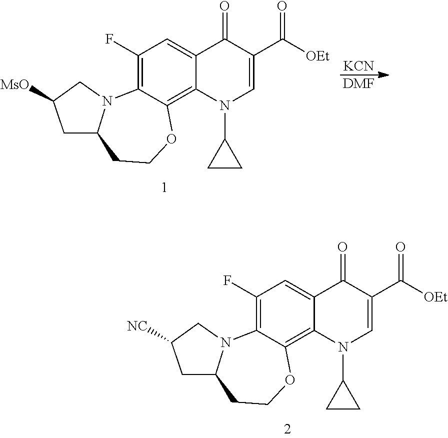

Mesylate (1) (180 mg; 0.375 mmol) was dissolved in 1.5 mL N-methylpyrrolidinone and treated with tetra-n-butyl ammonium cyanide (110 mg; 0.412 mmol; 1.1 eq); and the mixture was stirred at 70° C. for 2 hours. Finely ground potassium cyanide (100 mg) was added to the reaction mixture and temperature was increased to 80° C. overnight. Partitioned reaction mixture between ethyl acetate and saturated sodium chloride, ethyl acetate extract was washed with saturated sodium chloride and combined extracts were dried over sodium sulfate (anh.), filtered and filtrate evaporated. The crude product was purified by reversed phase C18 chromatography eluting with acetonitrile/water/0.01% trifluoroacetic acid gradient. Obtained 50 mg product (2). MH⁺412.1, MNa⁺434.1, M₂Na⁺845.

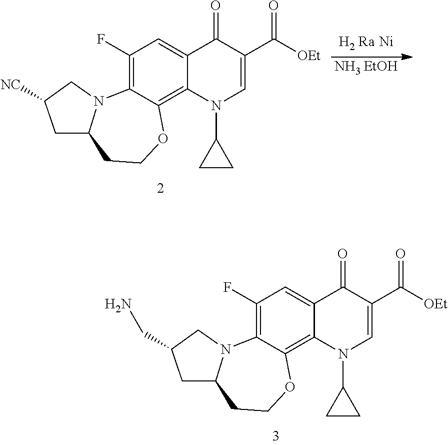

Compound (2) (50 mg) was dissolved in 5 mL absolute ethanol, treated with 0.050 mL 2 M ammonia in ethanol, added Raney Nickel (10 mg) and subjected to hydrogenation under balloon pressure for 3 hours. Removed Raney Nickel catalyst by filtration and evaporated solvent to give product (3). MH⁺ 416.1, MNa⁺: 438.2; M₂Na⁺: 853.0.

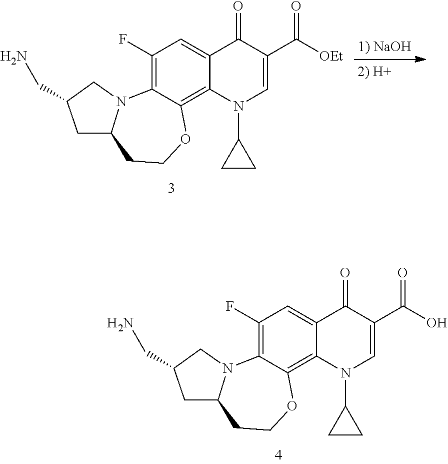

Compound (3) was suspended in 3 mL water, added 0.160 mL 3 M sodium hydroxide and heated mixture to 70° C. for 20 min, acidified mixture with glacial acetic acid. Product was purified by reversed phase C18 HPLC eluting with gradient of acetonitril/water/0.01% trifluoroacetic acid. Obtained 0.5 mg product (4). MH⁺ 388.1.

### Example 5

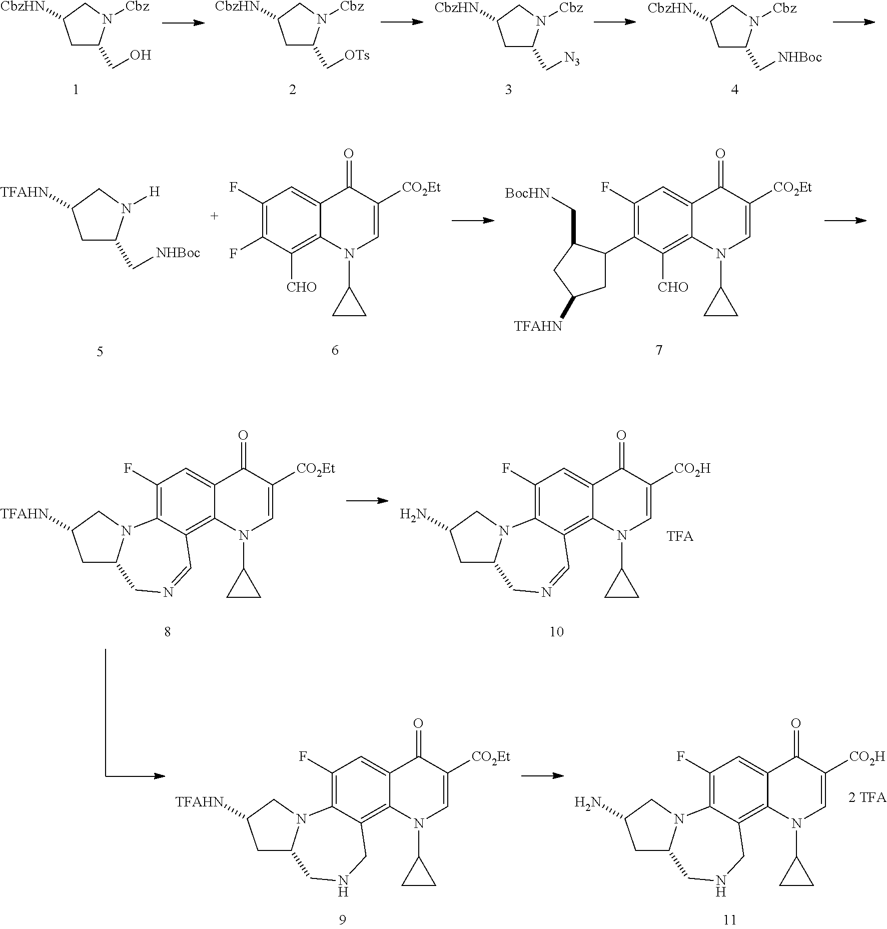

HPLC conditions: Agilent 1100 HPLC. Zorbax C8 150×4.6 mm column. Solvent A—Water (0.1% TFA); Solvent B—Acetonitrile (0.07% TFA). Flow rate, 1.50 mL/min. Gradient—10 min 95% A to 90% B; 2 min hold; then recycle. UV detection @ 214 and 254 nm. All reactions were carried out under an atmosphere of nitrogen.

### Benzyl-(2S,4S)-4-[(benzyloxy)carbonyl]amino-2-([(4-methylphenyl)sulfonyl]oxymethyl)pyrrolidine-1-carboxylate (2)

A solution of benzyl-(2S,4S)-4-[(benzyloxy)carbonyl]amino-2-(hydroxymethyl)pyrrolidine-1-carboxylate (1) (1.08 g, 2.81 mmol) in pyridine (5.2 mL) was cooled to 0° C. and treated with p-toluenesulfonyl chloride (0.573 g, 3.01 mmol) in one portion. The mixture was allowed to warm to room temperature and stir overnight. After 30 h at room temperature, starting material was present by TLC (50% EA/hex). Added an additional 150 mg of tosyl chloride and continued stirring. After 26 h, an additional 90 mg of tosyl chloride was added and stirring was continued. After an additional 44 h, the solvent was removed and the residue taken up in 40 mL 1N HCl. The aqueous phase was extracted twice with 30 mL portions of ethyl acetate. The combined organic extracts were washed with 30 mL portions of 0.5N HCl, saturated NaHCO₃ and brine and dried over Na₂SO₄. The solution was filtered and concentrated to yield the title compound (2) (1.20 g; 79%) as a slightly tan viscous oil. The material was found to be of satisfactory purity for use in the next step: MS (ESI+) for C₂₈H₃₀N₂O₇S m/z 539.2 (M+H)⁺; HPLC purity 74% (ret. time, 9.73 min); TLC 50% EA/Hex R_{f}=0.47.

### Benzyl-(2S,4S)-2-(azidomethyl)-4-[(benzyloxy)carbonyl]aminopyrrolidine-1-carboxylate (3)

A solution of benzyl-(2S,4S)-4-[(benzyloxy)carbonyl]amino-2-([(4-methylphenyl)sulfonyl]oxymethyl)pyrrolidine-1-carboxylate (2) (1.20 g, 2.23 mmol) in N,N-dimethylformamide (5.1 mL) was treated with sodium azide (0.156 g, 2.41 mmol) and heated at 50° C. After 67 h at 50° C., the reaction was found to be complete by TLC (50% EA/Hex). The reaction was allowed to cool to room temperature, diluted with 50 mL H₂O and extracted with two 40 mL portions of methyl t-butyl ether. The organic extracts were washed with two 40 mL portions of H₂O, dried over Na₂SO₄, filtered and concentrated to yield the title compound (3) (0.91 g; 100%) as a slightly tan viscous oil: MS (ESI+) for C₂₁H₂₃N₃O₄ m/z 410.2 (M+H)⁺; HPLC purity 78% (ret. time, 9.21 min); TLC 50% EA/Hex, R_{f}=0.57.

### Benzyl-(2S,4S)-4-[(benzyloxy)carbonyl]amino-2-[(tert-butoxycarbonyl)amino]methylpyrrolidine-1-carboxylate (4)

A solution of benzyl-(2S,4S)-2-(azidomethyl)-4-[(benzyloxy)carbonyl]aminopyrrolidine-1-carboxylate (3) (0.91 g, 2.2 mmol) in tetrahydrofuran (13 mL) was cooled at 0° C. in an ice bath and treated with triphenylphosphine (0.700 g, 2.67 mmol) in one portion. The solution was stirred for 1 h then treated dropwise with water (1.8 mL, 100 mmol) and stirred overnight. After 19 h at room temperature, the mixture was treated with di-tert-butyldicarbonate (0.58 g, 2.7 mmol) in one portion and allowed to stir at room temperature overnight. After 24 h at room temperature, the reaction was complete by LCMS and was concentrated to a tan viscous liquid. The material was purified by chromatography (80 g flash silica, 20-60% EA/heptane) to yield the title compound (4) (0.47 g, 44%): MS (ESI+) for C₂₆H₃₃N₃O₆ m/z 384.2 (M+H—C₅H₉O₂)⁺; HPLC purity 100% (ret. time, 9.54 min) TLC 50% EA/hex, R_{f}=0.49.

### tert-Butyl-((2S,4S)-4-[(trifluoroacetyl)amino]pyrrolidin-2-ylmethyl)carbamate (5)

A solution of benzyl-(2S,4S)-4-[(benzyloxy)carbonyl]amino-2-[(tert-butoxycarbonyl)amino]methylpyrrolidine-1-carboxylate (4) (307 mg, 0.635 mmol) in methanol (10 mL) was carefully treated with 10% palladium on carbon (40 mg). The reaction vessel was evacuated and filled with hydrogen gas three times and the reaction mixture was allowed to stir under an atmosphere of hydrogen. After 16 h at room temperature, the reaction was found to be complete by LCMS. The reaction mixture was filtered through a pad of celite and the pad was washed with 30 mL MeOH. The solution was concentrated to yield a colorless glass, which was placed on high vac. Yield of the crude diamine was 143 mg. The crude diamine was taken up in tetrahydrofuran (5 mL), treated dropwise with ethyl trifluoroacetate (75.7 uL, 0.635 mmol) and the mixture was stirred at room temperature. After 19 h at RT, the reaction mixture was concentrated to yield the title compound (5) (227 mg, 115%) as a colorless glass which was used as is in the next step: MS (ESI+) for C₁₂H₂₀F₃N₃O₃ m/z 212 (M+H—C₅H₉O₂)⁺.

### Ethyl-7-(2S,4S)-2-[(tert-butoxycarbonyl)amino]methyl-4-[(trifluoroacetyl)amino]pyrrolidin-1-yl-1-cyclopropyl-6-fluoro-8-formyl-4-oxo-1,4-dihydroquinoline-3-carboxylate (7)

A mixture of tert-butyl-((2S,4S)-4-[(trifluoroacetyl)amino]pyrrolidin-2-ylmethyl)carbamate (5) (227 mg, 0.729 mmol), ethyl-1-cyclopropyl-6,7-difluoro-8-formyl-4-oxo-1,4-dihydroquinoline-3-carboxylate (6) (156 mg, 0.486 mmol) and N,N-diisopropylethylamine (84.7 uL, 0.486 mmol) in N-methylpyrrolidinone (2.6 mL) was heated at 50° C. for 17 h, upon which the reaction was found to be complete by LCMS. The mixture was cooled to room temperature, diluted with 25 mL H₂O and extracted twice with 25 mL portions of 1/1 methyl tert-butyl ether/ethyl acetate. The combined organic phase was washed five times with 25 mL portions of H₂O, dried over Na₂SO₄, filtered and concentrated to a brownish yellow glass. The material was purified by chromatography (40 g silica gel, 4-6% MeOH/CH₂Cl₂) to yield the title compound (7) (207 mg, 70%) as a tannish yellow glass: MS (ESI+) for C₂₈H₃₂F₄N₄O₇ m/z 613.3 (M+H)⁺; HPLC purity 95-98% (ret. time, 7.91 min) TLC 40% EA/CH₂Cl₂ R_{f}=0.21.

### Ethyl-(3aS,5S)-13-cyclopropyl-8-fluoro-10-oxo-5-[(trifluoroacetyl)amino]-3a,4,5,6,10,13-hexahydro-3H-pyrrolo[2′,1′:3,4][1,4]diazepino[5,6-h]quinoline-11-carboxylate (8)

A solution of ethyl-7-(2S,4S)-2-[(tert-butoxycarbonyl)amino]methyl-4-[(trifluoroacetyl)amino]pyrrolidin-1-yl-1-cyclopropyl-6-fluoro-8-formyl-4-oxo-1,4-dihydroquinoline-3-carboxylate (7) (65 mg, 0.11 mmol) in methylene chloride (2 mL) was cooled at 0° C. and treated with trifluoroacetic acid (0.37 mL, 4.8 mmol) dropwise and the reaction was allowed to slowly warm to room temperature. After 2 h, the reaction was complete by LCMS. The reaction mixture was concentrated and the residue taken up in 10 mL CH₂Cl₂, reconcentrated and placed on high vac. The yellow glass was taken up in 20 mL CH₂Cl₂ and washed with 10 mL sat NaHCO₃. The aqueous phase was back extracted with 20 mL CH₂Cl₂, and the combined organic extracts were dried over Na₂SO₄, filtered and concentrated to a light yellow solid. The material was purified by prep TLC on a 20 cm×20 cm×0.5 mm prep TLC plate eluting with 5% MeOH/CH₂Cl₂, to yield the title compound (8) (27 mg, 51%) as a light yellow solid: MS (ESI+) for C₂₃H₂₂F₄N₄O₄ m/z 495.3 (M+H)⁺; HPLC purity 91% (ret. time, 4.61 min) TLC 5% MeOH/CH₂Cl₂, R_{f}=0.23.

### (3aS,5S)-5-amino-13-cyclopropyl-8-fluoro-10-oxo-3a,4,5,6,10,13-hexahydro-3H-pyrrolo[2′,′:3,4][1,4]diazepino[5,6-h]quinoline-11-carboxylic acid trifluoroacetate (10)

A mixture of ethyl-(3aS,5S)-13-cyclopropyl-8-fluoro-10-oxo-5-[(trifluoroacetyl)amino]-3a,4,5,6,10,13-hexahydro-3H-pyrrolo[2′,′:3,4][1,4]diazepino[5,6-h]quinoline-11-carboxylate (8) (27.0 mg, 0.0546 mmol) in methanol (2.32 mL) and water (0.9 mL) was treated with potassium carbonate (25 mg, 0.18 mmol) in one portion and the mixture was allowed to stir at room temperature for 47 h, at which point the reaction was determined to be complete by HPLC. The reaction mixture was concentrated to remove the methanol and the pH was adjusted to ˜7 with 10% aq HOAc. The mixture was subjected to preparative reverse phase HPLC with the following conditions: Phenomenex Luna 250×30 mm, 10 micron column. Flow rate was 20 mL/min. Gradient: solvent A=0.07% TFA in acetonitrile; solvent B=0.10% TFA in water; 26 minute run; 5% to 70% A over 14 minute ramp; 70% to 100% A over 3 minute ramp; hold 100% A over 3 minutes; ramp down from 100% to 5% A over 5 minutes; hold for 1 minute, then recycle. Detector wavelength was set to 290 nm. Product retention time=13.40 minutes. Product fractions were combined, concentrated to remove the acetonitrile and the water removed by lyophilization to yield the title compound (10) (10.5 mg, 40%) as a light yellow fluffy solid: MS (ESI+) for C₁₉H₁₈FN₄O₅ m/z 371.1 (M+H)⁺; HPLC purity 98% (ret time, 3.08 min).

### Ethyl-(3aS,5S)-13-cyclopropyl-8-fluoro-10-oxo-5-[(trifluoroacetyl)amino]-2,3,3a,4,5,6,10,13-octahydro-1H-pyrrolo[2′,1′:3,4][1,4]diazepino[5,6-h]quinoline-11-carboxylate (9)

A solution of ethyl-(3aS,5S)-13-cyclopropyl-8-fluoro-10-oxo-5-[(trifluoroacetyl)amino]-3a,4,5,6,10,13-hexahydro-3H-pyrrolo[2′,1′:3,4][1,4]diazepino[5,6-h]quinoline-11-carboxylate (8) (60.0 mg, 0.121 mmol) in methylene chloride (5.0 mL) was treated with sodium triacetoxyborohydride (51.4 mg, 0.243 mmol) in one portion and allowed to stir at room temperature. After 4 h, the reaction was found to complete by TLC (10% MeOH/CH₂Cl₂). The mixture was diluted with 15 mL CH₂Cl₂ and washed with 10 mL saturated NaHCO₃. The aqueous phase was washed with 5 mL CH₂Cl₂ and the combined organics were dried over Na₂SO₄, filtered and concentrated to a light yellow solid. The material was purified by prep TLC on a 20 cm×20 cm×1 mm prep TLC plate eluting with 10% MeOH/CH₂Cl₂, to yield the title compound (9) (55 mg, 91%) as a white solid: MS (ESI+) for C₂₃H₂₄F₄N₄O₄ m/z 497.0 (M+H)⁺; HPLC purity 100% (ret time, 5.03 min); TLC 10% MeOH/CH₂Cl₂ R_{f}=0.35.

(3aS,5S)-5-amino-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylic acid bis(trifluoroacetate) (11)

A mixture of ethyl-(3aS,5S)-13-cyclopropyl-8-fluoro-10-oxo-5-[(trifluoroacetyl)amino]-2,3,3a,4,5,6,10,13-octahydro-1H-pyrrolo[2′,1′:3,4][1,4]diazepino[5,6-h]quinoline-11-carboxylate (9) (55.0 mg, 0.111 mmol) in methanol (3.1 mL) and water (1.2 mL) was treated with potassium carbonate (50.5 mg, 0.366 mmol) in one portion and the mixture was allowed to stir at room temperature. After 120 h, the reaction was found to be complete by HPLC. The reaction mixture was concentrated to remove the methanol and the pH was adjusted to ˜7 with 10% aq HOAc. The mixture was allowed to stand at room temperature, during which time a white precipitate formed. The solid was filtered and washed with a little cold water followed by methyl t-butyl ether to give a light gray solid that was dried. The solid was treated with 0.1N TFA (6.5 mL), the solution was filtered and lyophilized to obtain the title compound (11) (50 mg, 81%) as a tan solid: MS (ESI+) for C₁₉H₂₁FN₄O₃ m/z 373.1 (M+H)⁺; HPLC purity 99% (ret. time, 3.40 min).

### Example 6

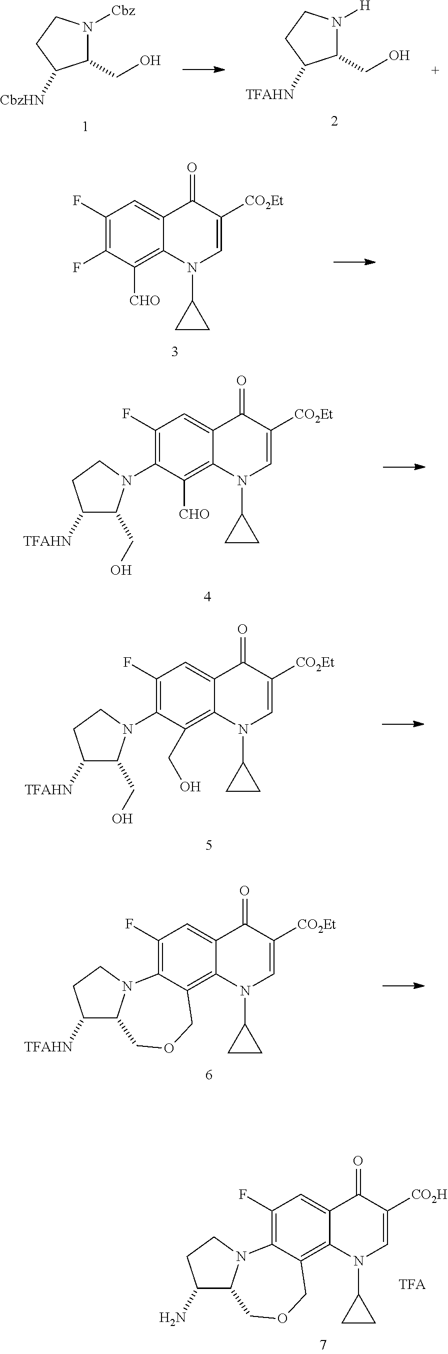

HPLC conditions: Agilent 1100 HPLC. Zorbax C8 150×4.6 mm column. Solvent A—Water (0.1% TFA); Solvent B—Acetonitrile (0.07% TFA). Flow rate, 1.50 mL/min. Gradient—10 min 95% A to 90% B; 2 min hold; then recycle. UV detection @ 214 and 254 nm or @ 214 and 290 nm. All reactions were conducted under an atmosphere of nitrogen.

### 2,2,2-Trifluoro-N-[(2S,3R)-2-(hydroxymethyl)pyrrolidin-3-yl]acetamide (2)

A solution of benzyl-(2S,3R)-3-[(benzyloxy)carbonyl]amino-2-(hydroxymethyl)pyrrolidine-1-carboxylate (1) (535 mg, 1.39 mmol) in methanol (30 mL) was carefully treated with 10% palladium on carbon (90 mg). The reaction vessel was evacuated and filled with hydrogen gas three times and the reaction mixture was allowed to stir under an atmosphere of hydrogen. After 2 h at room temperature, the reaction was found to be complete by TLC (50% EA/hex). The reaction mixture was filtered through a pad of Celite and the pad was washed with 20 mL MeOH. The filtrate was concentrated to a colorless glass. The crude diamine was taken up in tetrahydrofuran (3.4 mL) and methanol (2.6 mL), cooled at 0° C., and treated dropwise with ethyl trifluoroacetate (166 uL, 1.39 mmol). The mixture was allowed to warm to room temperature, stirred for 16 h and concentrated to yield the title compound (2) (307 mg, 104%) as a tan oil: MS (ESI+) for C₇H₁₁F₃N₂O₂MS m/z 213.1 (M+H)⁺; MS (ESI−) for C₇H₁₁F₃N₂O₂ m/z 211.1 (M−H)⁻.

### Ethyl-1-cyclopropyl-6-fluoro-8-formyl-7-(2S,3R)-2-(hydroxymethyl)-3-[(trifluoroacetyl)amino]pyrrolidin-1-yl-4-oxo-1,4-dihydroquinoline-3-carboxylate (4)

A mixture of 2,2,2-trifluoro-N-[(2S,3R)-2-(hydroxymethyl)pyrrolidin-3-yl]acetamide (2) (307 mg, 1.45 mmol), ethyl-1-cyclopropyl-6,7-difluoro-8-formyl-4-oxo-1,4-dihydroquinoline-3-carboxylate (3) (371 mg, 1.16 mmol) in N-methylpyrrolidinone (5 mL) was treated with N,N-diisopropylethylamine (0.28 mL) dropwise and the mixture was heated at 50° C. for 3 h, whereupon it was determined by LCMS that the reaction was complete. The reaction mixture was cooled, diluted with 50 mL ethyl acetate and washed with two 50 mL portions of H₂O and 50 mL brine. The organic phase was dried over Na₂SO₄, filtered and concentrated to a brown, sticky foam. The material was purified by flash chromatography (40 g flash silica gel; 2-6% MeOH/CH₂Cl₂) to yield the title compound (4) (340 mg, 57%) as a yellow stiff foam: MS (ESI+) for C₂₃H₂₃F₄N₃O₆MS m/z 514.2 (M+H)⁺; MS (ESI−) for C₂₃H₂₃F₄N₃O₆ m/z 512.2 (M−H)⁻; HPLC purity 86% (ret. time, 6.71 min).

### Ethyl-1-cyclopropyl-6-fluoro-8-(hydroxymethyl)-7-(2S,3R)-2-(hydroxymethyl)-3-[(trifluoroacetyl)amino]pyrrolidin-1-yl-4-oxo-1,4-dihydroquinoline-3-carboxylate (5)

A solution of ethyl-1-cyclopropyl-6-fluoro-8-formyl-7-(2S,3R)-2-(hydroxymethyl)-3-[(trifluoroacetyl)amino]pyrrolidin-1-yl-4-oxo-1,4-dihydroquinoline-3-carboxylate (4) (340 mg, 0.662 mmol) in methylene chloride (13 mL) was treated with sodium triacetoxyborohydride (281 mg, 1.32 mmol) in one portion and stirred at room temperature till complete. After 22 h, the reaction was complete by HPLC. The reaction mixture was diluted with 15 mL CH₂Cl₂ and 1 mL MeOH and washed with 20 mL sat NaHCO₃ solution. The aqueous phase was back extracted with 10 mL CH₂Cl₂ and the combined organic extracts were washed with 10 mL portions of H₂O and brine and dried over Na₂SO₄. The solution was filtered and concentrated to a light yellow stiff foam which was purified by flash chromatography (30 g flash silica gel, 3-10% EtOH/CH₂Cl₂) to yield the title compound (5) (270 mg, 79%) as a light yellow stiff foam: MS (ESI+) for C₂₃H₂₅F₄N₃O₆ m/z 516.0 (M+H)⁺; MS (ESI−) for C₂₃H₂₅F₄N₃O₆ m/z 514.2 (M−H)⁻; HPLC purity 96% (ret. time, 6.37 min); TLC 5% EtOH/CH₂Cl₂ R_{f}=0.31.

### Ethyl-(3aS,4R)-13-cyclopropyl-8-fluoro-10-oxo-4-[(trifluoroacetyl)amino]-3a,4,5,6,10,13-hexahydro-1H,3H-pyrrolo[2′,1′:3,4][1,4]oxazepino[5,6-h]quinoline-11-carboxylate (6)

A solution of ethyl-1-cyclopropyl-6-fluoro-8-(hydroxymethyl)-7-(2S,3R)-2-(hydroxymethyl)-3-[(trifluoroacetyl)amino]pyrrolidin-1-yl-4-oxo-1,4-dihydroquinoline-3-carboxylate (5) (115 mg, 0.223 mmol) in methylene chloride (12 mL) was treated dropwise with trifluoroacetic acid (0.43 mL, 5.6 mmol). The reaction mixture was stirred at room temperature till complete. After 70 h, the reaction was complete by HPLC. The reaction mixture was diluted with 5 mL CH₂Cl₂ and washed with 5 mL sat NaHCO₃ solution. The aqueous phase was washed with 5 mL CH₂Cl₂ and the combined organic phase was dried over Na₂SO₄. The solution was filtered and concentrated to yield a nearly colorless glass. The material was purified by flash chromatography (25 g flash silica gel, 2-6% EtOH/CH₂Cl₂) to yield the title compound (6) as a colorless glass which was not completely clean. The material was purified a second time by prep TLC using a 20 cm×20 cm×1 mm prep TLC plate, eluting twice with 6% MeOH/CH₂Cl₂, to yield the title compound (6) (47 mg, 42%) as a white amorphous solid: MS (ESI+) for C₂₃H₂₃F₄N₃O₅ m/z 498.0 (M+H)⁺; MS (ESI−) for C₂₃H₂₃F₄N₃O₅ m/z 496.1 (M−H)⁻; HPLC purity 100% (ret. time, 7.12 min); TLC 5% MeOH/CH₂Cl₂, R_{f}=0.38.

### (3aS,4R)-4-amino-13-cyclopropyl-8-fluoro-10-oxo-3a,4,5,6,10,13-hexahydro-1H,3H-pyrrolo[2′,1′:3,4][1,4]oxazepino[5,6-h]quinoline-1′-carboxylic acid trifluoroacetate (7)

A mixture of ethyl-(3aS,4R)-13-cyclopropyl-8-fluoro-10-oxo-4-[(trifluoroacetyl)amino]-3a,4,5,6,10,13-hexahydro-1H,3H-pyrrolo[2′,1′:3,4][1,4]oxazepino[5,6-h]quinoline-11-carboxylate (6) (45 mg, 0.090 mmol) in methanol (2.50 mL) and water (1 mL) was treated with potassium carbonate (38 mg, 0.27 mmol) in one portion and the mixture was stirred at room temperature for 108 h. An additional equivalent of K₂CO₃ was added and stirring was continued for 24 h, upon which HPLC indicated the reaction was complete. The reaction mixture was concentrated to remove the methanol and the aqueous phase was treated dropwise with 10% aq AcOH till the pH of the solution was 7. A white solid began to fall out of solution and the mixture was allowed to sit at room temperature overnight. The solid was filtered and washed with a little water followed by diethyl ether to produce a nearly white solid. The material was treated with 0.1 M of trifluoroacetic acid in water (1.08 mL) and 1 mL H₂O and the solution was lyophilized to yield the title compound (7) (32 mg, 77%) as a light yellow solid: MS (ESI+) for C₁₉H₂₀FN₃O₄ m/z 374.0 (M+H)⁺; MS (ESI−) for C₁₉H₂₀FN₃O₄ m/z 372.0 (M−H)⁻; HPLC purity 100% (ret time, 4.43 min).

### Example 7

### Example 7A

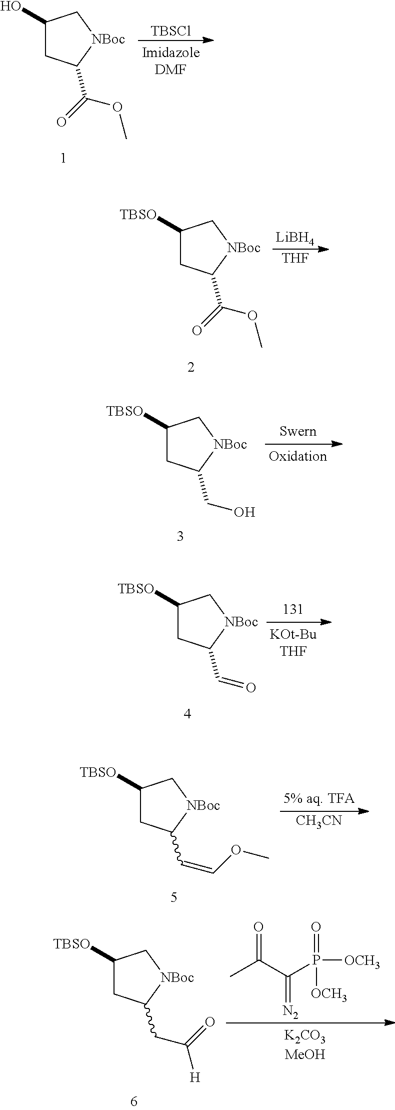

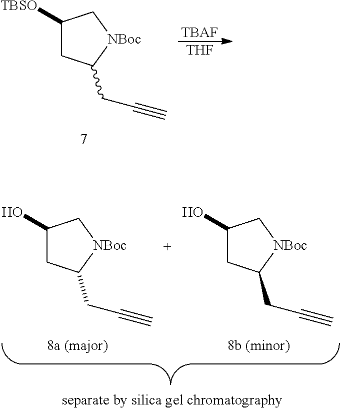

HPLC conditions (for final analysis and reaction monitoring) are as follows: Agilent 1100 HPLC. Agilent Scalar C18 150×4.6 mm 5 micron column. Solvent A—Water (0.1% TFA; Solvent B—Acetonitrile (0.07% TFA, Gradient—10 min 95% A to 95% B; 5 min hold; then recycle; UV Detection @ 214 and 250 nm.

Prep HPLC conditions used for final purification: Phenomenex Luna 250×21.20 mm, 10 micron; Gradient: solvent A is 0.07% TFA in acetonitrile; solvent B is 0.10% TFA in water; rate is 20 mL/min; 30 minute run; 5% to 70% A over 14 minute ramp; 3 minute ramp from 80% to 100% A; hold 100% A for 3 minutes; ramp down from 100% to 5% A over 5 minutes; hold 5 minutes then recycle.

### Methyl-(2S,4R)—N-tert-butoxycarbonyl-4-(tert-butyldimethylsilyloxy)-2-pyrrolidinecarboxylate (2)

Methyl-(2S,4R)—N-tert-butoxycarbonyl-4-hydroxy-2-pyrrolidinecarboxylate (1) (10.0 g, 40.8 mmol; Synthetec) was dissolved in N,N-dimethylformamide (200 mL) and the reaction was cooled at 0° C. (using an ice-water bath) and then 1H-imidazole (6.7 g, 98.0 mmol) was added. Then, tert-butyldimethylsilyl chloride (7.4 g, 49.0 mmol) in N,N-dimethylformamide (100 mL) was added dropwise via a pressure equalizing dropping funnel over a ˜20 min period of time. After the addition of tert-butyldimethylsilyl chloride was complete, the ice bath was removed and the reaction was stirred overnight at ambient temperature (˜20° C.). TLC analysis after this period of time showed a small amount of the starting material and so ˜20 mol % of each reagent was added with continued stirring for 2-3 hr. The reaction was checked again by TLC after this period of time and was determined to be complete. The reaction was quenched by pouring into water. The organic product was extracted with diethyl ether (3×100 mL) and the combined organic layers washed with water (3×100 mL) and brine, dried over MgSO₄, filtered and concentrated in vacuo to afford the TBS ether (2) (14.6 g, 99% yield) which was used without further purification; ¹H NMR (400 MHz, DMSO-d₆) δ ppm 0.05 (s, 3H), 0.06 (s, 3H), 0.84 (s, 9H), 1.32 (s, 4.5H, OtBu rotamer), 1.38 (s, 4.5H, OtBu rotamer), 1.89-2.01 (m, 1H), 2.06-2.16 (m, 1H), 3.19-3.30 (m, 1H), 3.40-3.48 (m, 1H), 3.63 (s, 1.5H, OMe rotamer), 3.66 (s, 1.5H, OMe rotamer), 4.14-4.26 (m, 1H), 4.39-4.47 (m, 1H); MS ES⁺360.1 m/z (M+1)⁺, 382.3 m/z (M+Na)⁺.

### tert-Butyl-(2S,4R)-4-[tert-butyl(dimethyl)silyl]oxy-2-(hydroxymethyl)pyrrolidine-1-carboxylate (31

1-Methyl-(2S,4R)—N-tert-butoxycarbonyl-4-(tert-butyldimethylsilyloxy)-2-pyrrolidinecarboxylate (2) (14.6 g, 40.6 mmol) was dissolved in tetrahydrofuran (300 mL) and then was cooled at 0° C. Then lithium tetrahydroborate (1.3 g, 61.0 mmol) was added in portions over a 10 minute period. The reaction was allowed to come to room temperature (˜22° C.) and stir for 2 hours after the addition was complete. After this period of time, the reaction was complete based on TLC analysis (30% ethyl acetate/hexanes). The reaction was quenched by pouring into a solution of ice water with 1M HCl (˜50 mL to give ˜300 mL volume total) and chloroform (˜200 mL). The reaction was stirred until the cloudy precipitate had dissolved, and then the organic product was extracted with chloroform (3×150 mL) and the combined organic layers washed with brine, dried over MgSO₄, filtered, and concentrated in vacuo to afford the crude product. The product was purified using a 90 g silica gel cartridge, eluting with 0 to 30% ethyl acetate in hexanes to afford the purified product (3) as a thick clear oil, 12.2 g, 91% yield; ¹H NMR and MS are consistent for the desired product; ¹H NMR (400 MHz, DMSO-d₆) δ ppm 0.04 (s, 3H), 0.05 (s, 3H), 0.84 (s, 9H), 1.38 (s, 9H), 1.76-1.84 (m, 1H), 1.91-2.01 (m, 1H), 3.24 (d, J=3.52 Hz, 2H), 3.39-3.45 (m, 2 H), 3.69-3.84 (m, 1H), 4.33-4.44 (m, 1H), 4.67 (t, J=5.60 Hz, 1H); MS: 354.3 m/z (M+Na)⁺.

### tert-Butyl-(2S,4R)-4-[tert-butyl(dimethyl)silyl]oxy}-2-formylpyrrolidine-1-carboxylate (4)

tert-Butyl-(2S,4R)-4-{[tert-butyl(dimethyl)silyl]oxy-2-(hydroxymethyl)pyrrolidine-1-carboxylate (3) (3.52 g, 10.6 mmol) was dissolved in methylene chloride (75 mL) and N-methylmorpholine N-oxide (1.4 g, 12.0 mmol) and 4 \acute{Å} molecular sieves (4.0 g) were added successively. Then, the reaction was cooled in a water bath and tetrapropylammonium perruthenate(VII) (93 mg, 0.26 mmol) was added in one portion. The reaction was stirred with the water bath for 10 minutes and then the water bath was removed. The reaction was monitored by thin layer chromatography and was determined to be complete after ˜40 min. After this period of time, the reaction was filtered through a short pad of Celite 545 with ˜20 g of silica gel on top. The desired product (4) eluted with DCM washes to afford a light colored oil (2.5 g, 71% yield); ¹H NMR was consistent with the proposed structure; ¹H NMR (400 MHz, DMSO-d₆) δ ppm 0.06 (s, 3H), 0.06 (s, 3H), 0.84 (s, 9H), 1.34 (s, 4.5H, N-tBu rotamer), 1.40 (s, 4.5H, N-tBu rotamer), 1.86-1.96 (m, 1H), 1.95-2.05 (m, 1H), 3.28 (dd, J=11.09, 7.98 Hz, 1H), 3.40-3.52 (m, 1H), 4.02-4.13 (m, 1H), 4.42 (br. s., 1 H), 9.40 (d, J=3.72 Hz, 0.5H, CHO rotamer), 9.41 (d, J=3.73 Hz, 0.5H, CHO rotamer).

The oxidation was also performed using Swern conditions as follows: Dimethyl sulfoxide (17.2 mL, 0.242 mol) was added to a solution of oxalyl chloride (10.3 mL, 0.121 mol) in 400 mL of CH₂Cl₂ that had been cooled to −78° C. under N₂. The mixture was stirred for 10 minutes, and then tert-butyl (2S,4R)-4-{[tert-butyl(dimethyl)silyl]oxy-2-(hydroxymethyl)pyrrolidine-1-carboxylate (3, 20.10 g, 60.6 mmol) was added as a solution in 50 mL of CH₂Cl₂. After 10 minutes triethylamine (33.8 mL, 0.242 mol) was added and the mixture was stirred at −78° C. for 15 minutes. Thin layer chromatography analysis at this time (5% EtOAc/CH₂Cl₂) shows no remaining alcohol. The reaction was quenched by addition of 300 mL of water and then allowed to warm to room temperature. The water layer was extracted with 300 mL of CH₂Cl₂ and the combined organic layers were dried over Na₂SO₄. Evaporation gave 19.9 g of a light yellow solid, 99% yield. No further purification was required.

### tert-Butyl-(4R)-4-{[tert-butyl(dimethyl)silyl]oxy}-2-[(Z)-2-methoxyvinyl]pyrrolidine-1-carboxylate (5)

Potassium tert-butoxide (9.46 g, 84.3 mmol) was added in portions to a 0° C. suspension of (methoxymethyl)triphenylphosphonium chloride (30.3 g, 88.4 mmol) in tetrahydrofuran (260 mL) and then the reaction was allowed to warm to room temperature and stir for 2 hours. After this period of time, the dark red solution was returned to the 0° C. cold bath and tert-butyl-(2S,4R)-4-{[tert-butyl(dimethyl)silyl]oxy}-2-formylpyrrolidine-1-carboxylate (4) (13.09 g, 39.7 mol) in tetrahydrofuran was added dropwise via a pressure equalizing dropping funnel and the reaction was stirred overnight during which time the cold bath slowly expired. Thin layer chromatography analysis after 16 hr shows complete consumption of the starting material and formation of a new, higher R_{f} product. The reaction was quenched by the addition of saturated sodium bicarbonate (200 mL) and the organic product was extracted with ethyl acetate (3×100 mL) and the combined organic layers were washed with brine, dried over magnesium sulfate, filtered and concentrated in vacuo to afford the crude product. The resulting slurry was triturated with ether to remove triphenylphosphine and triphenylphosphine oxide and the filtrate concentrated in vacuo and the trituration process was repeated until only a thick oil remained after concentrating the filtrate. Chromatography with 0 to 20% ethyl acetate in hexanes using a 120 g silica gel cartridge afforded 8.92 g (63-64%) of the purified product (5) that is a mixture of diastereomers. The trans-proline isomer is major and cis-proline isomer minor based on x-ray crystallography of the minor product. ¹H NMR shows only the cis-olefin based on coupling constants and is consistent with a 60:40 mixture of cis-proline and trans-proline diastereomeric products at C-2; ¹H NMR (400 MHz, CDCl₃) δ 6.38 (d, J=12 Hz, 0.6H), 5.801 (m, 0.4H), 4.66 (m, 0.4H), 4.55 (m, 0.6H), 4.28 (m, 2H), 3.53 (s, 1.2H), 3.46 (s, 1.8H), 3.37 (m, 1 H), 3.29 (m, 1H), 1.98 (m, 1H), 1.73 (m, 1H), 1.38 (s, 9H), 0.82 (s, 9H), 0.00 (s, 3 H), −0.01 (s, 3H).

### tert-Butyl-(4R)-4-{[tert-butyl(dimethyl)silyl]oxy}-2-prop-2-yn-1 carboxylate (7)

tert-Butyl-(4R)-4-{[tert-butyl(dimethyl)silyl]oxy}-2-[(E)-2-methoxyvinyl]pyrrolidine-1-carboxylate (5) (11.51 g, 32.2 mmol) was dissolved in acetonitrile (200 mL) and then 5% aqueous TFA (50 mL) was added. The reaction was stirred at ambient temperature for 45 min and then checked. TLC analysis at this time showed consumption of the starting material and formation of a new product. The reaction was quenched by the addition of saturated aqueous sodium bicarbonate until the pH was slightly basic. Then, the solvent was removed in vacuo and the organic product was extracted from the resulting aqueous layer (3×100 mL) CH₂Cl₂ to afford the crude product which contained a small amount of TBS hydrolysis product along with the TBS ether. The crude product was subjected directly to the Bestmann alkynylation conditions as follows: The aldehyde (6) was dissolved in methanol (100 mL) and potassium carbonate (8.9 g, 64.0 mmol) was added. Then, dimethyl (1-diazo-2-oxopropyl)phosphonate (7.4 g, 39.0 mmol) in methanol (30 mL) was added dropwise to the stirred solution at room temperature and the resulting mixture was stirred overnight at room temperature. After this period of time, the reaction was complete based on TLC analysis which shows formation of a new, less polar product. The reaction was quenched with saturated ammonium chloride and then the volatiles were removed in vacuo. The resulting aqueous layer was partitioned between water and ethyl acetate and then the aqueous layer was extracted with ethyl acetate two more times (˜50 mL each). The combined organic layers were washed with brine, dried over MgSO₄, filtered and concentrated in vacuo to afford a mixture of the TBS ether (7) and alcohol. (major diastereomer, trans-proline) ¹H NMR (400 MHz, DMSO-d₆) δ ppm 0.06 (s, 6 H), 0.84 (s, 9H), 1.40 (br. s., 9H), 1.85-2.00 (m, 2H), 2.47-2.63 (m, 2H), 2.77-2.88 (m, 1H), 3.16-3.32 (m, 2H), 3.79-3.91 (m, 1H), 4.29-4.49 (m, 1H); for mixture: ¹H NMR (400 MHz, CDCl₃) δ ppm 4.36 (m, 1H), 4.02 (m, 1H), 3.40 (m, 2 H), 2.57 (m, 2H), 2.03 (m, 3H), 1.48 (t, J=2.59 Hz, 9H), 0.88 (m, 9H), 0.08 (m, 6H).

### tert-Butyl-(2R,4R)-4-hydroxy-2-prop-2-yn-1-ylpyrrolidine-1-carboxylate and tert-butyl (2S,4R)-4-hydroxy-2-prop-2-yn-1-ylpyrrolidine-1-carboxylate (8a and 8b)

The crude product from above, tert-butyl-(4R)-4-{[tert-butyl(dimethyl)silyl]oxy}-2-prop-2-yn-1-ylpyrrolidine-1-carboxylate (7) (10.0 g, 29.4 mmol), was dissolved in tetrahydrofuran (200 mL) and 30.0 mL of 1.0 M of tetra-n-butylammonium fluoride in tetrahydrofuran was added dropwise via an addition funnel. The reaction was monitored by TLC for completion. After ˜2-3 hours, the reaction was complete and 200 mL of water was added and the reaction was concentrated to remove the THF. The alcohol was extracted with ethyl acetate (3×100 mL) and then the combined organic layers were washed with water and brine, dried over MgSO₄, filtered and concentrated in vacuo to afford the crude product as a mixture of cis- and trans-proline isomers. These were separated by silica gel chromatography-120 g silica gel column, eluting with 0 to 30% ethyl acetate/hexanes gradient (˜300-500 mL) in 5% increments to afford clean separation of the cis- and trans-proline isomers along with a small amount of mixed fractions. The mixed fractions were combined and subjected to the same chromatography conditions (120 g silica gel cartridge) to afford more clean-cis, clean-trans and a smaller amount of mixed fractions. The mixed fractions were subjected to one additional column—90 g silica gel column, eluting with 0 to 30% ethyl acetate/hexanes to separate completely the cis- and trans-isomers. From the combined lots, a total of 2.33 g of the (8b) cis-proline isomer (33% yield) and 4.35 g of the (8a) trans-proline isomer (62% yield) were obtained. ¹H NMR is consistent; trans-proline (8a, lower R_{f}): ¹H NMR (400 MHz, CDCl₃) δ ppm 4.40 (m, 1H), 4.01 (m, 1H), 3.54 (m, 1H), 3.41 (br. s., 1H), 2.62 (m, 1H), 2.47 (m, 1H), 2.07 (m, 2H), 1.86 (m, 1H), 1.74 (m, 1H), 1.41 (s, 9H); cis-proline (8b, higher R_{f}): ¹H NMR (400 MHz, CDCl₃) δ ppm 4.44 (m, 1H), 3.98 (m, 1H), 3.62 (m, 1H), 3.41 (d, J=11.82 Hz, 1H), 2.74 (m, 2 H), 2.27 (m, 1H), 2.13 (m, 1H), 2.02 (m, 2H), 1.48 (br. s., 9H).

### Example 7B

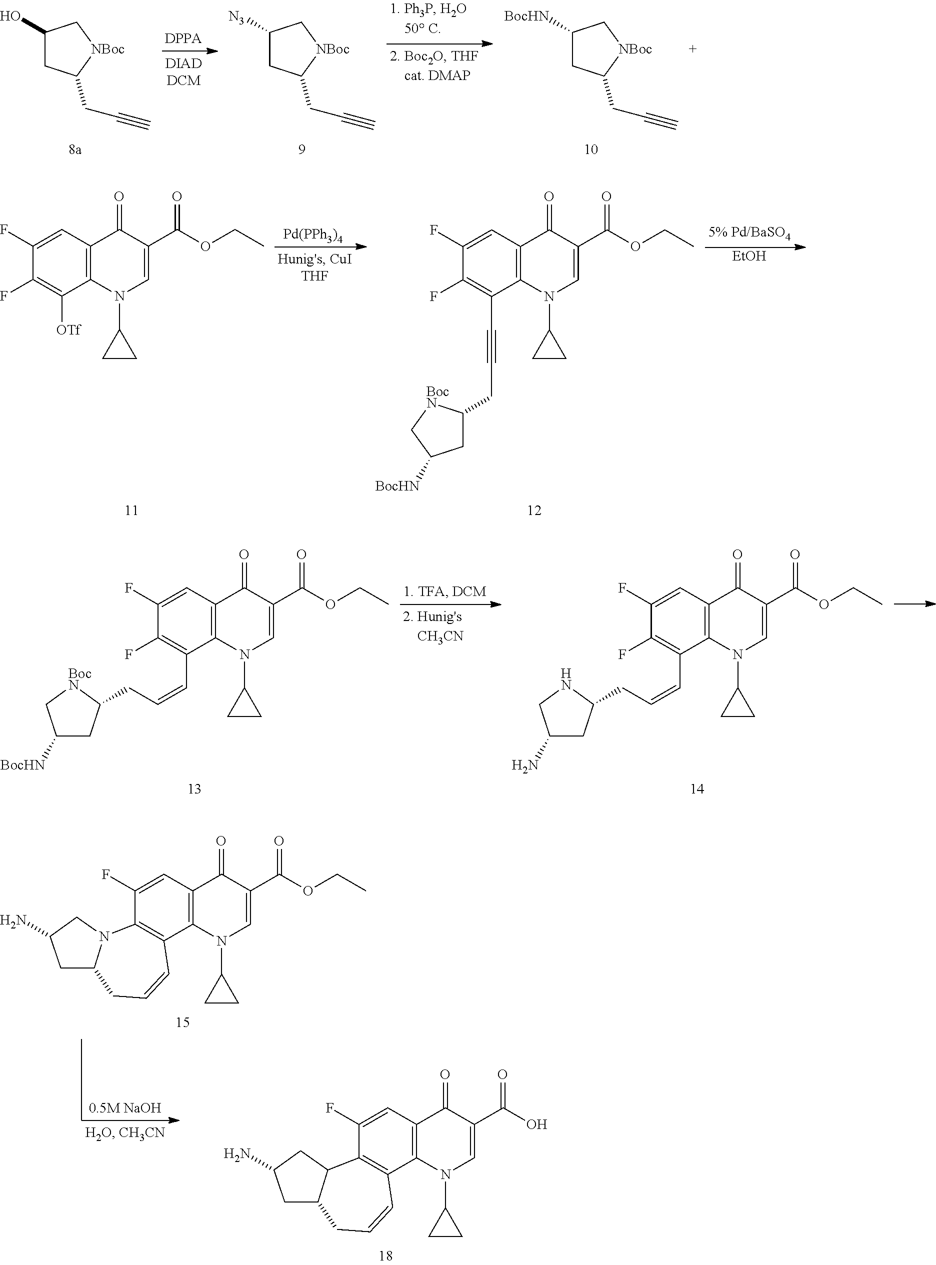

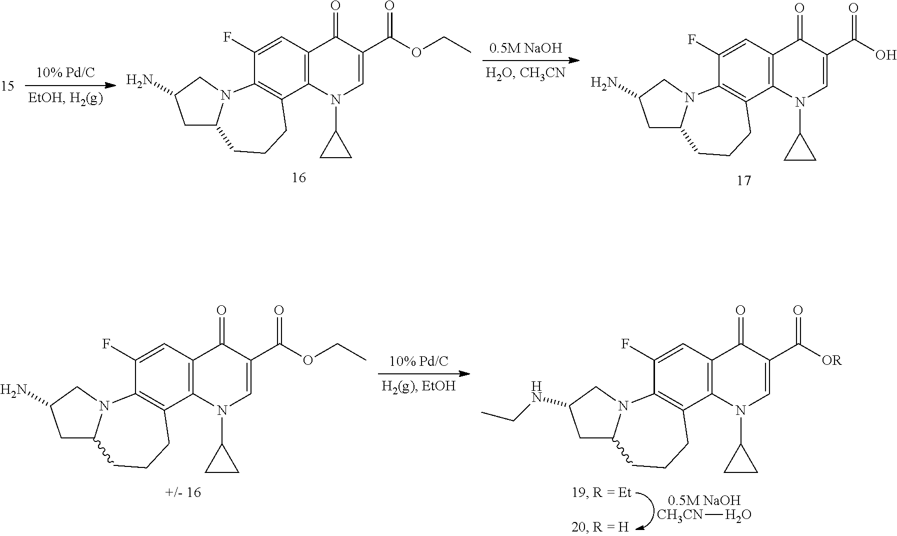

tert-Butyl-(2R,4S)-4-[(tert-butoxycarbonyl)amino]-2-prop-2-yn-1-ylpyrrolidine-1-carboxylate (10)

tert-Butyl-(2R,4R)-4-hydroxy-2-prop-2-yn-1-yl-pyrrolidine-1-carboxylate (8a) (0.25 g, 1.1 mmol) was dissolved in methylene chloride (10 mL) and triphenylphosphine (0.291 g, 1.11 mmol) was added and then the reaction vessel was cooled in an ice-water bath. Then, diisopropyl azodicarboxylate (0.330 mL, 1.68 mmol) (DIAD) was added dropwise via syringe and finally diphenylphosphonic azide (0.394 mL, 1.83 mmol) was added dropwise via syringe. The resulting solution was stirred overnight and the ice bath was allowed to slowly expire. The reaction was checked by TLC after this period of time and shows complete consumption of the starting alcohol and formation of a much higher R_{f} product. The reaction was transferred to a 150 mL separatory funnel, and diluted with ˜50 mL CH₂Cl₂ and washed twice with water (˜25 mL) and once with brine (˜25 mL). The organic layer was dried over MgSO₄, filtered and then, triphenylphosphine (0.87 g, 3.3 mmol) was added to the bright yellow solution—at which time the solution became clear and colorless. The reaction mixture was then concentrated in vacuo (in a 100 mL round bottom flask) and then taken up in tetrahydrofuran (20 mL). The reaction was checked by TLC at this time and most of the starting azide was gone and another, significantly lower-R_{f} product had formed, the iminophosphorane. Then, the vessel was equipped with a reflux condenser and water (2.4 mL, 130 mmol) was added and the reaction was heated at 55° C. for 5-6 hr before checking again. After this period of time, the hydrolysis of the iminophosphorane appeared to be complete based on TLC—the initial much significantly lower product had disappeared and a new, slightly less polar product had formed. The reaction was cooled to room temperature and concentrated to ½ volume and then transferred to a 30 mL scintillation vial that was under an atmosphere of nitrogen. Then, di-tert-butyldicarbonate (0.29 g, 1.3 mmol) and a catalytic amount of 4-dimethylaminopyridine (14 mg, 0.11 mmol) was added along with another 10 mL of THF to aid in solubility of all the reactants. The reaction was then allowed to stir overnight at ambient temperature. After this period of time, the formation of the N-Boc carbamate was complete based on TLC analysis. The reaction was transferred to a 100-mL round bottom flask and then the solvent was removed in vacuo. The crude film was taken up in ethyl acetate (˜100 mL) and washed once with water (˜25 mL) and once with brine (˜25 mL), dried over MgSO₄, filtered and concentrated in vacuo to afford the crude product. The crude material was purified using silica gel (40 g) chromatography, eluting with 0 to 25% ethyl acetate in hexanes to afford the desired compound (10), 262 mg, 73% yield for the three steps. ¹H NMR is consistent; NMR (400 MHz, CDCl₃) 1.46 (d, J=4.77 Hz, 18H), 1.83 (br. s., 1H), 1.99 (br. s., 2H), 2.35-2.89 (m, 3H), 3.07 (br. s., 1H), 3.88 (br. s., 2H), 4.12 (s, 1H), 4.77 (br. s., 1H); MS ES⁺347.22 m/z for [C₁₇H₂₈N₂O₄+Na]⁺.

### Ethyl-8-(3-{(2R,4S)-1-(tert-butoxycarbonyl)-4-[(tert-butoxycarbonyl)amino]pyrrolidin-2-yl}prop-1-yn-1-yl)-1-cyclopropyl-6,7-difluoro-4-oxo-1,4-dihydroquinoline-3-carboxylate (12)

tert-Butyl-(2R,4S)-4-[(tert-butoxycarbonyl)amino]-2-prop-2-yn-1-ylpyrrolidine-1-carboxylate (10) (332.9 mg, 1.026 mmol) and ethyl-1-cyclopropyl-6,7-difluoro-4-oxo-8-{[(trifluoromethyl)sulfonyl]oxy}-1,4-dihydroquinoline-3-carboxylate (11) (452.9 mg, 1.026 mmol) were combined in a 40 mL scintillation vial and then placed under nitrogen via partial vacuum evacuation and backfill with nitrogen. Then, triphenylphosphine (67 mg, 0.25 mmol) and tetrahydrofuran (10 mL) were added and the reaction was sparged with nitrogen for 3-4 minutes. Then, N,N-diisopropylethylamine (0.357 mL, 2.05 mmol) and tetrakis(triphenylphosphine)palladium(0) (120 mg, 0.10 mmol) were added with continued sparging (˜3 min) and then finally copper(I) iodide (39 mg, 0.20 mmol) was added and the resultant clear, yellow-colored reaction mixture was heated at 60° C. for 8-9 hr and then checked by HPLC/LCMS and TLC. Analysis after this period of time shows complete consumption of the starting triflate and formation of the desired coupled product, based on MS. The reaction appeared clean by TLC and HPLC with a major product formed. Other minor peaks are noted in the HPLC at longer retention times, these correspond to containing Pd in the LCMS. The crude reaction was diluted with ethanol (˜10 mL) and then stirred for 5 minutes before filtering through a short plug of Celite 545 to remove the precipitated salts. The filtrate was concentrated in vacuo and then subjected to regular phase silica gel chromatography on a 40 g silica gel cartridge, eluting with 0 to 30% ethyl acetate in DCM to afford the purified product (12), 493 mg, 78% yield, which contains a small amount of triphenylphosphine, ˜6% by HPLC at 250 nm; ¹H NMR (400 MHz, CDCl₃) δ ppm 8.62 (s, 1H), 8.24 (dd, J=9.74, 9.12 Hz, 1H), 4.71 (br. s., 1H), 4.40 (q, J=7.19 Hz, 2H), 4.15 (m, 2H), 4.02 (m, 1 H), 3.94 (m, 1H), 3.18 (m, 1H), 3.07 (m, 1H), 2.91 (m, 1H), 2.53 (m, 1H), 1.92 (m, 1 H), 1.47 (s, 9H), 1.43 (s, 9H), 1.41 (m, 3H), 1.27 (m, 2H), 1.13 (m, 2H); MS ES⁺ 616.5 m/z for [C₃₂H₃₉F₂N₃O₇+1]⁺.

### Ethyl-8-[(1Z)-3-{(2R,4S)-1-(tert-butoxycarbonyl)-4-[(tert-butoxycarbonyl)amino]pyrrolidin-2-yl}prop-1-en-1-yl]-1-cyclopropyl-6,7-difluoro-4-oxo-1,4-dihydroquinoline-3-carboxylate (13)

Ethyl-8-(3-{(2R,4S)-1-(tert-butoxycarbonyl)-4-[tert-butoxycarbonyl)amino]pyrrolidin-2-yl}prop-1-yn-1-yl)-1-cyclopropyl-6,7-difluoro-4-oxo-1,4-dihydroquinoline-3-carboxylate (12) (490.0 mg, 0.7959 mmol) was dissolved in ethanol (30 mL) and then the reaction was partially evacuated with vacuum and then back filled with nitrogen ×3 before adding palladium on barium sulfate, reduced (0.36 g). The reaction was then partially evacuated and back filled with hydrogen ×3, sparged with 3-1 L balloons filled with hydrogen, and then maintained under an atmosphere of hydrogen with a hydrogen filled balloon and checked after 30-45 minutes for completion. The reaction was checked at this time, showing no progress. More hydrogen was added (by sparging) and continued under an atmosphere of hydrogen for several hours with no change. The reaction mixture was filtered through a short plug of Celite 545 and then rinsed with 20 mL of ethanol. The filtrate (in a 100 mL flask) was sparged with nitrogen for 3 minutes and then placed under an atmosphere of nitrogen and 10% palladium on carbon (85 mg) was added. The reaction vessel was then partially evacuated and back-filled with hydrogen (×3) and then kept under an atmosphere of hydrogen with a balloon. The reaction was checked after 2-3 hrs and found to be complete, with no apparent over-reduction. The reaction was filtered through a short plug of Celite 545 and then the filtrate was concentrated in vacuo. Silica gel chromatography using 0 to 35% ethyl acetate in CH₂Cl₂ afforded purified product (13), 450 mg, 92% isolated yield; ¹H NMR is consistent; ¹H NMR (400 MHz, CDCl₃) δ ppm 8.64 (s, 1H), 8.27 (t, J=9.43 Hz, 1H), 6.84 (d, J=11.20 Hz, 1H), 5.95 (m, 1H), 4.41 (q, J=7.05 Hz, 2H), 4.02 (m, 1H), 3.87 (m, 2H), 3.74 (m, 1H), 2.87 (m, 1H), 2.39 (m, 2H), 2.00 (m, 1H), 1.65 (br. s., 2H), 1.45 (s, 18H), 1.42 (t, J=7.15 Hz, 3H), 1.27 (m, 1H), 1.17 (m, 2H), 0.98 (m, 1H); MS ES⁺618.4 m/z for [C₃₂H₄₁F₂N₃O₇+1]⁺.

### Ethyl-(7aR,9S)-9-amino-4-cyclopropyl-12-fluoro-1-oxo-4,7,7a,8,9,10-hexahydro-1H-pyrrolo[1′,2′:1,7]-azepino[2,3-h]quinoline-2-carboxylate (15)

Ethyl-8-[(1Z)-3-{(2R,4S)-1-(tert-butoxycarbonyl)-4-[(tert-butoxycarbonyl)-amino]pyrrolidin-2-yl}prop-1-en-1-yl]-1-cyclopropyl-6,7-difluoro-4-oxo-1,4-dihydroquinoline-3-carboxylate (13) (451 mg, 0.730 mmol) in a 40-mL scintillation vial, at room temperature and under an atmosphere of nitrogen, was dissolved in methylene chloride (25 mL) and trifluoroacetic acid (1.12 mL, 14.6 mmol) was added in one portion. The reaction was stirred overnight (˜10 hr) at ambient temperature and then checked by HPLC. After this period of time, the reaction was determined to be complete based on HPLC and the solvent was removed with a stream of nitrogen. Then, the resulting film was taken up in 2% MeOH/CHCl₃ and washed once with 10% aqueous ammonium hydroxide solution (˜20 mL) and then once with brine (˜20 mL), dried over MgSO₄, filtered and concentrated in vacuo. The crude product was transferred to a 40-mL scintillation vial and then placed under an atmosphere of nitrogen before dissolving in acetonitrile (20 mL) and adding N,N-diisopropylethylamine (0.32 mL, 1.8 mmol). The resulting solution was sparged with nitrogen for 2 minutes and then capped and heated at 55° C. overnight (˜10 hr). After this period of time, the reaction was checked by HPLC for completion. HPLC shows complete consumption of the intermediate di-amine (14) and formation of a new, slightly less polar product (15). The reaction mixture was concentrated in vacuo and then subjected to silica gel chromatography (40 g silica gel column), eluting with 0, 2.5, 5, 7.5 and 10% methanol in chloroform (˜300 mL each). The fractions containing product were combined to afford 210 mg of the cyclized product (15) in 72% isolated yield. ¹H NMR is consistent; ¹H NMR (400 MHz, CDCl₃) δ ppm 8.51 (s, 1H), 7.70 (d, J=14.93 Hz, 1H), 6.48 (d, J=12.23 Hz, 1H), 5.91 (dt, J=12.02, 4.15 Hz, 1H), 4.31 (qd, J=7.15, 1.97 Hz, 2H), 4.24 (m, 1H), 3.78 (m, 2H), 3.72 (m, 1H), 3.36 (m, 1H), 2.99 (m, 1H), 2.55 (m, 1H), 2.43 (ddd, J=13.06, 8.71, 6.63 Hz, 1H), 1.83 (br. s., 2 H), 1.53 (m, 1H), 1.33 (t, J=7.05 Hz, 3H), 1.18 (m, 1H), 0.88 (m, 2H), 0.68 (m, 1 H); MS ES⁺: 398.3 m/z for [C₂₂H₂₄FN₃O₃+1]⁺.

### (7aR,9S)-9-Amino-4-cyclopropyl-12-fluoro-1-oxo-4,7,7a,8,9,10-hexahydro-1H-pyrrolo[1′,2′:1,7]azepino[2,3h]-quinoline-2-carboxylic acid trifluoroacetate (18)

Ethyl-(7aR,9S)-9-amino-4-cyclopropyl-12-fluoro-1-oxo-4,7,7a,8,9,10-hexahydro-1H-pyrrolo[1′,2′:1,7]azepino[2,3-h]quinoline-2-carboxylate (15) (24.0 mg, 0.0604 mmol) was dissolved in tetrahydrofuran (5 mL) and then potassium trimethylsilanolate (10.3 mg, 0.0725 mmol) was added. The reaction was stirred for 30 minutes and then checked by TLC (20% methanol/CHCl₃) and a small amount of product (18) is formed. The reaction was stirred an additional two hours before checking again. After this period of time, the reaction has progress a small amount. Thus, an additional lot of potassium trimethylsilanolate (8.1 mg, 0.063 mmol) in acetonitrile (1 mL) was added with continued stirring for 2 hr before checking again. After this period of time, the reaction was complete. Acetic acid was added dropwise to reach pH was 6-7 and then the reaction was concentrated in vacuo and subjected to prep HPLC for purification using the conditions described above; ¹H NMR is consistent; ¹H NMR (400 MHz, DMSO-d₆) δ ppm 0.74-0.85 (m, 1H) 0.85-0.98 (m, 1H) 0.98-1.08 (m, 1H) 1.66-1.80 (m, 1H) 2.61-2.76 (m, 3H) 3.39-3.51 (m, 2H) 3.79-3.90 (m, 1H) 3.88-4.01 (m, 1H) 4.20-4.32 (m, 1H) 4.39-4.52 (m, 1H) 6.05 (dt, J=11.97, 4.07 Hz, 1H) 6.80 (d, J=12.44 Hz, 1H) 7.69 (d, J=14.30 Hz, 1H) 8.16 (br. s., 2H) 8.78 (s, 1H) MS ES⁺370.2 m/z for [C₂₀H₂₀FN₃O₃+1]⁺ and ES⁻ 368.2 m/z for [C₂₀H₂₀FN₃O₃−1]⁻.

### Ethyl-(7aR,9S)-9-amino-4-cyclopropyl-12-fluoro-1-oxo-4,5,6,7,7a,8,9,10-octahydro-1H-pyrrolo[1′,2′:1,7]-azepino[2,3-h]quinoline-2-carboxylate (16)

Ethyl-(7aR,9S)-9-amino-4-cyclopropyl-12-fluoro-1-oxo-4,7,7a,8,9,10-hexahydro-1H-pyrrolo[1′,2′:1,7]azepino[2,3-h]quinoline-2-carboxylate (15) (200.0 mg, 0.503 mmol) in a 50-mL round bottom flask was dissolved in ethanol (15 mL) and then the reaction vessel was partially evacuated with vacuum and back-filled with nitrogen and then the reaction solution was sparged with nitrogen for 2-3 minutes. After this, 10% palladium on carbon (54 mg) was added in one portion and the reaction vessel was partially evacuated and then back-filled with hydrogen gas (×3) and then the reaction mixture was sparged with 3-1 L hydrogen balloons and then finally maintained under an atmosphere of hydrogen with a hydrogen-filled balloon. The reaction was stirred for 10 hr before checking by HPLC. HPLC after this period of time shows that the reaction is about 50% complete. The reaction was charged with hydrogen once more and then the hydrogen atmosphere was maintained with a hydrogen-filled balloon. The reaction was stirred vigorously for 8 hr, after which time the reaction was complete and the starting material was consumed and a new product had formed based on HPLC and MS. The reaction was filtered through a short plug of Celite 545 and rinsed with 5% methanol in chloroform solution and then the filtrate was concentrated in vacuo. The crude product was purified by silica gel chromatography on a 40 g column, eluting with 0 to 15% methanol in chloroform to afford the purified product (16), 154.3 mg in 77% yield. ¹H NMR is consistent; ¹H NMR (400 MHz, CDCl₃) δ 8.53 (s, 1H), 7.83 (d, J=13.89 Hz, 1 H), 4.31 (m, 1H), 4.12 (m, 1H), 4.06 (m, 1H), 3.87 (m; 1H), 3.47 (m, 1H), 3.22 (m, 2 H), 2.50 (m, 1H), 2.42 (m, 1H), 2.04 (m, 1H), 1.80 (m, 1H), 1.67 (m, 2H), 1.51 (m, 2 H), 1.33 (t, J=7.15 Hz, 3H), 1.16 (m, 1H), 1.03 (m, 1H), 0.87 (m, 1H), 0.64 (m, 1H); ES⁺400.3 m/z for [C₂₂H₂₆FN₃O₃+1]⁺.

### (7aR,9S)-9-Amino-4-cyclopropyl-12-fluoro-1-oxo-4,5,6,7,7a,8,9,10-octahydro-1H-pyrrolo[1′,′:1,7]-azepino[2,3-h]quinoline-2-carboxylic acid trifluoroacetate (17)

Ethyl-(7aR,9S)-9-amino-4-cyclopropyl-12-fluoro-1-oxo-4,5,6,7,7a,8,9,10-octahydro-1H-pyrrolo[1′,2′:1,7]-azepino[2,3h]-quinoline-2-carboxylate (16) (134.6 mg, 0.337 mmol) was dissolved in acetonitrile (7 mL) and then 0.74 mL of a 0.500 M aqueous sodium hydroxide was added. The reaction was stirred for 2 hr at 50° C. and checked by HPLC. The reaction was ˜60% complete at this time. An additional 100 uL of 0.500 M of aqueous sodium hydroxide was added with continued heating. After 3 hr the reaction was complete and was neutralized to ˜pH 7 by the use of acetic acid (1-2 drops required). Then, the crude product was lyophilized and then purified by prep HPLC using conditions described above to afford the purified product (17), 88.6 mg, 54% yield. ¹H NMR is consistent; after D₂O exchange (in DMSO): ¹H NMR (400 MHz, DMSO-d₆) δ ppm 8.78 (s, 1H), 7.77 (d, J=13.48 Hz, 1 H), 4.33 (m, 1H), 4.16 (m, 1H), 3.79 (m, 1H), 3.66 (m, 1H), 3.44 (m, 2H), 2.63 (m, 2 H), 2.01 (m, 1H), 1.89 (m, 1H), 1.74 (m, 1H), 1.64 (m, 2H), 1.23 (m, 1H), 1.15 (m, 1 H), 0.95 (m, 1H), 0.72 (m, 1H); MS ES⁺372.4 m/z for [C₂₀H₂₂FN₃O₃+1]⁺.

### Ethyl-(95)-4-cyclopropyl-9-(ethylamino)-12-fluoro-1-oxo-4,5,6,7,7a,8,9,10-octahydro-1H-pyrrolo-[1′,2′:1,7]azepino[2,3-h]quinoline-2-carboxylate (19)

Ethyl-(95)-9-amino-4-cyclopropyl-12-fluoro-1-oxo-4,7,7a,8,9,10-hexahydro-1H-pyrrolo[1′,2′:1,7]azepino[2,3-h]quinoline-2-carboxylate-TFA salt (±16) (236 mg, 0.594 mmol) in a round bottom flask was placed under an atmosphere of nitrogen and then was dissolved in ethanol (20 mL). The resulting solution was partially evacuated and back filled ×1 with nitrogen and then 10% palladium on carbon (0.13 g) was added. The resultant black mixture was partially evacuated and back filled with hydrogen ×3 and then sparged with ˜500 mL of hydrogen gas. The reaction was maintained under an atmosphere of hydrogen with a balloon and checked after 3 hr, showing little or no progress. Another 50 mg of 10% palladium on carbon was added and then the mixture was sparged with 2 L of hydrogen gas and then maintained under an atmosphere of hydrogen with a balloon and stirred overnight (˜10 hr); LCMS/HPLC after this period of time shows some progress, so the reaction mixture was sparged again with another 2 L of hydrogen gas and maintained under an atmosphere of hydrogen with a balloon. This sparging process was repeated two additional times after 16 and 24 hr; however no additional catalyst was added. After this period of time, the starting material was consumed. The reaction was filtered through a short plug of Celite 545 and then concentrated in vacuo. The resulting solid was subjected to silica gel chromatography, eluting with 0 to 15% MeOH:CHCl₃ to afford the N-ethyl amine compound (19). MS ES⁺428.2 m/z for [C₂₄H₃₀FN₃O₃+1]⁺.

### (9S)-4-cyclopropyl-9-(ethylamino)-12-fluoro-1-oxo-4,5,6,7,7a,8,9,10-octahydro-1H-pyrrolo[1′,2′:1,7]-azepino[2,3-h]quinoline-2-carboxylic acid trifluoroacetate (20)

Ethyl-(9S)-4-cyclopropyl-9-(ethylamino)-12-fluoro-1-oxo-4,5,6,7,7a,8,9,10-octahydro-1H-pyrrolo[1′,2′:1,7]azepino[2,3-h]quinoline-2-carboxylate (19) (50.0 mg, 0.117 mmol) was dissolved in acetonitrile (2 mL) and then 0.500 M aqueous sodium hydroxide (0.26 mL) was added. The reaction was stirred for 2 hr at 50° C. and checked by HPLC. The reaction was ˜60% complete at this time. An additional 100 uL of 0.500 M aqueous sodium hydroxide was added with continued heating. After 3 hr the reaction was complete and was neutralized to ˜pH 7 by the use of acetic acid (1-2 drops required). Then, the crude product was lyophilized and then purified by prep HPLC using conditions described above to afford the purified product, 54.3 mg, 90% yield. ¹H NMR is consistent for a mixture of diastereomeric products (exchangeable hydrogens not observed); ¹H NMR (400 MHz, D₂O) δ ppm 8.63 (m, 1H), 7.07 (m, 1 H), 4.14 (m, 2H), 3.99 (m, 1H), 3.81 (m, 2H), 3.51 (m, 1H), 3.38 (m, 1H), 3.13 (m, 2 H), 2.68 (m, 1H), 2.48 (m, 1H), 2.25 (m, 1H), 2.00 (m, 1H), 1.77 (m, 1H), 1.60 (m, 2 H), 1.26 (t, J=6.74 Hz, 3H), 1.17 (m, 2H), 0.80 (m, 1H), 0.74 (m, 1H); MS ES⁺400.37 m/z for [C₂₂H₂₆FN₃O₃+1]⁺.

### Example 7C

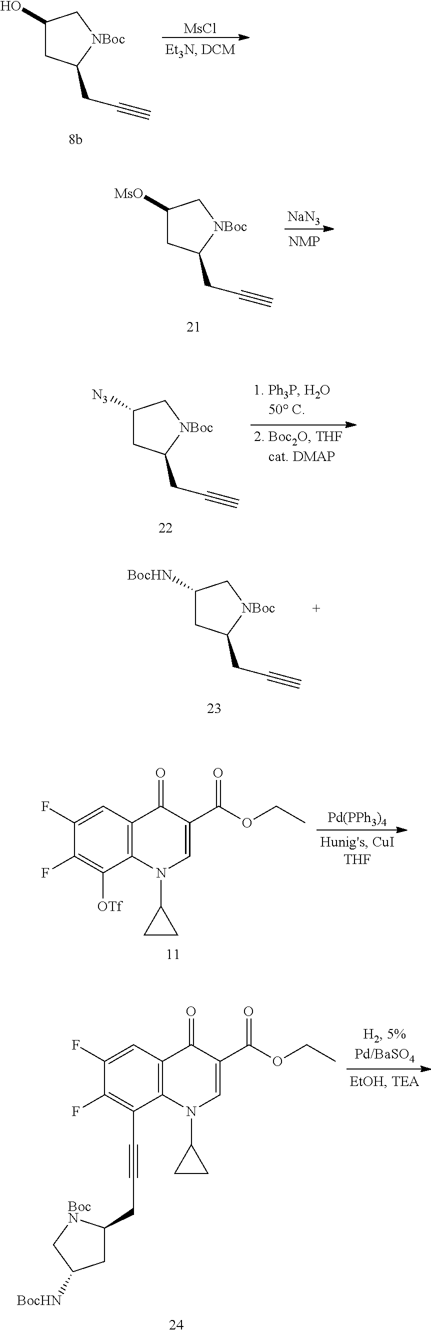

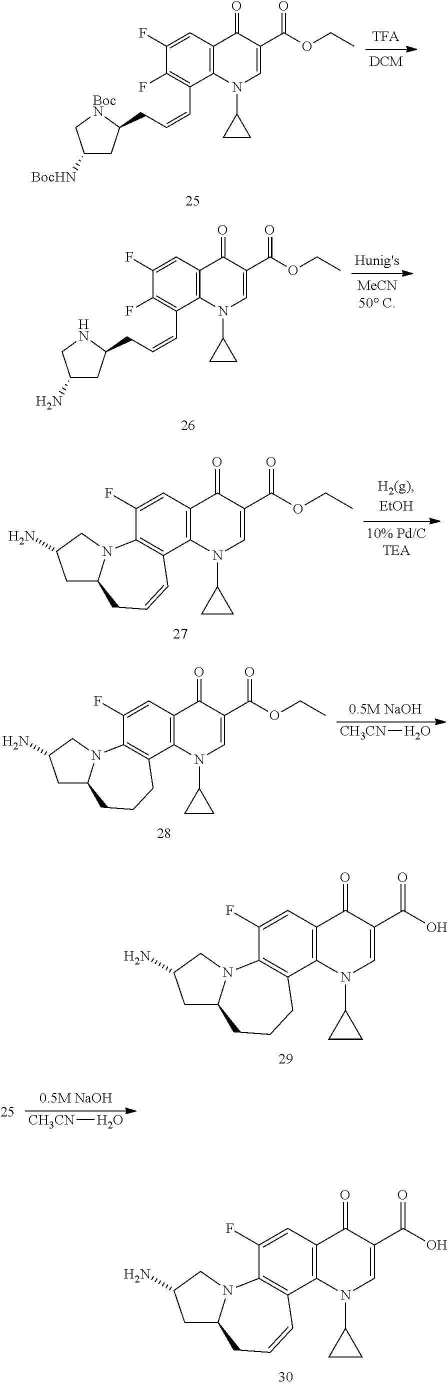

### tert-Butyl-(2S,4R)-4-[(methylsulfonyl)oxy]-2-prop-2-yn-1-ylpyrrolidine-1-carboxylate (21)

tert-Butyl-(2S,4R)-4-hydroxy-2-prop-2-yn-1-ylpyrrolidine-1-carboxylate (8b) (1.492 g, 6.62 mmol) was dissolved in methylene chloride (30 mL) and was cooled at 0° C. in an ice-water bath. Then, triethylamine (1.11 mL, 7.95 mmol) and methanesulfonyl chloride (0.564 mL, 7.28 mmol) were added successively and the reaction stirred at reduced temperature for 2 hr. TLC after this period of time shows incomplete consumption of the starting material and formation of a higher R_{f} product. An additional 0.1 equivalent of both triethylamine and methanesulfonyl chloride were added and stirred at 0° C. for 1 hr. After this period of time, the reaction was complete by TLC analysis. The reaction was quenched by pouring into ice-water/0.1M HCl (100 mL ea) and then the organic product was extracted with DCM (3×100 mL). The combined organic layers were washed with water and brine (50 mL ea), and then dried over sodium sulfate, filtered and concentrated in vacuo to afford the crude white solid (21) (2.05 g, 100%) that was used without further purification; ¹H NMR confirmed formation of the mesylate; ¹H NMR (400 MHz, CDCl₃) δ ppm 5.24 (m, 1H), 4.04 (m, 1 H), 3.78 (m, 1H), 3.66 (m, 1H), 3.08 (s, 3H), 2.83 (m, 1H), 2.44 (m, 3H), 2.00 (br. s., 1H), 1.48 (br. s., 9H).

### tert-Butyl-(2S,4S)-4-[(tert-butoxycarbonyl)amino]-2-prop-2-yn-1-ylpyrrolidine-1-carboxylate (23)

tert-Butyl-(2S,4R)-4-[(methylsulfonyl)oxy]-2-prop-2-yn-1-ylpyrrolidine-1-carboxylate (21) (2.05 g, 6.55 mmol) and N-methylpyrrolidinone (10 mL) were added to a 40 mL vial with stirring. Sodium azide (1.70 g, 0.0262 mol) was added and the reaction was stirred at 40° C. overnight. After this period of time, the reaction was complete based on TLC analysis (20% ethyl acetate in hexanes). The reaction was quenched by pouring into water and the organic product was extracted with diethyl ether (2×100 mL). The combined organic layers were washed with water (3×50 mL) and brine (1×50 mL), and then dried over sodium sulfate.

Then, the crude tert-butyl-(2S,4S)-4-azido-2-prop-2-yn-1-ylpyrrolidine-1-carboxylate (22) (1.64 g, 6.55 mmol) in diethyl ether (200 mL) and dichloromethane (20 mL) was treated with triphenylphosphine (5.16 g, 19.6 mmol). The solution was swirled to dissolve the solids and allowed to stand over night (some bubbles were evolved). LCMS and TLC analysis after this period of time showed complete consumption of the azide starting material and complete conversion to the iminophosphorane (MS ES+485.3 m/z, M+1). The solution was filtered to remove the sodium sulfate and concentrated to remove the solvent. Then, tetrahydrofuran (50 mL) and water (10 mL) were added successively and the reaction was heated to 50° C. for 4 hr and then cooled to room temperature. After this period of time, LCMS shows disappearance of the iminophosphorane. Subsequently, di-tert-butyldicarbonate (4.29 g, 19.6 mmol) was added followed by a catalytic amount of 4-dimethylaminopyridine (˜10 mol %). The reaction was stirred for 48 hr and then checked by TLC for completion and found to be complete after this period of time. The reaction was concentrated to remove the THF and the organic product was extracted with ethyl acetate (2×75 mL). The combined organic layers were washed with brine (50 mL), dried over sodium sulfate, filtered and concentrated in vacuo to afford the crude product. Silica gel chromatography, eluting with 15% ethyl acetate in hexanes afforded the purified product (23) (1.72 g, 81% yield) as a colorless oil; ¹H NMR is consistent; ¹H NMR (400 MHz, CDCl₃) [symbol] ppm 1.45 (s, 18H), 1.96 (br. s., 2H), 2.25 (br. s., 1H), 2.52 (br. s., 2H), 3.27 (br. s., 1H), 3.62 (dd, J=11.20, 6.22 Hz, 1H), 3.86-4.06 (m, 1 H), 4.30 (br. s., 1H), 4.55 (br. s., 1H); (ES⁺) 347.3 m/z for M+23 (M+Na)⁺; 291.3 m/z (M−99)⁺.

### Ethyl-8-(3-{(2S,4S)-1-(tert-butoxycarbonyl)-4-[(tert-butoxycarbonyl)amino]pyrrolidin-2-yl}prop-1-yn-1-yl)-1-cyclopropyl-6,7-difluoro-4-oxo-1,4-dihydroquinoline-3-carboxylate (24)

tert-Butyl-(2S,4S)-4-[(tert-butoxycarbonyl)amino]-2-prop-2-yn-1-ylpyrrolidine-1-carboxylate (23) (820.0 mg, 2.528 mmol) and ethyl-1-cyclopropyl-6,7-difluoro-4-oxo-8-{[(trifluoromethyl)sulfonyl]oxy}-1,4-dihydroquinoline-3-carboxylate (11) (1.12 g, 2.53 mmol) were combined in a 40 mL scintillation vial and then placed under nitrogen via vacuum evacuation and backfill with nitrogen. Then, triphenylphosphine (160 mg, 0.63 mmol) and tetrahydrofuran (25 mL) were added and the reaction was sparged with nitrogen for 3-4 minutes. Then, N,N-diisopropylethylamine (0.880 mL, 5.06 mmol) and tetrakis(triphenylphosphine)palladium(0) (290 mg, 0.25 mmol) were added successively with continued sparging (˜3 min) and then finally copper(I) iodide (120 mg, 0.63 mmol) was added and the resultant clear, yellow-colored reaction mixture was sealed with a cap and heated at 60° C. overnight. After heating for 16 hr, the reaction was checked by HPLC/LCMS and TLC. Analysis after this period of time shows complete consumption of the starting triflate and alkyne. The crude reaction was diluted with ethanol (˜10 mL) and then stirred for 5 minutes before concentrating in vacuo. The crude reaction was then subjected to silica gel chromatography on a 120 g cartridge, eluting with 15% ethyl acetate in DCM with 1% EtOH to afford the purified product (24), 1.49 g, 96% yield, which is ˜85 area % by HPLC. MS (ES⁺) 616.3 m/z (M+1) for [C₃₂H₃₉F₂N₃O₇+1]⁺; (ES⁻) 614.4 m/z (M−1) for [C₃₂H₃₉F₂N₃O₇−1]⁻.

### Ethyl-8-[(1Z)-3-{(2S,4S)-1-(tert-butoxycarbonyl)-4-[(tert-butoxycarbonyl)amino]pyrrolidin-2-yl}-prop-1-en-1-yl]-1-cyclopropyl-6,7-difluoro-4-oxo-1,4-dihydroquinoline-3-carboxylate (25)

Compound (25) was prepared from compound (24) according to the general procedure described above for compound (13) to afford the desired product; (ES⁺) 618.5 m/z (M+1) for [C₃₂H₄₁F₂N₃O₇+1]⁺; (ES⁻) 616.5 m/z (M−1) for [C₃₂H₄₁F₂N₃O₇−1]⁻.

Ethyl-(7aS,9S)-9-amino-4-cyclopropyl-12-fluoro-1-oxo-4,7,7a,8,9,10-hexahydro-1H-pyrrolo[1′,2′:1,7]-azepino[2,3-h]quinoline-2-carboxylate (27)

Compound (27) was prepared from compound (25) according to the general procedure described above for compounds (14) and (15) to afford the desired compound; ¹H NMR (400 MHz, CDCl₃) δ ppm 0.68-0.85 (m, 1H), 0.86-1.05 (m, 2 H), 1.18-1.28 (m, 2H), 1.41 (t, J=7.15 Hz, 3H), 1.73 (br. s., 2H), 1.93-2.11 (m, 2 H), 2.49-2.71 (m, 2H), 3.67-3.88 (m, 3H), 3.89-4.03 (m, 2H), 4.39 (qd, J=7.15, 7.05, 1.87 Hz, 2H), 5.94 (dt, J=12.34, 3.94, 3.84 Hz, 2H), 6.50 (d, J=12.23 Hz, 1 H), 7.77 (d, J=15.13 Hz, 1H), 8.58 (s, 1H); MS (ES⁺) 398.3 m/z (M+1) for [C₂₂H₂₄FN₃O₃+1]⁺; (ES⁻) 396.4 m/z (M−1) for [C₂₂H₂₄FN₃O₃−1]¹.

### (7aS,9S)-9-Amino-4-cyclopropyl-12-fluoro-1-oxo-4,7,7a,8,9,10-hexahydro-1H-pyrrolo[1′,2′:1,7]azepino-[2,3-h]quinoline-2-carboxylic acid trifluoroacetate (30)

Compound (30) was prepared from compound (27) according to the general procedure described above for compound (18); ¹H NMR and MS are consistent; ¹H NMR (400 MHz, DMSO-d₆) δ ppm 8.77 (s, 1H), 8.19 (br. s., 3H), 7.66 (d, J=14.51 Hz, 1H), 6.72 (d, J=12.44 Hz, 1H), 6.00 (dt, J=12.54, 3.94, 3.84 Hz, 1H), 4.25 (m, 1H), 3.98 (m, 4H), 2.63 (m, 2H), 2.26 (m, 1H), 2.09 (m, 1H), 1.27 (m, 1H), 0.98 (m, 2H), 0.82 (m, 1H); MS (ES⁺) 370.3 m/z (M+1) for [C₂₀H₂₂FN₃O₃+1]⁺; (ES⁻) 368.4 m/z (M−1) for [C₂₀H₂₂FN₃O₃-1]⁻.

### Ethyl-(7aS,9S)-9-amino-4-cyclopropyl-12-fluoro-1-oxo-4,5,6,7,7a,8,9,10-octahydro-1H-pyrrolo[1′,2′:1,7]-azepino-[2,3-h]quinoline-2-carboxylate (28)

Compound (28) was prepared from compound (27) according to the general procedure described above for preparation of compound (16) with ¹H NMR and MS consistent with the proposed structure; ¹H NMR (400 MHz, CDCl₃) δ 8.59 (s, 1H), 7.80 (d, J=14.30 Hz, 1H), 4.38 (m, 2H), 3.73 (m, 6H), 2.30 (m, 6H), 1.83 (m, 1H), 1.62 (m, 2H), 1.40 (t, J=7.15 Hz, 3H), 1.23 (m, 1H), 1.07 (m, 1H), 0.93 (m, 1H), 0.71 (m, 1H); MS (ES⁺) 400.3 m/z (M+1) for [C₂₂H₂₆FN₃O₃+1]⁺.

### (7aS,9S)-9-Amino-4-cyclopropyl-12-fluoro-1-oxo-4,5,6,7,7a,8,9,10-octahydro-1H-pyrrolo quinoline-2-carboxylic acid trifluoroacetate (29)

Compound (29) was prepared from compound (28) according to the general procedure described above for the synthesis of compound (17) with ¹H NMR and MS consistent with the proposed structure; ¹H NMR (400 MHz, DMSO-d₆) δ ppm 8.77 (s, 1H), 8.14 (br. s., 3H), 7.75 (d, J=14.10 Hz, 1H), 4.31 (dq, J=7.07, 3.68, 3.52 Hz, 1H), 3.97 (m, 2H), 3.77 (m, 2H), 3.64 (dd, J=15.13, 9.54 Hz, 1H), 2.21 (m, 1H), 2.04 (m, 2H), 1.84 (m, 1H), 1.63 (m, 2H), 1.23 (m, 2H), 1.02 (m, 1H), 0.68 (m, 1H); (ES⁺): 372.3 m/z (M+1) for [C₂₀H₂₄FN₃O₃+1]⁺.

### Example 7D

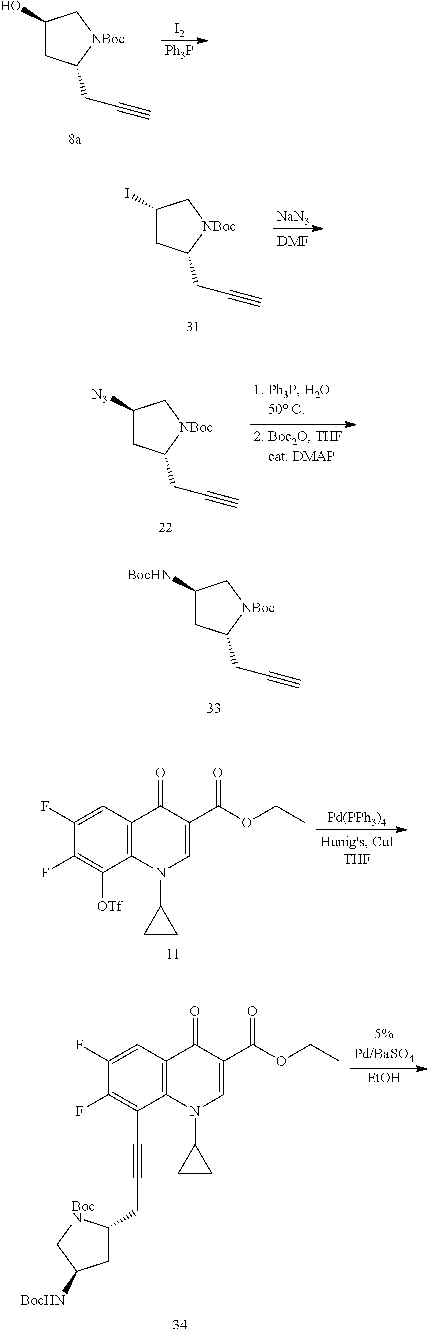

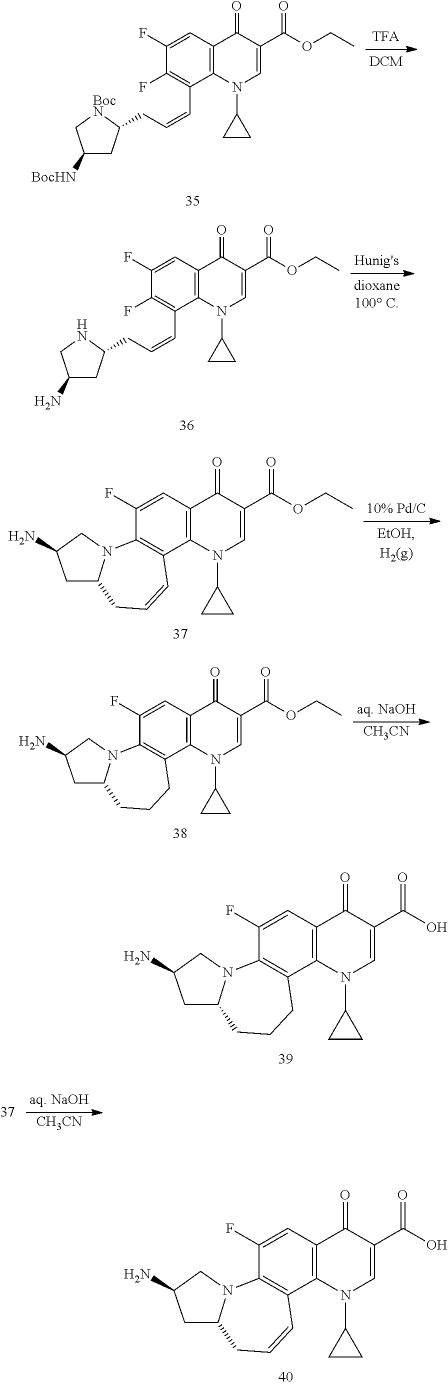

### tert-Butyl-(2R,4R)-4-[(tert-butoxycarbonyl)amino]-2-prop-2-yn-1-ylpyrrolidine-1-carboxylate (33)

A mixture of triphenylphosphine (2.6 g, 0.010 mol) and 1H-imidazole (0.69 g, 0.010 mol) in methylene chloride (100 mL) was stirred at room temperature under an atmosphere of nitrogen in a 250-mL round bottom flask. After 5 minutes iodine (2.6 g, 0.010 mol) was added and the mixture became warm and orange-brown colored. After the iodine had dissolved, a solution of tert-butyl-(2R,4R)-4-hydroxy-2-prop-2-yn-1-ylpyrrolidine-1-carboxylate (8a) (1.75 g, 7.77 mmol) in methylene chloride (30 mL) was added. The mixture was stirred overnight at ambient temperature and checked for completion after this period of time. TLC shows complete consumption of the starting material and formation of new, higher R_{f} product. The reaction was diluted with CH₂Cl₂ and washed 2×50 mL with an aqueous sodium thiosulfate solution, once with water (˜50 mL) and then with brine (50 mL). The organic layer was dried over MgSO₄, filtered and concentrated in vacuo to afford the crude product. The crude iodide (31) was immediately subjected to sodium azide conditions as follows.

The iodide (31) was placed under an atmosphere of nitrogen and then N, N-dimethylformamide (70 mL) was added followed by sodium azide (2.5 g, 0.039 mol). The reaction was heated overnight at 55° C. and then checked for completion. After this period of time, the reaction was complete based on TLC analysis. The reaction was quenched by the addition of water (˜50 mL) and then the organic product was extracted with diethyl ether (100 mL×2) and then the combined organic layers washed twice with water (25 mL), once with brine (25 mL), dried over MgSO₄, and filtered to afford the crude azide (32) (˜1.9 g, 97% yield). The azide was used directly without further characterization as follows:

tert-Butyl-(2R,4R)-4-azido-2-prop-2-yn-1-ylpyrrolidine-1-carboxylate (32) (1.90 g, 7.59 mmol), in a 40-mL scintillation vial equipped with a nitrogen balloon, was dissolved in tetrahydrofuran (25 mL) and then triphenylphosphine (10.0 g, 0.038 mol) was added and the reaction was stirred at ambient temperature for 30 minutes. After this period of time, water (4 mL, 0.2 mol) was added and the reaction was heated at 55° C. for 8 hr, at which time all of the azide (R_{f} ˜0.99 in 10% MeOH/CHCl₃ was consumed and the intermediate iminophosphorane (R_{f} ˜0.1 in 10% MeOH/CHCl₃) had been hydrolyzed to give the amine (R_{f} ˜0.35 in 10% MeOH/CHCl₃)—based on TLC analysis. The reaction was cooled to ambient temperature and then di-tert-butyldicarbonate (1.66 g, 7.59 mmol) was added as a solution in tetrahydrofuran (15 mL) and then 4-dimethylaminopyridine (0.093 g, 0.76 mmol) was added. The reaction was stirred overnight (˜12 hr) at ambient temperature. After this period of time, the reaction was complete and a single new product had formed (R_{f} ˜0.35 in 30% ethyl acetate/hexanes) based on TLC analysis. The reaction was diluted with 200 mL of ethyl acetate and then water was added (˜75 mL). The organic layer was washed 2-50 mL with water, 1-50 mL with saturated aqueous sodium bicarbonate, brine-50 mL, dried over MgSO₄, filtered and concentrated in vacuo to afford the crude product as a light colored solid. The solid (triphenylphosphine salts) was removed by trituration with diethyl ether and the resultant filtrate was concentrated in vacuo and then subjected to silica gel chromatography with 0 to 25% ethyl acetate in hexanes (40 g column) to afford 1.66 g of the alkyne (33), in 67% isolated yield over the three steps; ¹H NMR is consistent; ¹H NMR (400 MHz, CDCl₃) [symbol] ppm 4.57 (br. s., 1H), 4.30 (m, 1H), 3.96 (m, 1H), 3.62 (dd, J=11.30, 6.12 Hz, 1H), 3.29 (m, 1H), 2.59 (m, 1H), 2.52 (m, 1 H), 2.25 (m, 1H), 2.03 (m, 1H), 1.96 (br. s., 1H), 1.46 (s, 18H).

### Ethyl-8-(3-{(2R,4R)-1-(tert-butoxycarbonyl)-4-[(tert-butoxycarbonyl)amino]pyrrolidin-2-yl}prop-1-yn-1-yl)-1-cyclopropyl-6,7-difluoro-4-oxo-1,4-dihydroquinoline-3-carboxylate (34)

tert-Butyl-(2R,4R)-4-[(tert-butoxycarbonyl)amino]-2-prop-2-yn-1-ylpyrrolidine-1-carboxylate (33) (617.4 mg, 1.90 mmol) and ethyl-1-cyclopropyl-6,7-difluoro-4-oxo-8-{[(trifluoromethyl)sulfonyl]oxy}-1,4-dihydroquinoline-3-carboxylate (11) (839.9 mg, 1.90 mmol) and triphenylphosphine (120 mg, 0.48 mmol) were transferred to a 40-mL reactor vial equipped with a teflon screw cap and then the vessel was evacuated and back-filled with nitrogen ×3. Then, tetrahydrofuran (30 mL) was added and the reaction solution was sparged with nitrogen for 3 minutes. After this period of time, N,N-diisopropylethylamine (0.663 mL, 3.81 mmol) and tetrakis(triphenylphosphine)-palladium(0) (220 mg, 0.19 mmol) were added with continued sparging for 3 minutes. Finally, copper(I) iodide (91 mg, 0.48 mmol) was added and the resulting mixture was sparged for 2-3 minutes before the reaction was capped. The sealed vessel was heated at 55° C. for 8 hr in an oil bath and then checked. LCMS after this period of time shows the complete consumption of the starting alkyne and formation of a new product peak with correct mass. The reaction was cooled to room temperature and then 20 mL of ethanol was added with continued stirring for 20 minutes. The resultant precipitate was removed by vacuum filtration with a Buchner funnel and Whatman Grade 2 filter paper. The resulting filtrate was concentrated in vacuo and then subjected to silica gel chromatography with 0 to 30% ethyl acetate in DCM to afford the purified product (34) (still containing a small amount of Ph₃P, <5% by ¹H NMR), 1.07 g in 91% yield. ¹H NMR and MS are consistent with the proposed structure; ¹H NMR (400 MHz, CDCl₃) δ ppm 8.54 (s, 1H), 8.16 (t, J=9.33 Hz, 1H), 4.52 (m, 1H), 4.32 (q, J=7.05 Hz, 2H), 4.21 (m, 1H), 4.09 (m, 1H), 4.01 (m, 1H), 3.57 (dd, J=11.09, 5.91 Hz, 1H), 3.28 (m, 1H), 2.84 (m, 2H), 2.23 (m, 1H), 2.01 (m, 1H), 1.39 (br. s., 9H), 1.38 (s, 9H), 1.33 (t, J=7.05 Hz, 3H), 1.20 (m, 2H), 1.05 (m, 2H); (ES⁺): 616.4 m/z (M+1) for [C₃₂H₃₉F₂N₃O₇+1]⁺; (ES⁻) 614.5 m/z (M−1) for [C₃₂H₃₉F₂N₃O₇+1]⁺.

### Ethyl-8-[(1Z)-3-{(2R,4R)-1-(tert-butoxycarbonyl)-4-[(tert-butoxycarbonyl)amino]pyrrolidin-2-yl}prop-1-en-1-yl]-1-cyclopropyl-6,7-difluoro-4-oxo-1,4-dihydroquinoline-3-carboxylate (35)

Ethyl-8-(3-{(2R,4R)-1-(tert-butoxycarbonyl)-4-[(tert-butoxycarbonyl)amino]pyrrolidin-2-yl}prop-1-yn-1-yl)-1-cyclopropyl-6,7-difluoro-4-oxo-1,4-dihydroquinoline-3-carboxylate (34) (2.46 g, 4.00 mmol) was dissolved in ethanol (100 mL) and the reaction vessel was partially evacuated and then backfilled with nitrogen ×3. Then, palladium on barium sulfate, reduced (2.1 g) was added and the reaction vessel was partially evacuated and then back-filled with hydrogen gas (×3). Finally, the reaction was maintained under an atmosphere of hydrogen by the use of a balloon. The reaction was stirred overnight and checked after this period of time (˜12 hr) for completion. After this period of time, little progress was noted. The reaction was filtered through a short pad of Magnesol to remove the palladium salts and then the filter cake was rinsed with 10% MeOH/CHCl₃. The resultant filtrate was concentrated in vacuo and then resubjected to the same reaction conditions. HPLC after 6 hr shows that the reaction is approx 50% complete (280 nm). The reaction was charged again with hydrogen with a balloon and stirred overnight (˜10 hr). HPLC after this period of time shows that the reaction is complete. The reaction was filtered again through a short pad of Magnesol and rinsed with 5% MeOH:CHCl₃. The resultant filtrate was concentrated in vacuo and then dried overnight on the high vacuum pump. The crude product could be purified further by silica gel chromatography using a 90 g column, eluting with 0 to 35% ethyl acetate in DCM to afford the purified (35), 2.04 g, in 83% yield. ¹H NMR confirms; ¹H NMR (400 MHz, CDCl₃) δ ppm 8.56 (s, 1H), 6.74 (d, J=10.99 Hz, 1H), 5.87 (dt, J=11.25, 7.13 Hz, 1H), 4.44 (br. s., 1H), 4.32 (q, J=7.05 Hz, 2H), 4.02 (m, 1H), 3.82 (m, 1H), 3.65 (m, 1H), 3.37 (m, 1H), 3.18 (m, 1H), 2.19 (m, 1H), 1.93 (m, 1H), 1.77 (d, J=12.44 Hz, 1H), 1.69 (br. s., 2H), 1.36 (s, 18H), 1.33 (t, J=7.15 Hz, 3H), 1.09 (m, 2H), 0.90 (m, 2H); MS ES⁺618.6 m/z (M+1) for [C₃₂H₄₁F₂N₃O₇+1]⁺.

### Ethyl-(7aR,9R)-9-amino-4-cyclopropyl-12-fluoro-1-oxo-4,7,7a,8,9,10-hexahydro-1H-pyrrolo[1′,2′:1,7]-azepino-[2,3-h]quinoline-2-carboxylate (37)

Ethyl-8-[(1Z)-3-{(2R,4R)-1-(tert-butoxycarbonyl)-4-[(tert-butoxycarbonyl)amino]pyrrolidin-2-yl}prop-1-en-1-yl]-1-cyclopropyl-6,7-difluoro-4-oxo-1,4-dihydroquinoline-3-carboxylate (35) (2.04 g, 3.30 mmol) was dissolved in methylene chloride (40 mL) and trifluoroacetic acid (5 mL) was added. The reaction was stirred overnight (˜10 hr) and after this period of time, the reaction was complete based on HPLC analysis which showed a significantly more polar product. The reaction was diluted with 5% methanol/chloroform and then washed once with 10% aqueous ammonium hydroxide (˜25 mL) and brine (˜25 mL), dried over MgSO₄, filtered and concentrated in vacuo to afford the crude di-amine (36). This was treated directly with N,N-diisopropylethylamine (2.9 mL, 16 mmol) in acetonitrile (90 mL) with heating at 65° C. The reaction was stirred for ˜8 hr at elevated temperature and then checked by HPLC, which showed about 80% conversion based on HPLC analysis. The reaction was heated overnight (another 10 hr) at elevated temperature and then found to be complete after this period of time. The reaction mixture was concentrated in vacuo and then the product was partitioned between 5% methanol/chloroform and saturated sodium bicarbonate. The aqueous layer was checked to ensure pH >7 and then organic product extracted ×3 with 50 mL of 5% methanol/chloroform. The combined organic layers were washed with brine, dried over MgSO₄, and filtered to afford the crude product which was further purified by silica gel chromatography using a 90 g silica gel column, eluting with 0 to 15% methanol/chloroform to afford the desired product (37), 985 mg, 75%, over the two steps. ¹H NMR is consistent; ¹H NMR (400 MHz, CDCl₃) δ ppm 8.51 (s, 1H), 7.69 (d, J=15.13 Hz, 1H), 6.41 (d, J=12.44 Hz, 1H), 5.86 (dt, J=12.23, 3.94 Hz, 1H), 4.31 (m, 2H), 3.86 (m, 2H), 3.76 (m, 2H), 3.66 (m, 1H), 2.53 (m, 2H), 1.92 (m, 2H), 1.65 (br. s., 2H), 1.33 (t, J=7.15 Hz, 3H), 1.18 (m, 1H), 0.88 (m, 2H), 0.70 (m, 1H).

### (7aR,9R)-9-Amino-4-cyclopropyl-12-fluoro-1-oxo-4,7,7a,8,9,10-hexahydro-1H-pyrrolo[1′,2′:1,7]-azepino[2,3-h]quinoline-2-carboxylic acid trifluoroacetate (40)

Ethyl-(7aR,9R)-9-amino-4-cyclopropyl-12-fluoro-1-oxo-4,7,7a,8,9,10-hexahydro-1H-pyrrolo[1′,2′:1,7]azepino[2,3-h]quinoline-2-carboxylate (37) (109 mg, 0.274 mmol) was dissolved in acetonitrile (5 mL) and water (0.25 mL, 0.014 mol) and then 0.500 M of aqueous sodium hydroxide (1.10 mL) was added. Then, the reaction was gently heated at 55° C. for ˜12 hr. After this period of time, the ester was consumed and HPLC showed formation of a new, slightly more polar product. The reaction was quenched by the addition of acetic acid (2-3 drops) to neutral pH. The volatiles were removed in vacuo and then the resultant slurry taken up in water and lyophilized. The lyophilized solid was dissolved in water and then purified by prep HPLC using conditions described above to afford the desired product (40), 71 mg in 54% isolated yield. ¹H NMR is consistent; ¹H NMR (400 MHz, DMSO-d₆) δ ppm 8.76 (s, 1H), 8.30 (br. s., 3H), 7.65 (d, J=14.51 Hz, 1H), 6.71 (d, J=12.44 Hz, 1H), 6.00 (m, 1H), 4.26 (m, 1H), 4.12 (m, 1H), 4.05 (m, 1H), 4.00 (m, 1H), 3.93 (m, 1H), 4.09 (br. s., 1 H), 2.63 (m, 2H), 2.27 (m, 1H), 2.09 (dd, J=12.34, 6.12 Hz, 1H), 1.27 (m, 1H), 1.02 (m, 1H), 0.94 (m, 1H), 0.84 (m, 1H); MS ES⁺370.3 m/z (M+1) for [C₂₀H₂₀FNO₃+1]⁺ and ES⁻ 368.4 m/z (M−1) for [C₂₀H₂₀FN₃O₃−1]⁻.

### Ethyl-(7aR,9R)-9-amino-4-cyclopropyl-12-fluoro-1-oxo-4,5,6,7,7a,8,9,10-octahydro-1H-pyrrolo[1′,2′:1,7]-azepino[2,3-h]quinoline-2-carboxylate (38)

Ethyl-(7aR,9R)-9-amino-4-cyclopropyl-12-fluoro-1-oxo-4,7,7a,8,9,10-hexahydro-1H-pyrrolo[1′,2′:1,7]azepino[2,3-h]quinoline-2-carboxylate (37) (424 mg, 1.07 mmol) in a 50-mL round bottom flask was dissolved in ethanol (32 mL) and then the reaction vessel was partially evacuated with vacuum and back-filled with nitrogen and then the reaction solution was sparged with nitrogen for 2-3 minutes. After this, 10% palladium on carbon (110 mg) was added in one portion and the reaction vessel was partially evacuated and then back-filled with hydrogen gas (×3) and then the reaction mixture was sparged with 3-1 L hydrogen balloons and then finally maintained under an atmosphere of hydrogen with a hydrogen-filled balloon. The reaction was stirred for 10 hr before checking by HPLC/LCMS. HPLC/LCMS after this period of time shows consumption of the starting material and formation of a new product with mass corresponding to the desired reduced product. Residual hydrogen was removed from the reaction by a gentle stream of nitrogen and then the reaction mixture was filtered through a short plug of Celite 545 to remove the palladium salts and then the filtrate concentrated in vacuo. Finally, the product was purified by silica gel chromatography using a 40 g cartridge, eluting with 0 to 15% methanol/chloroform to afford the purified product (38), 205 mg in 48% yield. ¹H NMR is consistent with the proposed structure; ¹H NMR (400 MHz, CDCl₃) δ 8.50 (s, 1H), 7.60 (d, J=11.82 Hz, 1H), 4.30 (m, 2H), 3.90 (m, 1H), 3.82 (m, 2H), 3.71 (m, 1H), 3.47 (m, 1H), 3.40 (dd, J=14.93, 8.91 Hz, 1H), 2.61 (br. s., 2H), 2.37 (m, 1H), 2.17 (m, 1H), 1.99 (m, 2 H), 1.76 (m, 1H), 1.55 (m, 2H), 1.32 (t, ^{J}=7.15 Hz, 3H), 1.16 (m, 1H), 1.00 (m, 1 H), 0.86 (m, 1H), 0.63 (m, 1H); MS ES⁺400.3 m/z (M+1) for [C₂₂H₂₆FN₃O₃+1]⁺.

### (7aR,9R)-9-Amino-4-cyclopropyl-12-fluoro-1-oxo-4,5,6,7,7a,8,9,10-octahydro-1H-pyrrolo[1′,2′:1,7]-azepino[2,3-h]quinoline-2-carboxylic acid trifluoroacetate (39)

Compound (39) was prepared from compound (38) (166 mg, 0.42 mmol) according to the procedure described above for preparation of compound (17) in 37% isolated yield; ¹H NMR and MS are consistent with the proposed structure; ¹H NMR (400 MHz, D₂O) δ 8.55 (s, 1H), 7.03 (d, J=13.68 Hz, 1H), 4.11 (br. s., 1H), 4.00 (m, 2H), 3.77 (d, J=10.16 Hz, 1H), 3.63 (br. s., 1H), 3.50 (dd, J=14.31, 9.12 Hz, 1H), 2.29 (m, 2H), 2.12 (m, 1H), 1.93 (m, 1H), 1.82 (m, 1H), 1.65 (m, 1H), 1.53 (m, 1H), 1.23 (m, 1H), 1.12 (m, 1H), 0.78 (m, 1H), 0.69 (m, 1H); MS ES⁺372.36 m/z (M+1) for [C₂₀H₂₂FN₃O₃+1]⁺ and ES^{˜}370.35 m/z (M−1) for [C₂₀H₂₂FN₃O₃−1]⁻.

### Example 7E

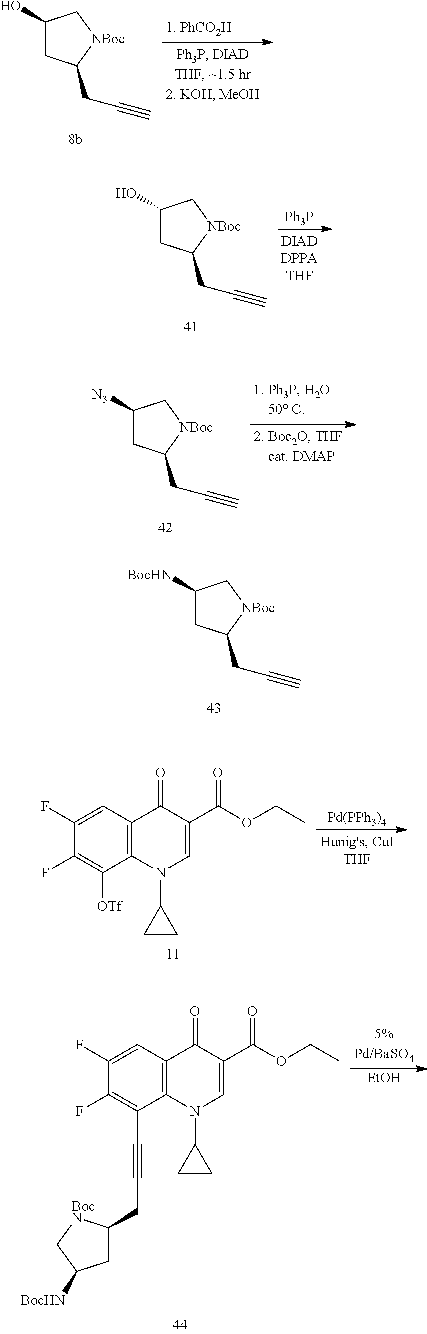

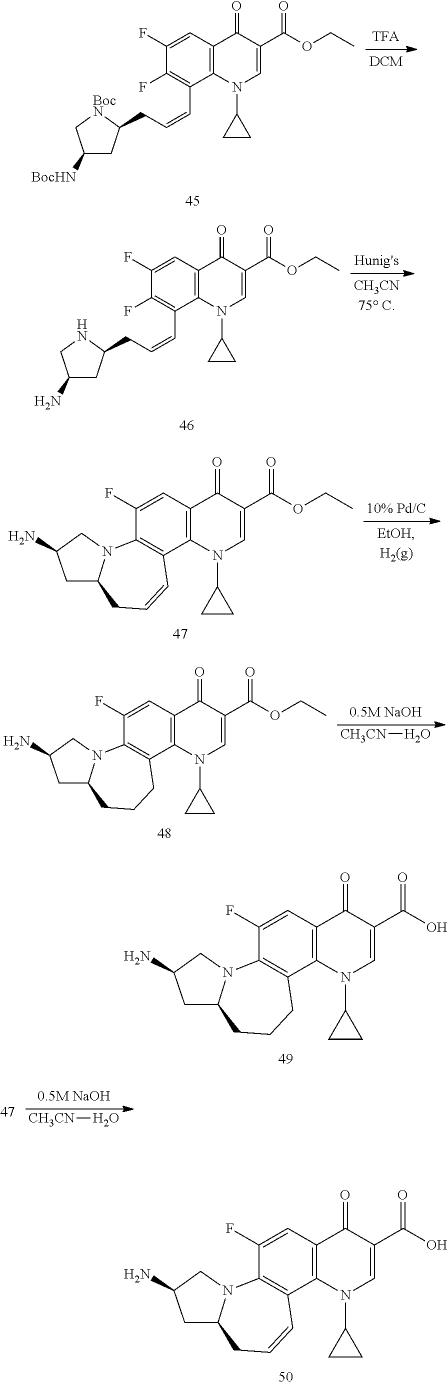

### tert-Butyl-(2S,4S)-4-hydroxy-2-prop-2-yn-1-ylpyrrolidine-1-carboxylate (41)

tert-Butyl-(2S,4R)-4-hydroxy-2-prop-2-yn-1-ylpyrrolidine-1-carboxylate (8b) (1.17 g, 5.19 mmol) was dissolved in tetrahydrofuran (50.0 mL) and triphenylphosphine (1.63 g, 6.22 mmol) and benzoic acid (0.760 g, 6.22 mmol) were added at room temperature. Then, the reaction vessel was cooled in an ice-water bath and diisopropyl azodicarboxylate (1.2 mL, 6.1 mmol) was added slowly dropwise in a solution of tetrahydrofuran (10 mL) and then the ice-water bath was removed. The resulting solution was stirred for 1.5 hr at room temperature and then checked by TLC. TLC (50% ethyl acetate in hexanes) after this period of time shows complete consumption of the starting alcohol and formation of a higher R_{f} product—the benzoate ester. The reaction was diluted with ethyl acetate (20 mL) and sodium bicarbonate (10 mL) was added. The organic layer was isolated and the resultant aqueous layer was washed once more with ˜50 mL of ethyl acetate. The combined organic layers were washed with water and brine and then dried over MgSO₄, and concentrated in vacuo to afford the crude product, which was subjected to hydrolysis conditions as follows:

The crude benzoate ester was placed under an atmosphere of nitrogen and then methanol (25 mL) was added and the reaction was cooled in an ice-water bath before a solution of potassium hydroxide (0.379 g, 6.75 mmol) in methanol (6.0 mL) was added slowly dropwise via syringe to the 0° C. cooled reaction mixture. The reaction was monitored by TLC and when complete (˜1-2 hr), the reaction was quenched (cold) with 1M HCl in ethyl acetate/dioxane added dropwise. The resultant mixture was partitioned between water and ethyl acetate (50 mL each) and the aqueous layer extracted ×2 (30 mL) with ethyl acetate. The combined organic layers were washed with water and brine, dried over MgSO₄, and filtered to afford the crude product after concentration in vacuo. The crude product was subjected to silica gel chromatography using a 40 g silica gel column, eluting with 0 to 30% ethyl acetate/hexanes to afford 1.08 g (92% isolated yield) of (41). ¹H NMR is consistent; ¹H NMR (400 MHz, CDCl₃) [symbol] ppm 4.49 (d, J=3.52 Hz, 1H), 4.10 (m, 1H), 3.59 (m, 2 H), 2.64 (m, 2H), 2.16 (br. s., 2H), 1.95 (s, 1H), 1.67 (m, 1H), 1.50 (m, 9H).

### tert-Butyl-(2S,4R)-4-[(tert-butoxycarbonyl)amino]-2-prop-2-yn-1-ylpyrrolidine-1-carboxylate (43)

tert-Butyl-(2S,4S)-4-hydroxy-2-prop-2-yn-1-ylpyrrolidine-1-carboxylate (41) (1.08 g, 4.79 mmol) was dissolved in methylene chloride (30 mL) and triphenylphosphine (1.26 g, 4.79 mmol) was added and then the reaction vessel was cooled in an ice-water bath. Then, diisopropyl azodicarboxylate (1.42 mL, 7.24 mmol) was added dropwise via syringe and then finally, diphenylphosphonic azide (1.70 mL, 7.91 mmol) was added, also dropwise via syringe. The reaction was stirred overnight and the ice-water bath was allowed to slowly expire. After this period of time, the reaction was analyzed by TLC, which indicated that the reaction was complete. The reaction was quenched by the addition of saturated aqueous sodium bicarbonate and then the organic product was extracted with CH₂Cl₂ (50-mL ×2). The combined organic layers were washed with water and brine and then dried over MgSO₄. Then, triphenylphosphine (3.77 g, 14.4 mmol) was added to the dried organic layer, at which time the light yellow solution turned clear. The solvent removed in vacuo and exchanged for tetrahydrofuran (80 mL), and the reaction vessel was equipped with a reflux condenser and stirred for 5 minutes. Then water (10.0 mL, 580 mmol) was added and the reaction was heated at 55-60° C. overnight. After this period of time, the very polar iminophosphorane was consumed and the slightly less polar amine was observed. The reaction was cooled to rt and then di-tert-butyldicarbonate (1.2 g, 5.8 mmol) was added along with a catalytic amount of 4-dimethylaminopyridine (58 mg, 0.48 mmol). The reaction was stirred at ambient temperature for 2-3 hr at which point the amine was consumed and a much higher R_{f} product was observed. The reaction was diluted with water and then concentrated in vacuo. The crude film was taken up in DCM (˜200 mL) and washed once with water and once with brine, dried over MgSO₄, filtered and then concentrated in vacuo to afford the crude product. Silica gel chromatography, eluting with 0, 5, 10, 15, and 20% ethyl acetate in hexanes afforded the desired product (43), 1.27 g in 82% yield for the 3-steps process. ¹H NMR is consistent with the proposed structure; ¹H NMR (400 MHz, CDCl₃) δ ppm 4.79 (br. s., 1H), 4.13 (m, 1H), 3.93 (m, 2H), 3.09 (m, 1H), 2.68 (m, 2H), 2.48 (m, 1H), 2.00 (br. s., 1H), 1.84 (br. s., 1H), 1.48 (s, 9H), 1.46 (s, 9H).

### Ethyl-8-(3-{(2S,4R-1-(tert-butoxycarbonyl)-4-[(tert-butoxycarbonyl)amino]pyrrolidin-2-yl}prop-1-yn-1-yl)-1-cyclopropyl-6,7-difluoro-4-oxo-1,4-dihydroquinoline-3-carboxylate (44)

tert-Butyl-(2S,4R)-4-[(tert-butoxycarbonyl)amino]-2-prop-2-yn-1-ylpyrrolidine-1-carboxylate (43) (1.27 g, 3.91 mmol), ethyl-1-cyclopropyl-6,7-difluoro-4-oxo-8-{[(trifluoromethyl)sulfonyl]oxy}-1,4-dihydroquinoline-3-carboxylate (11) (1.73 g, 3.91 mmol) and triphenylphosphine (0.26 g, 0.98 mmol) were transferred to a 40-mL scintillation vial and then the vessel was placed under an atmosphere of nitrogen by partial evacuation and back-filling with nitrogen. Then, tetrahydrofuran (25 mL) was added and the reaction mixture was sparged with nitrogen for 2-3 minutes before adding N,N-diisopropylethylamine (1.36 mL, 7.83 mmol) and tetrakis-(triphenylphosphine)palladium(0) (0.45 g, 0.39 mmol). The reaction mixture was sparged for 2-3 additional minutes and then copper(I) iodide (0.19 g, 0.98 mmol) was added and the reaction vessel was capped and allowed to stir at 60° C. overnight ˜10 hr before checking. HPLC and MS after this period of time show consumption of the triflate and alkyne and formation of a major product that is the desired Songogashira coupled product based on MS. After cooling to ambient temperature, ethanol (15 mL) was added to the reaction vessel with continued stirring for 10 minutes. The reaction was filtered to remove the precipitated salts and the filtrate concentrated in vacuo. Silica gel chromatography (90 g cartridge), eluting with 0 to 30% ethyl acetate in CH₂Cl₂ afforded the desired coupled product (44), 2.41 g in near quantitative yield. ¹H NMR is consistent; ¹H NMR (400 MHz, CDCl₃) δ ppm 8.63 (s, 1H), 8.25 (t, J=9.43 Hz, 1H), 4.75 (br. s., 1H), 4.40 (q, J=7.05 Hz, 2H), 4.19 (m, 2H), 4.02 (m, 1H), 3.96 (m, 1 H), 3.20 (m, 1H), 3.08 (dd, J=10.37, 8.50 Hz, 1H), 2.93 (m, 1H), 2.55 (m, 1H), 1.93 (m, 1H), 1.48 (s, 9H), 1.44 (s, 9H), 1.42 (m, 3H), 1.30 (m, 2H), 1.14 (m, 2H); MS ES⁺616.5 m/z (M+1) for [C₃₂H₃₉F₂N₃O₇+1]⁺.

### Ethyl-8-[(1Z)-3-{(2S,4R)-1-(tert-butoxycarbonyl)-4-[(tert-butoxycarbonyl)amino]pyrrolidin-2-yl}prop-1-en-1-yl]-1-cyclopropyl-6,7-difluoro-4-oxo-1,4-dihydroquinoline-3-carboxylate (45)

Ethyl-8-(3-{(2S,4R)-1-(tert-butoxycarbonyl)-4-[(tert-butoxycarbonyl)amino]pyrrolidin-2-yl}prop-1-yn-1-yl)-1-cyclopropyl-6,7-difluoro-4-oxo-1,4-dihydroquinoline-3-carboxylate (44) (2.56 g, 0.00416 mol) was dissolved in ethanol (50 mL, 0.8 mol) and the reaction vessel (a 250-mL round bottom) was partially evacuated and back-filled with nitrogen (×3). Then, palladium on barium sulfate, reduced (2.2 g) was added and the reaction was placed under an atmosphere of hydrogen by partial evacuation and back-filling with hydrogen gas (×3). Then, 2-L of hydrogen were bubbled through the reaction mixture and the reaction maintained under an atmosphere of hydrogen by balloon. The reaction was stirred ˜10 hr under hydrogen gas and then checked by HPLC, which showed little or no progress. The reaction was filtered through a short plug of Celite 545 and the resultant filtrate treated with 10% palladium on carbon (0.44 g) using the same process described above. The reaction was stirred for ˜10 hr under an atmosphere of hydrogen and then checked by HPLC. The reaction had progress slowly. Therefore, another lot of 10% palladium on carbon (0.25 g) was carefully added and the reaction placed under hydrogen once more. The reaction was stirred for 8-10 hr and then checked by HPLC and found to be ˜50% complete. The reaction vessel was recharged with additional hydrogen and then stirred overnight (˜10 hr); HPLC analysis after this period of time showed consumption of the starting material. The reaction was filtered through a short plug of Magnesol and the solvent removed in vacuo. The crude product (45), 2.03 g, 79% was subjected directly to treatment with TFA in CH₂Cl₂.

### Ethyl-(7aS,9R)-9-amino-4-cyclopropyl-12-fluoro-1-oxo-4,7,7a,8,9,10-hexahydro-1H-pyrrolo[1′,2′:1,7]-azepino[2,3-h]quinoline-2-carboxylate trifluoroacetate (47)

Ethyl-8-[(1Z)-3-{(2S,4R)-1-(tert-butoxycarbonyl)-4-[(tert-butoxycarbonyl)amino]pyrrolidin-2-yl}prop-1-en-1-yl]-1-cyclopropyl-6,7-difluoro-4-oxo-1,4-dihydroquinoline-3-carboxylate (45) (70.0 mg, 0.113 mmol) was dissolved in methylene chloride (10 mL) and trifluoroacetic acid (0.2 mL, 0.002 mol) was added in one portion. The reaction was allowed to stir overnight (˜10 hr) at ambient temperature. After this period of time, the reaction was complete and a single new product had formed, based on HPLC analysis, and the starting material was consumed. The reaction was quenched by diluting with 10% MeOH/CHCl₃ and then washed once with 10% aqueous ammonium hydroxide and then with brine, dried over MgSO₄, filtered and concentrated in vacuo to afford the crude amine (46). The crude amine was dissolved in acetonitrile (10 mL) and then N,N-diisopropylethylamine (0.49 mL, 2.8 mmol) was added and the reaction was heated at 60° C. for 10 hr after which time the reaction was complete based on HPLC analysis. The crude product was purified by prep HPLC as described above to afford the desired product (47), 58 mg, in near quantitative yield. ¹H NMR is consistent for the proposed structure; ¹H NMR (400 MHz, CDCl₃) δ ppm 8.59 (s, 1H), 7.78 (d, J=14.93 Hz, 1H), 6.55 (d, J=12.02 Hz, 1H), 5.99 (dt, J=12.02, 4.15 Hz, 1H), 4.39 (qd, J=7.12, 1.87 Hz, 2H), 4.32 (m, 1H), 3.86 (m, 2H), 3.79 (m, 1H), 3.44 (m, 1H), 3.06 (m, 1H), 2.63 (m, 1H), 2.52 (ddd, J=12.96, 8.60, 6.63 Hz, 1 H), 2.00 (br. s., 2H), 1.62 (m, 1H), 1.41 (t, J=7.15 Hz, 3H), 1.27 (m, 1H), 0.95 (m, 2 H), 0.77 (m, 1H).

### (7aS,9)-9-Amino-4-cyclopropyl-12-fluoro-1-oxo-4,7,7a,8,9,10-hexahydro-1H-pyrrolo[1′,2′:1,7]-azepino[2,3-h]quinoline-2-carboxylic acid trifluoroacetate (50)

Ethyl-(7aS,9R)-9-amino-4-cyclopropyl-12-fluoro-1-oxo-4,7,7a,8,9,10-hexahydro-1H-pyrrolo[1′,2′:1,7]-azepino[2,3-h]quinoline-2-carboxylate trifluoroacetate (47) (115 mg, 0.225 mmol) was dissolved in acetonitrile (10 mL) and water (0.90 mL, 0.050 mol) and then 0.500 M of an aqueous sodium hydroxide solution (0.90 mL) was added. The reaction solution was heated at 65° C. for 8 hr and then checked by HPLC. HPLC at this time shows disappearance of the starting ester and formation of a new product that is more polar. The reaction was neutralized by the addition of a few drops of acetic acid until pH 6-7 and then the reaction mixture was lyophilized. The lyophilized product was purified by prep HPLC using the method outlined above to afford the purified material (50) (6 mg), HPLC >95 area % at 254, 214, and 280 nm; ¹H NMR is consistent with the proposed structure; ¹H NMR (400 MHz, D₂O) δ ppm 8.70 (s, 1H), 7.13 (d, J=14.10 Hz, 1H), 6.44 (d, J=12.23 Hz, 1H), 6.10 (dt, J=11.82, 4.25 Hz, 1H), 4.36 (m, 1H), 4.08 (m, 1H), 3.91 (m, 2H), 3.53 (m, 1H), 2.68 (m, 2H), 2.51 (m, 1H), 1.78 (dt, J=13.73, 4.95 Hz, 1H), 1.24 (m, 1H), 0.92 (m, 1H), 0.81 (m, 1H), 0.67 (m, 1H), plus 4 exchangeable hydrogens not observed in D₂O; MS ES⁺370.3 m/z (M+1) for [C₂₀H₂₀FN₃O₃+1]⁺.

### Ethyl-(7aS,9R)-9-amino-4-cyclopropyl-12-fluoro-1-oxo-4,5,6,7,7a,8,9,10-octahydro-1H-pyrrolo[1′,2′:1,7]-azepino[2,3-h]quinoline-2-carboxylate (48)

Ethyl-(7aS,9R)-9-amino-4-cyclopropyl-12-fluoro-1-oxo-4,7,7a,8,9,10-hexahydro-1H-pyrrolo[1′,2′:1,7]azepino[2,3-h]quinoline-2-carboxylate (47) (165 mg, 0.415 mmol) in a 50-mL round bottom flask was dissolved in ethanol (12 mL) and then the reaction vessel was partially evacuated with vacuum and back-filled with nitrogen and then the reaction solution was sparged with nitrogen for 2-3 minutes. After this, 10% palladium on carbon (44 mg) was added in one portion and the reaction vessel was partially evacuated and then back-filled with hydrogen gas (×3) and then the reaction mixture was sparged with 3-1 L hydrogen balloons and then finally maintained under an atmosphere of hydrogen with a hydrogen-filled balloon. HPLC/LCMS after 10 hr shows consumption of the starting material and formation of a new product with correct mass. The residual hydrogen was removed from the reaction by a gentle stream of nitrogen and then the reaction mixture was filtered through a short plug of Celite 545 to remove the palladium salts and then the filtrate concentrated in vacuo. The product was purified by silica gel chromatography using a 40 g cartridge, eluting with 0 to 15% methanol/chloroform to afford the purified product (48), 105 mg in 63% yield; ¹H NMR is consistent with the proposed structure; ¹H NMR (400 MHz, CDCl₃) δ ppm 8.60 (s, 1 H), 7.82 (d, J=11.61 Hz, 1H), 4.40 (qd, J=7.15, 1.76 Hz, 2H), 4.24 (m, 1H), 3.96 (m, 1H), 3.75 (m, 1H), 3.54 (dd, J=14.93, 7.88 Hz, 1H), 3.28 (m, 2H), 2.60 (m, 2 H), 2.10 (m, 1H), 1.91 (m, 1H), 1.66 (m, 3H), 1.42 (t, J=7.15 Hz, 3H), 1.25 (m, 1 H), 1.11 (m, 1H), 0.94 (m, 1H), 0.73 (m, 1H).

### (7aS,9R)-9-Amino-4-cyclopropyl-12-fluoro-1-oxo-4,5,6,7,7a,8,9,10-octahydro-1H-pyrrolo[1′,2′:1,7]-azepino[2,3-h]quinoline-2-carboxylic acid trifluoroacetate (49)

Ethyl-(7aS,9R)-9-amino-4-cyclopropyl-12-fluoro-1-oxo-4,5,6,7,7a,8,9,10-octahydro-1H-pyrrolo[1′,2′:1,7]azepino[2,3-h]quinoline-2-carboxylate (48) (50 mg, 0.0001 mol) was dissolved in acetonitrile (8 mL) and water (1.25 mL) in a 40 mL vial. Then, a 1.0 M solution of aqueous sodium hydroxide (0.376 mL) was added. The vessel was capped and heated at 50° C. for 24 hr. HPLC after this period of time shows reaction to be complete. The solution was cooled to room temperature and acetic acid was added to reach pH˜5 (5-10 drops). The reaction was concentrated to remove the acetonitrile and water. The film was dissolved in a minimal amount of water and purified by prep HPLC using the conditions described above. A gummy solid was isolated after lyophilization, which was triturated from isopropanol and filtered to afford (49) as a light tan solid (15 mg) after drying under high vacuum. ¹H NMR and MS are consistent with the proposed structure; ¹H NMR (400 MHz, DMSO-d₆) δ ppm 8.78 (s, 1H), 8.17 (br. s., 2H), 7.79 (d, 1H), 4.35 (m, 1H), 4.15 (m, 1H), 3.80 (dq, J=6.74 Hz, 1H), 3.65 (m, 1H), 3.46 (m, 2H), 2.62 (m, 2H), 1.96 (m, 2H), 1.74 (m, 3H), 1.21 (m, 2H), 0.98 (m, 1H), 0.74 (m, 1H); MS ES⁺372.27 m/z (M+1) for [C₂₀H₂₂FN₃O₃+1]⁺.

### Example 8

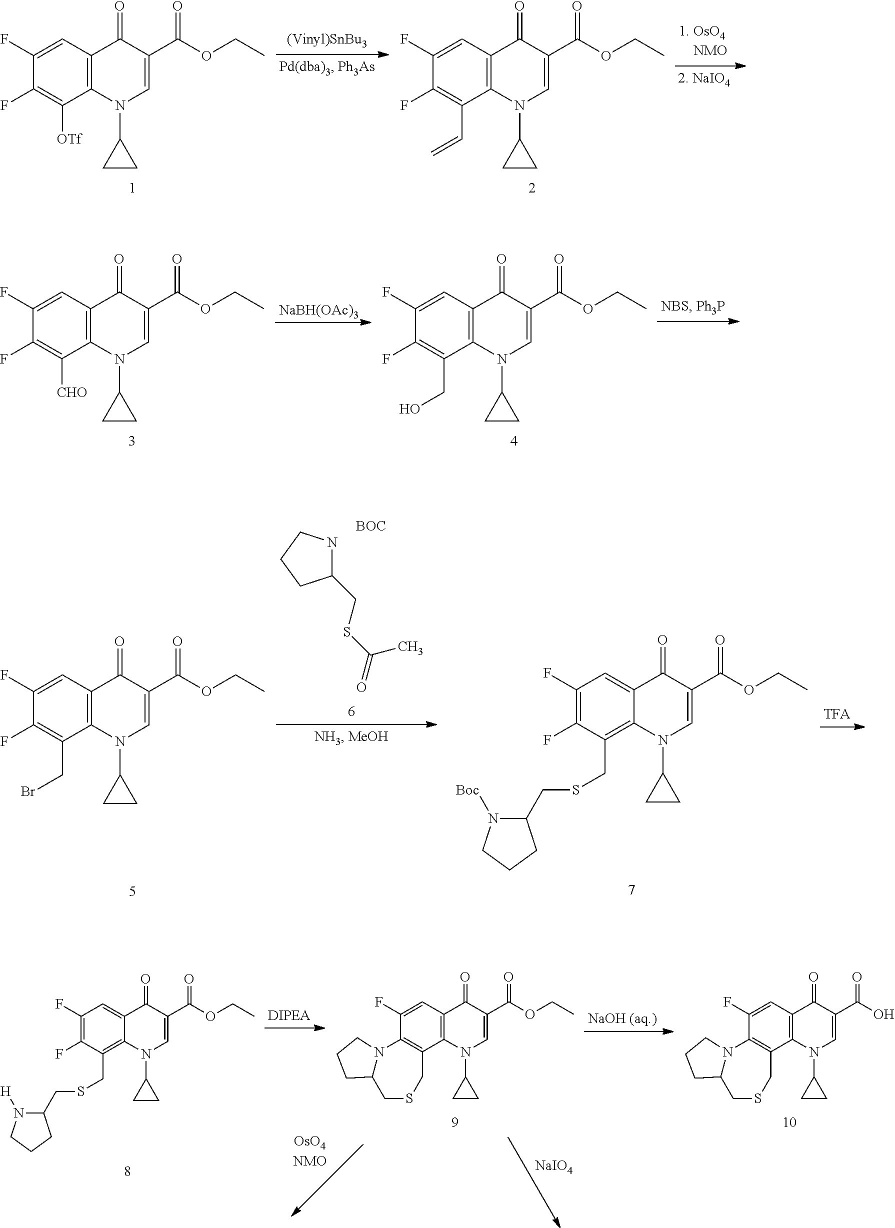

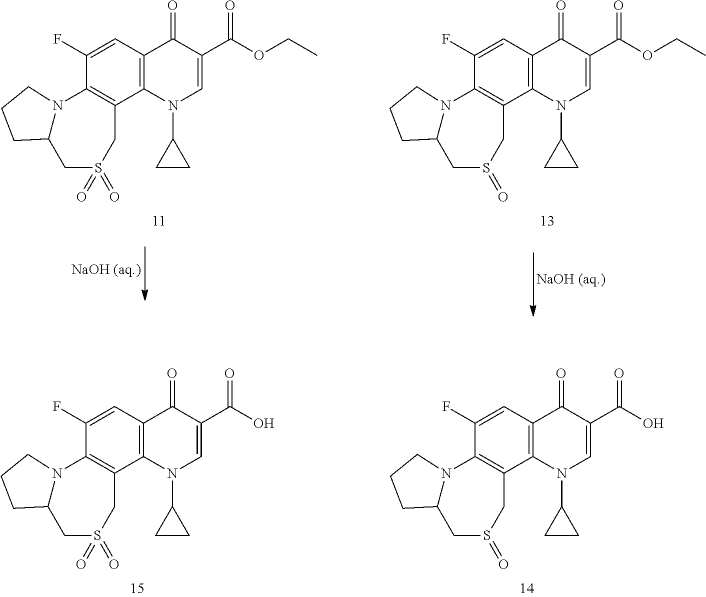

Analytical HPLC conditions for monitoring reactions and determining final product purities: Agilent 1100 HPLC. Zorbax C8 150×4.6 mm column. Solvent A—Water (0.1% TFA); Solvent B—Acetonitrile (0.07% TFA). Flow rate—1.50 mL/min. Gradient—10 min 95% A to 90% B, 2 min hold, then recycle. UV Detection @214 and 254 nm.

### Ethyl-1-cyclopropyl-6,7-difluoro-4-oxo-8-vinyl-1,4-dihydroquinoline-3-carboxylate (2)

A flame-dried flask was charged with ethyl 1-cyclopropyl-6,7-difluoro-4-oxo-8-[(trifluoromethyl)sulfonyl]oxy-1,4-dihydroquinoline-3-carboxylate (1) (7.19 g, 16.3 mmol), bis(dibenzylideneacetone)palladium(0) (190 mg, 0.32 mmol) and triphenylarsine (798 mg, 2.61 mmol). Anhydrous NMP (82 mL) was added, and the heterogeneous mixture was degassed and stirred under nitrogen for 10 mins. Vinyltributylstannane (6.66 mL, 22.8 mmol) was added, and the reaction was heated to 50° C. (internal temperature) and stirred at 50° C. for 10 h and at room temperature overnight. HPLC indicated complete consumption of starting material, so the reaction was diluted with water (80 mL) and extracted with EtOAc (2×120 mL), and the combined organic phase was washed with water (3×100 mL) and brine (50 mL), dried over MgSO₄, filtered and concentrated under reduced pressure. The oily residue was triturated with hexanes (2×200 mL) and EtOAc (30 mL)/hexanes (300 mL), pouring off and discarding the supernatant each time, and the remaining solid was purified by chromatography (120 g silica gel, 5-90% EtOAc/CH₂Cl₂) and the product retriturated with EtOAc (50 mL)/hexanes (450 mL) to give the title compound (2) (4.88 g, 94%) as a beige solid: R_{f} 0.35 (TLC, 40% EtOAc/CH₂Cl₂); MS (ESI+) for C₁₇H₁₅F₂NO₃ m/z 320 (M+H)⁺; HPLC purity, 100% (ret. time, 7.56 min).

### Ethyl-1-cyclopropyl-6,7-difluoro-8-formyl-4-oxo-1,4-dihydroquinoline-3-carboxylate (3)

A stirred homogeneous mixture of ethyl 1-cyclopropyl-6,7-difluoro-4-oxo-8-vinyl-1,4-dihydroquinoline-3-carboxylate (2) (5.93 g, 18.6 mmol) in 1,4-dioxane (140 mL) and water (55 mL) at 40° C. was treated with 2.5 wt % osmium tetroxide in 2-methyl-2-propanol (4.6 mL, 0.37 mmol), and the mixture was stirred for 20 min. NMO (2.28 g, 19.5 mmol) was added, and the mixture was stirred at 40° C. for 28 h, affording the diol intermediate (HPLC ret. time, 4.45 min). After cooling to room temperature, sodium metaperiodate (4.17 g, 19.5 mmol) was added, and the resulting cream-colored slurry was stirred at room temperature for 45 h, during which additional sodium metaperiodate (0.65 g each at 17 and 42 h, 6.08 mmol total) was added. HPLC indicated complete consumption of the diol intermediate, so the mixture was diluted with water (300 mL) and extracted with CH₂Cl₂ (2×300 mL), and the combined organic phase was washed with water (200 mL) and brine (100 mL), dried over Na₂SO₄ and concentrated under reduced pressure to give the title compound (3) (4.60 g, 72%) as a light brown solid: MS (ESI+) for C₁₆H₁₃F₂NO₄ m/z 322 (M+H)⁺; HPLC purity, 93% (ret. time, 6.51 min).

### Ethyl-1-cyclopropyl-6,7-difluoro-8-(hydroxymethyl)-4-oxo-1,4-dihydroquinoline-3-carboxylate (4)

A stirred homogeneous mixture of ethyl 1-cyclopropyl-6,7-difluoro-8-formyl-4-oxo-1,4-dihydroquinoline-3-carboxylate (3) (1.20 g, 3.74 mmol) in CH₂Cl₂ (75 mL) under nitrogen was treated with sodium triacetoxyborohydride (1.58 g, 7.47 mmol), and the resulting heterogeneous mixture was heated to 40° C. and monitored by HPLC for the disappearance of starting material. After stirring for 2 days at 40° C. and ˜3 days at room temperature, the reaction mixture was diluted with water (50 mL), the layers were separated, and the aqueous phase was extracted with CH₂Cl₂ (4×50 mL). The combined organic phase was washed with saturated aqueous NaHCO₃ (100 mL) and brine (50 mL), dried over Na₂SO₄ and concentrated under reduced pressure. The residue was triturated with MeOH (˜20 mL)/diethyl ether (˜30 mL), and the solid was isolated by filtration to give the title compound (4) (615 mg, 51%) as an off-white solid: R_{f} 0.18 (TLC, 40% EtOAc/CH₂Cl₂); MS (ESI+) for C₁₆H₁₅F₂NO₄ m/z 324 (M+H)⁺; HPLC purity, 95% (ret. time, 5.60 min).

### Ethyl-8-(bromomethyl)-1-cyclopropyl-6,7-difluoro-4-oxo-1,4-dihydroquinoline-3-carboxylate (5)

Method A: A stirred mixture of ethyl-1-cyclopropyl-6,7-difluoro-8-(hydroxymethyl)-4-oxo-1,4-dihydroquinoline-3-carboxylate (4) (200 mg, 0.557 mmol) and NBS (248 mg, 1.39 mmol) in CH₂Cl₂ (11 mL) under nitrogen in a flame-dried flask was cooled in an ice bath and treated with PPh₃ (365 mg, 1.39 mmol) portionwise over 10 mins. The color changed from yellow to orange soon after, and the mixture became homogeneous. The mixture was stirred at 0° C. and monitored by HPLC and TLC for the disappearance of starting material. At 2.5 h, the reaction was diluted with CH₂Cl₂ (40 mL), washed with water (25 mL), saturated aqueous NaHCO₃ (25 mL) and brine (20 mL), dried over Na₂SO₄ and concentrated under reduced pressure. The crude product mixture was triturated with hexanes (˜50 mL) with sonication, the supernatant was discarded, and the residue was purified by chromatography (90 g silica gel, 5-20% EtOAc/CH₂Cl₂) to give the title compound (5) (147 mg, 68%) as a pale yellow solid: R_{f} 0.43 (TLC, 40% EtOAc/CH₂Cl₂); MS (ESI+) for C₁₆H₁₄BrF₂NO₃ m/z 386 (M+H)⁺; HPLC purity, 94% (ret. time, 7.81 min).

Method B: A stirred mixture of ethyl-1-cyclopropyl-6,7-difluoro-8-methyl-4-oxo-1,4-dihydroquinoline-3-carboxylate (4) (85 mg, 75% purity, 0.21 mmol), NBS (44.3 mg, 0.249 mmol) and (E)-azobis(isobutyronitrile) (2 mg, 0.01 mmol) in benzene (2 mL) under nitrogen was heated to 75° C., and the resulting homogeneous mixture was stirred at this temperature and monitored by HPLC. At 4 h, the mixture was cooled to room temperature, diluted with EtOAc (15 mL), washed with water (15 mL), saturated aqueous NaHCO₃ (15 mL) and brine (10 mL), dried over MgSO₄ and concentrated under reduced pressure. The residue was purified by chromatography (40 g silica gel, 10-20% EtOAc/CH₂Cl₂) to give the title compound (5) (35 mg, 44%) as a white solid: HPLC purity, >98% (ret. time, 7.80 min).

### tert-Butyl-2-[(acetylthio)methyl]pyrrolidine-1-carboxylate (6)

Method A: A stirred solution of tert-butyl-2-([(4-methylphenyl)sulfonyl]oxymethyl)pyrrolidine-1-carboxylate (0.81 g, 2.3 mmol) [prepared as described in J. Org. Chem. 2002, 67(25), 9111, for the (S)-isomer] in dry DMF (23 mL) under nitrogen was treated with potassium thioacetate (0.286 g, 2.51 mmol), and the homogeneous mixture was stirred at room temperature for 4 days, during which additional potassium thioacetate (0.053 g each at 72 and 76 h, 0.92 mmol total) was added. TLC indicated complete consumption of starting material, so the reaction mixture was diluted with water (25 mL) and extracted with EtOAc (2×40 mL), and the combined organic phase was washed with water (4×40 mL) and brine (20 mL), dried over MgSO₄ and concentrated under reduced pressure. The oily residue was purified by chromatography (40 g silica gel, 10% EtOAc/hexanes) to give the title compound (6) (0.47 g, 80%) as a pale amber oil: R_{f} 0.49 (TLC, 25% EtOAc/hexanes); MS (ESI+) for C₁₂H₂₁NO₃S m/z 282 (M+Na)⁺.

Method B: A flame-dried flask was charged with tert-butyl-2-(hydroxymethyl)pyrrolidine-1-carboxylate (1.00 g, 4.97 mmol) [prepared as described in J. Org. Chem. 2002, 67(25), 9111, for the (S)-isomer], PPh₃ (1.95 g, 7.45 mmol) and dry THF (20 mL), and the stirred mixture was cooled to 0° C. Diisopropyl azodicarboxylate (1.47 mL, 7.45 mmol) was added slowly dropwise, allowing the color to dissipate between drops, and the resulting faint yellow mixture was stirred at 0° C. for 10 mins to give a cream-colored slurry. Thioacetic acid (0.533 mL, 7.45 mmol) was added slowly dropwise, affording a faint yellow near-homogeneous mixture soon after. The cooling bath was allowed to slowly expire, the reaction was stirred at room temperature over the weekend at which point TLC indicated complete consumption of starting material, and solvent was removed under reduced pressure. Purification by chromatography (120 g silica gel, 5% EtOAc/CH₂Cl₂) afforded the title compound (6) (1.39 g, 102%) as a faint amber oil: R_{f} 0.18 (TLC, 10% EtOAc/hexanes).

### Ethyl-8-[([1-(tert-butoxycarbonyl)pyrrolidin-2-yl]methylthio)methyl]-1-cyclopropyl-6,7-difluoro-4-oxo-1,4-dihydroquinoline-3-carboxylate (7)

A stirred solution of tert-butyl-2-[(acetylthio)methyl]pyrrolidine-1-carboxylate (6) (30 mg, 0.116 mmol) in 7 M ammonia in methanol (0.8 mL) was treated with ethyl-8-(bromomethyl)-1-cyclopropyl-6,7-difluoro-4-oxo-1,4-dihydroquinoline-3-carboxylate (5) (30 mg, 0.0777 mmol), and the resulting heterogeneous mixture was stirred in a sealed vial at room temperature and monitored by HPLC for the disappearance of starting material 5. At 1.5 h, the mixture was concentrated under reduced pressure and the residue purified by chromatography (40 g silica gel, 10-20% EtOAc/CH₂Cl₂) to give the title compound (7) (38 mg, 94%) as a glassy solid: R_{f} 0.44 (TLC, 40% EtOAc/CH₂Cl₂); MS (ESI+) for C₂₆H₃₂F₂N₂O₅S m/z 523 (M+H)⁺; HPLC purity, 97% (ret. time, 9.44 min).

### Ethyl-1-cyclopropyl-6,7-difluoro-4-oxo-8-[(pyrrolidin-2-ylmethyl)thio]methyl-1,4-dihydroquinoline-3-carboxylate (8)

A solution of ethyl-8-[([1-(tert-butoxycarbonyl)pyrrolidin-2-yl]methylthio)methyl]-1-cyclopropyl-6,7-difluoro-4-oxo-1,4-dihydroquinoline-3-carboxylate (7) (174 mg, 0.316 mmol) in CH₂Cl₂ (6 mL) was treated with trifluoroacetic acid (0.49 mL, 6.3 mmol), and the resulting mixture was stirred at room temperature for 6 h. HPLC and TLC indicated complete consumption of starting material, so the reaction mixture was concentrated under reduced pressure, and the oily residue was taken up in CH₂Cl₂ (˜25 mL), washed with half-saturated aqueous K₂CO₃ (15 mL) and brine (10 mL), dried over Na₂SO₄ and concentrated under reduced pressure to give the title compound (8) (123 mg, 86%) as a faint yellow crystalline film: R_{f} 0.16 (TLC, 10% MeOH/CH₂Cl₂); MS (ESI+) for C₂₁H₂₄F₂N₂O₃S m/z 423 (M+H)⁺; HPLC purity, 93% (ret. time, 5.14 min).

### Ethyl-13-cyclopropyl-8-fluoro-10-oxo-3a,4,5,6,10,13-hexahydro-1H,3H-pyrrolo[2′,1′:3,4][1,4]thiazepino[5,6-h]quinoline-11-carboxylate (9)

A stirred solution of the ethyl-1-cyclopropyl-6,7-difluoro-4-oxo-8-[(pyrrolidin-2-ylmethyl)thio]methyl-1,4-dihydroquinoline-3-carboxylate (8) (250 mg, 0.544 mmol) in dry NMP (12 mL) was evacuated and purged with nitrogen several times. N,N-Diisopropylethylamine (190 μL, 1.09 mmol) was added, and the reaction mixture was placed in an oil bath at 115° C. under nitrogen and carefully monitored by HPLC. At 3 h, ˜10% starting material remained, so the mixture was cooled to room temperature, diluted with water (20 mL) and extracted with EtOAc (2×20 mL). The combined organic phase was washed with water (3×20 mL) and brine (20 mL), dried over Na₂SO₄ and concentrated under reduced pressure. The residue was purified by chromatography (40 g silica gel, 10-60% EtOAc/CH₂Cl₂) to give the title compound (9) (137 mg, 62%) as an off-white solid: R_{f} 0.42 (TLC, 40% EtOAc/CH₂Cl₂); MS (ESI+) for C₂₁H₂₃FN₂O₃S m/z 403 (M+H)⁺; HPLC purity, >99% (ret. time, 8.47 min).

### 13-Cyclopropyl-8-fluoro-10-oxo-3a,4,5,6,10,13-hexahydro-1H,3H-pyrrolo[2′,1′:3,4][1,4]thiazepino[5,6-h]quinoline-11-carboxylic acid (10)

A stirred solution of ethyl-13-cyclopropyl-8-fluoro-10-oxo-3a,4,5,6,10,13-hexahydro-1H,3H-pyrrolo[2′,1′:3,4][1,4]thiazepino[5,6-h]quinoline-11-carboxylate (9) (40.0 mg, 0.0994 mmol) in THF (10 mL) was treated with 1.0 M aqueous NaOH (119 μL), and the mixture was sparged with nitrogen for 5 mins, placed in an oil bath at 35° C. and stirred at this temperature under nitrogen for 2 days, during which additional 1.0 M aqueous NaOH (20 μL) and water (180 μL) were added at 21 h. At this point, HPLC indicated <6% starting material remaining, so the mixture was cooled to room temperature, diluted with CH₂Cl₂ (40 mL), washed with 0.1 M aqueous HCl (20 mL), dried over Na₂SO₄ and concentrated under reduced pressure. The crude product was purified by preparative reverse phase HPLC [Phenomenex Luna 250×21.20 mm, 10 micron column. Solvent A—Acetonitrile (0.07% TFA); Solvent B—Water (0.10% TFA). Flow rate—20 mL/min. Gradient—5% to 80% A over 10 min; 80% to 100% A over 5 min; hold 100% A 5 min; 100% to 5% A over 5 min; 1 min hold; then recycle. Product retention time=13.2 min; starting material retention time=12.7 min.], and the product fractions were pooled and concentrated. The solid was taken up in CH₂Cl₂ (25 mL), washed with water (2×10 mL), dried over Na₂SO₄, and concentrated under reduced pressure to give the title compound (10) (27 mg, 72%) as a beige solid: R_{f} 0.09 (TLC, 40% EtOAc/CH₂Cl₂); ¹H NMR (400 MHz, DMSO-d₆) δ ppm 14.97 (s, 1H), 8.75 (s, 1H), 7.81 (d, J=13.7 Hz, 1H), 4.67 (d, J=15.2 Hz, 1H), 4.33 (m, 1H), 3.95 (m, 1H), 3.68 (d, J=15.2 Hz, 1H), 3.45 (m, 1H), 3.39 (m, 1H), 2.74 (m, 2H), 2.28 (m, 1H), 1.98 (m, 2H), 1.84 (m, 1H), 1.27 (m, 2H), 1.04 (m, 1H), 0.84 (m, 1H); MS (ESI+) for C₁₉H₁₉FN₂O₃S m/z 375 (M+H)⁺; MS (ESI−) for C₁₉H₁₉FN₂O₃S m/z 373 (M−H)⁻; HPLC purity, >99% (ret. time, 8.76 min).

### Ethyl-13-cyclopropyl-8-fluoro-10-oxo-3a,4,5,6,10,13-hexahydro-1H,3H-pyrrolo[2′,1′:3,4][1,4]thiazepino[5,6-h]quinoline-11-carboxylate 2,2-dioxide (11)

A slurry of ethyl-13-cyclopropyl-8-fluoro-10-oxo-3a,4,5,6,10,13-hexahydro-1H,3H-pyrrolo[2′,1′:3,4][1,4]thiazepino[5,6-h]quinoline-11-carboxylate (9) (75 mg, 0.19 mmol) and NMO (55 mg, 0.466 mmol) in acetone (3.0 mL) and water (1.0 mL) was treated with the 2.5 wt % osmium tetroxide in 2-methyl-2-propanol (120 μL, 0.0093 mmol), and the mixture was stirred at room temperature under nitrogen and monitored by HPLC and TLC. At 45 h, the mixture was treated with half-saturated aqueous NaHSO₃ (˜3 mL dropwise), diluted with water (5 mL) and extracted with CH₂Cl₂ (2×15 mL). The combined organic phase was dried over Na₂SO₄ and concentrated under reduced pressure, and the residue was purified by chromatography (40 g silica gel, 20-60% EtOAc/CH₂Cl₂) to give the title compound (11) (54 mg, 67%) as a white solid: R_{f} 0.15 (TLC, 40% EtOAc/CH₂Cl₂); MS (ESI+) for C₂₁H₂₃FN₂O₅S m/z 435 (M+H)⁺; HPLC purity, 100% (ret. time, 6.53 min).

### 13-Cyclopropyl-8-fluoro-10-oxo-3a,4,5,6,10,13-hexahydro-1H,3H-pyrrolo[2′,1′:3,4][1,4]thiazepino[5,6-h]quinoline-11-carboxylic acid 2,2-dioxide (12)

A stirred mixture of ethyl-13-cyclopropyl-8-fluoro-10-oxo-3a,4,5,6,10,13-hexahydro-1H,3H-pyrrolo[2′,1′:3,4][1,4]thiazepino[5,6-h]quinoline-11-carboxylate 2,2-dioxide (11) (47 mg, 0.11 mmol) in THF (10 mL) and water (1.1 mL) under nitrogen was treated with 1.0 M aqueous NaOH (0.119 mL), warmed to 35° C. (oil bath) and stirred for 24 h, at which point HPLC indicated complete consumption of starting material. The mixture was cooled to room temperature, diluted with CH₂Cl₂ (50 mL), washed with 0.1 M aqueous HCl (25 mL) and brine (10 mL), dried over Na₂SO₄ and concentrated under reduced pressure. The residue was triturated with diethyl ether (25 mL) with stirring for 1 h, and the solid was isolated by filtration to give the title compound (12) (38 mg, 86%) as a beige solid: R_{f} 0.23 (TLC, 10% MeOH/CH₂Cl₂); ¹H NMR (400 MHz, DMSO-d₆) δ ppm 14.82 (s, 1H), 8.72 (s, 1H), 7.95 (d, J=13.4 Hz, 1H), 5.58 (d, J=16.1 Hz, 1H), 5.05 (d, J=16.1 Hz, 1H), 4.30 (m, 1 H), 4.03 (m, 1H), 3.88 (m, 1H), 3.62 (m, 1H), 3.48 (m, 2H), 2.22 (m, 1H), 2.09 (m, 1 H), 2.02 (m, 1H), 1.78 (m, 1H), 1.33 (m, 2H), 1.17 (m, 2H); MS (ESI+) for C₁₉H₁₉FN₂O₅S m/z 407 (M+H)⁺; MS (ESI−) for C₁₉H₁₉FN₂O₅S m/z 405 (M−H)⁻; HPLC purity, >98% (ret. time, 6.84 min).

### Ethyl-13-cyclopropyl-8-fluoro-10-oxo-3a,4,5,6,10,13-hexahydro-1H,3H-pyrrolo[2′,1′:3,4][1,4]thiazepino[5,6-h]quinoline-1′-carboxylate 2-oxide (13)

A slurry of ethyl-13-cyclopropyl-8-fluoro-10-oxo-3a,4,5,6,10,13-hexahydro-1H,3H-pyrrolo[2′,1′:3,4][1,4]thiazepino[5,6-h]quinoline-11-carboxylate (9) (105 mg, 0.261 mmol) in MeOH (10 mL) was treated with a solution of NaIO₄ (58.6 mg, 0.274 mmol) in water (2 mL) dropwise over ˜30 sec (slight exotherm). The resulting white heterogeneous mixture was stirred at room temperature under nitrogen and monitored by TLC and HPLC for the disappearance of starting material. At 24 h, the reaction was concentrated down to a small volume, diluted with brine (15 mL) and extracted with CH₂Cl₂ (2×25 mL). The combined organic phase was dried over Na₂SO₄, concentrated down to a small volume and diluted with diethyl ether (˜20 mL) with stirring to give a slurry. The solid was isolated by filtration to give the title compound (13) (92 mg, 84%) as a white solid: R_{f} 0.51 (TLC, 10% MeOH/CH₂Cl₂); MS (ESI+) for C₂₁H₂₃FN₂O₄S m/z 419 (M+H)⁺; HPLC purity, 96% (ret. time, 5.51 min; no separation of stereoisomers). NMR indicates a 85:15 to 90:10 mixture of sulfoxide diastereomers (each as the racemate).

### 13-Cyclopropyl-8-fluoro-10-oxo-3a,4,5,6,10,13-hexahydro-1H,3H-pyrrolo[2′,1′:3,4][1,4]thiazepino[5,6-h]quinoline-1′-carboxylic acid 2-oxide (14)

A stirred solution of ethyl-13-cyclopropyl-8-fluoro-10-oxo-3a,4,5,6,10,13-hexahydro-1H,3H-pyrrolo[2′,1′:3,4][1,4]thiazepino[5,6-h]quinoline-1′-carboxylate 2-oxide (13) (50.0 mg, 0.119 mmol) in THF (10 mL) was treated with 1.0 M aqueous NaOH (0.130 mL) and water (1.2 mL), sparged with nitrogen for 5 mins, placed in an oil bath at 35° C. and stirred under nitrogen for 24 h, at which point HPLC indicated complete consumption of starting material. The mixture was cooled to room temperature, diluted with 0.1 M aqueous HCl (10 mL) and extracted with EtOAc (3×20 mL). The combined organic phase was washed with brine (15 mL), dried over Na₂SO₄ and concentrated under reduced pressure. The residue was taken up in a minimum of CH₂Cl₂ (˜5 mL) and diluted with diethyl ether (˜15 mL) with stirring to give a slurry, and the solid was isolated by filtration to give the title compound (14) (39 mg, 84%) as a faint yellow solid: R_{f} 0.21 (TLC, 10% MeOH/CH₂Cl₂); ¹H NMR (400 MHz, DMSO-d₆) δ ppm 14.85 (s, 1H), 8.77 (s, 1H), 7.87 (d, J=13.6 Hz, 1H), 4.82 (d, J=13.4 Hz, 1H), 4.45 (m, 1H), 4.39 (d, J=13.7 Hz, 1H), 3.94 (m, 1H), 3.66 (m, 1H), 3.45 (m, 2H), 2.93 (t, J=11.8 Hz, 1H), 2.26 (m, 1H), 2.11 (m, 1H), 1.96 (m, 1H), 1.83 (m, 1H), 1.33 (m, 2H), 1.12 (m, 1H), 0.92 (m, 1H) for the major sulfoxide diastereomer [NMR indicates a 93:7 to 95:5 mixture of sulfoxide diastereomers (each as the racemate)]; MS (ESI+) for C₁₉H₁₉FN₂O₄S m/z 391 (M+H)⁺; MS (ESI−) for C₁₉H₁₉FN₂O₄S m/z 389 (M−H)⁻; HPLC purity, >97% (ret. time, 6.00 min; no separation of stereoisomers).

### Example 9

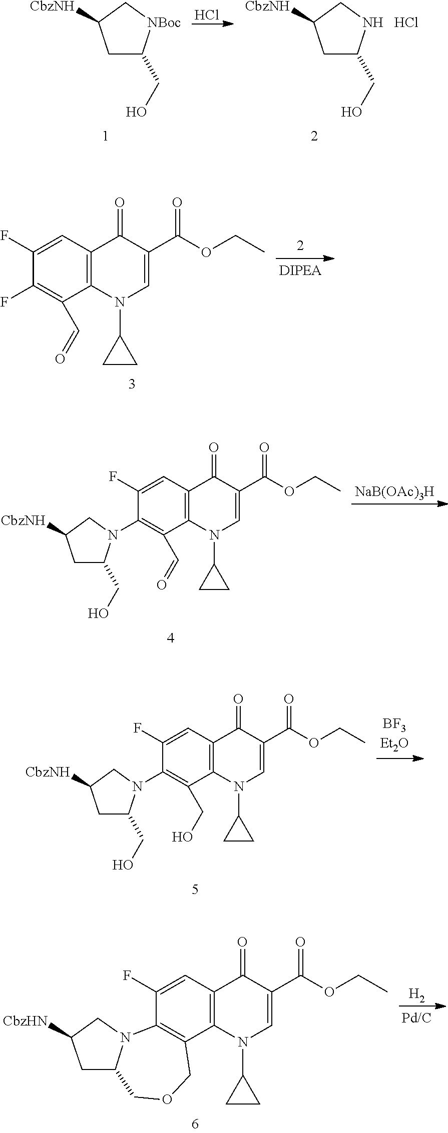

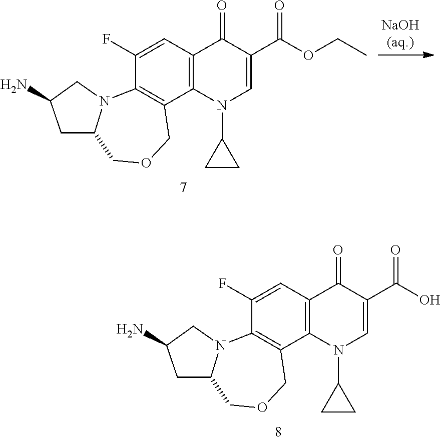

Analytical HPLC conditions for monitoring reactions and determining final product purities: Agilent 1100 HPLC. Zorbax C8 150×4.6 mm column. Solvent A—Water (0.1% TFA); Solvent B—Acetonitrile (0.07% TFA). Flow rate—1.50 mL/min. Gradient—10 min 95% A to 90% B, 2 min hold, then recycle. UV Detection @ 214 and 254 nm (290 nm for final product).

### Benzyl-[(3R,5S)-5-(hydroxymethyl)pyrrolidin-3-yl]carbamate hydrochloride (2)

A stirred solution of tert-butyl-(2S,4R)-4-[(benzyloxy)carbonyl]amino-2-(hydroxymethyl)pyrrolidine-1-carboxylate (1) (0.270 g, 0.770 mmol) in diethyl ether (6 mL) was treated dropwise with 4M HCl in 1,4-dioxane (0.96 mL) and stirred at room temperature for 1.8 d, during which additional 4M HCl in 1,4-dioxane (1.9 mL each) was added at 22 h and 27 h. HPLC and LC-MS indicated complete consumption of starting material, so the resulting white precipitate was isolated by filtration, washed with diethyl ether, and dried in the vacuum oven at 40° C. to give the title compound (2) (165 mg, 75%) as a white solid: MS (ESI+) for C₁₃H₁₈N₂O₃ m/z 251 (M+H)⁺ for free base; HPLC purity, 100% (ret. time, 4.52 min).

### Ethyl-7-[(2S,4R)-4-[(benzyloxy)carbonyl]amino-2-(hydroxymethyl)pyrrolidin-1-yl]-1-cyclopropyl-6-fluoro-8-formyl-4-oxo-1,4-dihydroquinoline-3-carboxylate (4)

A stirred mixture of benzyl-[(3R,5S)-5-(hydroxymethyl)pyrrolidin-3-yl]carbamate hydrochloride (2) (100 mg, 0.349 mmol) and ethyl 1-cyclopropyl-6,7-difluoro-8-formyl-4-oxo-1,4-dihydroquinoline-3-carboxylate (3) (56 mg, 0.174 mmol) in CH₃CN (2.0 mL) was treated with N,N-diisopropylethylamine (121 uL, 0.697 mmol), and the heterogeneous mixture was heated to 50° C. under nitrogen, affording a homogeneous mixture, and monitored by HPLC. At 2 h, the mixture was cooled to room temperature, diluted with water (10 mL) and extracted with EtOAc (2×15 mL), and the combined organic phase was washed with brine (10 mL), dried over Na₂SO₄ and concentrated under reduced pressure. The residue was triturated with EtOAc (˜1-2 mL) with sonication and diluted with diethyl ether (˜2-3 mL), and the solid was isolated by filtration, washed with diethyl ether and dried in the vacuum oven at 40° C. to give the title compound (4) (80 mg, 83%) as a yellow solid: R_{f} 0.10 (TLC, 40% EtOAc/CH₂Cl₂); MS (ESI+) for C₂₉H₃₀FN₃O₇ m/z 552 (M+H)⁺; HPLC purity, 95% (ret. time, 6.75 min).

### Ethyl-7-[(2S,4R)-4-[(benzyloxy)carbonyl]amino-2-(hydroxymethyl)pyrrolidin-1-yl]-1-cyclopropyl-6-fluoro-8-(hydroxymethyl)-4-oxo-1,4-dihydroquinoline-3-carboxylate (5)

A stirred solution of ethyl-7-[(2S,4R)-4-[(benzyloxy)carbonyl]amino-2-(hydroxymethyl)pyrrolidin-1-yl]-1-cyclopropyl-6-fluoro-8-formyl-4-oxo-1,4-dihydroquinoline-3-carboxylate (4) (350 mg, 0.634 mmol) in CH₂Cl₂ (6.5 mL) under nitrogen was treated with sodium triacetoxyborohydride (269 mg, 1.27 mmol) in one portion, and the resulting heterogeneous mixture was stirred at room temperature and monitored by HPLC for the disappearance of starting material. After 5 d, the mixture was diluted with CH₂Cl₂ (35 mL), washed with saturated aqueous NaHCO₃ (20 mL) and brine (10 mL), dried over Na₂SO₄, and concentrated under reduced pressure. The residue (357 mg) was combined with that from a previous reaction (165 mg), triturated with EtOAc (5-6 mL) with sonication, diluted with diethyl ether (5-6 mL), and the product filtered, washed with diethyl ether and dried in the vacuum oven at 40° C. to give the title compound (5) (400 mg, 78% avg. yield for two reactions) as a light yellow solid: R_{f} 0.50 (TLC, 10% MeOH/CH₂Cl₂); MS (ESI+) for C₂₉H₃₂FN₃O₇ m/z 554 (M+H)⁺; HPLC purity (214 nm), 94% (ret. time, 6.63 min).

### Ethyl-(3aS,5R)-5-[(benzyloxy)carbonyl]amino-13-cyclopropyl-8-fluoro-10-oxo-3a,4,5,6,10,13-hexahydro-1H,3H-pyrrolo[2′,1′:3,4][1,4]oxazepino[5,6-h]quinoline-11-carboxylate (6)

A stirred solution of ethyl-7-[(2S,4R)-4-[(benzyloxy)carbonyl]amino-2-(hydroxymethyl)pyrrolidin-1-yl]-1-cyclopropyl-6-fluoro-8-(hydroxymethyl)-4-oxo-1,4-dihydroquinoline-3-carboxylate (5) (390 mg, 0.704 mmol) in CH₂Cl₂ (14 mL) at −40° C. under nitrogen was treated with boron trifluoride etherate (178 uL, 1.41 mmol) dropwise, and the resulting mixture was allowed to warm to room temperature over 2.5 h and stir overnight. At 18 h, HPLC indicated consumption of starting material, so the mixture was quenched slowly with 1M aqueous HCl (15 mL) and stirred for 30 mins. The layers were separated, the aqueous phase was extracted with CH₂Cl₂ (25 mL), and the combined organic phase was washed with saturated aqueous NaHCO₃ (20 mL) and brine (10 mL), dried over Na₂SO₄ and concentrated under reduced pressure. The residue was purified by chromatography (40 g silica gel, 30-70% EtOAc/CH₂Cl₂ followed by 5-10% MeOH/DCM) to give the title compound (6) (118 mg, 31%) as an off-white solid: R_{f} 0.22 (TLC, 40% EtOAc/CH₂Cl₂); MS (ESI+) for C₂₉H₃₀FN₃O₆ m/z 536 (M+H)⁺; HPLC purity, >99% (ret. time, 7.85 min).

### Ethyl-(3aS,5R)-5-amino-13-cyclopropyl-8-fluoro-10-oxo-3a,4,5,6,10,13-hexahydro-1H,3H-pyrrolo[2′,1′:3,4][1,4]oxazepino[5,6-h]quinoline-11-carboxylate (7)

A solution of ethyl-(3aS,5R)-5-[(benzyloxy)carbonyl]amino-13-cyclopropyl-8-fluoro-10-oxo-3a,4,5,6,10,13-hexahydro-1H,3H-pyrrolo[2′,1′:3,4][1,4]oxazepino[5,6-h]quinoline-11-carboxylate (6) (113 mg, 0.211 mmol) in EtOH (10 mL) under nitrogen in a Parr bottle was treated with Et₃N (5.88 uL, 0.0422 mmol) followed by 10% Pd-on-carbon (45 mg, 0.042 mmol). The mixture was evacuated and filled with nitrogen several times and then with hydrogen several times and then shaken on the Parr apparatus under 20 psi hydrogen. At 6 hrs, HPLC and TLC showed complete consumption of starting material. The catalyst was removed by filtration through Celite 545, and the filtrate was concentrated under reduced pressure to give the title compound (7) (68 mg, 80%) as a faint yellow glassy solid: R_{f} 0.10 (TLC, 10% MeOH/CH₂Cl₂); MS (ESI+) for C₂₁H₂₄FN₃O₄ m/z 402 (M+H)⁺; HPLC purity, >95% (ret. time, 4.45 min).

### (3aS,5R)-5-Amino-13-cyclopropyl-8-fluoro-10-oxo-3a,4,5,6,10,13-hexahydro-1H,3H-pyrrolo[2′,1′:3,4][1,4]oxazepino[5,6-h]quinoline-11-carboxylic acid trifluoroacetate (8)

A solution of ethyl-(3aS,5R)-5-amino-13-cyclopropyl-8-fluoro-10-oxo-3a,4,5,6,10,13-hexahydro-1H,3H-pyrrolo[2′,1′:3,4][1,4]oxazepino[5,6-h]quinoline-11-carboxylate (7) (64.0 mg, 0.159 mmol) in THF (9.5 mL) and water (1.7 mL) under nitrogen was treated with 1.0 M aqueous NaOH (190 uL), and the light yellow mixture was stirred at 35° C. for 5 h and at room temperature overnight. At this point, HPLC indicated <10% starting material remaining, so the mixture was adjusted to pH˜5.5 (wet pH paper) with glacial AcOH and concentrated under reduced pressure. The crude product was purified by preparative reverse phase HPLC [Phenomenex Luna 250×21.20 mm, 10 micron column. Solvent A—Acetonitrile (0.07% TFA); Solvent B—Water (0.10% TFA). Flow rate—20 mL/min. Gradient—5% to 80% A over 10 min; 80% to 100% A over 5 min; hold 100% A 5 min; 100% to 5% A over 5 min; 1 min hold; then recycle. Product retention time=8.4 min; starting material retention time=9.2 min.], and the product fractions were pooled, concentrated and lyophilized to give the title compound (8) (54 mg, 69%) as a faint yellow solid: ¹H NMR (400 MHz, DMSO-d₆) δ ppm 8.78 (s, 1H), 8.20 (br s, 3H), 7.88 (d, J=13.7 Hz, 1H), 5.59 (d, J=14.1 Hz, 1H), 4.32 (m, 1H), 4.14 (m, 1H), 3.86 (m, 5H), 3.29 (m, 1H), 2.26 (m, 1 H), 1.98 (m, 1H), 1.26 (m, 2H), 0.99 (m, 1H), 0.71 (m, 1H); MS (ESI+) for C₁₉H₂₀FN₃O₄ m/z 374 (M+H)⁺ for amino acid; HPLC purity, 96.6% (290 nm)-97.5% (214 nm) (ret. time, 4.07 min).

### Example 10

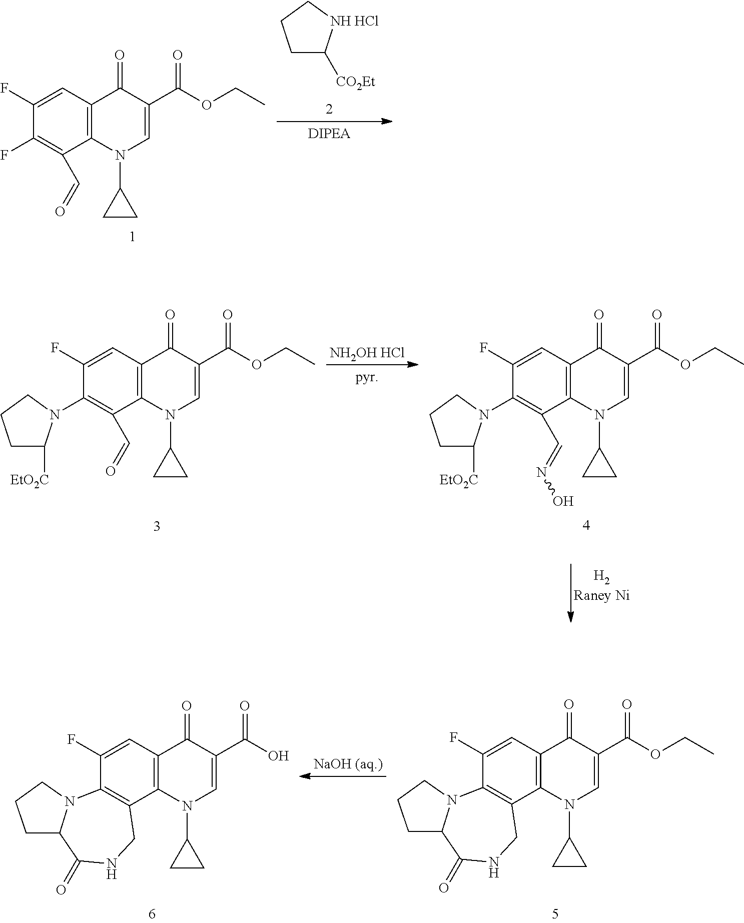

Analytical HPLC conditions for monitoring reactions and determining product purities: Agilent 1100 HPLC. Zorbax C8 150×4.6 mm column. Solvent A—Water (0.1% TFA); Solvent B—Acetonitrile (0.07% TFA). Flow rate—1.50 mL/min. Gradient—10 min 95% A to 90% B, 2 min hold, then recycle. UV Detection @ 214 and 254 nm (290 nm for final product).

### Ethyl-1-cyclopropyl-7-[2-(ethoxycarbonyl)pyrrolidin-1-yl]-6-fluoro-8-formyl-4-oxo-1,4-dihydroquinoline-3-carboxylate (3)

A stirred mixture of ethyl prolinate hydrochloride (2) (391 mg, 2.18 mmol) and ethyl-1-cyclopropyl-6,7-difluoro-8-formyl-4-oxo-1,4-dihydroquinoline-3-carboxylate (1) (350 mg, 1.09 mmol) in CH₃CN (10 mL) was treated with N,N-diisopropylethylamine (0.759 mL, 4.36 mmol), and the heterogeneous mixture was placed in a 50° C. oil bath and stirred under nitrogen at this temperature for 2 h, at which point HPLC indicated reaction near completion, and at room temperature for 18 h. The mixture was diluted with water (20 mL) and extracted with EtOAc (2×30 mL), and the combined organic phase was washed with water (20 mL) and brine (10 mL), dried over Na₂SO₄ and concentrated under reduced pressure. The residue was triturated with EtOAc (˜5 mL) to give a tan slurry and diluted with diethyl ether (˜10 mL), and the solid was isolated by filtration, washed with diethyl ether and dried in the vacuum oven at room temperature to give the title compound (3) (345 mg, 71%) as a faint yellow solid: MS (ESI+) for C₂₃H₂₅FN₂O₆ m/z 445 (M+H)⁺; HPLC purity, >99% (ret. time, 7.46 min).

### Ethyl-1-cyclopropyl-7-[2-(ethoxycarbonyl)pyrrolidin-1-yl]-6-fluoro-8-[(hydroxyimino)methyl]-4-oxo-1,4-dihydroquinoline-3-carboxylate (4)

A stirred mixture of ethyl-1-cyclopropyl-7-[2-(ethoxycarbonyl)pyrrolidin-1-yl]-6-fluoro-8-formyl-4-oxo-1,4-dihydroquinoline-3-carboxylate (3) (295 mg, 0.664 mmol) in abs. EtOH (4.0 mL) was treated with pyridine (4.0 mL) to give a yellow homogeneous mixture. Hydroxylamine hydrochloride (231 mg, 3.32 mmol) was added, and the mixture was stirred at room temperature and monitored by HPLC for the disappearance of starting material. At 1.75 h, the mixture was concentrated under reduced pressure, and the residue was diluted with water (˜25 mL) with sonication and stirring to give a slurry. The solid product was isolated by filtration, rinsing with several portions of water, and dried in the vacuum oven at 40° C. to give the title compound (4) (295 mg, 97%) as a white solid: R_{f} 0.22 (TLC, 40% EtOAc/CH₂Cl₂); MS (ESI+) for C₂₃H₂₆FN₃O₆ m/z 460 (M+H)⁺; HPLC purity, 100% (ret. time, 6.92 min).

### Ethyl-13-cyclopropyl-8-fluoro-3,10-dioxo-2,3,3a,4,5,6,10,13-octahydro-1H-pyrrolo[2′,1′:3,4][1,4]diazepino[5,6-h]quinoline-11-carboxylate (5)

A solution of ethyl-1-cyclopropyl-7-[2-(ethoxycarbonyl)pyrrolidin-1-yl]-6-fluoro-8-[(hydroxyimino)methyl]-4-oxo-1,4-dihydroquinoline-3-carboxylate (4) (97.0 mg, 0.211 mmol) in abs. EtOH (20 mL) was transferred to a Parr bottle containing wet Raney Nickel (62.0 mg) under nitrogen. The mixture was evacuated and filled with nitrogen several times and then with hydrogen several times and shaken on the Parr apparatus under 45 psi hydrogen for 24 h. HPLC showed >95% starting material, so the catalyst was removed by filtration through Celite, and the filtrate was concentrated under reduced pressure. The residue was resubmitted to the reaction conditions [wet Raney Nickel (300 mg), EtOH (20 mL), 45 psi hydrogen] and shaken on the Parr apparatus for 2 days, additional wet Raney Nickel (300 mg) being added at 28 h. HPLC showed <5% starting material remaining at this point and the product was precipitating out of solution, so the mixture was diluted with EtOH (˜100 mL) and filtered through Celite, rinsing the filter pad with EtOAc (˜150 mL). The filtrate was concentrated under reduced pressure, and the residue was purified by radial chromatography [2000 micron silica gel rotor; 5-10% MeOH/CH₂Cl₂ eluent] to give the title compound (5) (47 mg, 56%) as a faint yellow solid: R_{f} 0.49 (TLC, 10% MeOH/CH₂Cl₂); MS (ESI+) for C₂₁H₂₂FN₃O₄ m/z 400 (M+H)⁺; HPLC purity, 89% (ret. time, 5.59 min), with the methyl ester as the single impurity (formed during the purification).

### 13-Cyclopropyl-8-fluoro-3,10-dioxo-2,3,3a,4,5,6,10,13-octahydro-1H-pyrrolo[2′,1′:3,4][1,4]diazepino[5,6-h]quinoline-1′-carboxylic acid (6)

A stirred mixture of ethyl-13-cyclopropyl-8-fluoro-3,10-dioxo-2,3,3a,4,5,6,10,13-octahydro-1H-pyrrolo[2′,1′:3,4][1,4]diazepino[5,6-h]quinoline-1′-carboxylate (5) (60.0 mg, 0.150 mmol) in THF (10 mL) and water (1.5 mL) was treated with 1.0 M aqueous NaOH (195 uL), and the yellow mixture was stirred at room temperature for 18 h. HPLC showed a 2:1 ratio of product/starting material, but by-products were also forming. The mixture was diluted with EtOAc (˜25 mL), washed with 0.1 M aqueous HCl (20 mL) and brine (10 mL), dried over Na₂SO₄ and concentrated under reduced pressure. The residue was purified by preparative reverse phase HPLC [Phenomenex Luna 250×21.20 mm, 10 micron column. Solvent A—Acetonitrile (0.07% TFA); Solvent B—Water (0.10% TFA). Flow rate—20 mL/min. Gradient—5% to 80% A over 10 min; 80% to 100% A over 5 min; hold 100% A 5 min; 100% to 5% A over 5 min; 1 min hold; then recycle. Product retention time=12.0 min; starting material retention time=11.3 mind, and the product fractions were pooled and concentrated to a small volume. This aqueous residue was extracted with CH₂Cl₂ (2×20 mL), and the combined organic phase was washed with water (10 mL), dried over Na₂SO₄, concentrated under reduced pressure and dried under high vacuum to give the title compound (6) (6 mg, 10%) as a pale yellow solid: ¹H NMR (400 MHz, DMSO-d₆) δ ppm 8.74 (s, 1H), 7.93 (br. s, 1H), 7.73 (d, J=14.5 Hz, 1H), 5.28 (dd, J=16.2, 3.5 Hz, 1H), 5.02 (m, 1H), 4.39 (dd, J=16.4, 6.6 Hz, 1H), 4.18 (m, 1H), 3.88 (m, 1H), 3.65 (m, 1H), 2.32 (m, 1H), 1.94 (m, 3H), 1.24 (m, 2H), 0.90 (m, 1H), 0.71 (m, 1H); MS (ESI+) for C₁₉H₁₈FN₃O₄ m/z 372 (M+H)⁺, MS (ESI−) for C₁₉H₁₈FN₃O₄ m/z 370 (M−HPLC purity, 96% (ret. time, 6.03 min).

### Example 11

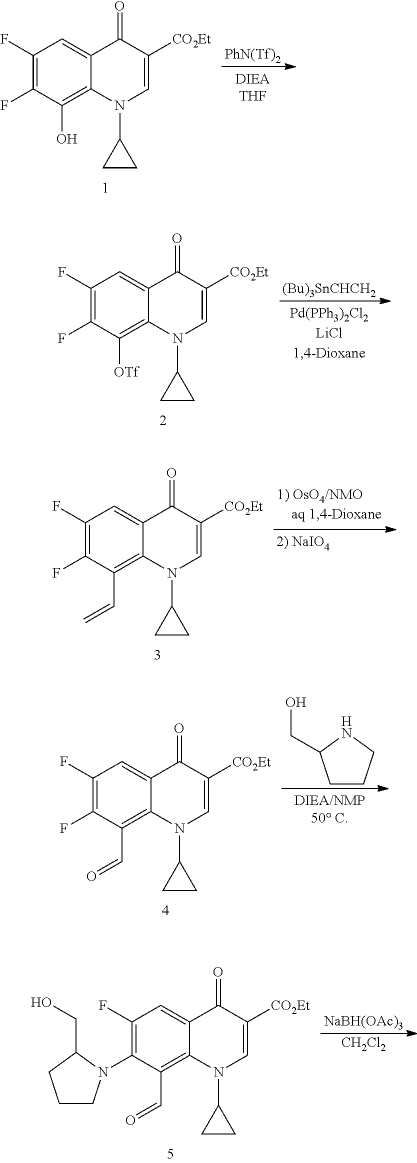

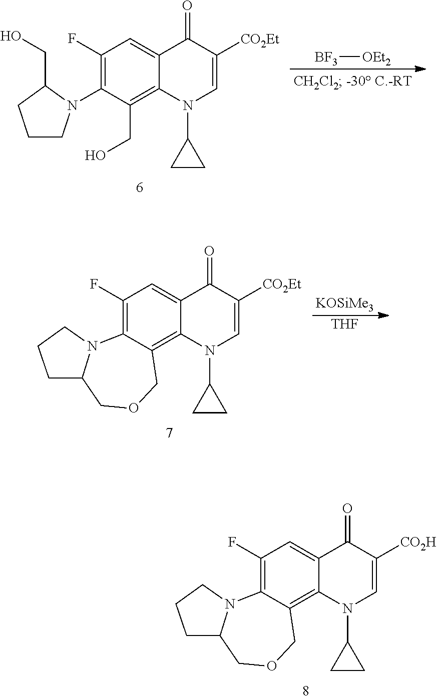

Analytical HPLC conditions: Agilent 1100 HPLC. Zorbax C8 150×4.6 mm column. Solvent A—Water (0.1% TFA); Solvent B—Acetonitrile (0.07% TFA). Flow rate, 1.50 mL/min. Gradient—10 min 95% A to 90% B; 2 min hold; then recycle. UV detection @ 214 and 254 nm, or @ 214 and 290 nm. All reactions were conducted under an atmosphere of nitrogen.

### Ethyl-1-cyclopropyl-6,7-difluoro-4-oxo-8-[(trifluoromethyl)sulfonyl]oxy-1,4-dihydroquinoline-3-carboxylate (2)

A mixture of ethyl-1-cyclopropyl-6,7-difluoro-8-hydroxy-4-oxo-1,4-dihydroquinoline-3-carboxylate (1) (38.8 g, 125 mmol), and N,N-diisopropylethylamine (43.7 mL, 251 mmol) in tetrahydrofuran (600 mL) was treated with N-phenylbis(trifluoromethanesulphonimide) (47.1 g, 132 mmol) in one portion at room temperature and the mixture was stirred for 24 h at which point it was determined by HPLC that the starting material was consumed. The reaction mixture was concentrated to a tan solid and the material was taken up in 700 mL ethyl acetate. The organic phase was successively washed with 500 mL each of 1N citric acid, saturated NaHCO₃ solution, and brine and dried over Na₂SO₄. The solution was filtered and concentrated to a light tan sticky solid. Further solvent was removed under high vacuum. The solid was taken up in 700 mL of boiling 2-propanol and the solution was allowed to cool slowly to room temperature during which time the triflate crystallized out. The solid was filtered, washed with 400 mL ice cold 2-propanol and dried in a vacuum oven at 85° C. overnight. The yield of the title compound (2) was 36.9 g (66%) as a light tan solid. MS (ESI+) for C₁₆H₁₂F₅NO₆S m/z 442 (M+H)⁺. HPLC purity 100% (ret. time, 9.10 min).

### Ethyl-1-cyclopropyl-6,7-difluoro-4-oxo-8-vinyl-1,4-dihydroquinoline-3-carboxylate (3)

A mixture of ethyl-1-cyclopropyl-6,7-difluoro-4-oxo-8-[(trifluoromethyl)sulfonyl]oxy-1,4-dihydroquinoline-3-carboxylate (2) (200 mg, 0.4 mmol), (2-ethenyl)tri-n-butyltin (0.20 mL, 0.68 mmol), and lithium chloride (60 mg, 1 mmol) in 1,4-dioxane (3.2 mL, 41 mmol) was sparged for 10 min with nitrogen gas. Bis(triphenylphosphine)palladium(II) chloride (30 mg, 0.04 mmol) was added and the mixture was sparged again for 10 min, after which, the reaction mixture was heated at 80° C. overnight. After 15 h at 80° C., TLC (40% ethyl acetate/CH₂Cl₂) indicated triflate was consumed and a new product had formed. The mixture was diluted with 5 mL ethyl acetate, filtered through a fine frit and the solids washed with 10 mL ethyl acetate. The solution was concentrated to a brown oil that was subjected to flash chromatography (50 g flash silica gel; 10-40% EA/CH₂Cl₂), which yielded 25 mg (25%) of the title compound (3) as a tan solid. MS (ESI+) for C₁₇H₁₅F₂NO₃ m/z 320 (M+H)⁺. HPLC purity 100% (ret. time, 7.56 min).

### Ethyl-1-cyclopropyl-6,7-difluoro-8-formyl-4-oxo-1,4-dihydroquinoline-3-carboxylate

A mixture of ethyl-1-cyclopropyl-6,7-difluoro-4-oxo-8-vinyl-1,4-dihydroquinoline-3-carboxylate (3) (77 mg, 0.24 mmol) in 1,4-dioxane (2 mL) and water (0.77 mL) (mixture was warmed to 40° C. in order to obtain a homogeneous solution) was treated with osmium tetroxide, 2.5 wt. % solution in 2-methyl-2-propanol (0.081 mL, 0.005 mmol) and the mixture was stirred for 30 minutes. N-methylmorpholine N-oxide (28 mg, 0.24 mmol) was added and the reaction was stirred for 20 h at 40° C. HPLC after 20 h indicated the reaction was complete. The now dark solution was treated with sodium metaperiodate (52 mg, 0.24 mmol) in one portion and the mixture was stirred at room temperature. After 6 h, an additional 50 mg of sodium metaperiodate and 1 mL of 1,4-dioxane was added and stirring was continued. Reaction was found to be complete by HPLC after 24 h. The reaction mixture was diluted with 7 mL H₂O and extracted with three 10 mL portions of CH₂Cl₂. The combined organic phase was washed with 15 mL portions of H₂O and brine and dried over Na₂SO₄. The organic phase was filtered and concentrated to yield 67 mg of the title compound (4) as a slightly tan solid which was of sufficient purity to be used in the next step. m/z (M+H)⁺. MS (ESI+) for C₁₆H₁₃F₂NO₄ m/z 322 (M+H)⁺. HPLC purity 100% (ret. time, 6.51 min).

### Ethyl-1-cyclopropyl-6-fluoro-7-(pyrrolidin-2-ylmethanol)-8-formyl-4-oxo-1,4-dihydroquinoline-3-carboxylate (5)

A mixture of ethyl-1-cyclopropyl-6,7-difluoro-8-formyl-4-oxo-1,4-dihydroquinoline-3-carboxylate (4) (75 mg, 0.23 mmol), N,N-diisopropylethylamine (89.4 uL, 0.514 mmol) and pyrrolidin-2-ylmethanol (0.045 g, 0.44 mmol) in N-methylpyrrolidinone (2 mL) was heated at 50° C. overnight. After 13 h at 50° C., the reaction was complete by HPLC. The mixture was cooled to room temperature, poured into 20 mL H₂O and extracted with three 15 mL portions of 1/1 ethyl acetate/methyl tert-butyl ether. The combined organic phase was washed twice with 15 mL H₂O, dried over Na₂SO₄, filtered and concentrated to a yellow solid. The crude material was purified by chromatography (40 g flash silica; 2-5% MeOH/CH₂Cl₂), to yield 80 mg of the title compound (5) as a yellow solid. MS (ESI+) for C₂₁H₂₃FN₂O₅ m/z 403 (M+H)⁺. HPLC purity 100% (ret. time, 5.88 min).

### Ethyl-1-cyclopropyl-6-fluoro-7-[2-(hydroxymethyl)pyrrolidin-1-yl]-8-hydroxymethyl-4-oxo-1,4-dihydroquinoline-3-carboxylic acid (6)

A solution of ethyl-1-cyclopropyl-6-fluoro-7-(pyrrolidin-2-ylmethanol)-8-formyl-4-oxo-1,4-dihydroquinoline-3-carboxylate (5) (1.20 g, 2.98 mmol) in methylene chloride (59 mL) was treated with sodium triacetoxyborohydride (1.26 g, 5.96 mmol) in one portion and stirred at room temperature for 9 days after which the reaction was found to be complete by HPLC. The reaction mixture was diluted with 20 mL CH₂Cl₂ and washed with 50 mL portions of H₂O and sat NaHCO₃. The organic phase was dried over Na₂SO₄, filtered and concentrated to a light yellow solid. The material was purified by chromatography (80 g flash silica; 4-9% EtOH/CH₂Cl₂), to yield 810 mg of the title compound (6) as a white solid. MS (ESI+) for C₂₁H₂₅FN₂O₅MS m/z 405 (M+H)⁺. HPLC purity 100% (ret. time, 5.72 min).

### Ethyl-13-cyclopropyl-8-fluoro-10-oxo-3a,4,5,6,10,13-hexahydro-1H,3H-pyrrolo[2′,1′:3,4][1,4]oxazepino[5,6-h]quinoline-1′-carboxylate (7)

The starting diol was azeotroped twice from toluene and placed on high vac before being subjected to the reaction conditions. A solution of ethyl-1-cyclopropyl-6-fluoro-7-[2-(hydroxymethyl)pyrrolidin-1-yl]-8-hydroxymethyl-4-oxo-1,4-dihydroquinoline-3-carboxylic acid (6) (211 mg, 0.522 mmol) in methylene chloride (7.0 mL, 110 mmol) was cooled at −30° C. and treated with boron trifluoride etherate (140 uL, 1.1 mmol) dropwise. The reaction mixture was stirred and allowed to slowly warm to room temperature. The reaction mixture began as a white slurry that became a homogenous yellow solution upon treatment with the BF₃—OEt₂. After 17 h the reaction was found to be complete by HPLC. The reaction mixture was diluted with 25 mL CH₂Cl₂ and washed with 20 mL sat NaHCO₃. The aqueous phase was washed once with 10 mL CH₂Cl₂ and the organic phases were combined and dried over Na₂SO₄. The solution was filtered and concentrated to a light yellow solid. The material was purified by chromatography (40 g flash silica; 30-50% EA/CH₂Cl₂), to yield 114 mg of the title compound (7) as a white solid. MS (ESI+) for C₂₁H₂₃FN₂O₄MS m/z 387 (M+H)⁺. HPLC purity 100% (ret. time, 7.41 min).

### 13-Cyclopropyl-8-fluoro-10-oxo-3a,4,5,6,10,13-hexahydro-1H,3H-pyrrolo[2′,1′:3,4][1,4]oxazepino[5,6-h]quinoline-11-carboxylic acid (8)

A solution of ethyl-13-cyclopropyl-8-fluoro-10-oxo-3a,4,5,6,10,13-hexahydro-1H,3H-pyrrolo[2′,1′:3,4][1,4]oxazepino[5,6-h]quinoline-11-carboxylate (7) (30.0 mg, 0.0776 mmol) in tetrahydrofuran (3 mL, 40 mmol) was treated with potassium trimethylsilanolate (13 mg, 0.093 mmol) and the mixture was stirred at room temperature for 72 h. HPLC indicated the reaction was complete. The reaction mixture was concentrated, the residue diluted with 5 mL H₂O, and made acidic to pH˜2 with 0.5N HCl. The aqueous phase was extracted with three 10 mL portions of ethyl acetate which were combined and dried over Na₂SO₄, filtered and concentrated to yield a tan solid. The solid was taken up in 0.5 mL NMP and purified by prep reverse phase HPLC: Phenomenex Luna 250×21.20 mm, 10 micron column. Gradient: solvent A is 0.07% TFA in acetonitrile; solvent B is 0.10% TFA in water; 26 minute run; 5% to 80% A over 10 minute ramp; 5 minute ramp from 80% to 100% A; hold 100% A for 5 minutes; ramp down from 100% to 5% A over 5 minutes; hold 1 minute, then recycle. Detector wavelength set to 290 nm. Product retention time=12.3 min. The yield of the title compound (8) was 15 mg as a tan solid. MS (ESI+) for C₁₉H₁₉FN₂O₄ m/z 359 (M+H)⁺. HPLC purity 100% (ret. time, 7.81 min).

### Example 12

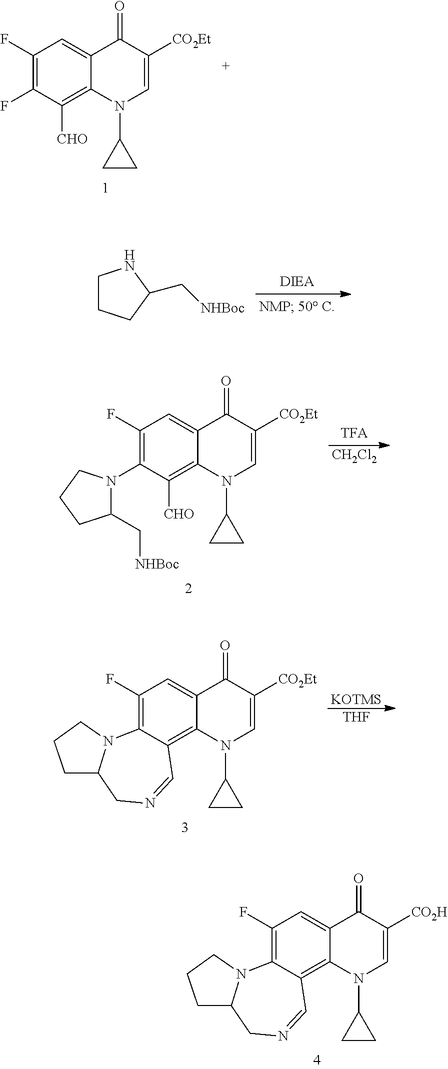

HPLC conditions: Agilent 1100 HPLC. Zorbax C8 150×4.6 mm column. Solvent A—Water (0.1% TFA); Solvent B—Acetonitrile (0.07% TFA). Flow rate, 1.50 mL/min. Gradient—10 min 95% A to 90% B; 2 min hold; then recycle. UV detection @ 214 and 254 nm or 214 and 290 nm. All reactions were conducted under an atmosphere of nitrogen.

### Ethyl-7-(2-[(tert-butoxycarbonyl)amino]methylpyrrolidin-1-yl)-1-cyclopropyl-6-fluoro-8-formyl-4-oxo-1,4-dihydroquinoline-3-carboxylate (2)

A mixture of ethyl-1-cyclopropyl-6,7-difluoro-8-formyl-4-oxo-1,4-dihydroquinoline-3-carboxylate (1) (653 mg, 2.03 mmol), N,N-diisopropylethylamine (0.779 mL, 4.47 mmol) and 2-N-Boc-aminomethylpyrrolidine (570 mg, 2.8 mmol) in N-methylpyrrolidinone (6 mL) was heated at 50° C. After about 1 h, reaction was complete by HPLC. Reaction mixture was cooled to room temperature and diluted with 100 mL H₂O, which produced a greenish yellow precipitate. The mixture was extracted with two 80 mL portions of 10% CH₂Cl₂/EA. The combined organic phase was washed twice with 50 mL portions of H₂O and dried over Na₂SO₄. The solution was filtered and concentrated to a brownish yellow solid. The material was purified by chromatography (80 g flash silica, 25-65% EA/CH₂Cl₂), to yield the title compound (2) (0.87 g, 85%) as a light yellow solid: MS (ESI+) for C₂₆H₃₂FN₃O₆ m/z 446.3 (M+H)⁺; HPLC purity 100% (ret. time, 7.77 min).

### Ethyl-13-cyclopropyl-8-fluoro-10-oxo-3a,4,5,6,10,13-hexahydro-3H-pyrrolo[2′,1′:3,4][1,4]diazepino[5,6-h]quinoline-11-carboxylate (3)

A solution of ethyl-7-(2-[(tert-butoxycarbonyl)amino]methylpyrrolidin-1-yl)-1-cyclopropyl-6-fluoro-8-formyl-4-oxo-1,4-dihydroquinoline-3-carboxylate (2) (380 mg, 0.758 mmol) in methylene chloride (10 mL, 200 mmol) was cooled at 0° C. and treated with trifluoroacetic acid (2.6 mL) dropwise. The reaction was allowed to slowly warm to room temperature. After about 1.5 h, the reaction appeared complete by HPLC. The reaction mixture was concentrated and the residue was taken up in 10 mL CH₂Cl₂ and reconcentrated. The residue was taken up in 25 mL CH₂Cl₂ and washed with 20 mL sat NaHCO₃. The bicarb phase was back extracted with 20 mL CH₂Cl₂, and the combined organic extracts were dried over Na₂SO₄, filtered and concentrated to give the title compound (3) (300 mg, in near quantitative yield) as a light yellow solid which was found to be of suitable purity for use in the next step: MS (ESI+) for C₂₁H₂₂FN₃O₃ m/z 384 (M+H)⁺; HPLC purity 100% (ret. time, 4.37 min).

### 13-Cyclopropyl-8-fluoro-10-oxo-3a,4,5,6,10,13-hexahydro-3H-pyrrolo[2′,1′:3,4][1,4]diazepino[5,6-h]quinoline-11-carboxylic acid (4)

A solution of ethyl-13-cyclopropyl-8-fluoro-10-oxo-3a,4,5,6,10,13-hexahydro-3H-pyrrolo[2′,1′:3,4][1,4]diazepino[5,6-h]quinoline-11-carboxylate (37 mg, 0.096 mmol) in tetrahydrofuran (3.0 mL) was treated with potassium trimethylsilanolate (16 mg, 0.12 mmol) in one portion and allowed to stir at room temperature for 5 h, upon which the reaction was found to be complete by HPLC. The reaction mixture was diluted with 7 mL 0.5N HCl and extracted three times with 10 mL portions of CH₂Cl₂. The aqueous phase was neutralized to pH ˜7 with saturated NaHCO₃ and extracted with three 10 mL portions of CH₂Cl₂. The organic phase was dried over Na₂SO₄, filtered and concentrated to a yellow solid. The solid was treated with 3 mL methyl t-butyl ether (MTBE), filtered and washed with additional MTBE to give the title compound (4) (19 mg, 44%) as a tan solid: MS (ESI+) for C₁₉H₁₈FN₃O₃ m/z 356 (M+H)⁺, 354 (M+H)⁻; HPLC purity 97% (ret. time, 4.57 min).

### Example 13

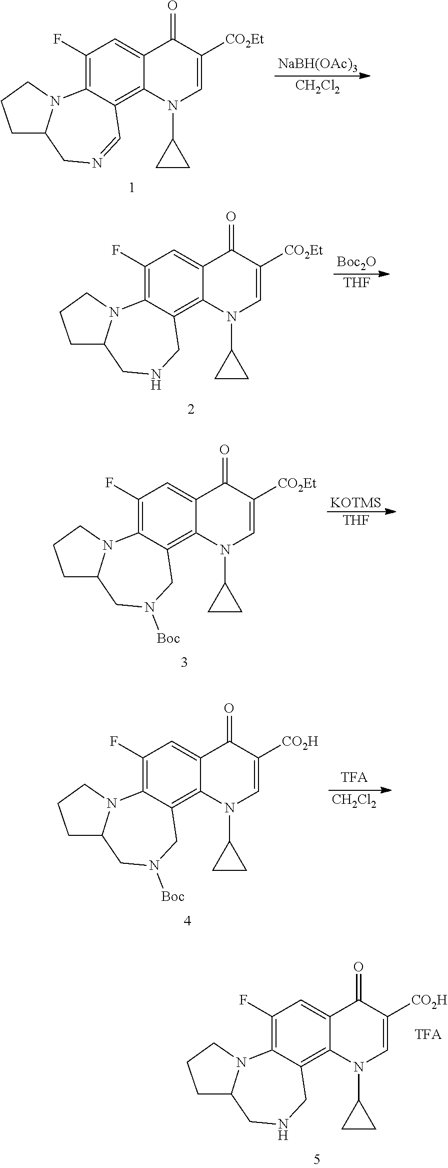

### Ethyl-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylate (2)

A solution of ethyl-13-cyclopropyl-8-fluoro-10-oxo-3a,4,5,6,10,13-hexahydro-3H-pyrrolo[2′,1′:3,4][1,4]diazepino[5,6-h]quinoline-11-carboxylate (1) (287 mg, 0.748 mmol) in methylene chloride (15 mL) was treated with sodium triacetoxyborohydride (317 mg, 1.50 mmol) in one portion and the mixture was stirred at room temperature. After 6 h, reaction was complete by HPLC. The reaction mixture was diluted with 30 mL CH₂Cl₂ and was washed with 25 mL saturated NaHCO₃. The aqueous phase was back extracted with 10 mL CH₂Cl₂ and the combined organic phase was dried over Na₂SO₄. The solution was filtered and concentrated to a yellow glass. The crude product was dissolved in 30 mL CH₂Cl₂ and washed with 20 mL sat NaHCO₃ solution. The organic phase was dried over Na₂SO₄, filtered and concentrated to yield the title compound (2) (266 mg, 92%) as a crude yellow solid; MS (ESI+) for C₂₁H₂₄FN₃O₃ m/z 386 (M+H)⁺; HPLC purity, 71% (ret. time, 4.66 min).

### 2-tert-Butyl-11-ethyl 13-cyclopropyl-8-fluoro-10-oxo-3a,4,5,6,10,13-hexahydro-1H-pyrrolo[2′,1′:3,4][1,4]diazepino[5,6-h]quinoline-2,11(3H)-dicarboxylate 3)

A mixture of ethyl-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylate (2) (314 mg, 0.692 mmol) in tetrahydrofuran (10 mL) was treated dropwise with triethylamine (0.106 mL, 0.762 mmol) followed by di-tert-butyldicarbonate (0.166 g, 0.762 mmol). The mixture was stirred at room temperature for 17 h upon which the reaction was determined to be complete by HPLC. The reaction mixture was diluted with 30 mL CH₂Cl₂, washed with 20 mL portions of H₂O, 0.01N HCl and H₂O and dried over Na₂SO₄. The solution was filtered and concentrated to yield a yellow glass. The material was purified by chromatography (40 g flash silica, 40-60% EA/CH₂Cl₂) to yield the title compound (3) (308 mg, 91%) as a light yellow solid; MS (ESI+) for C₂₆H₃₂FN₃O₅ m/z 486 (M+H)⁺; HPLC purity 94% (ret. time, 7.97 min).

### 2-(tert-Butoxycarbonyl)-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylic acid (4)

A solution of 2-tert-butyl-11-ethyl-13-cyclopropyl-8-fluoro-10-oxo-3a,4,5,6,10,13-hexahydro-1H-pyrrolo[2′,1′:3,4][1,4]diazepino[5,6-h]quinoline-2,11(3H)-dicarboxylate (3) (57 mg, 0.12 mmol) in tetrahydrofuran (5 mL) was treated with the potassium trimethylsilanolate (28 mg, 0.031 mmol) in one portion and the mixture was allowed to stir at room temperature for 3 h, upon which the reaction was determined complete by HPLC. The reaction mixture was diluted with 5 mL H₂O and made acidic to pH˜3 with 0.5N HCl solution. The aqueous phase was extracted with two 20 mL portions of ethyl acetate and the combined organic phase was washed with 15 mL brine. The organic phase was dried over Na₂SO₄, filtered and concentrated to an orange solid. The material was treated with 2 mL MTBE and filtered to give the title compound (4) (51 mg, 89%) as a tan solid; MS (ESI+) for C₂₄H₂₈FN₃O₅ m/z 458 (M+H)⁺; HPLC purity 100% (ret. time, 8.43 min).

### 13-Cyclopropyl-8-fluoro-10-oxo-2,3,3a,4,5,6,10,13-octahydro-1H-pyrrolo[2′,1′:3,4][1,4]diazepino[5,6-h]quinoline-11-carboxylic acid trifluoroacetate (5)

A solution of 2-(tert-butoxycarbonyl)-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylic acid (4) (50 mg, 0.11 mmol) in methylene chloride (2.0 mL, 31 mmol) was cooled to 0° C. in an ice bath and treated dropwise with trifluoroacetic acid (0.5 mL, 6 mmol). The mixture was allowed to stir and warm up to room temperature. After 2 h, starting material was consumed by HPLC. The reaction mixture was concentrated to a yellow-orange oil, dissolved in 5 mL CH₂Cl₂ and concentrated, followed by concentration from CH₂Cl₂/MTBE. The material was placed on high vac, upon which a tan solid formed. The product was isolated by preparative reverse phase HPLC with the following conditions: Phenomenex Luna 250×21.20 mm, 10 micron column. Gradient: solvent A=0.07% TFA in acetonitrile; solvent B=0.10% TFA in water; 26 minute run; 5% to 70% A over 14 minute ramp; 70% to 100% A over 3 minute ramp; hold 100% A over 3 minutes; ramp down from 100% to 5% A over 5 minutes; hold for 1 minute, then recycle. Detector wavelength was set to 290 nm. Product retention time=11.35 minutes. Product fractions were combined, concentrated to remove the acetonitrile and the water removed by lyophilization to yield the title compound (5) (27 mg, 52%) as a light yellow solid (as the TFA salt); MS (ESI+) for C₁₉H₂₀FN₃O₃ m/z 358 (M+H)⁺; HPLC purity 95% (ret. time, 4.95 min).

### Example 14

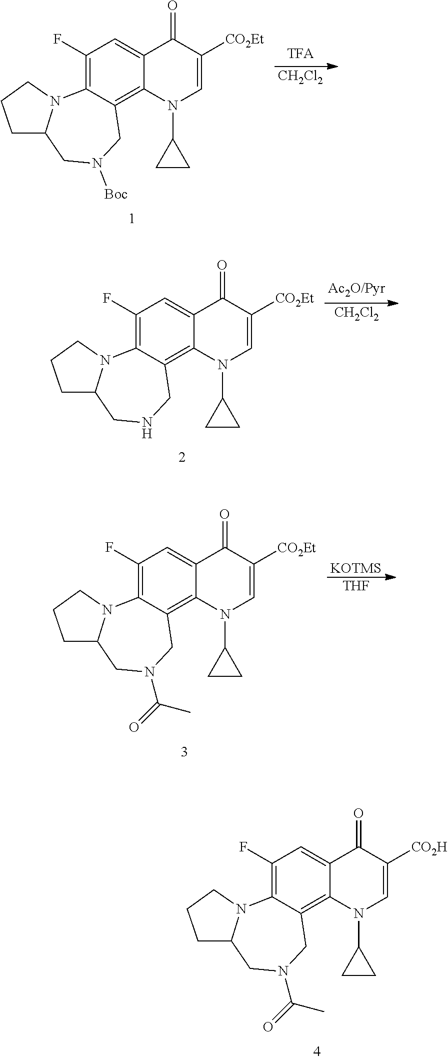

HPLC conditions: Agilent 1100 HPLC. Zorbax C8 150×4.6 mm column. Solvent A—Water (0.1% TFA); Solvent B—Acetonitrile (0.07% TFA). Flow rate, 1.50 mL/min. Gradient—10 min 95% A to 90% B; 2 min hold; then recycle. UV detection @ 214 and 254 nm or @290 and 214 nm. All reactions were conducted under an atmosphere of nitrogen.

### Ethyl-2-acetyl-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylate (3)

A solution of 2-tert-butyl-11-ethyl-13-cyclopropyl-8-fluoro-10-oxo-3a,4,5,6,10,13-hexahydro-1H-pyrrolo[2′,1′:3,4][1,4]diazepino[5,6-h]quinoline-2,11(3H)-dicarboxylate (1) (0.070 g, 0.14 mmol) in methylene chloride (3.0 mL) was cooled to 0° C. in an ice bath and treated dropwise with trifluoroacetic acid (0.5 mL, 6 mmol). The mixture was allowed to stir and warm up to room temperature. After 2.5 h, starting material was consumed as indicated by HPLC. The reaction mixture was concentrated to a yellow-orange oil, dissolved in 5 mL CH₂Cl₂, concentrated and the material was placed on high vac for about 30 minutes. The resultant crude amine TFA salt (2) was dissolved in methylene chloride (3.0 mL) and treated with pyridine (26 uL, 0.32 mmol) followed by acetic anhydride (16 uL, 0.17 mmol) dropwise and the mixture was allowed to stir at room temperature overnight. HPLC indicated the reaction was complete after 18 h. The reaction mixture was diluted with 15 mL CH₂Cl₂, washed with 10 mL H₂O and dried over Na₂SO₄. The solution was filtered and concentrated to yield a tan solid. The material was purified by chromatography (30 g silica gel, 3-6% EtOH/CHCl₃), to yield the title compound (3) (45 mg, 73%) as a light yellow glass: MS (ESI+) for C₂₃H₂₆FN₃O₄ m/z 428 (M+H)⁺; HPLC purity, 100% (ret. time, 5.88 min).

### 2-Acetyl-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylic acid (4)

A solution of ethyl-2-acetyl-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylate (3) (45 mg, 0.10 mmol) in tetrahydrofuran (3 mL) was treated with potassium trimethylsilanolate (26 mg, 0.18 mmol) in one portion and the mixture was allowed to stir at room temperature for 20 h, upon which the reaction was found to be complete by HPLC. The reaction mixture was diluted with 5 mL H₂O and made acidic to pH˜3 with 0.5N HCl solution. The aqueous phase was extracted with four 10 mL portions of ethyl acetate and the organic extracts were dried over Na₂SO₄. The solution was filtered and concentrated to yield a tan solid. The crude product (13 mg) was taken up in 0.7 mL NMP and purified by preparative reverse phase HPLC, using the following conditions: Phenomenex Luna 250×21.20 mm, 10 micron column. Gradient: solvent A=0.07% TFA in acetonitrile; solvent B=0.10% TFA in water; 26 minute run; 5% to 70% A over 14 minute ramp; 70% to 100% A over 3 minutes ramp; hold 100% A over 3 minutes; ramp down from 100% to 5% A over 5 minutes; hold for 5 minutes then recycle. Detector wavelength set to 290 nm. Product retention time=13.05 min. Product fractions were combined, concentrated to remove the acetonitrile, and the water was removed by lyophilization to yield the desired compound (4) (5 mg, 10% yield) as a yellow fluffy solid: MS (ESI+) for C₂₁H₂₂FN₃O₄ m/z 400 (M+H)⁺; HPLC purity, 97% (ret. time, 6.20 min).

### Example 15

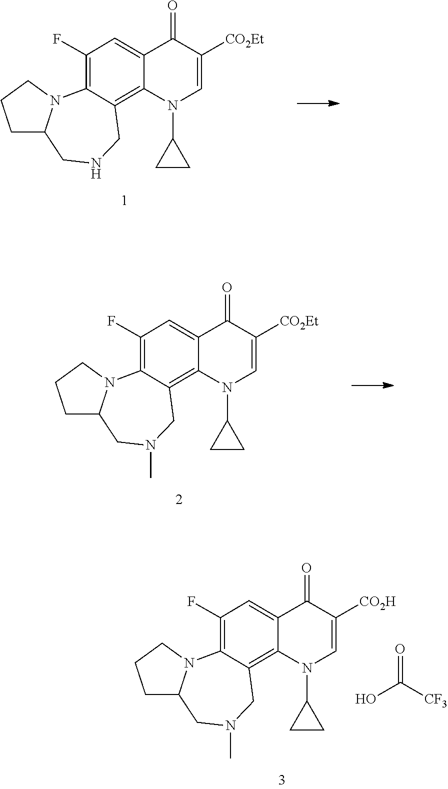

### Ethyl-13-cyclopropyl-8-fluoro-2-methyl-10-oxo-2,3,3a,4,5,6,10,13-octahydro-1H-pyrrolo[2′,1′:3,4][1,4]diazepino[5,6-h]quinoline-11-carboxylate (2)

A mixture of ethyl-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylate (1) (113 mg, 0.293 mmol) in formic acid (2 mL, 60 mmol) was treated with 37% formaldehyde (1.00 g) and heated to 80° C. After about 60 min, LC-MS indicated starting material was consumed. The reaction mixture was cooled to room temperature and concentrated to about one third the original volume. The residue was diluted with 10 mL H₂O and treated with saturated NaHCO₃ to give a solution of pH˜8. The aqueous phase was extracted with three 20 mL portions of CH₂Cl₂. The combined organic phase was dried over Na₂SO₄, filtered and concentrated to a yellow glass. The material was purified by chromatography (40 g silica gel, 1-7% EtOH/CHCl₃) to yield 79 mg of a yellow solid. The material was not completely clean by LC-MS and was subjected to prep TLC using two 20 cm×20 cm×1 mm silica gel TLC plates eluting with 5% EtOH/CHCl₃, yielding the title compound (2) (51 mg, 44%) as a light yellow solid: MS (ESI+) for C₂₂H₂₆FN₃O₃ m/z 400 (M+H)⁺; HPLC purity, 97% (ret. time, 4.72 min).

### 13-Cyclopropyl-8-fluoro-2-methyl-10-oxo-2,3,3a,4,5,6,10,13-octahydro-1H-pyrrolo[2′,1′:3,4][1,4]diazepino[5,6-h]quinoline-11-carboxylic acid trifluoroacetate (3)

A solution of ethyl-13-cyclopropyl-8-fluoro-2-methyl-10-oxo-2,3,3a,4,5,6,10,13-octahydro-1H-pyrrolo[2′,1′:3,4][1,4]diazepino[5,6-h]quinoline-1′-carboxylate (2) (49 mg, 0.12 mmol) in acetonitrile (6 mL, 100 mmol) was treated with 1.35 mL 0.1 M aqueous NaOH solution and heated at 35° C. for 18 h. An additional 0.25 mL of 0.1N NaOH was added and heating was continued for 48 h. The reaction mixture was treated a second time with 0.25 mL of 0.1N NaOH and heating was continued for 24 h where upon the reaction was determined to be 90% complete by HPLC. The reaction was cooled to room temperature, reduced in volume, diluted with 5 mL H₂O and extracted once with 5 mL ethyl acetate. The aqueous phase was treated with 0.1N HCl to a pH˜7 which produced a fine precipitate. The precipitate was filtered, washed with water and diethyl ether to give a light yellow solid. The solid was taken up in 6 mL 0.1% aqueous TFA and lyophilized to obtain the title compound (3) (13 mg, 22%) as a yellow solid: MS (ESI+) for C₂₂H₂₆FN₃O₃ m/z 372 (M+H)⁺; HPLC purity 98.8% (ret. time, 4.84 min).

### Example 16

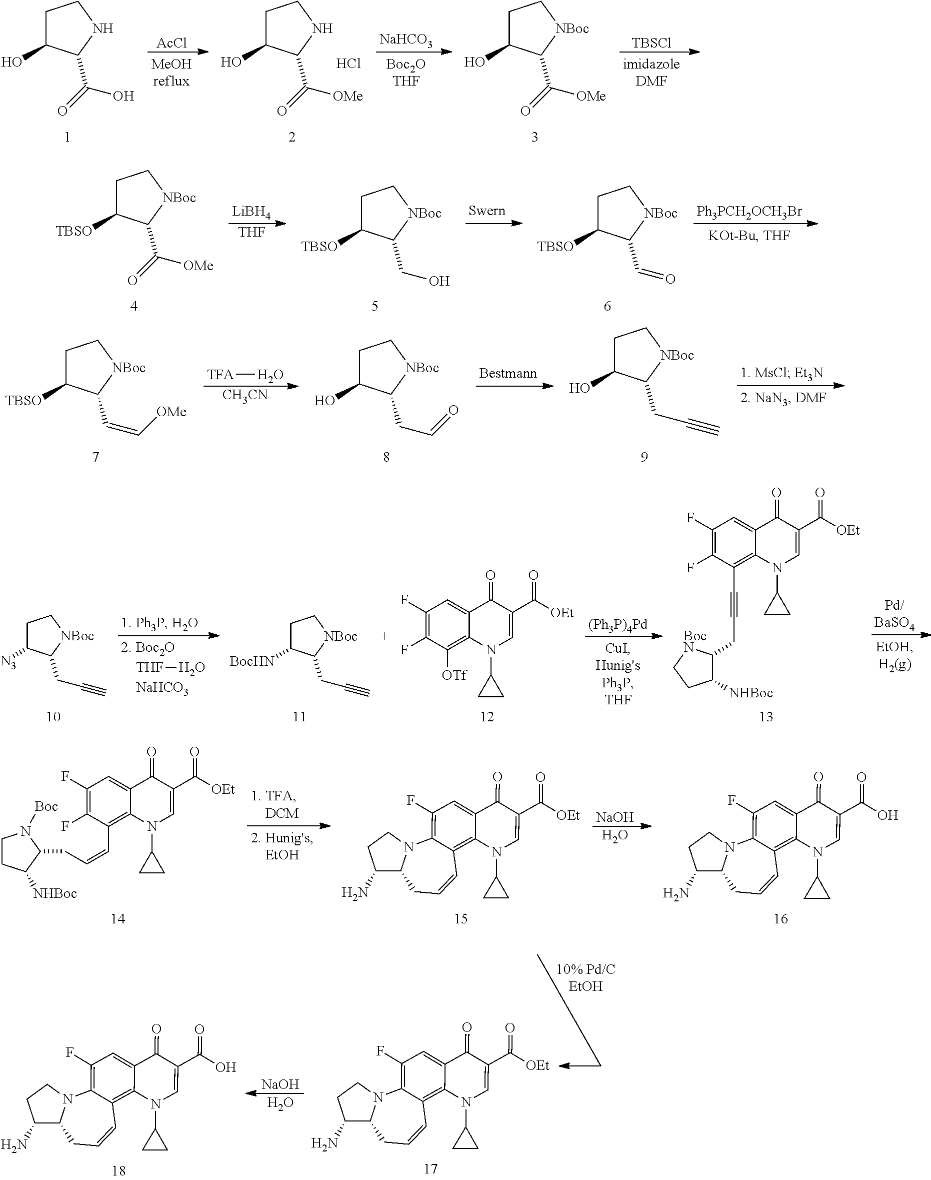

All reactions were performed under an atmosphere of N₂ (g). Unless otherwise indicated, the reaction flask was evacuated with vacuum and then back-filled with N₂ (g) via a balloon (×3) and the reaction kept under N₂ (g) via balloon for the duration of the reaction. Analytical HPLC was performed using an Agilent 1100 HPLC with one of the following methods:

Method A: Agilent Scalar C18 150×4.6 mm 5 micron column; 1.5 mL/min; solvent A—water (0.1% TFA); solvent B—acetonitrile (0.07% TFA, gradient: 10 min 95% A to 95% B; 5 min hold; then recycle; UV detection @ 214, 250 and 280 nm.

Method B: Agilent XDB C18 50×4.6 mm/1.8 micron column; 1.5 mL/min; solvent A—water (0.1% TFA), solvent B—acetonitrile (0.07% TFA); gradient: 5 min 95% A to 95% B then 1 min hold, 1 min 95% B to 95% A then 30 sec hold; UV detection @ 210, 254, and 280 nm.

Method C: Agilent Eclipse XBD C8 column; solvent A—water (0.1% TFA); solvent B—acetonitrile (0.07% TFA, gradient: 10 min 95% A to 95% B; 5 min hold; then recycle; UV detection @ 214, 250 and 280 nm.

Preparative HPLC conditions: Phenomenex Luna 250×21.20 mm, 10 micron; solvent A is 0.07% TFA in acetonitrile; solvent B is 0.10% TFA in water; 26 minute run; gradient: 5% to 80% A over 10 minutes; from 80% to 100% A over 5 minutes; hold 100% A for 5 minutes; 100% to 5% A over 5 minutes; hold 1 minute then recycle; detection at 285 nm. Thin layer chromatography (TLC) was performed using Analtech TLC plates GHLF, 250 microns, order #21521. Regular phase silica gel chromatography was done using R10030B 40-63 μM 60 Å silica gel from Silicycle. ¹H NMR was obtained on a Brucker Avance 400 MHz instrument in the stated solvent. Mass spectral data was obtained on a Micromass instrument using electrospray ionization.

### Methyl-(3S)-3-hydroxy-L-prolinate hydrochloride (2)

(3S)-3-Hydroxy-L-proline (1) (10.0 g, 76.3 mmol) was added, in one portion, to a stirred solution (at 0° C./ice-water bath) of acetyl chloride (7.6 mL, 110 mmol) in methanol (70 mL). After the addition, the ice-water bath was removed and the reaction warmed to ambient temperature and then heated at 65° C. for 5-6 hr. After this period of time, the reaction was complete based on TLC (20% MeOH/CHCl₃) and was cooled to ambient temperature. The cooled reaction mixture was diluted with ether (150 mL) and a white precipitate formed. The white precipitate was collected by filtration and the solid washed ×2 with 25-mL of cold diethyl ether and then dried overnight under high vacuum to afford 12.6 g of (2) in 91% yield as a white crystalline solid. ¹H NMR is consistent; ¹H NMR (400 MHz, DMSO-d₆ with CDCl₃ added) δ ppm 9.67 (br. s., 2H), 5.93 (br. s., 1H), 4.50 (br. s., 1H), 4.18 (d, J=2.07 Hz, 1H), 3.77 (s, 3H), 3.35 (m, 2H), 1.94 (m, 2H).

### 1-tert-Butyl-2-methyl-(2S,3S)-3-hydroxypyrrolidine-1,2-dicarboxylate (3)

Methyl-(3S)-3-hydroxy-L-prolinate hydrochloride (2) (12.62 g, 69.4 mmol) was dissolved in tetrahydrofuran (500 mL) and water (20 mL) and then cooled at 0° C. in an ice-water bath. Then, sodium bicarbonate (14.6 g, 0.174 mol) was added followed by di-tert-butyldicarbonate (22.7 g, 0.104 mol) in tetrahydrofuran (75 mL)—added dropwise via an addition funnel. The ice-water bath was removed and the reaction was stirred at ambient temperature overnight (˜10 hr). After this period of time, the reaction was complete based on TLC analysis (30% ethyl acetate/CH₂Cl₂; R_{f} for product ˜0.40; starting material R_{f} ˜0.05). The reaction was concentrated to remove the THF and then the resulting aqueous layer was partitioned between ethyl acetate (100 mL) and water (200 mL) and then separated. The aqueous layer was extracted (3×50 mL) with ethyl acetate. The combined organic layers were washed with H₂O and brine, dried over MgSO₄, and filtered to afford the crude product. The crude product was purified by silica gel chromatography (120 g column) eluting with 0 to 30% ethyl acetate in CH₂Cl₂ to afford purified product (3), 16.6 g, in 97% yield; ¹H NMR confirms; ¹H NMR (400 MHz, CDCl₃) δ ppm 4.46 (m, 1H), 4.31 (s, 0.40H, CH rotamer next to BOC), 4.19 (s, 0.60H, CH rotamer next to BOC), 3.76 (s, 3H), 3.64 (m, 2H), 2.16 (m, 1H), 2.12 (m, 1H), 1.93 (m, 1H), 1.49 (s, 4H, BOC rotamer), 1.43 (s, 5 H, BOC rotamer).

### 1-tert-Butyl-2-methyl-(2S,3S)-3-{[tert-butyl(dimethyl)silyl]oxy}pyrrolidine-1,2-dicarboxylate (4)

1-tert-Butyl-2-methyl-(2S,3S)-3-hydroxypyrrolidine-1,2-dicarboxylate (3) (16.6 g, 67.7 mmol) was dissolved in N,N-dimethylformamide (100 mL) and 1H-imidazole (6.91 g, 102 mmol) was added followed by tert-butyldimethylsilyl chloride (12.2 g, 81.2 mmol). The reaction was stirred for 2-3 hr at ambient temperature and then checked for completion by silica gel TLC (30% ethyl acetate/CH₂Cl₂). TLC at this time indicates a very small amount of the alcohol. The reaction was treated with additional reagent, 1H-imidazole (1.4 g, 20.0 mmol) and tert-butyldimethylsilyl chloride (1.5 g, 10.0 mmol) added successively. The reaction was stirred for an additional hour and then quenched by the addition of 200 mL of water. The organic product was extracted with diethyl ether (2×200 mL) and the combined organic layers washed with water (3×100 mL), 1 M aqueous HCl (100 mL), saturated sodium bicarbonate (1×100 mL), and brine (1×100 mL) and then dried over MgSO₄. The filtered product was concentrated in vacuo to afford a light clear oil that was purified using silica gel chromatography (120 g silica) eluting with 0, 5, and 10% ethyl acetate in hexanes to afford 23.4 g of the TBS ether (4) in 96% yield; ¹H NMR confirms; ¹H NMR (400 MHz, CDCl₃) δ ppm 4.25 (m, 1H), 4.08 (s, 0.4H, CH rotamer), 3.94 (d, J=2.28 Hz, 0.6H, CH rotamer), 3.64 (s, 1H, CH₃ rotamer), 3.63 (s, 2H, CH₃ rotamer), 3.46 (m, 2H), 1.91 (m, 1H), 1.71 (m, 1H), 1.37 (s, 4H, t-Bu rotamer), 1.31 (s, 5H, t-Bu rotamer), 0.78 (s, 9H), −0.01 (s, 3H), −0.02 (s, 3H).

### tert-Butyl-(2R,3S)-3-{[tert-butyl(dimethyl)silyl]oxy}-2-(hydroxymethyl)pyrrolidine-1-carboxylate (5)

tert-Butyl-2-methyl-(2S,3S)-3-{[tert-butyl(dimethyl)silyl]oxy}pyrrolidine-1,2-dicarboxylate (4) (23.4 g, 65.1 mmol) dissolved in tetrahydrofuran (400 mL) and was cooled at 0° C. in an ice-water bath. Then, lithium tetrahydroborate (2.13 g, 97.6 mmol) was added in portions and the ice bath allowed to expire overnight with continued stirring for approximately 18 hr. After this period of time, the reaction was checked for completion by TLC analysis (30% ethyl acetate:hexanes), which revealed the complete consumption of the starting material (R_{f} ˜0.70) and formation of a major product (R_{f} ˜0.5). The reaction was concentrated to remove the THF and then partitioned between chloroform (200 mL) and 0.1 M aqueous HCl (˜150 mL) with ice/water (˜300 mL). Then, 1M HCl was added until the pH was slightly acidic and the two layers separated. The organic layer was saved and then the aqueous layer was extracted twice more with chloroform (100 mL each) and then the organic layers were combined and washed once with water (˜200 mL) and once with brine (˜200 mL), dried over MgSO₄, filtered and then concentrated in vacuo to afford a thick clear oil. Silica gel chromatography (120 g) eluting with 0, 5, 10, 15, 20, 25, and 30% ethyl acetate in hexanes (˜200 mL each) afforded the purified alcohol (5), 20.7 g in 96% yield; ¹H NMR confirms; ¹H NMR (400 MHz, CDCl₃) δ ppm 4.24 (m, 1H), 3.92 (m, 1H), 3.65 (m, 2H), 3.53 (br. s., 1H), 3.46 (t, J=8.60 Hz, 1H), 3.27 (m, 1H), 1.85 (m, 1H), 1.69 (m, 1H), 1.39 (s, 9H), 0.80 (s, 9H), −0.00 (s, 3 H), −0.01 (s, 3H).

### tert-Butyl-(2S,3S)-3-{[tert-butyl(dimethyl)silyl]oxy}-2-formylpyrrolidine-1-carboxylate (6)

A 2-L 3-N round bottom flask equipped with a thermometer and addition funnel (and under an atmosphere of N₂ (g)) was charged with CH₂Cl₂ (930 mL) and then was cooled at −78° C. in a CO₂(s)-acetone bath. Then, oxalyl chloride (10.6 mL, 0.125 mol) was added and the reaction stirred an additional 5 minutes before adding dimethyl sulfoxide (17.7 mL, 0.250 mol) dropwise in portions, slow enough to maintain the reaction temperature below −65° C. After the addition was complete, the reaction was stirred an additional 10 minutes before adding tert-butyl-(2R,3S)-3-{[tert-butyl(dimethyl)silyl]oxy}-2-(hydroxymethyl)pyrrolidine-1-carboxylate (5) (20.7 g, 62.5 mmol) as a solution in CH₂Cl₂ (200 mL) dropwise—at a rate so as to keep the reaction mixture at less than −65° C. After the addition was complete, the turbid reaction mixture was stirred an additional 20 minutes and then triethylamine (34.8 mL, 0.250 mol) was added via syringe and the reaction mixture became clear and translucent. After one hour, the reaction was quenched by pouring into water and the organic product was extracted with CH₂Cl₂ (3×200 mL) and the combined organic layers washed with water (2×50 mL), 1 M HCl (1×100 mL), and brine (2×50 mL), dried over MgSO₄, filtered and concentrated in vacuo to afford the crude product. TLC of the crude reaction mixture shows the complete consumption of the starting material (R_{f} ˜0.70 in 30% ethyl acetate/hexanes) and formation of a new, higher R_{f} product (R_{f} ˜0.80 in 30% ethyl acetate/hexanes). Silica gel chromatography using a 120 g silica gel cartridge, eluting with 0 to 30% ethyl acetate in hexanes afforded the purified product (6), 17.7 g in 86% yield; ¹H NMR confirms; ¹H NMR (400 MHz, CDCl₃) δ ppm 9.49 (s, 0.4H, CHO rotamer), 9.39 (d, J=2.70 Hz, 0.6H, CHO rotamer), 4.29 (m, 1H), 4.05 (s, 0.4H, rotamer), 3.84 (t, J=2.70 Hz, 0.6H, rotamer), 3.50 (m, 2H), 1.80 (m, 2H), 1.39 (m, 4H, t-Bu rotamer), 1.34 (s, 5H, t-Bu rotamer), 0.80 (s, 9H), 0.01 (s, 3H), −0.00 (s, 3 H).

### tert-Butyl-(2R,3S)-3-hydroxy-2-(2-oxoethyl)pyrrolidine-1-carboxylate (8)

(Methoxymethyl)triphenylphos-phonium chloride (46.0 g, 0.134 mol) was suspended in tetrahydrofuran (400 mL) that had been cooled at 0° C. in an ice-water bath. Then, potassium tert-butoxide (13.9 g, 0.124 mol) was added in portions over about 5-10 minutes, and the ice bath removed and the reaction warmed to ambient temperature and stirred for 2 hr. After this period of time, the reaction was cooled at 0° C. in an ice-water bath and tert-butyl-(2S,3S)-3-{[tert-butyl(dimethyl)silyl]oxy}-2-formylpyrrolidine-1-carboxylate (6) (17.7 g, 53.7 mmol) in tetrahydrofuran (200 mL) was added dropwise via a pressure-equalizing dropping funnel over a 20 minute period of time. The resultant reaction was stirred overnight (˜14 hr) at ambient temperature and then checked for completion by TLC. TLC at this time shows complete consumption of the starting material and formation of a new, slightly higher R_{f} product (40% ethyl acetate/hexanes, R_{f} ˜0.80). The reaction was quenched by pouring into ice-water and then the organic product was extracted with ethyl acetate (2×150 mL) and the combined organic layers washed with brine, dried over MgSO₄, filtered and concentrated in vacuo to afford the crude product. The crude product (dark red) was purified by silica gel chromatography using a 90 g column eluting with 0 to 15% ethyl acetate in hexanes to afford the crude enol ether (7).

The crude enol ether (7) was dissolved in acetonitrile (200 mL) and a 5% aqueous TFA solution (100 mL) was added and the reaction was stirred with continued monitoring by TLC every 30 minutes until complete (40% ethyl acetate/hexanes; product R_{f} ˜0.60). After approximately 2 hours, the reaction was complete and was quenched by the addition of saturated aqueous sodium bicarbonate (˜200 mL). The reaction mixture was concentrated to remove the volatiles and then the product was extracted (3×100 mL) with ethyl acetate. The combined organic layers were washed with water and brine (150 mL each), dried over MgSO₄, filtered and concentrated in vacuo to afford the crude product. Silica gel chromatography using 0 to 30% ethyl acetate in hexanes afforded the purified product (8), 7.11 g in 58% yield for the two steps; ¹H NMR of the final aldehyde confirms; ¹H NMR (400 MHz, CDCl₃) δ ppm 9.72 (s, 1H), 4.06 (m, 1H), 4.01 (m, 1H), 3.52 (m, 1H), 3.33 (m, 1H), 3.01 (d, J=18.45 Hz, 0.5H), 2.79 (d, J=18.87 Hz, 0.5H), 2.37 (m, 1H), 2.10 (br. s., 1H), 1.96 (m, 1 H), 1.83 (m, 1H), 1.38 (s, 9H).

### tert-Butyl-(2R,3S)-3-hydroxy-2-prop-2-yn-1-ylpyrrolidine-1-carboxylate (9)

Dimethyl-2-oxopropylphosphonate (6.0 mL, 43 mmol) was dissolved in acetonitrile (30 mL) and then was cooled at 0° C. in an ice-water bath. Potassium carbonate (12 g, 86 mmol) was then added in one portion and the reaction was stirred for 5 minutes before adding 4-methylbenzenesulfonyl azide (8.5 g, 43 mmol) in acetonitrile (20 mL) via a pressure equalizing dropping funnel over a 10 minute period of time. After the addition was complete, the reaction was warmed to room temperature and allowed to stir for 2 hr. After this period of time, the reaction was checked for formation of the desired diazo-intermediate (TLC solvent 30% ethyl acetate/CH₂Cl₂). When the diazo-intermediate had completely formed, the reaction was cooled in a water bath and then tert-butyl-(2R,3S)-3-hydroxy-2-(2-oxoethyl)pyrrolidine-1-carboxylate (8) (6.60 g, 28.8 mmol) in CH₃OH (500 mL) was added dropwise via an addition funnel over a 20 minute period of time. The reaction was allowed to stir overnight at ambient temperature (˜10 hours) and then checked by TLC. After this period of time the starting aldehyde was consumed, and the reaction was quenched by the addition of water. The resultant mixture was concentrated to remove the volatiles and then the slurry was partitioned between ethyl acetate (150 mL) and water (100 mL). The organic product was extracted further with 2-100 mL portions of ethyl acetate and then the combined organic layers were washed with water (100 mL) and brine (100 mL), dried over MgSO₄, filtered and concentrated in vacuo to afford the crude product. Silica gel chromatography (120 g) using 0 to 20% ethyl acetate in CH₂Cl₂ afforded the purified product (9), 5.6 g in 86% yield; ¹H NMR confirms; ¹H NMR (400 MHz, CDCl₃) δ ppm 4.40 (br. s., 1H), 3.76 (m, 1H), 3.57 (m, 1H), 3.44 (m, 1H), 2.75 (d, J=16.59 Hz, 1 H), 2.64 (d, J=17.00 Hz, 1H), 2.24 (m, 1H), 2.15 (m, 1H), 2.02 (m, 1H), 1.88 (m, 1 H), 1.75 (m, 1H), 1.47 (s, 9H); HSQC (CDCl₃) coordinates: 4.42 ppm/74.83 ppm (CH); 3.82, 3.75 ppm (CH, rotamers)/64.15 ppm; 3.44, 3.59 ppm/44.23 ppm (CH₂); 2.26 ppm/21.40 ppm (CH of propargylic CH₂), 2.66 ppm, 2.76 ppm (CH of propargylic CH₂, rotamers)/21.89 ppm; 2.03 ppm/69.98 ppm (alkyne C—H); 1.98 ppm/30.63 ppm (CH of CH₂), 2.17 ppm/31.12 ppm (CH of CH₂); 1.48 ppm/27.72 ppm (t-Bu).

### tert-Butyl-(2R,3R)-3-[(tert-butoxycarbonyl)amino]-2-prop-2-yn-1-ylpyrrolidine-1-carboxylate (11)

tert-Butyl-(2R,3S)-3-hydroxy-2-prop-2-yn-1-ylpyrrolidine-1-carboxylate (9) (1.53 g, 6.79 mmol) was dissolved in CH₂Cl₂ (60 mL) and triphenylphosphine (1.78 g, 6.79 mmol) was added and then the reaction vessel was cooled in an ice-water bath. Then, diisopropyl azodicarboxylate (DIAD, 2.02 mL, 10.3 mmol) was added dropwise via syringe in 0.25 mL increments (allowing the bright orange color to dissipate between additions) and finally diphenylphosphonic azide (DPPA, 2.41 mL, 11.2 mmol) was added dropwise via syringe (in portions, as above). The resulting solution was stirred overnight (˜12 hours) and the ice bath was allowed to slowly expire. TLC analysis after this period of time shows some starting alcohol (9) and formation of a much higher R_{f} product (10), along with a few unidentified side-product. The reaction was transferred to a 250 mL separatory funnel, and diluted with ˜75 mL CH₂Cl₂ and washed twice with H₂O (˜25 mL) and once with brine (˜25 mL). Then triphenylphosphine (5.3 g, 20.0 mmol) was added to the bright yellow solution—at which time the solution became clear and colorless. The reaction mixture was then concentrated in vacuo and then taken up in tetrahydrofuran (100 mL). The reaction was checked by TLC (30% ethyl acetate:hexanes) at this time and most of the starting azide was gone and a lower-R_{f} product (R_{f} is baseline in 30% ethyl acetate:hexanes, R_{f} ˜0.15 in ˜20% methanol:CHCl₃) had formed. Then, the reaction vessel was equipped with a condenser and H₂O (15 mL, 810 mmol) was added and the reaction was heated at 55° C. for 5-6 hr. After this period of time, the reaction appeared to be complete based on TLC (baseline spot disappears and slightly higher R_{f} product forms—R_{f} 0.30 in 20% MeOH:CHCl₃). The reaction was cooled to room temperature and then di-tert-butyldicarbonate (1.8 g, 8.1 mmol) and a catalytic amount of 4-dimethylaminopyridine (83 mg, 0.68 mmol) were added and along with another 10 mL of THF to aid in solubility of all the reactants. The reaction was then allowed to stir overnight at ambient temperature. After this period of time the solvent was removed in vacuo. The crude film was taken up in ethyl acetate (˜175 mL) and washed once with water (˜25 mL) and once with brine (˜25 mL), dried over MgSO₄, filtered and concentrated in vacuo to afford the crude product. The crude material was purified using silica gel (40 g) chromatography, eluting with 0 to 25% ethyl acetate in hexanes to afford the desired compound (11), 610 mg, 29% yield for the three steps; ¹H NMR confirms; ¹H NMR (400 MHz, CDCl₃) δ ppm 4.95 (m, 1H), 4.24 (br. s., 1H), 3.96 (t, J=5.39 Hz, 1H), 3.46 (m, 1H), 3.24 (m, 1H), 2.68 (m, 1H), 2.25 (m, 1H), 2.07 (m, 2H), 1.92 (br. s., 1 H), 1.40 (s, 9H), 1.39 (s, 9H).

### Ethyl-8-(3-{(2R,3R)-1-(tert-butoxycarbonyl)-3-[(tert-butoxycarbonyl)amino]pyrrolidin-2-yl}prop-1-yn-1-yl)-1-cyclopropyl-6,7-difluoro-4-oxo-1,4-dihydroquinoline-3-carboxylate (13)

tert-Butyl-(2R,3R)-3-[(tert-butoxycarbonyl)amino]-2-prop-2-yn-1-ylpyrrolidine-1-carboxylate (11) (0.61 g, 1.9 mmol) and ethyl 1-cyclopropyl-6,7-difluoro-4-oxo-8-{[trifluoromethyl)sulfonyl]oxy}-1,4-dihydroquinoline-3-carboxylate (12) (0.83 g, 1.9 mmol) were transferred to a 50-mL, 3-neck round bottom flask equipped with a reflux condenser. Then, the reaction vessel was placed under an atmosphere of N₂ (g) by partial evacuation and then back-fill with N₂ (g) ×3. Then, anhydrous tetrahydrofuran (20 mL) was added and the reaction mixture was sparged with N₂ (g) for 2-3 minutes. Then, triphenylphosphine (0.12 g, 0.47 mmol), tetrakis(triphenylphosphine)palladium(0) (0.22 g, 0.19 mmol) and N,N-diisopropylethylamine (0.655 mL, 3.76 mmol) were added successively with continued N₂ (g) sparge for 2-3 minutes and then finally copper(I) iodide (0.12 g, 0.66 mmol) was added. The bright yellow reaction mixture was sparged with N₂ (g) an additional 2-3 minutes and then the reaction was heated at 55° C. for 12 hr (overnight) with a N₂ (g) atmosphere maintained by a balloon. After this period of time, the reaction was complete and the starting material (alkyne and triflate) had been consumed (40% ethyl acetate/CH₂Cl₂) and no triflate hydrolysis was observed based on LCMS. The reaction was cooled to room temperature and then ethanol (˜50 mL) was added, with continued stirring for 30 minutes, and then the reaction was vacuum filtered to remove the precipitated salts and the filtrate was concentrated in vacuo. Silica gel chromatography (40 g), using 0 to 40% ethyl acetate in CH₂Cl₂ afforded purified product (13), 690 mg, in 60.0% yield. ¹H NMR confirms; ¹H NMR (400 MHz, CDCl₃) δ ppm 8.54 (s, 1H), 8.17 (t, J=9.43 Hz, 1H), 4.95 (m, 1H), 4.32 (q, J=7.19 Hz, 2H), 4.28 (m, 1H), 4.06 (m, 2H), 3.45 (m, 1H), 3.26 (m, 1H), 3.10 (m, 0.5H), 2.89 (m, 0.5H), 2.58 (m, 1H), 2.12 (m, 1H), 2.04 (m, 1H), 1.39 (s, 9H), 1.34 (s, 9H), 1.33 (m, 3H), 1.19 (m, 2H), 1.08 (m, 2H); MS: ES⁺616.2 m/z (M+1) for [C₃₂H₃₉F₂N₃O₇+H]⁺; ES⁻ 614.2 m/z (M−1) for [C₃₂H₃₉F₂N₃O₇−H]⁻; HPLC retention time 8.291 min; Method A.

### Ethyl-8-[(1Z)-3-{(2R,3R)-1-(tert-butoxycarbonyl)-3-[(tert-butoxycarbonyl)amino]pyrrolidin-2-yl}prop-1-en-1-yl]-1-cyclopropyl-6,7-difluoro-4-oxo-1,4-dihydroquinoline-3-carboxylate (14)

Ethyl-8-(3-{(2R,3R)-1-(tert-butoxycarbonyl)-3-[(tert-butoxycarbonyl)amino]pyrrolidin-2-yl}prop-1-yn-1-yl)-1-cyclo-propyl-6,7-difluoro-4-oxo-1,4-dihydroquinoline-3-carboxylate (13) (0.61 g, 0.99 mmol) was placed under an atmosphere of N₂ (g) and then triethylamine (˜0.05 mL, to insure basic pH ˜8 with wet pH paper), quinoline (0.053 mL, 0.45 mmol) and ethanol (50 mL) were added followed by 5% palladium on barium sulfate (0.48 g). Then, the reaction vessel was partially evacuated with vacuum and backfilled with H₂ (g) ×3. The reaction mixture was sparged with H₂ (g) gas (˜1 L) and the maintained under an atmosphere of H₂ (g) with a balloon, overnight. After this period of time, the reaction had progressed very little (<20 area % product by HPLC). The reaction was filtered through a short pad of Celite 545 and then rinsed with chloroform. The filtrate was concentrated in vacuo and taken up in 50 mL of ethanol. The solution was placed under an atmosphere of N₂ (g) and then treated with 0.02 mL of triethylamine and 10% palladium on carbon (0.24 g) using the same evacuation and back-fill procedure noted above. After ˜4-5 hr, the reaction was complete with no apparent over-reduction product. The palladium salts were removed by filtration through a short plug of Celite 545 and concentrated in vacuo. Silica gel chromatography using a gradient of 0 to 40% ethyl acetate in CH₂Cl₂ over a 40 minute period of time afforded the purified product (14), 290 mg in 47% yield; ¹H NMR (400 MHz, CDCl₃) δ ppm 8.65 (s, 1H), 8.26 (t, J=9.33 Hz, 1H), 6.76 (d, J=11.40 Hz, 1 H), 6.09 (m, 1H), 4.45 (m, 1H), 4.40 (q, J=7.12 Hz, 2H), 4.19 (m, 1H), 4.07 (m, 1 H), 3.79 (m, 1H), 3.34 (m, 1H), 3.18 (t, J=9.74 Hz, 1H), 2.06 (m, 2H), 1.82 (m, 1 H), 1.69 (m, 1H), 1.44 (s, 9H), 1.41 (m, 3H), 1.39 (s, 9H), 1.17 (m, 2H), 0.98 (m, 2 H); MS: ES⁺618.4 m/z (M+1) for [C₃₂H₄₁F₂N₃O₇+1]⁺; 640.3 m/z (M+Na) for [C₃₂H₄₁F₂N₃O₇+23]⁺; ES⁻ 616.4 m/z for [C₃₂H₄₁F₂N₃O₇−1]⁻; HPLC retention time 8.268 min; Method A.

### Ethyl-(7aR,8R)-8-amino-4-cyclopropyl-12-fluoro-1-oxo-4,7,7a,8,9,10-hexahydro pyrrolo[1′,2′:1,7]azepino[2,3-h]quinoline-2-carboxylate (15)

Ethyl-8-[(1Z)-3-{(2R,3R)-1-(tert-butoxycarbonyl)-3-[(tert-butoxy-carbonyl)amino]pyrrolidin-2-yl}prop-1-en-1-yl]-1-cyclopropyl-6,7-difluoro-4-oxo-1,4-dihydroquinoline-3-carbox-ylate (14) (0.290 g, 0.469 mmol) was dissolved in CH₂Cl₂ (20 mL) and trifluoroacetic acid (1.5 mL) was added. The reaction was stirred overnight (˜12 hr) at ambient temperature and then checked by HPLC for completion. HPLC reveals the complete consumption of the starting material. The reaction was diluted with CHCl₃ (˜200 mL) and then washed with 25 mL of 10% aqueous NH₄OH. The aqueous layer was extracted two additional times with 10% methanol in CHCl₃. The combined organic layers were washed with brine and then dried over magnesium sulfate, filtered and concentrated in vacuo to afford a light yellow foam; HPLC retention time 4.479 min (Method A, diamine). The light yellow foam was transferred to a 50 mL round bottom flask and placed under an atmosphere of N₂ (g). Then, acetonitrile (10 mL) was added at which time the foam became a yellow precipitate; however, on addition of N,N-diisopropylethylamine (0.409 mL, 2.35 mmol) the reaction became a homogeneous translucent yellow. The reaction was stirred at ambient temperature for 2 hr and then checked by HPLC for completion. HPLC after this period of time shows that the reaction was nearly complete (˜75% area % by HPLC). The reaction was stirred overnight at ambient temperature, and HPLC after this period of time shows complete consumption of the starting material. The reaction was concentrated in vacuo and then subjected to silica gel chromatography using a 40 g silica gel cartridge, eluting from 0 to 15% methanol in chloroform in 2% intervals (˜200 mL solvent each) to afford 166 mg of (15) in 89% yield. The purified product has ¹H NMR and MS consistent with the proposed structure; ¹H NMR (400 MHz, DMSO-d₆) δ ppm 8.52 (s, 1H), 7.60 (d, J=15.13 Hz, 1H), 6.48 (d, J=12.23 Hz, 1H), 6.01 (td, J=8.09, 4.15 Hz, 1H), 4.26 (d, J=3.94 Hz, 2H), 4.11 (m, 1H), 3.83 (t, J=9.95 Hz, 2H, 2-methine H), 3.69 (m, 2H), 2.92 (br. s., 2H), 2.65 (m, 1H), 2.47 (m, 1H), 2.20 (m, 1H), 1.93 (m, 1H), 1.32 (t, J=6.95 Hz, 3H), 1.22 (m, 1H), 0.95 (m, 1H), 0.86 (m, 1H), 0.71 (m, 1H); MS ES⁺: 398.2 m/z for [C₂₂H₂₄FN₃O₃+1]⁺; HPLC retention time 5.028 min, Method A; 100 area % at 214, 254, and 280 nm.

### (7aR,8R)-8-Amino-4-cyclopropyl-12-fluoro-1-oxo-4,7,7a,8,9,10-hexahydro-1H-pyrrolo[1′,2′:1,7]azepino[2,3-h]quinoline-2-carboxylic acid trifluoroacetate (16)

Ethyl-(7aR,8R)-8-amino-4-cyclopropyl-12-fluoro-1-oxo-4,7,7a,8,9,10-hexahydro-1H-pyrrolo[1′,2′:1,7]azepino[2,3-h]quinoline-2-carboxylate (15) (53.1 mg, 0.134 mmol) was dissolved in acetonitrile (10 mL) and water (0.5 mL) and 0.35 mL of 0.500 M aqueous sodium hydroxide was added via syringe. The reaction was heated at 60° C. for 3 hr and monitored for completion by HPLC analysis. After this period of time, HPLC shows the complete consumption of the starting ester (retention time 5.028 min, Method A) and formation of a new product peak (retention time 5.093 min) with mass consistent with the desired product. The reaction was cooled to ambient temperature, acidified with glacial acetic acid (few drops to pH 5-6) and then lyophilized to afford a fine yellow powder. The powder was taken up in a 3:1 solution of water and CH₃CN and then filtered through a 13 mm syringe filter (0.45 μm PTFE, VWR) and the filtrate purified by reverse phase preparative HPLC to afford 38 mg of the desired acid (16) in 62% yield as the TFA salt after a second lyophilization; ¹H NMR (400 MHz, D₂O) δ ppm 8.67 (s, 1H), 6.91 (m, 1H), 6.20 (m, 1H), 6.00 (m, 1H), 3.92 (m, 4H), 2.47 (m, 2H), 2.36 (m, 1H), 2.11 (m, 1H), 1.22 (m, 1H), 0.92 (m, 1H), 0.79 (m, 1H), 0.64 (m, 1H); ¹H NMR (400 MHz, DMSO-d₆) δ ppm 8.76 (s, 1H), 8.41 (br. s., 3H), 7.62 (d, J=14.72 Hz, 1H), 6.73 (d, J=12.23 Hz, 1H), 6.08 (m, 1H), 4.26 (m, 1H), 4.15 (m, 1H), 4.09 (m, 1H), 3.95 (m, 1H), 3.83 (m, 1H), 2.65 (m, 1H), 2.55 (m, 1H), 2.30 (m, 1H), 2.16 (m, 1H), 1.29 (m, 1H), 1.04 (m, 1H), 0.91 (m, 1H), 0.81 (m, 1H); ES⁺370.2 m/z for [C₂₀H₂₀FN₃O₃+H]⁺; 384.2 m/z for [C₂₀H₂₀FN₃O₃+Na]⁺; HPLC retention time 5.093 min, Method A, 100 area % at 254 and 280 nm.

### Ethyl-(7aR,8R)-8-amino-4-cyclopropyl-12-fluoro-1-oxo-4,5,6,7,7a,8,9,10-octahydro-1H-pyrrolo[1′,2′:1,7]aze-pino[2,3-h]quinoline-2-carboxylate (17)

Ethyl-(7aR,8R)-8-amino-4-cyclopropyl-12-fluoro-1-oxo-4,7,7a,8,9,10-hexahydro-1H-pyrrolo[1′,2′:1,7]azepino[2,3-h]quinoline-2-carboxylate (15) (107 mg, 0.269 mmol) was dissolved in ethanol (20 mL) and was placed under an atmosphere of N₂ (g) by partial evacuation and back-filling with N₂ (g) (via balloon, ×3). Then, the reaction was sparged with N₂ (g) for 1 minute before adding 10% palladium on carbon (29 mg) and triethylamine (0.05 mL) to achieve basic pH (>8 on wet pH paper). Then, the reaction was placed under an atmosphere of H₂ (g) by partial evacuation and back-filling with H₂ (g) (via balloon, ×3). The reaction was maintained under an atmosphere of H₂ (g) by the use of a balloon and was checked for completion after 3-4 hr. HPLC after this period of time shows no progress. The reaction was treated once more with 25 mg of 10% palladium on carbon and 0.05 mL of triethylamine and maintained under an atmosphere of H₂ (g) overnight. HPLC after this period of time shows complete consumption of the starting material. The reaction was filtered through a short plug of silica gel and then rinsed with 15-20% methanol/chloroform (˜1 L) until no product eluted based on TLC (10% methanol/chloroform). Then the crude product was subjected to preparative HPLC for purification. The combined fractions were neutralized with sodium bicarbonate to pH 9 and then the organic product extracted with 5% methanol/chloroform (3×100 mL) and the combined organic layers washed with brine, dried over MgSO₄, filtered and concentrated in vacuo to afford 50.1 mg of the saturated compound (17) in 46% yield; MS: ES⁺400.2 m/z for [C₂₂H₂₆FN₃O₃+1]⁺; HPLC retention time 5.028 min, Method A, 100 area % at 254 and 280 nm.

### (7aR,8R)-8-Amino-4-cyclopropyl-12-fluoro-1-oxo-4,5,6,7,7a,8,9,10-octahydro-1H-pyrrolo[1′,2′:1,7]azepino[2,3-h]quinoline-2-carboxylic acid trifluoroacetate (18)

Ethyl-(7aR,8R)-8-amino-4-cyclopropyl-12-fluoro-1-oxo-4,5,6,7,7a,8,9,10-octahydro-1H-pyrrolo[1′,2′:1,7]azepino[2,3-h]quinoline-2-carboxylate (17) (0.050 g, 0.12 mmol) was dissolved in acetonitrile (5 mL) and water (1 mL) and then 0.500 M aqueous sodium hydroxide (0.25 mL) was added via syringe. The reaction was heated at 60° C. for 2 hr and then checked by HPLC. HPLC at this time shows little progress and no mass for the desired acid is observed. The reaction was treated with another 0.25 mL aliquot of 0.50 M aqueous sodium hydroxide with continued heating. HPLC after approximately 1 hr shows progress. The reaction was then stirred overnight (˜12 hr) at the elevated temperature. HPLC after this period of time shows consumption of the starting material and formation of the desired product. The reaction was neutralized with 1-2 drops of glacial acetic acid to pH 5-6 and then concentrated to ˜3 mL volume. The aqueous mixture was filtered through a 13 mm syringe filter (0.45 μm PTFE, VWR) and the filtrate purified by reverse phase preparative HPLC to afford 4.5 mg (18) (˜10% yield) of the TFA salt after lyophilization; ¹H NMR in D₂O is consistent for the proposed structure—the 4 acidic hydrogens have exchanged with deuterium and are not observed; ¹H NMR (400 MHz, D₂O) δ ppm 8.62 (s, 1H), 6.95 (d, J=12.65 Hz, 1H), 4.00 (m, 4H), 3.66 (m, 1H), 3.50 (m, 1H), 2.56 (m, 1H), 2.33 (m, 1H), 2.13 (m, 1H), 1.95 (m, 1H), 1.79 (m, 3H), 1.24 (m, 1H), 1.11 (m, 1H), 0.79 (m, 2H); ES⁺372.1 m/z for [C₂₀H₂₂FN₃O₃+H]⁺; HPLC retention time 5.082 min, Method A; 100 area % at 254 and 280 nm.

### Example 17

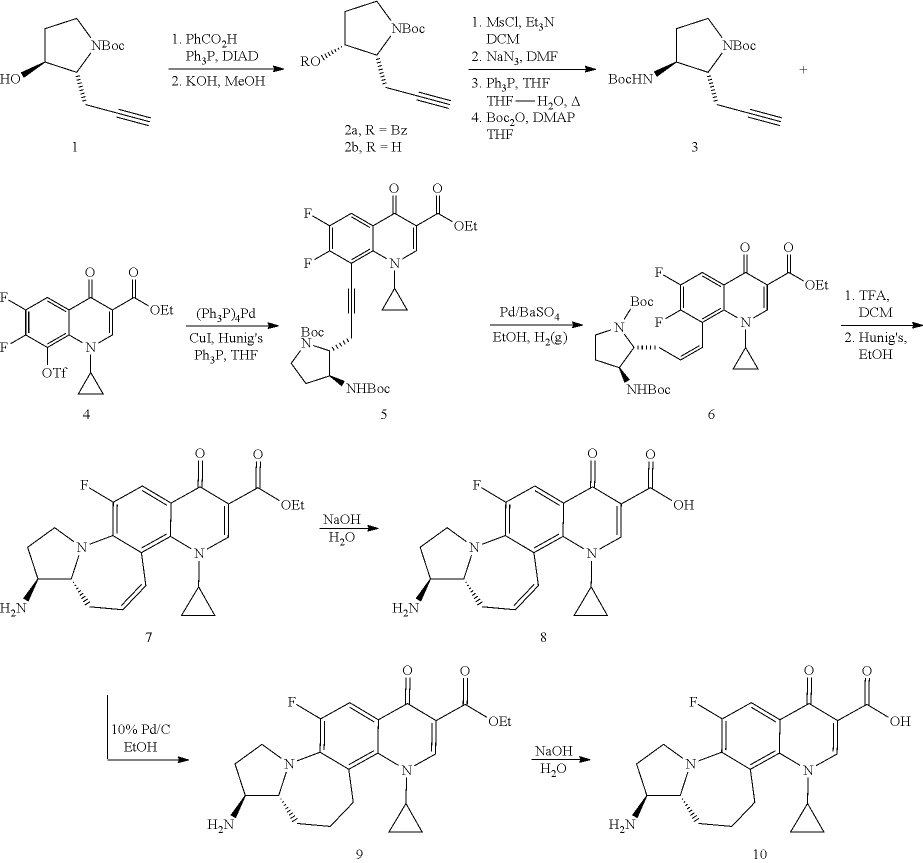

### tert-Butyl-(2R,3R)-3-(benzoyloxy)-2-prop-2-yn-1-ylpyrrolidine-1-carboxylate (2a)

tert-Butyl-(2R,3S)-3-hydroxy-2-prop-2-yn-1-ylpyrrolidine-1-carboxylate (1) (2.50 g, 11.1 mmol) was dissolved in tetrahydrofuran (101 mL) and then triphenylphosphine (3.49 g, 13.3 mmol) and benzoic acid (1.63 g, 13.3 mmol) were added at room temperature. Then, the reaction vessel was cooled in an ice-water bath and diisopropyl azodicarboxylate (2.62 mL, 13.3 mmol) was added slowly dropwise as a solution in tetrahydrofuran (20 mL) and then the ice-water bath was removed. The resulting solution was stirred for 2 hr at ambient temperature and then checked by TLC for completion. TLC at this time (50% ethyl acetate in hexanes) showed incomplete reaction. Therefore an additional 25 mol % of each reagent was added, benzoic acid (0.34 g, 2.8 mmol), triphenylphosphine (0.73 g, 2.8 mmol) and diisopropyl azodicarboxylate (0.55 mL, 2.8 mmol) and the reaction was stirred overnight. After this period of time, the reaction was complete. The reaction was concentrated to a thin film and then partitioned between water (100 mL) and ethyl acetate (50 mL) and the layers were separated. Then, the aqueous layer was washed two additional times with ethyl acetate (50 mL each). The combined organic layers were washed with brine (50 mL), dried over MgSO₄, filtered and concentrated in vacuo to afford the crude product. Silica gel chromatography using a 90 g silica gel cartridge, eluting with 0, 5, 10, 15 and 20% ethyl acetate in CH₂Cl₂ afforded the purified benzoate ester (2a), 0.92 g in low yield (25%); ¹H NMR is consistent; ¹H NMR (400 MHz, CDCl₃) δ ppm 8.03 (m, 2H), 7.52 (m, 1H), 7.39 (m, 2H), 5.55 (m, 1H), 4.13 (m, 1H), 3.48 (m, 2H), 2.75 (m, 2H), 2.19 (m, 2H), 1.80 (t, J=2.70 Hz, 1H), 1.43 (s, 9H).

### tert-Butyl-(2R,3R)-3-hydroxy-2-prop-2-yn-1-ylpyrrolidine-1-carboxylate (2b)

tert-Butyl-(2R,3R)-3-(benzoyloxy)-2-prop-2-yn-1-ylpyrrolidine-1-carboxylate (2a) (1.02 g, 3.10 mmol) was dissolved in CH₃OH (10 mL) and then cooled at 0° C. in an ice-water bath before potassium hydroxide (0.22 g, 4.0 mmol) in methanol (6 mL) was added. The reaction was stirred at reduced temperature for 2 hr and then checked by TLC for completion. TLC shows consumption of the starting benzoate ester and formation of a new, lower R_{f} product. The reaction was neutralized with 0.05 M HCl (cold) and then concentrated in vacuo and the resultant film was partitioned between ethyl acetate and water (˜50 mL:10 mL). The aqueous layer was extracted twice more with 5 mL of ethyl acetate (each time) and then the combined organic layers washed with brine, dried over MgSO₄, filtered and concentrated in vacuo to afford the crude product. Silica gel chromatography (40 g) using a gradient 0 to 35% ethyl acetate/CH₂Cl₂ over 40 min afforded purified product (2b), 0.69 g in quantitative yield. ¹H NMR (400 MHz, CDCl₃) [symbol] ppm 4.56 (q, J=5.60 Hz, 1H), 3.89 (m, 1H), 3.49 (m, 2 H), 2.97 (m, 0.5H), 2.77 (m, 0.5H), 2.55 (ddd, J=16.64, 8.97, 2.59 Hz, 1H), 2.04 (m, 3H), 1.49 (s, 9H), OH not observed.

### tert-Butyl-(2R,3S)-3-[(tert-butoxycarbonyl)amino]-2-prop-2-yn-1-ylpyrrolidine-1-carboxylate (3)

tert-Butyl-(2R,3R)-3-hydroxy-2-prop-2-yn-1-ylpyrrolidine-1-carboxylate (2b) (0.69 g, 3.1 mmol) was dissolved in CH₂Cl₂ (20 mL) and was cooled at 0° C. in an ice-water bath. Then, triethylamine (1.1 mL, 7.6 mmol) and methanesulfonyl chloride (0.31 mL, 4.0 mmol) were added successively. The resultant reaction was stirred at reduced temperature for 2 hr and then checked for completion by TLC (30% ethyl acetate/CH₂Cl₂). TLC at this time shows the complete consumption of the starting alcohol and formation of a higher R_{f} product, the mesylate. The reaction mixture was diluted with 150 mL of CH₂Cl₂, transferred to a 250 mL separatory funnel and washed once with cold 0.05 M HCl and then with brine. The organic layer was dried over MgSO₄, filtered and concentrated in vacuo to afford the crude mesylate which was treated directly with azide as outlined below.

The crude mesylate was transferred to a 100 mL round bottom flask and placed under an atmosphere of N₂ (g). Then, N,N-dimethylformamide (50 mL) was added followed by sodium azide (0.70 g, 11 mmol) and the resultant reaction mixture was stirred vigorously at 40° C. overnight and then checked after this period of time (TLC) for completion. TLC analysis after this period of time shows a small amount of progress, a higher R_{f} product is observed by TLC (30% ethyl acetate/hexanes). The reaction was heated at 55° C. for 48 hr and checked again at which time TLC shows that the reaction is ˜70% complete. An additional lot of sodium azide (0.50 g, 7.6 mmol) was added with continued stirring for ˜16 hr at 60° C. After this period of time, the reaction was ˜95% complete. The reaction was cooled to room temperature and then 50 mL of water was added to dissolve the azide salts. The organic product was extracted with diethyl ether (3×50 mL) and then the combined organic layers were washed with water and brine, dried over MgSO₄, filtered and concentrated in vacuo to afford the crude azide.

The azide was then taken up in tetrahydrofuran (50 mL) and triphenylphosphine (4 g, 20 mmol) was added and the reaction solution stirred at ambient temperature until the azide had disappeared based on TLC analysis (30% ethyl acetate/hexanes). After the azide was consumed, water (3 mL, 200 mmol) was added and the reaction was heated at 50° C. for 6-8 hr before checking. After the iminophosphorane was consumed, the reaction was cooled to ambient temperature and then di-tert-butyldicarbonate (0.80 g, 3.7 mmol) and 4-dimethylaminopyridine (0.094 g, 0.76 mmol) were added with continued stirring overnight. After this period of time, the reaction was diluted with 50 mL of water and the organic product extracted with ethyl acetate (3×50 mL). The combined organic layers were washed with water (100 mL) and brine (100 mL), dried over MgSO₄, filtered and concentrated in vacuo to afford the crude product. Silica gel chromatography using 40 g of silica and eluting with 0 to 15% ethyl acetate in hexanes afforded the purified product (3), 0.48 g in 48% yield for the five steps. The product was used directly in the next step.

### Ethyl-8-(3-{(2R,3S)-1-(tert-butoxycarbonyl)-3-[(tert-butoxycarbonyl)amino]pyrrolidin-2-yl}prop-1-yn-1-yl)-1-cyclopropyl-6,7-difluoro-4-oxo-1,4-dihydroquinoline-3-carboxylate (5)

tert-Butyl-(2R,3S)-3-[(tert-butoxy-carbonyl)amino]-2-prop-2-yn-1-ylpyrrolidine-1-carboxylate (3) (0.480 g, 1.48 mol) and ethyl-1-cyclopropyl-6,7-difluoro-4-oxo-8-{[(trifluoromethyl)sulfonyl]oxy}-1,4-dihydroquinoline-3-carboxylate (4) (0.664 g, 1.50 mmol) were transferred to a 100-mL, 3-neck round bottom flask equipped with a reflux condenser. The reaction vessel was placed under an atmosphere of N₂ (g) by partial evacuation and then back-filled with N₂ (g) three times. Then, tetrahydrofuran (20 mL) was added and the reaction mixture was sparged with N₂ (g) for 2-3 minutes. Then, triphenylphosphine (99 mg, 0.38 mmol), tetrakis(triphenylphosphine)palladium(0) (0.17 g, 0.15 mmol) and N,N-diisopropylethylamine (0.524 mL, 3.01 mmol) were added with continued sparging with N₂ (g) for 2-3 minutes and then finally copper(I) iodide (0.10 g, 0.53 mmol) was added. The bright yellow reaction mixture was sparged with N₂ (g) an additional 2-3 minutes and then the reaction was heated at 60° C. for 12 hr (overnight). After this period of time, the reaction was complete (based on TLC, 30% ethyl acetate:hexanes) with both the starting alkyne and triflate consumed—no triflate hydrolysis was evident by LCMS or TLC either. The reaction was cooled to ambient temperature and then ethanol (˜50 mL) was added, with continued stirring for 30 minutes, and then the reaction was vacuum filtered to remove the precipitated salts and the filtrate was concentrated in vacuo. Silica gel chromatography, using 0 to 30% ethyl acetate in CH₂Cl₂ afforded purified product (5), 640 mg, in 60% yield. ¹H NMR confirms; ¹H NMR (400 MHz, CDCl₃) δ ppm 8.55 (s, 1H), 8.16 (t, J=9.33 Hz, 1H), 4.60 (br. s., 1 H), 4.32 (q, J=7.05 Hz, 2H), 4.18 (m, 2H), 3.66 (m, 1H), 3.45 (m, 2H), 3.06 (m, 1 H), 2.89 (m, 1H), 2.26 (m, 1H), 1.71 (m, 1H), 1.40 (s, 9H), 1.37 (s, 9H), 1.33 (t, J=7.15 Hz, 3H), 1.24 (m, 2H), 1.03 (m, 2H); HSQC (400 MHz, CHCl₃) coordinates: 1.18 ppm/11.70 ppm, 1.42 ppm/12.94 ppm, 1.46 ppm/27.27 ppm, 1.77 ppm/29.13 ppm, 2.35 ppm/29.76 ppm, 2.95 ppm/22.91 ppm, 3.20 ppm/23.53 ppm, 3.51 ppm/44.08 ppm, 3.74 ppm/62.14 ppm, 4.25 ppm/54.66 ppm, 4.29 ppm/38.47 ppm, 4.40 ppm/60.27 ppm, 8.26 pp/n/114.44 ppm, 8.63 ppm/150.56 ppm; HPLC: 8.291 min, Method A; MS: ES⁺616.3 m/z (M+1) for [C₃₂H₃₉F₂N₃O₇+H]⁺; ES⁻ 614.2 m/z (M−1) for [C₃₂H₃₉F₂N₃O₇−H]⁻.

### Ethyl-8-[(1Z)-3-{(2R,3S)-1-(tert-butoxycarbonyl)-3-[(tert-butoxycarbonyl)amino]pyrrolidin-2-yl}prop-1-en-1-yl]-1-cyclopropyl-6,7-difluoro-4-oxo-1,4-dihydroquinoline-3-carboxylate (6)

Ethyl-8-(3-{(2R,3S)-1-(tert-butoxycarbonyl)-3-[(tert-butoxycarbonyl)amino]pyrrolidin-2-yl}prop-1-yn-1-yl)-1-cyclopropyl-6,7-difluoro-4-oxo-1,4-dihydroquinoline-3-carboxylate (5) (0.64 g, 1.0 mmol) was dissolved in ethanol (50 mL) and then quinoline (0.05 mL, 0.4 mol) and triethylamine (0.1 mL, 0.7 mmol), (to insure basic pH) were added. The reaction was partially evacuated and then backfilled with N₂ (g) (via balloon) three times before adding 0.33 g of 5% palladium on barium sulfate. After the palladium was added, the reaction vessel was partially evacuated and backfilled with H₂ (g) three times and then maintained under an atmosphere of H₂ (g) with a balloon. The reaction was stirred overnight at ambient temperature. HLPC and MS at this time show no product. Consequently, the reaction was filtered through a short plug of Celite 545 (and rinsed twice with 50 mL portions of ethanol) to remove the palladium salts and then the filtrate was transferred to a 250 mL round bottom flask and placed under an atmosphere of N₂ (g) before adding 10% palladium on carbon (0.15 g) and triethylamine (0.05 mL, 0.4 mmol). The reaction vessel was partially evacuated and back filled with H₂ (g) three times and then 2 L of H₂ (g) were bubbled through the reaction mixture and finally the reaction was maintained under an atmosphere of H₂ (g) with a balloon. The reaction was stirred for 6 hr at ambient temperature after which time HPLC and MS show complete consumption of the starting alkyne (HPLC rt=8.514 min, Method A; ES⁺MS: 616.2 m/z, M+1) and formation of the desired alkene (HPLC rt=8.195 min; Method A; ES⁺MS: 618.2 m/z, M+1). The reaction was filtered through a short plug of Celite 545 to remove the palladium salts and the filter cake wash washed with 100 mL of 10% MeOH/CHCl₃. The filtrate was concentrated in vacuo and then purified by silica gel chromatography eluting with 0 to 50% ethyl acetate/CH₂Cl₂ to afford the desired product (6), 0.40 g in 62% isolated yield. ¹H NMR confirms. ¹H NMR (400 MHz, CDCl₃) δ ppm 8.65 (s, 1 H), 8.25 (t, J=9.23 Hz, 1H), 6.85 (d, J=10.16 Hz, 1H), 6.11 (m, 1H), 4.60 (m, 1H), 4.40 (q, J=7.12 Hz, 2H), 3.76 (m, 3H), 3.34 (m, 2H), 2.21 (m, 1H), 1.87 (m, 2H), 1.68 (m, 1H), 1.44 (s, 9H), 1.44 (s, 9H), 1.42 (m, 3H), 1.19 (m, 2H), 1.00 (m, 2H); MS ES⁺618.2 m/z for [C₃₂H₄₁F₂N₃O₇+H]⁺.

### Ethyl-(7aR,8S)-8-amino-4-cyclopropyl-12-fluoro-1-oxo-4,7,7a,8,9,10-hexahydro-1H-pyrrolo[1′,2′:1,7]aze-pino[2,3-h]quinoline-2-carboxylate (7)

Ethyl-8-[(1Z)-3-{(2R,3S)-1-(tert-butoxycarbonyl)-3-[(tert-butoxy-carbonyl)amino]pyrrolidin-2-yl}prop-1-en-1-yl]-1-cyclopropyl-6,7-difluoro-4-oxo-1,4-dihydroquinoline-3-carboxylate (6) (0.40 g, 0.65 mmol) was dissolved in CH₂Cl₂ (20 mL) and trifluoroacetic acid (1.00 mL, 13.0 mmol) was added. The reaction was stirred for 14 hr at ambient temperature. After this period of time, HPLC shows complete consumption of the starting bis-Boc compound (20, HPLC retention time=8.204 min; Method A) and formation of a new, more polar product (diamine, HPLC retention time=4.419 min; Method A). The reaction was diluted in 300 mL of chloroform and then 40 mL of a 10% ammonium hydroxide solution was added. The solution was transferred to a 250 mL separatory funnel and the organic product partitioned between the two layers. The aqueous layer was washed twice more with 100-mL portions of 10% methanol/CHCl₃ and then the combined organic layers were combined. TLC analysis of the aqueous layer shows no more UV active material present. The combined organic layers were washed with brine (˜40 mL), dried over MgSO₄, filtered and concentrated in vacuo to afford the crude diamine intermediate. The intermediate was taken up in acetonitrile (10 mL) and N,N-diisopropylethylamine (0.56 mL, 3.2 mmol) was added. The reaction was stirred at ambient temperature for 6 hr and then checked for completion by HPLC. HPLC after this period of time shows that the diamine is consumed and a new, less polar compound (21, HPLC retention time=5.296 min; Method A) had formed. The reaction was concentrated in vacuo and then subjected to silica gel chromatography using a 12 g regular phase silica gel cartridge, eluting with 0 to 50% ethyl acetate in CH₂Cl₂ to afford the desired product (7), 210 mg in 82% isolated yield. ¹H NMR confirms; ¹H NMR (400 MHz, CDCl₃) δ ppm 8.49 (s, 1H), 7.63 (d, J=14.93 Hz, 1H), 6.41 (d, J=12.23 Hz, 1H), 5.86 (dt, J=12.08, 3.91 Hz, 1 H), 4.36 (m, 1H), 4.30 (m, 2H), 3.75 (m, 1H), 3.66 (m, 1H), 3.53 (d, J=9.95 Hz, 1 H), 3.38 (br. s., 1H), 2.60 (m, 1H), 2.38 (m, 1H), 2.19 (m, 3H), 1.79 (dd, J=12.65, 6.22 Hz, 1H), 1.32 (t, J=7.05 Hz, 3H), 1.17 (m, 1H), 0.91 (m, 1H), 0.82 (m, 1H), 0.69 (m, 1H); MS: ES⁺398.2 m/z for [C₂₂H₂₅F₂N₃O₃+1]⁺.

### (7aR,8S)-8-Amino-4-cyclopropyl-12-fluoro-1-oxo-4,7,7a,8,9,10-hexahydro-1H-pyrrolo[1′,2′:1,7]azepino[2,3-h]quinoline-2-carboxylic acid trifluoroacetate (8)

Ethyl-(7aR,8S)-8-amino-4-cyclopropyl-12-fluoro-1-oxo-4,7,7a,8,9,10-hexahydro-1H-pyrrolo[1′,2′:1,7]azepino[2,3-h]quinoline-2-carboxylate (7) (47.5 mg, 0.120 mmol) was dissolved in acetonitrile (5 mL) and water (1 mL) and then the reaction was placed under an atmosphere of N₂ (g). Then, 0.29 mL of 0.500 M aqueous sodium hydroxide was added and the reaction was heated at 55° C. for 6 hr. HPLC after this period of time shows consumption of the starting material (HPLC rt=5.296 min; Method A) and formation of a more polar product (HPLC rt=5.210 min; Method A). The reaction was cooled to ambient temperature and then made acidic (pH ˜5) by the addition of glacial acetic acid (added dropwise), and the solvent was removed by lyophilization. The lyophilized product was taken up in water (˜5-6 mL) and then filtered through Grade 1 Whatman filter paper and the filtrate was subjected to preparative HPLC purification to afford the desired TFA salt, 20.0 mg (8) in 35% isolated yield; ¹H NMR is consistent; ¹H NMR (400 MHz, DMSO-d₆) δ ppm 8.75 (s, 1 H), 8.26 (br. s., 3H), 7.62 (d, J=14.5 Hz, 1H), 6.68 (d, J=12.2 Hz, 1H), 6.00 (dt, J=12.2, 3.7 Hz, 1H), 4.35 (m, 1H), 4.25 (m, 1H), 3.95 (dd, J=9.3, 1.8 Hz, 1H), 3.87 (m, 1H), 3.72 (d, J=4.7 Hz, 1H), 2.63 (m, 2H), 2.42 (m, 1H), 2.12 (dd, J=14.0, 6.1 Hz, 1H), 1.29 (m, 1H), 1.07 (m, 1H), 0.96 (m, 1H), 0.74 (m, 1H); HSQC coordinates: 0.75/9.83 ppm, 0.94/9.83 ppm, 1.07/12.94 ppm, 1.28/12.94 ppm, 2.12/28.51 ppm, 2.42/28.51 ppm, 2.50/40.02 ppm, 2.65/35.36 ppm, 3.72/54.44 ppm, 3.86/48.44 ppm, 3.97/68.99 ppm, 4.24/41.16 ppm, 4.35/48.44 ppm, 6.00/126.3 ppm, 6.67/126.9 ppm, 7.60/108.2 ppm, 7.63/108.5 ppm, 8.75/151.7 ppm; MS: 370.2 m/z (M+1) for [C₂₀H₂₀FN₃O₃+1]⁺.

### Ethyl-(7aR,8S)-8-amino-4-cyclopropyl-12-fluoro-1-oxo-4,5,6,7,7a,8,9,10-octahydro-1H-pyrrolo[1′,2′:1,7]aze-pino[2,3-h]quinoline-2-carboxylate (9)

Ethyl-(7aR,8S)-8-amino-4-cyclopropyl-12-fluoro-1-oxo-4,7,7a,8,9,10-hexahydro-1H-pyrrolo[1′,2′:1,7]azepino[2,3-h]quinoline-2-carboxylate (7) (176 mg, 0.443 mmol) was dissolved in ethanol (10 mL) and was placed under an atmosphere of N₂ (g) by partial evacuation and back-fill with N₂ (g) (×3). Then, 10% palladium on carbon (0.12 g) and triethylamine (0.03 mL, 0.2 mmol, to maintain a basic reaction mixture) were added and the reaction was placed under an atmosphere of H₂ (g) by partial evacuation and back-fill with H₂ (g) (×3). The reaction mixture was sparged with H₂ (g) (2 L) and then maintained under an atmosphere of H₂ (g) with a balloon. After ˜6 hr, the reaction was checked by HPLC and found to be −30% complete. The reaction atmosphere was exchanged with N₂ (g) by partial evacuation and back-fill with N₂ (g) before charging the reaction vessel with an additional lot of 10% palladium on carbon (55 mg) and triethylamine (0.10 mL, 0.72 mmol) and then the H₂ (g) atmosphere was restored by partial evacuation and backfill with H₂ (g) gas (as above), the reaction was sparged with 2-1 L balloons filled with H₂ (g) and then maintained under an atmosphere of H₂ (g) with a balloon. The reaction was stirred for 10 hr and then checked by HPLC. HPLC at this period of time shows consumption of the starting olefin (HPLC retention time=5.297 min; Method A) and formation of a new peak. The reaction was filtered through a short plug of Celite 545 to remove the palladium salts and then concentrated in vacuo and (9) was used directly in the next step without further purification. HPLC retention time=5.149 min (Method A); MS ES⁺: 400.2 m/z M+1 for [C₂₂H₂₆FN₃O₃+1]⁺.

### (7aR,8S)-8-Amino-4-cyclopropyl-12-fluoro-1-oxo-4,5,6,7,7a,8,9,10-octahydro-1H-pyrrolo[1′,2′:1,7]azepino-[2,3-h]quinoline-2-carboxylic acid trifluoroacetate (10)

Ethyl-(7aR,8S)-8-amino-4-cyclopropyl-12-fluoro-1-oxo-4,5,6,7,7a,8,9,10-octahydro-1H-pyrrolo[1′,2′:1,7]azepino[2,3-h]quinoline-2-carboxylate (9) (0.177 g, 0.443 mmol) was dissolved in acetonitrile (10 mL) and water (1.0 mL) and then 1.1 mL of 0.500 M aqueous sodium hydroxide was added. The reaction was heated at 60° C. for 5 hr and then checked by HPLC at which time the reaction was determined to be complete. The reaction was neutralized with glacial acetic acid (dropwise) to pH 5 and then the solvent removed by lyophilization. The lyophilized sample was taken up in water/CH₃CN, filtered, and then subjected to preparative HPLC purification to afford ˜25 mg (10) (12% isolated) after lyophilization of the pure fractions; ¹H NMR is consistent; HPLC retention time 5.143 min (Method A); ¹H NMR (400 MHz, DMSO-d₆) δ ppm 8.74 (s, 1H), 8.04 (br. s., 3H), 7.68 (d, J=14.31 Hz, 1H), 4.24 (m, 1H), 4.18 (m, 1H), 3.93 (m, 1H), 3.72 (m, 1H), 3.58 (m, 3H), 3.42 (br. s., 1H), 2.75 (dd, J=15.45, 7.98 Hz, 1H), 2.41 (m, 1H), 2.06 (dd, J=13.99, 6.95 Hz, 1H), 1.91 (m, 1H), 1.80 (m, 1H), 1.68 (m, 1H), 1.26 (m, 1H), 1.14 (m, 1H), 1.04 (m, 1H), 0.67 (m, 1H); MS ES⁺: 372.2 m/z M+1 for [C₂₀H₂₂FN₃O₃+1]⁺.

### Example 18

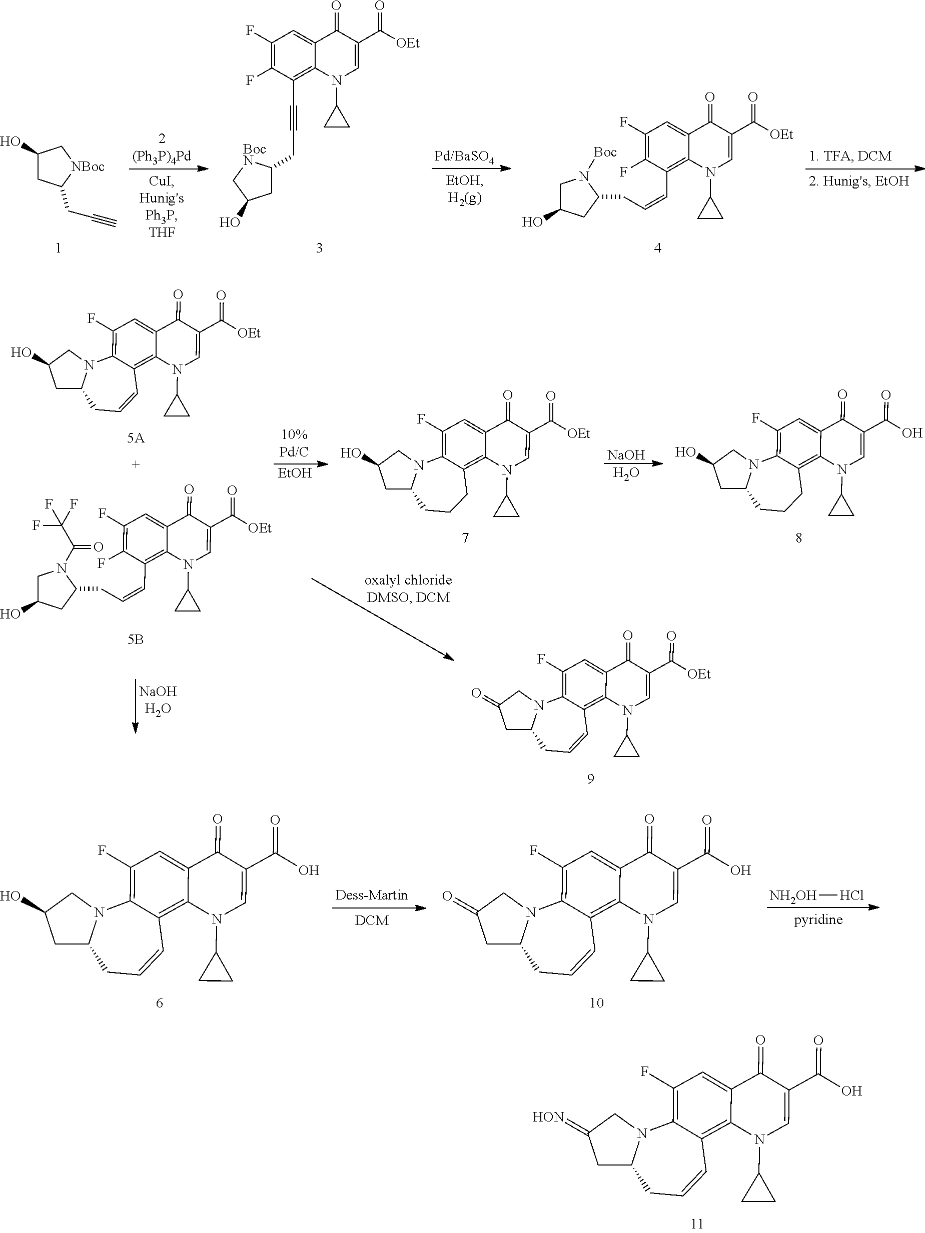

### Ethyl-8-{3-[(2R,4R)-1-(tert-butoxycarbonyl)-4-hydroxypyrrolidin-2-yl]prop-1-yn-1-yl}-1-cyclopropyl-6,7-difluoro-4-oxo-1,4-dihydroquinoline-3-carboxylate (3)

tert-Butyl-(2R,4R)-4-hydroxy-2-prop-2-yn-1-ylpyrroli-dine-1-carboxylate (1) (1.52 g, 6.75 mmol) and ethyl-1-cyclopropyl-6,7-difluoro-4-oxo-8-{[(trifluoromethyl)-sulfonyl]oxy}-1,4-dihydroquinoline-3-carboxylate (2) (3.03 g, 6.86 mmol) were transferred to a 250 mL, 3-neck round bottom flask and then placed under an atmosphere of N₂ (g) by partial evacuation and then back-fill with N₂ (g) three times. Then, tetrahydrofuran (70 mL) was added and the reaction mixture was sparged with N₂ (g) for 2-3 minutes. Then, triphenylphosphine (450 mg, 1.7 mmol), tetrakis(triphenylphosphine)palladium(0) (0.79 g, 0.68 mmol) and N,N-diisopropylethylamine (2.39 mL, 13.7 mmol) were added with continued sparging (N₂) for 2-3 minutes and then finally copper(I) iodide (0.46 g, 2.4 mmol) was added. The bright yellow reaction mixture was sparged with N₂ (g) an additional 2-3 minutes and then the reaction was heated at 60° C. for 12 hr (overnight). After this period of time, the reaction was complete and the starting material (8A and 12) had been consumed. No triflate hydrolysis was evident by HPLC or TLC (40% ethyl acetate:hexanes). The reaction was cooled to room temperature and then ethanol (˜50 mL) was added, with continued stirring for 30 minutes, and then the reaction was vacuum filtered to remove the precipitated salts and the filtrate was concentrated in vacuo. Silica gel chromatography, using 0 to 50% ethyl acetate in CH₂Cl₂ (1.5 hr gradient) afforded purified product (3), 2.64 g, in 76% yield; ¹H NMR confirms; HPLC: 7.364 min (Method A); ¹H NMR (400 MHz, CDCl₃) δ ppm 8.54 (s, 1H), 8.16 (t, J=9.43 Hz, 1 H), 4.44 (m, 1H), 4.32 (q, J=7.19 Hz, 2H), 4.09 (m, 2H), 3.64-3.42 (m, 2H), 3.12-2.73 (m, 2H), 2.14 (dd, J=7.77, 3.84 Hz, 2H), 1.64 (br. s., 1H), 1.40 (s, 9H), 1.33 (t, J=7.15 Hz, 3H), 1.20 (m, 2H), 1.05 (m, 2H); MS: ES⁺517.1 m/z (M+1) for [C₂₇H₃₀F₂N₂O₆+H]⁺.

### Ethyl-8-{(1Z)-3-[(2R,4R)-1-(tert-butoxycarbonyl)-4-hydroxypyrrolidin-2-yl]prop-1-en-1-yl}-1-cyclopropyl-6,7-difluoro-4-oxo-1,4-dihydroquinoline-3-carboxylate (4)

Ethyl-8-{3-[(2R,4R)-1-(tert-butoxycarbonyl)-4-hydroxypyrrolidin-2-yl]prop-1-yn-1-yl}-1-cyclopropyl-6,7-difluoro-4-oxo-1,4-dihydroquinoline-3-carboxylate (3) (0.871 g, 1.69 mmol) was dissolved in ethanol (100 mL) and the reaction was placed under an atmosphere of N₂ (g) by partial evacuation and back fill with N₂ (g) three times. Then, 5% palladium on barium sulfate, (0.36 g), quinoline (0.040 mL, 0.34 mmol) and triethylamine (0.059 mL, 0.42 mmol) were added successively and the reaction mixture was placed under an atmosphere of H₂ (g) by partial evacuation and then back fill with H₂ (g) three times. Then, the reaction vessel was sparged with H₂ (g) gas using 2-1 L H₂ (g) balloons and then maintained under an atmosphere of H₂ (g) with a H₂ (g) filled balloon. The reaction was checked after ˜8 hr and HPLC showed no progress. The reaction atmosphere was exchanged for N₂ (g) and then the reaction mixture was filtered through a short plug of Celite 545 and the filter cake was washed with 50 mL of ethanol (in 10 mL portions). Then, the filtrate was sparged with N₂ (g) and the reaction vessel placed under an atmosphere of N₂ (g) by partial evacuation and back fill with N₂ (g) (via balloon as above). Then, 10% palladium on carbon (150 mg) and another aliquot of triethylamine (0.10 mL, 0.72 mmol) was added. The reaction was placed under an atmosphere of H₂ (g) by partial evacuation and back fill with H₂ (g) (via balloon) and then sparged with 2-1 L H₂ (g) balloons. The reaction was maintained under an atmosphere of H₂ (g) with a balloon and allowed to stir overnight (˜12 hr). HPLC after this period of time shows consumption of the starting alkyne (HPLC rt=7.359 min; (Method A); MS: 517.1 m/z, M+1 for [C₂₇H₃₀F₂N₂O₆+1]⁺) and formation of a new slightly more polar product (HPLC rt=7.020 min; (Method A); MS: 519.2 m/z M+1 for [C₂₇H₃₂F₂N₂O₆+1]⁺. No evidence of over-reduction product is noted. The reaction atmosphere was exchanged for N₂ (g) and then filtered through a short plug of Celite 545 and the filter cake rinsed with several 20 mL portions of 5% methanol/chloroform. The filtrate was concentrated in vacuo and then subjected to silica gel chromatography (40 g silica gel) eluting with 0 to 50% ethyl acetate in CH₂Cl₂ to afford the desired product (4), 0.61 g in 70% isolated yield; ¹H NMR confirms; ¹H NMR (400 MHz, CDCl₃) δ ppm 8.64 (s, 1H), 8.26 (t, J=9.43 Hz, 1H), 6.83 (d, J=11.61 Hz, 1H), 5.96 (dt, J=11.20, 7.15 Hz, 1H), 4.41 (q, J=7.19 Hz, 2H), 4.30 (m, 1H), 4.03 (m, 1H), 3.74 (m, 1H), 3.42 (m, 1H), 3.31 (dd, J=12.02, 4.35 Hz, 1H), 2.37 (m, 1H), 1.99 (m, 2H), 1.63 (s, 1H), 1.61 (m, 1H), 1.42 (m, 9H), 1.43 (t, J=7.05 Hz, 3H), 1.17 (br. s., 2H), 0.96 (m, 2H).

### Ethyl-(7aR,9R)-4-cyclopropyl-12-fluoro-9-hydroxy-1-oxo-4,7,7a,8,9,10-hexahydro-1H-pyrrolo[1′,2′:1,7]aze-pino[2,3-h]quinoline-2-carboxylate (5A) and ethyl-1-cyclopropyl-6,7-difluoro-8-{(1Z)-3-[(2R,4R)-4-hydroxy-1-(trifluoroacetyl)pyrrolidin-2-yl]prop-1-en-1-yl}-4-oxo-1,4-dihydroquinoline-3-carboxylate (5B)

Ethyl-8-{(1Z)-3-[(2R,4R)-1-(tert-butoxycarbonyl)-4-hydroxypyrrolidin-2-yl]prop-1-en-1-yl}-1-cyclopropyl-6,7-difluoro-4-oxo-1,4-dihydroquinoline-3-carboxylate (4) (0.61 g, 1.2 mmol) was dissolved in CH₂Cl₂ (15 mL) and trifluoroacetic acid (2.0 mL, 0.026 mol) was added. The reaction was stirred at ambient temperature for 8 hr after which time HPLC shows consumption of the starting N-Boc compound (HPLC retention time=7.020 min; (Method A)) and formation of a slightly lower R_{f} product (HPLC retention time=5.095 min; (Method A)). The reaction was diluted with CHCl₃ (˜200 mL) and then ˜40 mL of 10% aqueous NH₄OH was added. The product was partitioned between the two layers and then the layers were separated. The aqueous layer was extracted (×3, 100 mL) with 5% methanol in chloroform. Then the combined organic layers were washed with brine (50 mL), dried over MgSO₄, filtered and concentrated in vacuo to afford the crude product (MS: 399.2 m/z for [C₂₂H₂₄F₂N₂O₄−F]⁺ and 418.2 m/z for [C₂₂H₂₄F₂N₂O₄+1]⁺) that was used directly in the next step.

The crude amino alcohol was suspended in acetonitrile (20 mL) and then N,N-diisopropylethylamine (2.0 mL, 11 mmol) was added at which time the reaction became homogeneous. The reaction was stirred for 2 hr at ambient temperature and then checked for completion by HPLC. At this point, the reaction was not complete. An additional lot of N,N-diisopropylethylamine (2.0 mL, 11 mmol) was added with continued heating and stirring overnight (˜14 hr). HPLC after this period of time shows the complete consumption of the intermediate and formation of a new, less polar product. The reaction was concentrated in vacuo and then purified by silica gel chromatography (40 g) eluting with 0 to 15% CH₃OH/CHCl₃ to afford the purified product (5A), 394 mg, 84% yield; ¹H NMR is consistent; HPLC retention time=6.290 min (Method A); ¹H NMR (400 MHz, CDCl₃, 27A) δ ppm 8.51 (s, 1H), 7.68 (d, J=14.93 Hz, 1H), 6.45 (d, J=12.44 Hz, 1H), 5.86 (dt, J=12.18, 3.96 Hz, 1H), 4.54 (m, 1H), 4.31 (m, 2H), 4.03 (m, 1H), 3.87 (m, 1H), 3.77 (m, 1H), 3.69 (m, 1H), 2.50 (m, 2H), 2.20 (m, 1H), 1.95 (m, 1H), 1.65 (br. s., 1H), 1.33 (t, J=7.15 Hz, 3H), 1.19 (m, 1H), 0.89 (m, 2H), 0.70 (m, 1H); MS: ES⁺399.1 m/z for [C₂₂H₂₃FN₂O₄+1]⁺. On larger scale, another product was also observed, which is believed to be 5B based on ¹H NMR and MS data; ¹H NMR (400 MHz, CDCl₃, 27B) δ ppm 8.54 (s, 1H), 8.18 (t, J=9.33 Hz, 1H), 6.86 (d, J=11.20 Hz, 1H), 5.83 (dt, J=11.20, 7.26 Hz, 1H), 4.76 (m, 1 H), 4.32 (q, J=7.05 Hz, 2H), 4.05 (m, 1H), 3.79 (d, J=18.66 Hz, 1H), 3.61 (m, 1H), 2.71 (dd, J=18.56, 9.64 Hz, 1H), 2.23 (m, 2H), 2.10 (m, 1H), 1.58 (br. s., 2H), 1.34 (t, J=7.15 Hz, 3H), 1.19 (m, 1H), 1.11 (m, 1H), 0.93 (m, 2H); MS ES⁺515.1 m/z M+1 for [C₂₄H₂₃F₅N₂O₅+H]⁺.

### (7aR,9R)-4-Cyclopropyl-12-fluoro-9-hydroxy-1-oxo-4,7,7a,8,9,10-hexahydro-1H-pyrrolo[1′,2′:1,7]azepino-[2,3-h]quinoline-2-carboxylic acid (6)

Ethyl-(7aR,9R)-4-cyclopropyl-12-fluoro-9-hydroxy-1-oxo-4,7,7a,8,9,10-hexahydro-1H-pyrrolo[1′,2′:1,7]azepino[2,3-h]quinoline-2-carboxylate (5A, 78.9 mg, 0.198 mmol) was dissolved in acetonitrile (10 mL) and water (3 mL) and then 0.87 mL of 0.500 M aqueous sodium hydroxide was added. The reaction was heated at 60° C. for 4 hr and then checked by HPLC. HPLC after this period of time shows consumption of the starting material and formation of a new product that is the desired carboxylic acid based on MS (ES⁺371.2 m/z for [C₂₀H₁₉FN₂O₄+H]⁺); HPLC retention time 6.193 min; (Method A). The reaction was removed from heat and then neutralized to pH ˜5 with acetic acid (added dropwise) and then the solvent was removed by lyophilization. The lyophilized product was taken up in water and filtered to remove water soluble salts. The solid was dried for 24 hr on high vacuum to afford 56.2 mg of (6) in 77% yield; HPLC is 100 area % at 214, 254, and 280 nm. NMR (400 MHz, CDCl₃) δ ppm 8.76 (s, 1H), 7.65 (d, J=14.31 Hz, 1H), 6.47 (d, J=12.02 Hz, 1H), 5.92 (m, 1H), 4.59 (m, 1H), 4.12 (m, 1H), 3.92 (m, 2H), 3.76 (m, 1H), 2.56 (m, 2H), 2.22 (m, 1H), 1.99 (m, 1H), 1.59 (br. s., 2H), 1.27 (m, 1H), 0.96 (m, 2H), 0.78 (m, 1H).

### Ethyl-(7aR,9R)-4-cyclopropyl-12-fluoro-9-hydroxy-1-oxo-4,5,6,7,7a,8,9,10-octahydro-1H-pyrrolo[1′,2′:1,7]-azepino[2,3-h]quinoline-2-carboxylate (7)

Ethyl-(7aR,9R)-4-cyclopropyl-12-fluoro-9-hydroxy-1-oxo-4,7,7a,8,9,10-hexahydro-1H-pyrrolo[1′,2′:1,7]azepino[2,3-h]quinoline-2-carboxylate (5A) (294.9 mg, 0.7402 mmol) under an atmosphere of N₂ (g) and then was dissolved in ethanol (20 mL). Then, the reaction flask was partially evacuated and backfilled with N₂ (g) three times before adding triethylamine (0.05 mL, 0.4 mmol) and 10% palladium on carbon (79 mg). The reaction was then partially evacuated and backfilled with H₂ (g) three times, sparged with two 1 L H₂ (g) balloons and then maintained under an atmosphere of H₂ (g) with a H₂ (g) filled balloon. The reaction was stirred vigorously at ambient pressure and then checked for completion after 8 hr. HPLC after this period of time shows consumption of the starting olefin (HPLC retention time 6.290 min; Method A) and formation of a slightly less polar product (HPLC retention time 6.426 min; Method A). MS confirms formation of the desired saturated product; ES⁺401.2 m/z (M+1) for [C₂₂H₂₅FN₂O₄+1]⁺. The reaction mixture was filtered through a short plug of Celite 545 and rinsed several times (5×20 mL) with 5% CH₃OH in CHCl₃. The filtrated was concentrated in vacuo and then subjected to silica gel chromatography, (40 g silica gel), eluting with 0 to 10% CH₃OH in CHCl₃ with a 1% gradient over 1.25 hr in 20 mL fractions. Concentration of the fractions containing product afforded 275 mg of (7) in 93% yield; ¹H NMR confirms; ¹H NMR (400 MHz, CHCl₃) δ ppm 8.53 (s, 1H), 7.81 (d, J=14.31 Hz, 1H), 4.46 (m, 1H), 4.31 (m, 2H), 3.93 (m, 1H), 3.85 (m, 1H), 3.57 (ddd, J=11.25, 4.30, 2.70 Hz, 1H), 3.46 (m, 2H), 2.42 (dd, J=13.99, 10.47 Hz, 1H), 2.21 (m, 1H), 1.99 (m, 1H), 1.84 (m, 2H), 1.60 (m, 2H), 1.46 (m, 1H), 1.33 (t, J=7.15 Hz, 3H), 1.17 (m, 1H), 1.02 (m, 1H), 0.87 (m, 1H), 0.65 (m, 1H).

### (7aR,9R)-4-Cyclopropyl-12-fluoro-9-hydroxy-1-oxo-4,5,6,7,7a,8,9,10-octahydro-1H-pyrrolo-[1′,2′:1,7]aze-pino[2,3-h]quinoline-2-carboxylic acid (8)

Ethyl-(7aR,9R)-4-cyclopropyl-12-fluoro-9-hydroxy-1-oxo-4,5,6,7,7a,8,9,10-octahydro-1H-pyrrolo[1′,2′:1,7]azepino[2,3-h]quinoline-2-carboxylate (7) (72.9 mg, 0.182 mmol) was dissolved in acetonitrile (9 mL) and water (3 mL) and then 0.500 M of aqueous sodium hydroxide in water (0.80 mL) was added. The reaction was heated at 60° C. for 4 hr and then checked by HPLC. HPLC after this period of time shows ˜70% conversion to the desired acid. The reaction was stirred overnight at 45° C. and then checked again. HPLC after this period of time shows consumption of the starting material and complete conversion to the desired acid. The reaction was neutralized to pH 5 with dropwise addition of acetic acid and then the solvent removed by lyophilization. The lyophilized product was taken up in water and filtered to remove water soluble salts and then dried overnight on high vacuum. HPLC and ¹H NMR after this period of time are clean and consistent with the desired product (8) (40 mg in 60% yield); HPLC retention time 6.325 min; Method A; ¹H NMR (400 MHz, CDCl₃) δ ppm 15.02 (s, 1H), 8.83 (s, 1H), 7.84 (d, J=14.10 Hz, 1H), 4.60 (m, 1H), 4.04 (m, 2H), 3.76 (m, 2H), 3.59 (dd, J=14.82, 9.02 Hz, 1H), 2.55 (dd, J=14.41, 9.85 Hz, 1H), 2.29 (m, 1H), 2.09 (m, 1H), 1.98 (m, 2H), 1.76 (m, 1H), 1.64 (m, 1H), 1.34 (m, 1H), 1.18 (m, 1H), 1.02 (m, 1H), 0.83 (m, 1H); MS: 373.2 m/z for [C₂₀H₂₁FN₂O₄+H]⁺.

### Ethyl-(7aR)-4-cyclopropyl-12-fluoro-1,9-dioxo-4,7,7a, 8,9,10-hexahydro-1H-pyrrolo[1′,2′:1,7]aze-pino[2,3-h]quinoline-2-carboxylate (9)

Methylene chloride (10 mL) was cooled at −78° C. using a dry ice/acetone cold bath. After cooling for 10 minutes, oxalyl chloride (0.106 mL, 1.25 mmol) was added and then dimethyl sulfoxide (0.178 mL, 2.51 mmol) was added dropwise via syringe over a 5 minute period of time. The reaction was stirred for 30 minutes at reduced temperature and then ethyl-(7aR,9R)-4-cyclopropyl-12-fluoro-9-hydroxy-1-oxo-4,7,7a,8,9,10-hexahydro-1H-pyrrolo[1′,2′:1,7]azepino[2,3-h]quinoline-2-carboxylate (5A) (250.0 mg, 0.6275 mmol) in CH₂Cl₂ (2 mL) was added dropwise via syringe. The reaction was stirred for 1 hr at reduced temperature and then triethylamine (0.350 mL, 2.51 mmol) was added dropwise via syringe over a 2-3 minute period of time and the reaction was stirred at reduced temperature for 30 minutes and the cold bath allowed to slowly expire with continued stirring overnight. HPLC analysis at this time shows consumption of the alcohol and formation of a new product. The reaction was quenched by the addition of water and the organic product extracted with ethyl acetate (3×100 mL). The combined organic layers were washed with water and brine, dried over MgSO₄, filtered and concentrated in vacuo to afford the crude product. Purification using preparative HPLC afforded the desired product (9) (50 mg, 20% yield), ¹H NMR (400 MHz, CDCl₃) δ ppm 8.56 (s, 1H), 7.82 (d, J=13.68 Hz, 1H), 6.66 (d, J=12.02 Hz, 1H), 5.98 (dt, J=11.87, 4.64 Hz, 1H), 4.62 (d, J=19.49 Hz, 1 H), 4.32 (m, 2H), 4.26 (m, 1H), 3.79 (m, 1H), 3.62 (d, J=19.49 Hz, 1H), 2.85 (dd, J=17.41, 8.09 Hz, 1H), 2.63 (m, 1H), 2.51 (m, 1H), 2.39 (m, 1H), 1.34 (t, J=7.15 Hz, 3H), 1.22 (m, 1H), 0.91 (m, 2H), 0.72 (m, 1H); MS ES⁺397.1 m/z M+1 for [C₂₂H₂₁FN₂O₄+H]⁺.

### (7aR)-4-Cyclopropyl-12-fluoro-1,9-dioxo-4,7,7a,8,9,10-hexahydro-1H-pyrrolo[1′,2′:1,7]azepino[2,3-h]quino-line-2-carboxylic acid (10)

(7aR,9R)-4-Cyclopropyl-12-fluoro-9-hydroxy-1-oxo-4,7,7a,8,9,10-hexahydro-1H-pyrrolo[1′,2′:1,7]azepino[2,3-h]quinoline-2-carboxylic acid (6) (37.5 mg, 0.101 mmol) was dissolved in CH₂Cl₂ (9.0 mL) and then Dess-Martin periodinane (49.2 mg, 0.116 mmol) was added with continued stirring for 1.5 hr before checking by HPLC/TLC for completion. HPLC after this period of time shows that the reaction is complete and a new, major product is formed, HPLC rt=6.546 min (Method A). The reaction was quenched with saturated aqueous sodium bisulfate and then the organic product was extracted with chloroform (3×50 mL) and the combined organic layers washed with brine (25 mL), dried over sodium sulfate, filtered and concentrated in vacuo to afford the crude product. The crude material was purified on a 12 g reverse phase C-18 silica gel column, eluting with 0 to 30% acetonitrile in water (1 hr gradient in 5% increments with 20 mL fractions) to afford the desired keto-acid (10), 15 mg, in low yield 40%). ¹H NMR and MS are consistent; ¹H NMR (400 MHz, CDCl₃) δ ppm 8.91 (s, 1H), 7.89 (d, J=13.48 Hz, 1H), 6.74 (d, J=12.23 Hz, 1H), 6.10 (m, 1H), 4.79 (d, J=19.49 Hz, 1H), 4.38 (m, 1H), 4.02 (m, 1H), 3.77 (d, J=19.28 Hz, 1H), 2.99 (dd, J=17.41, 8.29 Hz, 1H), 2.74 (m, 1H), 2.65 (m, 1H), 2.49 (dd, J=17.52, 1.97 Hz, 1H); ES⁺369.11 m/z for [C₂₀H₁₇FN₂O₄+H]⁺; ES⁻ 367.10 m/z for [C₂₀H₁₇FN₂O₄−H]⁻; HPLC: 6.546 min; 214 nm (94 area %), 250 nm (98 area %), and 280 nm (100 area %); (Method A).

### (7aR,9E)-4-Cyclopropyl-12-fluoro-9-(hydroxyimino)-1-oxo-4,7,7a,8,9,10-hexahydro-1H-pyrrolo[1′,2′:1,7]aze-pino[2,3-h]quinoline-2-carboxylic acid (11)

(7aR)-4-Cyclopropyl-12-fluoro-1,9-dioxo-4,7,7a,8,9,10-hexahydro-1H-pyrrolo[1′,2′:1,7]azepino[2,3-h]quinoline-2-carboxylic acid (10) (80 mg, 0.2 mmol) was taken up into pyridine (3.6 mL) and then hydroxylamine hydrochloride (237 mg, 3.41 mmol) was added in one portion at room temperature. The reaction mixture was stirred for 30 min at which point LCMS indicated that the reaction was complete. Water (3.6 mL) was added followed by sodium bicarbonate (286 mg, 3.41 mmol) at which time, pH of the yellow solution was 7-8 by Merck pH paper. The reaction mixture was frozen and then the solvent removed by lyophilization to afford a cream colored fluffy solid. The crude product was purified by reverse phase chromatography using a 40 g reverse phase C18 cartridge, eluting with 0 to 75% acetonitrile in 5% increments, eluting for 10 minutes at each step, to afford the desired oxime (11) (15 mg) in low yield (˜15%). The side product in this reaction is some of the alcohol and some of the N-oxide—these were removed by careful chromatography; ¹H NMR (400 MHz, D₂O) δ ppm 9.20 (br. s., 1H), 8.08 (d, J=13.06 Hz, 1H), 7.12 (br. s., 1H), 6.48 (br. s., 1H), 5.35 (m, 1H), 4.52 (m, 3H), 3.35 (m, 1H), 3.02 (m, 3H), 1.70 (m, 1H), 1.39 (m, 2H), 1.20 (m, 1H); ES⁺384.08 m/z for [C₂₀H₁₈FN₃O₄+H⁺]⁺; ES⁻ 382.09 m/z for [C₂₀H₁₈FN₃O₄−H⁺]⁻; HPLC: 6.103, 6.110 min (E- and Z-oxime diastereomers) 214 nm (100%), 250 nm (100%), and 280 nm (100%) (Method A).

### Example 19

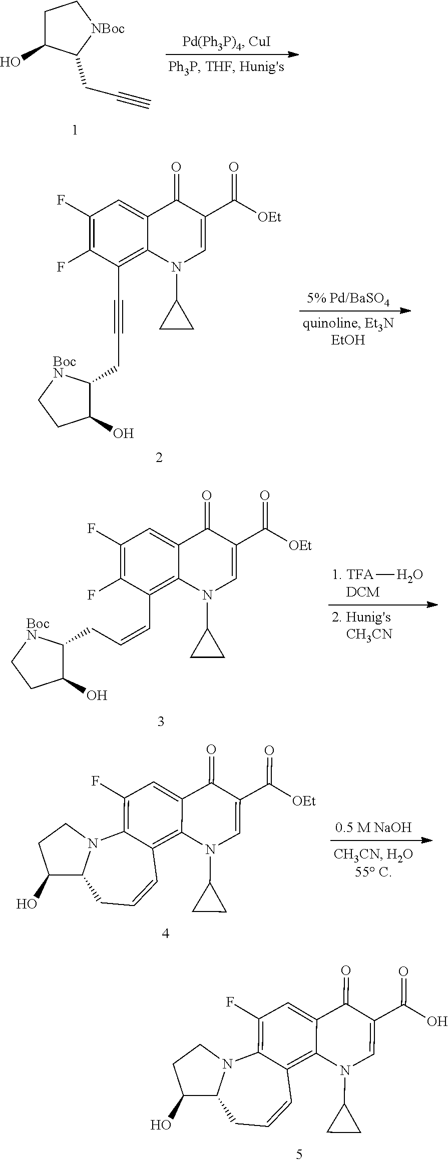

All reactions were performed under an atmosphere of nitrogen. Unless otherwise indicated, the reaction flask was evacuated with vacuum and then back-filled with nitrogen via a balloon (×3) and the reaction kept under nitrogen via balloon for the duration of the reaction.

Analytical HPLC was performed using an Agilent 1100 HPLC with one of the following methods:

Method A: Agilent Scalar C18 150×4.6 mm 5 micron column; 1.5 mL/min; solvent A—water (0.1% TFA); solvent B—acetonitrile (0.07% TFA, gradient: 10 min 95% A to 95% B; 5 min hold; then recycle; UV detection @ 214, 250 and 280 nm.

Method B: Agilent XDB C18 50×4.6 mm/1.8 micron column; 1.5 mL/min; solvent A—water (0.1% TFA), solvent B—acetonitrile (0.07% TFA); gradient: 5 min 95% A to 95% B then 1 min hold, 1 min 95% B to 95% A then 30 sec hold; UV detection @ 210, 254, and 280 nm.

Method C: Agilent Eclipse XBD C8 column; solvent A—water (0.1% TFA); solvent B—acetonitrile (0.07% TFA, gradient: 10 min 95% A to 95% B; 5 min hold; then recycle; UV detection @ 214, 250 and 280 nm.

Preparative HPLC condition—Method D: Phenomenex Luna 250×21.20 mm, 10 micron; solvent A is 0.07% TFA in acetonitrile; solvent B is 0.10% TFA in water; 26 minute run; gradient: 5% to 80% A over 10 minutes; from 80% to 100% A over 5 minutes; hold 100% A for 5 minutes; 100% to 5% A over 5 minutes; hold 1 minute then recycle; detection at 285 nm.

Prep HPLC conditions—Method E: Phenomenex Luna 250×30.00 mm, 10 micron; solvent A is 0.07% TFA in acetonitrile; solvent B is 0.10% TFA in water; rate is 20 mL/min; 30 minute run; 5% to 70% A over 14 minute ramp; 3 minute ramp from 80% to 100% A; hold 100% A for 3 minutes; ramp down from 100% to 5% A over 5 minutes; hold 10 minutes then recycle.

Thin layer chromatography (TLC) was performed using Analtech TLC plates GHLF, 250 microns, order #21521.

### Ethyl-8-{3-[(2R,3S)-1-(tert-butoxycarbonyl)-3-hydroxypyrrolidin-2-yl]prop-1-yn-1-yl}-1-cyclopropyl-6,7-di-fluoro-4-oxo-1,4-dihydroquinoline-3-carboxylate (2)

tert-Butyl-(2R,3S)-3-hydroxy-2-prop-2-yn-1-ylpyrroli-dine-1-carboxylate (1) (1.50 g, 6.66 mmol) and ethyl-1-cyclopropyl-6,7-difluoro-4-oxo-8-{[(tri-fluoromethyl)sulfonyl]oxy}-1,4-dihydroquinoline-3-carboxylate (2.94 g, 6.66 mol) were transferred to a 100-mL round bottom flask equipped with a reflux condenser and side-arm capped with a rubber septum. Then, the reaction vessel was evacuated and back filled with N₂ (×3) before adding triphenylphosphine (0.44 g, 1.7 mmol) and tetrahydrofuran (40 mL). The resulting solution was sparged with N₂ for 3-5 minutes before adding tetrakis(triphenylphosphine)palladium(0) (0.77 g, 0.66 mmol) and N,N-diisopropylethylamine (2.32 mL, 13.3 mmol) with continued sparging for another 3-5 minutes before, finally, adding copper(I) iodide (0.32 g, 1.7 mmol). The resultant clear yellow solution was stirred at 60° C. for 12 hr before checking. After this period of time, the reaction had become dark, and HPLC showed the complete consumption of the starting triflate and formation of a major product that is the desired Sonogashira coupled product, 35. The reaction was cooled to ambient temperature and then ethanol (20 mL) was added with continued stirring for 15 minutes. After this period of time, the reaction was filtered through a short plug of Celite 545 and rinsed with several portion of ethanol (3×50 mL) and the filtrate was concentrated in vacuo to afford the crude product as a dark oil. The crude material was purified by silica gel chromatography (90 g) eluting with 0 to 50% ethyl acetate in CH₂Cl₂ (1.5 hr gradient, ˜30 mL/min) to afford 3.5 g of a dark solid foam. This material was purified once more by silica gel chromatography (90 g) eluting with the following gradient (25-30 mL/min flow): 0-5 min, 0% ethyl acetate/CH₂Cl₂, 5-40 min (0 to 20% ethyl acetate/CH₂Cl₂, linear gradient), 40 min to 1 hr (20 to 30% ethyl acetate/CH₂Cl₂, linear gradient), 1 hr to 1.25 hr (30 to 40% ethyl acetate/CH₂Cl₂, linear gradient), and 1.25 hr to 1.5 hr (40 to 50% ethyl acetate/CH₂Cl₂, linear gradient) to afford the purified product (2), 3.34 g in 97% yield after solvent removal; ¹H NMR (400 MHz, DMSO-d₆) δ ppm 8.55 (s, 1H), 8.06 (t, J=9.54 Hz, 1H), 5.13 (dd, J=7.46, 3.32 Hz, 1H), 4.23 (q, J=7.05 Hz, 2H), 4.18 (m, 2H), 3.64 (m, 1H), 3.34 (m, 2H), 2.87 (m, 1H), 2.73 (m, 1H), 2.13 (m, 1H), 1.74 (m, 1H), 1.41 (s, 9H), 1.28 (t, J=7.15 Hz, 3H), 1.18 (m, 4H); HPLC: 7.249 min (Method A: 100area % at 214, 254, and 280 nm); MS ES⁺517.1 m/z for [C₂₇H₃₀F₂N₂O₆+1]⁺; ES 561.1 m/z for [C₂₇H₃₀F₂N₂O₆+formate]⁻.

### Ethyl-8-{(1Z)-3-[(2R,3S)-1-(tert-butoxycarbonyl)-3-hydroxypyrrolidin-2-yl]prop-1-en-1-yl}-1-cyclopropyl-6,7-difluoro-4-oxo-1,4-dihydroquinoline-3-carboxylate (3)

Ethyl-8-{3-[(2R,3S)-1-(tert-butoxycarbonyl)-3-hydroxypyrrolidin-2-yl]prop-1-yn-1-yl}-1-cyclopropyl-6,7-difluoro-4-oxo-1,4-dihydroquinoline-3-carboxylate (2) (3.00 g, 5.81 mmol) was placed under an atmosphere of N₂ (g) by partial evacuation and then back-fill with N₂ (balloon). Then, the substrate was dissolved in ethanol (100 mL) and the solution sparged with N₂ (via syringe needle and outlet needle) for 5 minutes before adding 5% palladium on barium sulfate (1.8 g), quinoline (0.15 mL, 1.3 mmol), and triethylamine (0.15 mL, 1.1 mmol). Then, the reaction was partially evacuated and back filled with H₂ (×3) and then 2-L of hydrogen was bubbled through the mixture. After this, the reaction was maintained under an atmosphere of H₂ with a balloon. The reaction was stirred overnight and then checked after ˜12 hr. HPLC after this period of time shows complete consumption of the starting material and formation of the desired olefin (HPLC 6.850 min, Method A). The reaction mixture was filtered through a short plug of Celite 545 (˜30 mL) and then the filter cake was washed several times with 50-mL portions of ethanol. The filtrate was concentrated in vacuo and then purified by silica gel chromatography (90 g) eluting with 0 to 10% CH₃OH in CHCl₃ to afford the desired hydroxy-olefin (3), 2.30 g in 76% yield; ¹H NMR (400 MHz, CDCl₃) δ ppm 8.65 (s, 1H), 8.25 (t, J=9.33 Hz, 1H), 6.83 (d, J=11.20 Hz, 1H), 6.06 (m, 1H), 4.40 (q, J=7.12 Hz, 2H), 3.97 (m, 1H), 3.75 (m, 2H), 3.49 (m, 1H), 3.24 (m, 1H), 2.18 (m, 1H), 1.96 (m, 2H), 1.76 (m, 2H), 1.44 (br. s., 9H), 1.41 (m, 3H), 1.19 (m, 2H), 1.03 (m, 1H), 0.91 (m, 1H); MS: ES⁺519.1 m/z (M+1) for [C₂₇H₃₂F₂N₂O₆+1]⁺.

### Ethyl-(7aR,8S)-4-cyclopropyl-12-fluoro-8-hydroxy-1-oxo-4,7,7a,8,9,10-hexahydro-1H-pyrrolo[1′,2′:1,7]azepi-no[2,3-h]quinoline-2-carboxylate (4)

Ethyl-8-{(1Z)-3-[(2R,3S)-1-(tert-butoxycarbonyl)-3-hydroxypyrrolidin-2-yl]prop-1-en-1-yl}-1-cyclopropyl-6,7-difluoro-4-oxo-1,4-dihydroquinoline-3-carboxylate (3) (1.30 g, 2.51 mmol) was dissolved in CH₂Cl₂ (60 mL) and then water (20 mL) and trifluoroacetic acid (5 mL) were added. The reaction was stirred vigorously overnight at ambient temperature and then checked by HPLC/LCMS, which showed roughly equal amounts of the desired deprotected product, some TFA ester, and remaining starting material. The reaction was charged with additional reagent: trifluoroacetic acid (20 mL) and water (20 mL) with continued stirring another 24 hr. HPLC after this period of time shows a small amount of progress. The reaction was concentrated in vacuo and then 50 mL of 10% aqueous ammonium hydroxide was added and the organic product extracted with chloroform (4×100 mL). The combined organic layers were washed with 20 mL of saturated sodium chloride, dried over MgSO₄, filtered and concentrated in vacuo to afford the crude amine. The crude product was treated with N,N-diisopropylethylamine (7.5 mL, 43 mmol) in acetonitrile (50 mL) at 60° C. for 8 hr at which time the reaction was complete. The reaction mixture was concentrated in vacuo. HPLC/LCMS shows that there is product (4) and remaining starting material (3). The mixture was purified by preparative HPLC to afford the desired product (4) (454 mg, 45%). The remaining starting material was re-subjected to the reaction conditions described above to afford more of the desired tricyclic-ester; ¹H NMR (400 MHz, DMSO-d₆) δ ppm 8.50 (s, 1H), 7.46 (d, J=15.34 Hz, 1H), 6.62 (d, J=12.02 Hz, 1H), 5.95 (m, 1H), 4.23 (m, 3H), 4.07 (m, 2H), 3.72 (m, 1H), 3.63 (d, J=9.33 Hz, 1H), 2.63 (d, J=15.96 Hz, 1H), 2.35 (m, 1H), 2.09 (m, 1H), 1.86 (dd, J=12.96, 6.12 Hz, 1H), 1.27 (t, J=7.15 Hz, 3H), 1.24 (m, 1H), 0.92 (m, 2H), 0.71 (d, J=8.50 Hz, 1H), acidic H not observed; MS: ES⁺399.0 m/z (M+1) for [C₂₂H₂₃FN₂O₄+1]⁺; HPLC: 3.172 min (Method B).

### (7aR,8S)-4-Cyclopropyl-12-fluoro-8-hydroxy-1-oxo-4,7,7a,8,9,10-hexahydro-1H-pyrrolo[1′,2′:1,7]azepino[2,3-h]quinoline-2-carboxylic acid (5)

Ethyl-(7aR,8S)-4-cyclopropyl-12-fluoro-8-hydroxy-1-oxo-4,7,7a,8,9,10-hexahydro-1H-pyrrolo[1′,2′:1,7]azepino[2,3-h]quinoline-2-carboxylate (4) (454 mg, 1.14 mmol) was dissolved in acetonitrile (25 mL) and Water (2.0 mL) and then 2.73 mL of a 0.500 M aqueous solution of sodium hydroxide was added and the reaction was heated at 60° C. for 2 hr. HPLC at this time showed about 50% conversion to product. The reaction was charged once more with 1.50 mL of a 0.500 M solution of aqueous sodium hydroxide with continued stirring for 2 hr. HPLC after this period of time shows complete conversion to the desired product >97% by HPLC at 280 and 254 nm. The reaction was cooled to room temperature and neutralized (to pH 7) by the addition of acetic acid dropwise and then the reaction was concentrated in vacuo. The crude product was triturated with water to afford (5), 337 mg, in 79% yield; ¹H NMR (400 MHz, DMSO-d₆) δ ppm 8.73 (s, 1H), 7.59 (d, J=14.93 Hz, 1H), 6.64 (d, J=12.44 Hz, 1H), 5.99 (dt, J=12.08, 3.81 Hz, 1H), 5.15 (d, J=2.70 Hz, 1H), 4.25 (m, 2H), 4.10 (br. s., 1H), 3.82 (m, 1H), 3.69 (d, J=9.95 Hz, 1H), 2.66 (m, 1H), 2.38 (m, 1H), 2.11 (m, 1H), 1.89 (dd, J=12.85, 6.01 Hz, 1H), 1.27 (m, 1H), 1.05 (m, 1H), 0.96 (m, 1H), 0.79 (m, 1H), acidic hydrogen not observed; MS: ES⁺371.06 m/z (M+1)⁺ for [C₂₀H₁₉FN₂O₄+H]⁺ and ES⁻ 369.10 m/z (M−1)⁻ for [C₂₀H₁₉FN₂O₄−H]⁻ and 415.08 m/z (M+formate)⁻ for [C₂₀H₁₉FN₂O₄+CHO₂—]⁻; HPLC: 3.227 min (Method B).

### Example 20

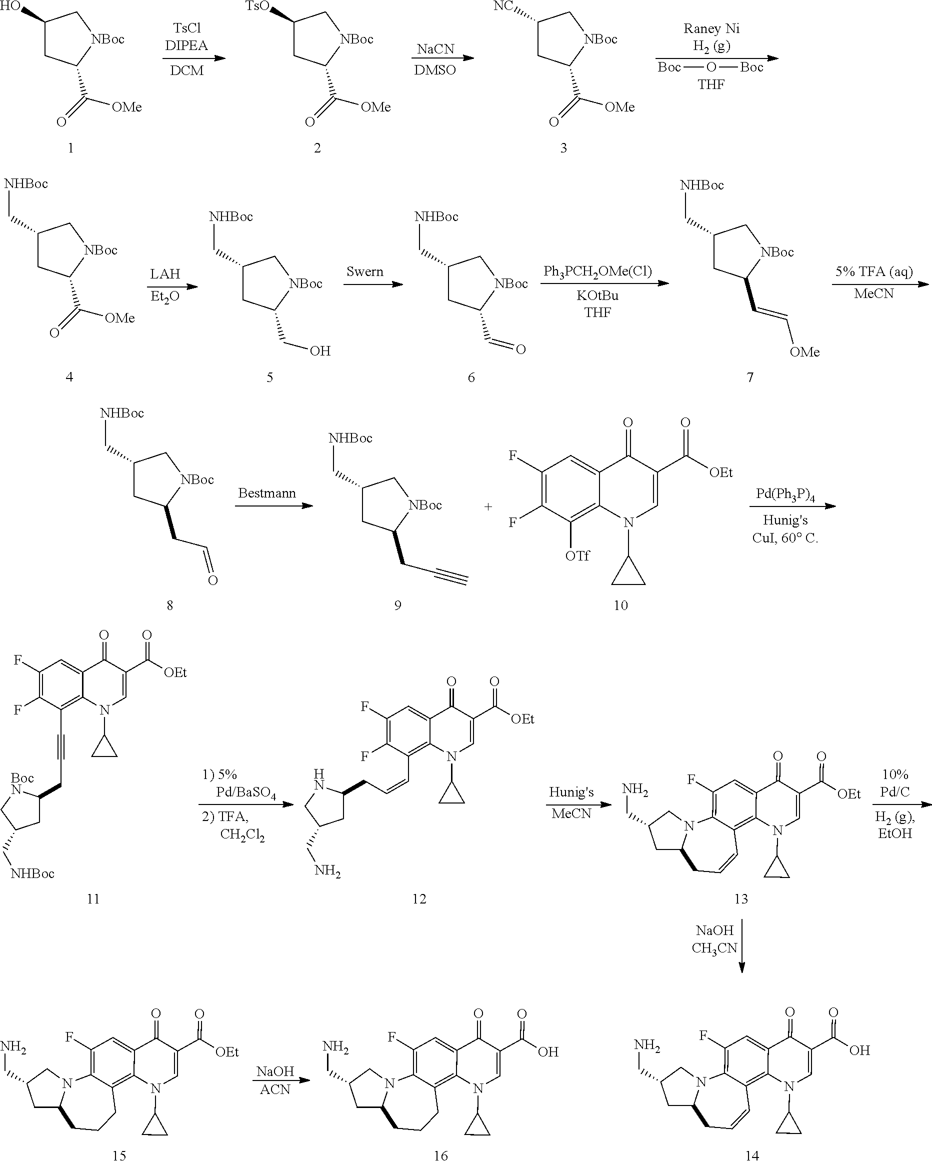

HPLC conditions (for final analysis and reaction monitoring) are as follows: Agilent 1100 HPLC. Agilent Scalar C18 150×4.6 mm 5 micron column. Solvent A—Water (0.1% TFA; Solvent B—Acetonitrile (0.07% TFA, Gradient—10 min 95% A to 95% B; 5 min hold; then recycle; UV Detection @ 214, 250, and 280 nm.

Prep HPLC conditions used for final purification: Phenomenex Luna 250×21.20 mm, 10 micron; Gradient: solvent A is 0.07% TFA in acetonitrile; solvent B is 0.10% TFA in water; rate is 20 mL/min; 30 minute run; 5% to 70% A over 14 minute ramp; 3 minute ramp from 80% to 100% A; hold 100% A for 3 minutes; ramp down from 100% to 5% A over 5 minutes; hold 5 minutes then recycle.

### 1-tert-Butyl-2-methyl-C2S,4R)-4-{[(4-methylphenyl)sulfonyl]oxy}pyrrolidine-1,2-dicarboxylate (2)

p-Toluenesulfonyl chloride (28.0 g, 147 mmol) was added to a solution of 1-tert-butyl-2-methyl-(2S,4R)-4-hydroxypyrrolidine-1,2-dicarboxylate (1) (30.00 g, 122.3 mmol) and triethylamine (51.1 mL, 367 mmol) in CH₂Cl₂ (500 mL). Then, 4-dimethylaminopyridine (1.00 g, 8.18 mmol) was added and the reaction was stirred overnight. TLC in 50% ethyl acetate in hexanes shows incomplete reaction; consequently, 0.5 eq (14 g) of p-toluenesulfonyl chloride was added to the mixture and the reaction was stirred overnight at ambient temperature. TLC after this period of time shows no remaining starting material. The reaction was diluted with 300 mL of CH₂Cl₂ and then the organic layer was washed with 1.0 N HCl (2×100 mL), 1.0 N NaOH (2×100 mL), brine (1×100 mL) and then dried over Na₂SO₄. Evaporation of the solvent afforded a dark oil with some crystalline solid present. Purification by silica gel chromatography using 25% ethyl acetate in hexanes on 450 g of silica gel afforded 47.4 g (97%) of a light brown oil (2); ¹H NMR (400 MHz, CDCl₃) δ ppm 1.32-1.48 (m, 9 H), 2.07-2.23 (m, 1H), 2.34-2.62 (m, 4H), 3.52-3.66 (m, 2H), 3.72 (s, 3H), 4.28-4.46 (m, 1H), 4.96-5.12 (m, 1H), 7.33-7.41 (m, 2H), 7.79 (d, J=8.29 Hz, 2H); MS ES⁺300.3 m/z (M−99) and 344.3 m/z (M−55) for [C₁₈H₂₅NO₇S]⁺.

### 1-tert-Butyl-2-methyl-(2S,4S)-4-cyanopyrrolidine-1,2-dicarboxylate (3)

A mixture of 1-tert-butyl-2-methyl-(2S,4R)-4-{[(4-methylphenyl)sulfonyl]oxy}pyrrolidine-1,2-dicarboxylate (2) (47.4 g, 0.119 mol) and sodium cyanide (18.2 g, 0.370 mol) (finely ground in a mortar and pestle) in 150 mL of DMSO was heated in an 55° C. oil bath for 16 hr then allowed cool to room temperature for 2 days. LC/MS after this period of time shows a peak for the starting material. The reaction was heated at 55° C. for an additional 10 hours and then allowed to cool to room temperature overnight. LC/MS after this period of time shows complete consumption of the starting tosylate (2). The reaction was diluted with 200 mL of 1:1 brine/water and extracted with EtOAc (3×100 mL). The combined organics were washed with water (2×100 mL) and then dried over Na₂SO₄, filtered and evaporated to give a dark oil. The residue was purified by silica gel chromatography (Biotage 65M, 450 g) eluting with 8% EtOAc in CH₂Cl₂ to give 18.6 g (62%) of a light brown solid (3); ¹H NMR (400 MHz, CDCl₃) δ ppm 1.34-1.52 (m, 9H), 2.22-2.43 (m, 1H), 2.58-2.79 (m, 1H), 2.97-3.34 (m, 1H), 3.60-3.84 (m, 4H), 3.84-4.04 (m, 1H), 4.24-4.54 (m, 1H); MS ES⁺277.3 m/z for [C₁₂H₁₈N₂O₄+Na]⁺;

### 1-tert-Butyl-2-methyl-(2S,4S)-4-cyanopyrrolidine-1,2-dicarboxylate (4)

Raney nickel (8.59 g, 146 mmol) was added to a mixture of 1-tert-butyl-2-methyl-(2S,4S)-4-cyanopyrrolidine-1,2-dicarboxylate (3) (18.6 g, 73.1 mmol) and di-tert-butyldicarbonate (31.9 g, 146 mmol) in tetrahydrofuran (400 mL). The mixture was stirred at room temperature under an H₂ atmosphere overnight. After this period of time, the reaction was not complete by TLC analysis (5% ethyl acetate in CH₂Cl₂). The reaction vessel was charged again with hydrogen and then a fresh H₂ balloon was added and the reaction and stirred overnight at ambient temperature. After this period of time, the reaction was still not complete. Additional Raney nickel (4 g) was added along with another fresh H₂ balloon with continued stirring overnight (˜16 hr). The reaction mixture was filtered through Celite 545 to remove catalyst and the filtrate was concentrated to remove the solvent. The crude material was purified by silica gel chromatography on a Biotage 65M (450 g) using 5% ethyl acetate in CH₂Cl₂ as the elutant to afford 11.2 g (43%) of the desired product (4) as a colorless oil along with 12 g of the recovered starting material. MS ES⁺381.4 m/z for [C₁₇H₃₀N₂O₆+Na]⁺.

### tert-Butyl-(2S,4R)-4-{[tert-butoxycarbonyl)amino]methyl}-2-(hydroxymethyl)pyrrolidine-1-carboxylate (5)

1-tert-butyl-2-methyl-(2S,4R)-4-[(tert-butoxycarbonyl)amino]methyl)pyrrolidine-1,2-dicarboxylate (4) (4.72 g, 13.2 mmol) in 75 mL of Et₂O was added dropwise via addition funnel to a suspension of lithium aluminum hydride (1.00 g, 0.0263 mol) in 200 mL of diethyl ether which was cooled in an ice/water bath under N₂. The mixture was stirred for 1 hour. TLC (5% ethyl acetate in CH₂Cl₂) after this period of time showed no remaining starting material. The reaction was quenched by the sequential addition of 1.00 mL of water, 1.00 mL of 15% NaOH (aq) and 3 mL of water and then the mixture was allowed to warm to room temperature. The solid was removed by filtration through a pad of Celite 545 and the resulting filtrate was evaporated to afford a light yellow oil. The residue was purified by silica gel chromatography using 120 g of silica gel eluting with 50% ethyl acetate in hexanes to afford 3.9 g (88%) of the product (5) as a white foam; ¹H NMR (400 MHz, CDCl₃) δ ppm 1.36-1.54 (m, 18H), 2.09-2.22 (m, 1H), 2.28 (br. s., 1H), 2.93 (t, J=10.57 Hz, 1H), 3.14 (br. s., 2H), 3.53-3.81 (m, 3H), 3.94 (br. s., 1H), 4.62 (br. s., 1H), 5.18 (br. s., 1H); MS ES⁺353.4 m/z for [C₁₆H₃₀N₂O₅+Na]⁺; ES⁻ 329.4 m/z for [C₁₆H₃₀N₂O₄−H]⁻.

### tert-Butyl-(2S,4R)-4-{[(tert-butoxycarbonyl)amino]methyl}-2-formylpyrrolidine-1-carboxylate (6)

Dimethyl sulfoxide (6.01 mL, 84.7 mmol) was added to a solution of oxalyl chloride (3.58 mL, 42.4 mmol) in CH₂Cl₂ (500 mL) which had been cooled to −78° C. under a nitrogen atmosphere. The mixture was stirred at −78° C. for 15 minutes, then tert-butyl-(2S,4R)-4-{[(tert-butoxycarbonyl)amino]methyl}-2-(hydroxymethyl)pyrrolidine-1-carboxylate (5) (7.0 g, 21 mol) was added as a solution in 50 mL of CH₂Cl₂. The addition was done at a rate to keep the reaction temp <−75° C. When the addition was complete the mixture was stirred for 30 minutes and then triethylamine (11.8 mL, 84.7 mmol) was added. The mixture was stirred at −78° C. for 1 hour. The reaction was allowed to warm to −40° C. over ˜30 min. TLC (50% ethyl acetate in hexanes) after this period of time indicated complete consumption of the starting material. The reaction was quenched by addition of 100 mL of H₂O and the mixture was warmed to room temperature. The layers were separated and the aqueous phase was extracted with 100 mL of CH₂Cl₂. The combined organic layers were washed with water (1×100 mL) and brine (1×50 mL), dried over Na₂SO₄, and concentrated in vacuo to afford 7.2 g (100%) of a viscous yellow oil (6). ¹H NMR (400 MHz, CDCl₃) δ ppm 9.46 (m, 1H), 4.07 (m, 2H), 3.70 (m, 1H), 3.13 (m, 2H), 2.32 (m, 2H), 1.64 (m, 2H), 1.45 (m, 18H).

### tert-Butyl-(2R,4R)-4-{[(tert-butoxycarbonyl)amino]methyl}-2-[(E)-2-methoxyvinyl]pyrrolidine-1-carboxylate (7)

Potassium tert-butoxide (7.41 g, 66.1 mmol) was added to a suspension of (methoxymethyl)triphenyl-phosphonium chloride (24.1 g, 70.3 mmol) in tetrahydrofuran (200 mL) and the reaction was allowed stir for 1 hr. After this period of time, tert-butyl-(2S,4R)-4-{[(tert-butoxycarbonyl)amino]methyl}-2-formylpyrrolidine-1-carboxylate (6) (7.0 g, 21 mmol) in tetrahydrofuran (100 mL) was added drop wise via cannula and the reaction was stirred overnight. TLC analysis after 16 hr shows a new, higher R_{f} product. The reaction was quenched by the addition of saturated sodium bicarbonate solution (100 mL) and concentrated in vacuo. The organic product was extracted with ethyl acetate (3×100 mL) and the combined organic layers were washed with brine (1×50 mL), dried over sodium sulfate, filtered and concentrated in vacuo to afford the crude product. Purification by silica gel chromatography (Biotage 65M, 450 g) eluting with 25% ethyl acetate in hexanes afforded 6.46 g (85%) of the purified product (7) as a colorless oil; ¹H NMR (400 MHz, CDCl₃) δ ppm 1.44 (s, 18H), 2.13-2.38 (m, 2H), 2.86-3.36 (m, 3H), 3.42-3.90 (m, 4H), 4.01-4.77 (m, 3H), 5.71-6.59 (m, 1H); MS ES⁺379.2 m/z for [C₁₈H₃₂N₂O₅+Na]⁺;

### tert-Butyl-(2R,4R)-4-{[(tert-butoxycarbonyl)amino]methyl}-2-(2-oxoethyl)pyrrolidine-1-carboxylate (8)

tert-Butyl-(2R,4R)-4-{[(tert-butoxycarbonyl)amino]methyl}-2-[(E)-2-methoxyvinyl]pyrrolidine-1-carboxylate (7) (3.30 g, 9.26 mmol) was dissolved in acetonitrile (100 mL) in a 100-mL round bottom flask. Then 0.4 M trifluoroacetic acid in water (2 mL) was added to the reaction mixture and the reaction was stirred overnight at ambient temperature. After this period of time, the reaction was determined to be complete by TLC analysis (50% ethyl acetate in hexanes). A saturated solution of sodium bicarbonate (10 mL) was added to quench the reaction and then the solvent was evaporated. The resultant aqueous layer was then extracted with ethyl acetate (3×50 mL), and the combined organic layers were washed with brine (1×25 mL), dried with sodium sulfate, filtered and concentrated in vacuo to afford the crude product. Silica gel chromatography of the crude material (Biotage, 40 g silica gel cartridge) eluting with 30% ethyl acetate in hexanes afforded 2.75 g (87%) of the product (8) as a colorless oil. This material was carried on to the next step without further characterization.

### tert-Butyl-(2R,4R)-4-{[tert-butoxycarbonyl)amino]methyl}-2-prop-2-yn-1-ylpyrrolidine-1-carboxylate (9)

Tosyl azide (2.74 g, 13.9 mmol), acetonitrile (50 mL) and potassium carbonate (3.84 g, 27.8 mmol) were added to a 500 mL flask. Dimethyl 2-oxopropylphosphonate (2.02 mL, 0.0139 mol) was added to the stirred suspension and then the reaction was stirred for 2 hr at ambient temperature. TLC (5% methanol in dichloromethane) analysis after this period of time shows complete formation of the diazophosphonate. Next, tert-Butyl-(2R,4R)-4-{[(tert-butoxycarbonyl)amino]methyl}-2-(2-oxoethyl)pyrrolidine-1-carboxylate (8) (2.75 g) was dissolved in methanol (100 mL) and added dropwise via addition funnel and the resultant mixture was allowed to stir overnight. TLC (50% ethyl acetate in hexanes) after this period of time shows the complete consumption of starting material and formation of a large spot corresponding to product. The reaction was concentrated to remove the solvents, and the resultant residue was partitioned between diethyl ether and water (100 mL each). The layers were separated and the aqueous layer was extracted with diethyl ether (2×50 mL) before the combined organic layers were washed with water (2×50 mL) and brine (1×25 mL), dried with sodium sulfate, filtered and concentrated in vacuo to afford the crude product. Silica gel chromatography on a 40 g silica gel cartridge, eluting with 20% ethyl acetate in hexanes afforded 1.80 g (57%) of a colorless oil that crystallized upon standing. The product was dissolved in hot hexanes (20 mL) and allowed to cool. The crystallized product (9) was filtered to give 700 mg of a white solid; ¹H NMR (400 MHz, CDCl₃) δ ppm 4.61 (br. s., 1H), 3.77 (m, 2H), 3.18 (br. s., 2H), 2.94 (t, J=10.16 Hz, 1H), 2.62 (m, 2H), 2.29 (m, 2H), 1.94 (br. s., 1H), 1.63 (m, 1H), 1.46 (m, 18H); MS ES⁺361.2 m/z [C₁₈H₃₀N₂O₄+Na]⁺;

### Ethyl-8-{3-[(2R,4R)-1-(tert-butoxycarbonyl)-4-{[(tert-butoxycarbonyl)amino]methyl}pyrrolidin-2-yl]prop-1-yn-1-yl}-1-cyclopropyl-6,7-difluoro-4-oxo-1,4-dihydroquinoline-3-carboxylate (11)

tert-Butyl-(2R,4R)-4-{[(tert-butoxycarbonyl)amino]methyl}-2-prop-2-yn-1-ylpyrrolidine-1-carboxylate (9) (700 mg, 2.07 mmol) and ethyl-1-cyclopropyl-6,7-difluoro-4-oxo-8-{[(trifluoromethyl)sulfonyl]oxy}-1,4-dihydroquinoline-3-carboxylate (10) (913 mg, 2.07 mmol) were combined in a 40 mL scintillation vial and then placed under nitrogen via vacuum evacuation and backfill with nitrogen. Triphenylphosphine (140 mg, 0.52 mmol) and tetrahydrofuran (20 mL) were added and the reaction was sparged with nitrogen for 3-4 minutes. Then, N,N-Diisopropylethylamine (0.720 mL, 4.14 mmol) and tetrakis(triphenylphosphine)palladium(0) (240 mg, 0.21 mmol) were added with continued sparging (˜3 min) and finally copper(I) iodide (143 mg, 0.751 mmol) was added and the resultant translucent yellow reaction mixture was capped and heated at 60° C. overnight. After heating for 16 hr, the very dark reaction was checked by HPLC/LCMS and TLC. Analysis after this period of time shows complete consumption of the starting triflate and alkyne. The crude reaction was diluted with ethanol (5 mL) and then stirred for 5 minutes, filtered (Buchner funnel/filter paper) to remove the precipitated solids. The filtrate was concentrated in vacuo and then purified by silica gel chromatography on a 120 g cartridge, eluting with 15% ethyl acetate in dichloromethane with 1% ethanol to afford the purified product (11), 1.16 g, 89% yield, which was ˜96% pure by HPLC; HPLC: 6.976 min; ¹H NMR (400 MHz, CDCl₃) δ ppm 8.62 (s, 1H), 8.23 (t, J=9.43 Hz, 1H), 4.65 (br. s., 1H), 4.39 (q, J=7.26 Hz, 2 H), 4.13 (m, 3H), 3.09 (m, 4H), 2.33 (m, 2H), 1.73 (m, 1H), 1.44 (m, 21H), 1.28 (m, 3H), 1.12 (m, 2H). MS ES⁺630.3 m/z (M+1) for [C₃₃H₄₁F₂N₃O₇+H]⁺.

### Ethyl-8-{(1Z)-3-[(2S,4R)-4-(aminomethyl)pyrrolidin-2-yl]prop-1-en-1-yl}-1-cyclopropyl-6,7-difluoro-4-oxo-1,4-dihydroquinoline-3-carboxylate (12)

Ethyl-8-{3-[(2R,4R)-1-(tert-butoxycarbonyl)-4-{[(tert-butoxy-carbonyl)amino]methyl}pyrrolidin-2-yl]prop-1-yn-1-yl}-1-cyclopropyl-6,7-difluoro-4-oxo-1,4-dihydroquinoline-3-carboxylate (11) (1.16 g, 1.84 mmol) was dissolved in ethanol (100 mL). The reaction was sparged with nitrogen before adding 5% palladium on barium sulfate (0.784 g, 0.368 mmol). Then, triethylamine (0.050 mL, 0.36 mmol) was added to ensure that the reaction was basic (wet pH paper). The reaction was sparged with nitrogen and then with hydrogen and maintained under an atmosphere of hydrogen with a balloon. The reaction was checked after 2.5 hr and found to be ˜50% complete. After an additional 3 hr, the reaction is ˜65% complete. Additional catalyst (˜0.7 g) was added along with a fresh balloon of H₂ and the reaction was stirred overnight. After this period of time, HPLC/LCMS showed the presence of the desired cis-alkene along with over-reduction products—both the alkane and cyclopropyl cleaved products. The reaction was sparged with nitrogen for 3 minutes and then vacuum filtered through a short plug of Celite 545 and the filtrate was concentrated in vacuo. The crude material was purified by silica gel chromatography on a Biotage 40 L using 60% ethyl acetate in hexanes with 1% ethanol as the elutant. The compound without the cyclopropyl group (240 mg) was separated from the other products. The remaining mixture (840 mg) was carried on to the next step without further separation.

The mixture containing ethyl-8-{(1Z)-3-[(2S,4R)-1-(tert-butoxycarbonyl)-4-{[(tert-butoxycarbonyl)amino]-methyl}-pyrrolidin-2-yl]prop-1-en-1-yl}-1-cyclopropyl-6,7-difluoro-4-oxo-1,4-dihydroquinoline-3-carboxylate (0.424 g, 0.671 mmol) was dissolved in CH₂Cl₂ (50 mL) in a 500 mL round bottom flask. Trifluoroacetic acid (2 mL, 0.02 mol) was added and the reaction was stirred at room temperature for 4 hr. After this period of time, the reaction was checked for completion by HPLC and no starting material was observed. The reaction was concentrated to remove the solvents, and then the residue was partitioned between dichloromethane (100 mL) and 10% aqueous ammonium hydroxide solution (25 mL). The layers were separated and the aqueous was extracted with dichloromethane (2×50 mL). The combined organic layers were washed with brine (1×50 mL), dried over sodium sulfate, filtered and concentrated in vacuo. The resultant crude product (12) was carried on to the next step without further purification.

### Ethyl-(7aS,9R)-9-(aminomethyl)-4-cyclopropyl-12-fluoro-1-oxo-4,7,7a,8,9,10-hexahydro-1H-pyrrolo-[1′,2′:1,7]azepino[2,3-h]quinoline-2-carboxylate (13)

A mixture containing ethyl-8-{(1Z)-3-[(2S,4R)-4-(aminomethyl)pyrrolidin-2-yl]prop-1-en-1-yl}-1-cyclopropyl-6,7-difluoro-4-oxo-1,4-dihydroquinoline-3-carboxylate (12) in acetonitrile (25 mL) was treated with N,N-diisopropylethylamine (1.67 mL, 9.58 mmol). The reaction mixture was stirred at 50° C. for 4 hr. HPLC after this period of time revealed the formation of a new product. The reaction was concentrated to remove the volatiles and then purified by silica gel chromatography using a 90 g silica gel cartridge, eluting with 20% ethyl acetate in CH₂Cl₂ to afford the purified product (13), 179 mg (yellow foam) in 24% yield over the three steps (from 10); ¹H NMR (400 MHz, CDCl₃) δ ppm 0.69-0.86 (m, 1H), 0.84-1.05 (m, 2H), 1.18-1.34 (m, 1H), 1.33-1.53 (m, 4H), 2.29-2.60 (m, 3H), 2.60-2.74 (m, 1H), 2.85 (br. s., 2H), 3.16 (ddd, J=11.20, 6.95, 3.84 Hz, 1H), 3.78-3.91 (m, 2H), 4.31-4.48 (m, 3H), 5.98 (dt, J=11.87, 4.33 Hz, 1H), 6.60 (d, J=11.82 Hz, 1H), 7.81 (d, J=14.72 Hz, 1H), 8.59 (s, 1H); MS ES⁺412.2 m/z (M+1) for [C₂₃H₂₆FN₃O₃₊H]⁺ and ES″ 410.2 m/z (M−1) for [C₂₃H₂₆FN₃O₃−H]⁻.

### (7aS,9R)-9-(Aminomethyl)-4-cyclopropyl-12-fluoro-1-oxo-4,7,7a,8,9,10-hexahydro-1H-pyrrolo[1′,2′:1,7]azepino[2,3-h]quinoline-2-carboxylic acid trifluoroacetate (14)

Ethyl-(7aS,9R)-9-(aminomethyl)-4-cyclopropyl-12-fluoro-1-oxo-4,7,7a,8,9,10-hexahydro-1H-pyrrolo[1′,2′:1,7]azepino-[2,3-1H]quinoline-2-carboxylate (13) (52 mg, 0.13 mmol) was dissolved in acetonitrile (7 mL) in a 40 mL scintillation vial. The, water (1 mL) and 1.0 M sodium hydroxide (0.379 mL) were added to the reaction. The vial was capped and heated at 50° C. for 4 hr. After this period of time, HPLC and LC/MS show reaction to be complete (>95 area % purity). The reaction was removed from the oil bath and allowed to cool to room temperature and quenched with acetic acid until a pH of 5 was attained (10 drops). The solvent was evaporated under a stream of nitrogen, and the residue was purified by preparative HPLC. Two very clean fractions of the product were isolated from preparative HPLC and the fractions were combined and lyophilized overnight to give 53 mg (84%) of (14) as a yellow solid as the trifluoroacetate salt; ¹H NMR (400 MHz, DMSO-d₆) δ ppm 8.76 (s, 1H), 7.85 (br. s., 3H), 7.67 (d, J=14.51 Hz, 1H), 6.77 (d, J=12.02 Hz, 1H), 6.01 (dt, J=11.66, 4.22 Hz, 1H), 4.34 (m, 1H), 4.25 (m, 1H), 3.89 (br. s., 1H), 3.30 (m, 2H), 2.98 (m, 2 H), 2.62 (m, 3H), 1.51 (m, 1H), 1.00 (m, 2H), 0.93 (m, 3H); MS ES⁺384.2 m/z (M+1) for [C₂₁H₂₂FN₃O₃+H]⁺ and ES^{˜}382.2 m/z (M−1) for [C₂₁H₂₂FN₃O₃−H]⁻;

### Ethyl-(7aS,9R)-9-(aminomethyl)-4-cyclopropyl-12-fluoro-1-oxo-4,5,6,7,7a,8,9,10-octahydro-1H-pyrrolo-[1′,2′:1,7]azepino[2,3-h]quinoline-2-carboxylate (15)

Ethyl-(7aS,9R)-9-(aminomethyl)-4-cyclopropyl-12-fluoro-1-oxo-4,7,7a,8,9,10-hexahydro-1H-pyrrolo[1′,2′:1,7]azepino[2,3-h]quinoline-2-carboxylate (13) (0.124 g, 0.301 mmol), ethanol (10 mL), and triethylamine (0.010 mL, 0.072 mmol) were added to a 250 mL flask. Nitrogen was bubbled through the solution and 10% palladium on carbon (0.032 g) was added with continued bubbling for 5 min. The pH of the reaction was checked with wet pH paper to ensure it was basic. The nitrogen line was replaced with a hydrogen balloon (bubbled through solution for 2-3 min). The vent needle was removed and the reaction mixture was stirred overnight under an atmosphere of hydrogen. After this period of time, HPLC analysis shows the reaction to be complete. Nitrogen was bubbled through the reaction mixture for 5 min to remove residual hydrogen, and then the reaction was filtered through Celite 545 and the filtrate was concentrated to remove ethanol. The reaction was dissolved in CH₂Cl₂ and evaporated twice (azeotropic removal of residual water) to afford 397 mg (94%) of a bright yellow (fluorescent) glass; MS ES⁺414.2 m/z (M+1) for [C₂₃H₂₈FN₃O₃+H]⁺.

### (7aS,9R)-9-(Aminomethyl)-4-cyclopropyl-12-fluoro-1-oxo-4,5,6,7,7a,8,9,10-octahydro-1H-pyrrolo[1′,2′:1,7]-azepino[2,3-h]quinoline-2-carboxylic acid trifluoroacetate (16)

Compound (16) was prepared from compound (15) using the same procedure outlined for the synthesis of compound (14) to afford 74 mg (58%) of a yellow solid as the trifluoroacetate salt; ¹H NMR (400 MHz, DMSO-d₆) δ ppm 8.77 (s, 1H), 7.79 (m, 4H), 4.34 (m, 1H), 4.07 (m, 1H), 3.68 (m, 1H), 3.24 (m, 1H), 2.97 (br. s., 2H), 2.41 (m, 2H), 2.01 (m, 1H), 1.90 (m, 1H), 1.67 (m, 1H), 1.49 (m, 2H), 1.18 (m, 2H), 0.98 (m, 1H), 0.74 (m, 1H); MS ES⁺386.2 m/z (M+1) for [C₂₁H₂₄FN₃O₃+H]⁺ and ES^{˜}384.2 m/z (M−1) for [C₂₁H₂₄FN₃O₃−H]⁻.

### Example 21

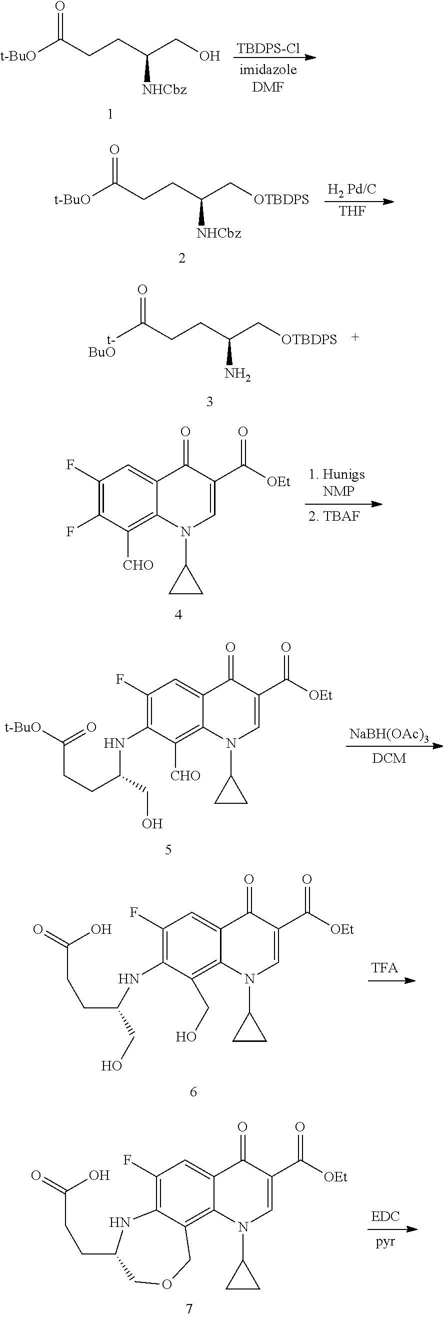

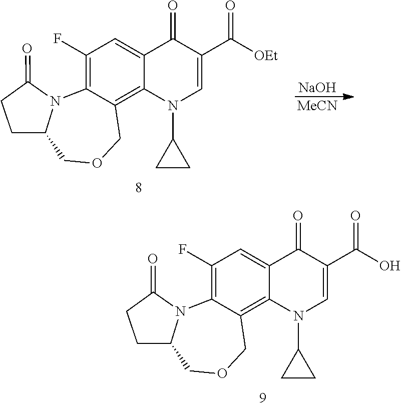

All reactions were performed under an atmosphere of nitrogen. Unless otherwise indicated, the reaction flask was evacuated with vacuum and then back-filled with nitrogen via a balloon (×3) and the reaction kept under nitrogen via balloon for the duration of the reaction.

Analytical HPLC was performed using an Agilent 1100 HPLC with one of the following methods:

Method A: Agilent Scalar C18 150×4.6 mm 5 micron column; 1.5 mL/min; solvent A—water (0.1% TFA); solvent B—acetonitrile (0.07% TFA, gradient: 10 min 95% A to 95% B; 5 min hold; then recycle; UV detection @ 214, 250 and 280 nm.

Method B: Agilent XDB C18 50×4.6 mm/1.8 micron column; 1.5 mL/min; solvent A—water (0.1% TFA), solvent B—acetonitrile (0.07% TFA); gradient: 5 min 95% A to 95% B then 1 min hold, 1 min 95% B to 95% A then 30 sec hold; UV detection @ 210, 254, and 280 nm.

Method C: Agilent Eclipse XBD C8 column; solvent A—water (0.1% TFA); solvent B—acetonitrile (0.07% TFA, gradient: 10 min 95% A to 95% B; 5 min hold; then recycle; UV detection @ 214, 250 and 280 nm.

Preparative HPLC condition—Method D: Phenomenex Luna 250×21.20 mm, 10 micron; solvent A is 0.07% TFA in acetonitrile; solvent B is 0.10% TFA in water; 26 minute run; gradient: 5% to 80% A over 10 minutes; from 80% to 100% A over 5 minutes; hold 100% A for 5 minutes; 100% to 5% A over 5 minutes; hold 1 minute then recycle; detection at 285 nm.

Prep HPLC conditions—Method E: Phenomenex Luna 250×30.00 mm, 10 micron; solvent A is 0.07% TFA in acetonitrile; solvent B is 0.10% TFA in water; rate is 20 mL/min; 30 minute run; 5% to 70% A over 14 minute ramp; 3 minute ramp from 80% to 100% A; hold 100% A for 3 minutes; ramp down from 100% to 5% A over 5 minutes; hold 10 minutes then recycle.

Thin layer chromatography (TLC) was performed using Analtech TLC plates GHLF, 250 microns, order #21521.

### tert-Butyl-(4S)-4-{[(benzyloxy)carbonyl]amino}-5-{[tert-butyl(diphenyl)silyl]oxy}pentanoate (2)

tert-Butyl-(4S)-4-{[(benzyloxy)carbonyl]amino}-5-hydroxypentanoate (1) (1.00 g, 3.09 mmol) was dissolved in N,N-dimethylformamide (5 mL) and 1H-imidazole (0.253 g) was added followed by tert-butylchlorodiphenylsilane (0.965 mL, 3.71 mmol). The reaction was stirred overnight at ambient temperature and then checked for completion by silica gel TLC (30% ethyl acetate:dichloromethane). TLC at this time indicates the reaction is complete. Methanol (1 mL) was added to quench the remaining tert-butylchlorodiphenylsilane, and the reaction was partitioned between diethyl ether and water (100 mL each). The organic product was extracted with diethyl ether (2×200 mL) and the combined organic layers washed with water (2×100 mL) and saturated sodium bicarbonate (100 mL) and brine (100 mL). The organic layer was then dried over sodium sulfate, filtered, and concentrated in vacuo. This afforded 1.82 g (105%) of the desired product (2) as a white solid (contains less than 10% of the TBDPS methyl ether). This was used in the next step without further purification.

### tert-Butyl-(4S)-4-amino-5-{[tert-butyl(diphenyl)silyl]oxy}pentanoate (3)

tert-Butyl-(4S)-4-{[(benzyloxy)carb-onyl]amino}-5-{[tert-butyl(diphenyl)silyl]oxy}pentanoate (2) (0.530 g, 0.943 mmol) and tetrahydrofuran (10 mL) were added to a 100 mL flask. The stirring solution was sparged with nitrogen for 5 minutes, and then 5% Pd on carbon (0.201 g) was added to the mixture with continued sparging. The nitrogen line was replaced with a balloon of hydrogen gas, and the reaction was sparged for 3 minutes, the vent needle was removed, and the reaction was stirred under a hydrogen atmosphere overnight. TLC (25% ethyl acetate in hexanes) after this period of time showed the reaction to be complete. The mixture was sparged with nitrogen, filtered through a short plug Celite 545 and concentrated to remove the solvent. The crude material was purified by silica gel chromatography on a Biotage 40M (90 g) using 0 to 10% methanol in dichloromethane as the elutant. The fractions containing the product were combined and concentrated to give 290 mg (72%) of the desired product (3) was isolated as a yellow oil; ¹H NMR (400 MHz, CDCl₃) δ ppm 1.07 (s, 18H), 1.42 (s, 18H), 1.64-1.85 (m, 2H), 2.20-2.41 (m, 2H), 3.04 (br. s., 3H), 3.53 (dd, J=10.26, 6.95 Hz, 1H), 3.70 (dd, J=10.26, 4.04 Hz, 1H), 7.33-7.50 (m, 6H), 7.60-7.71 (m, 4H).

### Ethyl-7-{[(1S)-4-tert-butoxy-1-(hydroxymethyl)-4-oxobutyl]amino}-1-cyclopropyl-6-fluoro-8-formyl-4-oxo-1,4-dihydroquinoline-3-carboxylate (5)

tert-Butyl-(4S)-4-amino-5-{[tert-butyl(diphenyl)silyl]oxy}pentanoate (3) (0.154 g, 0.000360 mol) and N-methylpyrrolidinone (5 mL) were added to a 100 mL flask. Ethyl-1-cyclopropyl-6,7-difluoro-8-formyl-4-oxo-1,4-dihydroquinoline-3-carboxylate (4) (231 g, 0.000719 mol) and N,N-diisopropylethylamine (0.250 mL, 0.00144 mol) were added with heating at 50° C. overnight. LCMS after this period of time shows complete consumption of the starting material. The reaction was cooled and partitioned between ethyl acetate and water (50 mL each). The layers were separated and the aqueous layer was extracted with ethyl acetate (3×50 mL), and the combined organic layers were washed with water (3×25 mL) and brine (1×10 mL), dried over sodium sulfate, filtered, and concentrated in vacuo. The crude residue was dissolved in tetrahydrofuran (10 mL), and then 1 equivalent (each) of acetic acid and tetrabutylammonium fluoride (1M in tetrahydrofuran) were added to remove the silyl protecting group. The reaction was concentrated to remove the tetrahydrofuran, diluted with water (20 mL) and the product was extracted with ethyl acetate (3×25 mL). The combined organic layers were washed with water, saturated sodium bicarbonate, and brine (25 mL each). The organic layer was then dried over sodium sulfate, filtered and concentrated in vacuo to afford the crude product which was purified by silica gel chromatography on a Biotage 40S (40 g) using 25% ethyl acetate in CH₂Cl₂ as the elutant. The fractions containing the product were combined and concentrated to give 0.112 g (63%) of the desired product (5) as a white solid; MS ES⁺491.1 m/z for [C₂₅H₃₁FN₂O₇+H]⁺; MS ES^{˜}489.2 m/z for [C₂₅H₃₁FN₂O₇−H]⁻.

### Ethyl-7-{[(1S)-4-tert-butoxy-1-(hydroxymethyl)-4-oxobutyl]amino}-1-cyclopropyl-6-fluoro-8-(hydroxyl-methyl)-4-oxo-1,4-dihydroquinoline-3-carboxylate (6)

Methylene chloride (5 mL) was added to ethyl-7-{[(1S)-4-tert-butoxy-1-(hydroxymethyl)-4-oxobutyl]amino}-1-cyclopropyl-6-fluoro-8-formyl-4-oxo-1,4-dihydroquino-line-3-carboxylate (5) (0.112 g, 0.000228 mol) in a 100 mL flask. Then, sodium triacetoxyborohydride (0.0968 g, 0.457 mmol) was added in one portion and the reaction was allowed to stir overnight under nitrogen. The LC/MS after this period of time shows the reaction to be complete. The reaction was diluted with water (10 mL), and the mixture was extracted with CH₂Cl₂ (3×25 mL), dried over sodium sulfate, filtered and concentrated in vacuo to give 110 mg (98%) of the desired product (6) as a white solid with no need for further purification; MS ES⁺493.0 m/z for [C₂₅H₃₃FN₂O₇+H]⁺; MS ES⁻ 491.1 m/z for [C₂₅H₃₃FN₂O₇−H]⁻.

### 3-[(8S)-1-Cyclopropyl-3-(ethoxycarbonyl)-6-fluoro-4-oxo-1,4,7,8,9,11-hexahydro[1,4]oxazepino[5,6-h]quinolin-8-yl]propanoic acid (7)

Trifluoroacetic Acid (2.00 mL, 0.0260 mol) was added to a solution of ethyl-7-{[(1S)-4-tert-butoxy-1-(hydroxymethyl)-4-oxobutyl]amino}-1-cyclopropyl-6-fluoro-8-(hydroxymethyl)-4-oxo-1,4-dihydroquinoline-3-carboxylate (6) (0.110 g, 0.223 mmol) in CH₂Cl₂ (10 mL). The reaction was monitored over 2 days until complete, and then the solvent was evaporated and the crude mixture was used in the next step without further purification; MS ES⁺419.1 m/z for [C₂₁H₂₃FN₂O₆+H]⁺; MS ES⁻ 417.1 m/z for [C₂₁H₂₃FN₂O₆−H]⁻.

### Ethyl-(3aS)-13-cyclopropyl-8-fluoro-6,10-dioxo-3a,4,5,6,10,13-hexahydro-1H,3H-pyrrolo[2′,1′:3,4][1,4]oxa-zepino[5,6-h]quinoline-11-carboxylate (8)

Pyridine (10 mL) was added to a 2 dram vial containing 3-[(8S)-1-cyclopropyl-3-(ethoxycarbonyl)-6-fluoro-4-oxo-1,4,7,8,9,11-hexahydro[1,4]oxazepino[5,6-h]quinolin-8-yl]propan-oic acid (7) (0.093 g, 0.22 mmol), and the reaction was stirred until the reactants were dissolved. Then, N-(3-dimethylaminopropyl)-N′-ethylcarbodiimide hydrochloride (0.213 g, 1.11 mmol) was added and the reaction was allowed to stir at room temperature overnight. After this time period, the LC/MS indicated that the reaction was complete. The reaction was concentrated to remove the pyridine. The residue was suspended between water and ethyl acetate (50 mL each), and the aqueous layer was separated and extracted with ethyl acetate (3×50 mL). The combined organic layers were washed with ice cold 0.5N HCl (3×25 mL) to remove the excess pyridine, and then the organic layer was washed with water (50 mL) and brine (50 mL). The organic layer was then dried over sodium sulfate, filtered and concentrated in vacuo to give 15 mg (17%) of the product (8) as a white solid; ¹H NMR (400 MHz, CDCl₃) [symbol] ppm 0.80-0.98 (m, 2H), 1.12-1.33 (m, 2 H), 1.41 (t, J=7.15 Hz, 3H), 1.81 (dd, J=6.95, 1.55 Hz, 1H), 2.53 (ddd, J=13.01, 9.74, 9.59 Hz, 1H), 2.59-2.74 (m, 2H), 3.60 (t, J=11.40 Hz, 1H), 3.82-3.94 (m, 2 H), 4.03 (dd, J=11.92, 3.21 Hz, 1H), 4.24 (d, J=14.10 Hz, 1H), 4.39 (qd, J=7.08, 2.59 Hz, 1H), 5.74 (dd, J=13.89, 2.07 Hz, 1H), 8.21 (d, J=9.74 Hz, 1H), 8.67 (s, 1 H); MS ES⁺401.0 m/z for [C₂₁H₂₁FN₂O₅+H]⁺.

### (3aS)-13-Cyclopropyl-8-fluoro-6,10-dioxo-3a,4,5,6,10,13-hexahydro-1H,3H-pyrrolo[2′,1′:3,4]oxazepino-[5,6-h]quinoline-11-carboxylic acid (9)

Acetonitrile (3 mL) was added to a 2 dram vial containing ethyl-(3aS)-13-cyclopropyl-8-fluoro-6,10-dioxo-3a,4,5,6,10,13-hexahydro-1H,3H-pyrrolo[2′,1′: 3,4]-[1,4]oxazepino[5,6-h]quinoline-11-carboxylate (8) (0.015 g, 0.037 mmol), and then water (0.340 mL) and 0.112 mL of 1.0 M aqueous sodium hydroxide were added. The reaction was heated at 50° C. in a reaction block, and after 1 hr, the reaction was complete. The reaction was concentrated to remove the acetonitrile under a nitrogen stream, and then quenched with a minimal amount of 10% acetic acid, at which time a white precipitate formed. The solid was filtered, washed with water and then hexanes and dried under high vacuum to give 12 mg (86%) of (9) as a white solid; ¹H NMR (400 MHz, CDCl₃) δ ppm 14.36 (s, 1H), 8.97 (s, 1H), 8.28 (d, J=9.33 Hz, 1 H), 5.88 (dd, J=13.99, 2.18 Hz, 1H), 4.30 (d, J=14.10 Hz, 1H), 4.06 (m, 2H), 3.92 (m, 1H), 3.62 (t, J=11.51 Hz, 1H), 2.70 (m, 2H), 1.88 (m, 1H), 1.48 (m, 1H), 1.38 (m, 1H), 0.96 (m, 2H); MS ES⁺373.0 m/z for [C₁₉H₁₇FN₂O₅+H]⁺; MS ES⁻ 371.0 m/z for [C₁₉H₁₇FN₂O₅−H]⁻.

### Example 22

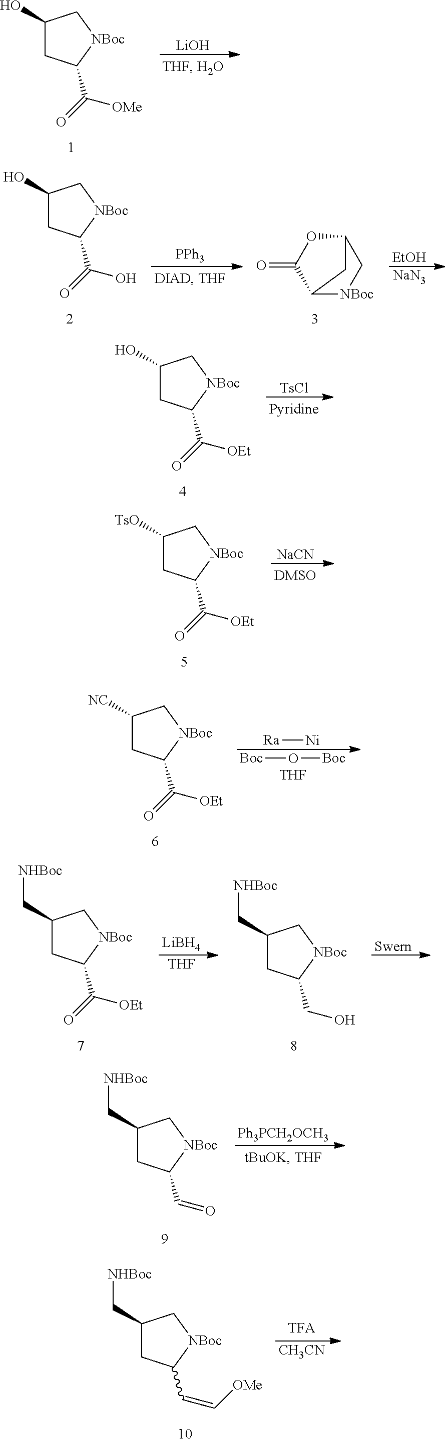

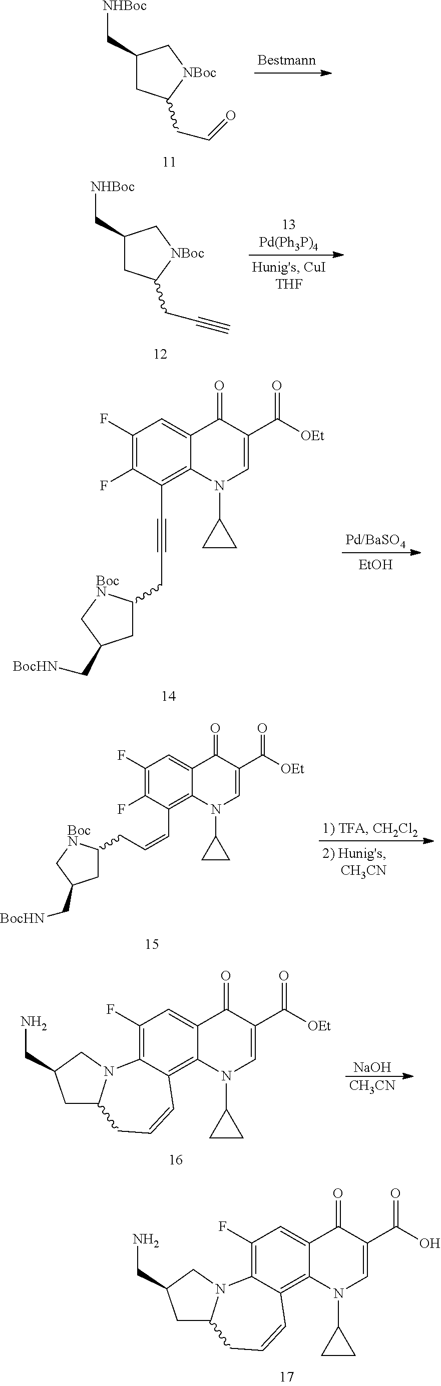

All reactions were performed under an atmosphere of nitrogen. Unless otherwise indicated, the reaction flask was evacuated with vacuum and then back-filled with nitrogen via a balloon (×3) and the reaction kept under nitrogen via balloon for the duration of the reaction.

Analytical HPLC was performed using an Agilent 1100 HPLC with one of the following methods:

Method A: Agilent Scalar C18 150×4.6 mm 5 micron column; 1.5 mL/min; solvent A—water (0.1% TFA); solvent B—acetonitrile (0.07% TFA, gradient: 10 min 95% A to 95% B; 5 min hold; then recycle; UV detection @ 214, 250 and 280 nm.

Method B: Agilent XDB C18 50×4.6 mm/1.8 micron column; 1.5 mL/min; solvent A—water (0.1% TFA), solvent B—acetonitrile (0.07% TFA); gradient: 5 min 95% A to 95% B then 1 min hold, 1 min 95% B to 95% A then 30 sec hold; UV detection @ 210, 254, and 280 nm.

Method C: Agilent Eclipse XBD C8 column; solvent A—water (0.1% TFA); solvent B—acetonitrile (0.07% TFA, gradient: 10 min 95% A to 95% B; 5 min hold; then recycle; UV detection @ 214, 250 and 280 nm.

Preparative HPLC conditions: Phenomenex Luna 250×21.20 mm, 10 micron; solvent A is 0.07% TFA in acetonitrile; solvent B is 0.10% TFA in water; 26 minute run; gradient: 5% to 80% A over 10 minutes; from 80% to 100% A over 5 minutes; hold 100% A for 5 minutes; 100% to 5% A over 5 minutes; hold 1 minute then recycle; detection at 280 nm.

Thin layer chromatography (TLC) was performed using Analtech TLC plates GHLF, 250 microns, order #21521. Regular phase silica gel chromatography was done using R10030B 40-63 μM 60 Å silica gel from Silicycle. ¹H NMR was obtained on a Brucker Avance 400 MHz instrument in the stated solvent. Mass spectral data was obtained on a Micromass instrument using electrospray ionization.

### (2S,4R)-1-(tert-Butoxycarbonyl)-4-hydroxypyrrolidine-2-carboxylic acid (2)

1-tert-Butyl-2-methyl-(2S,4R)-4-hydroxypyrrolidine-1,2-dicarboxylate (1) (10.00 g, 40.8 mmol, Synthetech Inc.) in 100 mL of MeOH was treated with 100 mL of 1.0 N LiOH at room temperature overnight. TLC after this period of time (20% EtOAc/CH₂Cl₂) shows no remaining starting material. The solvent was evaporated and then the residual water was removed by azeotrope with toluene (2×300 mL). The remaining solid was taken up in 200 mL of CH₃CN and cooled in an ice bath before adding concentrated HCl (8.2 mL) and then the mixture was stirred with cooling for 10 minutes. The residual water was removed by repeated addition and evaporation of toluene (3×150 mL) to give a clear oil. Finally, the oil was taken up in 150 mL of EtOAc and dried over Na₂SO₄. Evaporation of the volatiles gave 9 g of a clear oil which was dried at room temperature under high vacuum overnight to afford 7 g (95% yield) of (2) as a white foam; MS confirms desired product: ES⁻ m/z 230 (M−1) for [C₁₀H₁₇NO₅−1]⁻

### tert-Butyl-(1S,4S)-3-oxo-2-oxa-5-azabicyclo[2.2.1]heptane-5-carboxylate (3)

Diisopropyl azodicarboxylate (11.8 mL, 0.0602 mol) was added drop wise to a solution of (2S,4R)-1-(tert-butoxycarbonyl)-4-hydroxypyrrolidine-2-carboxylic acid (2) (11.60 g, 50.16 mmol) and triphenylphosphine (15.8 g, 60.2 mmol) in 100 mL of THF which was cooled in an ice bath under N₂. When the addition was complete the ice bath was removed and the mixture was stirred at room temperature overnight. TLC in 50% EtOAc/hexane after this period of time shows no starting material. The solvent was evaporated and the remaining oil was stirred in 200 mL of Et₂O at room temperature for 1 hour. The solid was removed by filtration and the filtrate was evaporated. The residue was taken up in 200 mL of 50% EtOAc/hexane. The solid was removed by filtration and the filtrate was chromatographed on 300 g of silica gel in 10% EtOAc/CH₂Cl₂ to afford 7.2 g (67% yield) of (3); ¹H NMR (400 MHz, CDCl₃) δ ppm 1.31 (br. s., 12H), 1.50 (br. s., 56H), 2.04 (br. s., 7H), 2.22 (br. s., 6H), 3.50 (br. s., 12H), 5.05 (br. s., 7H).

### 1-tert-Butyl-2-ethyl (2S,4S)-4-hydroxypyrrolidine-1,2-dicarboxylate (4)

A mixture of tert-butyl(1S,4S)-3-oxo-2-oxa-5-azabicyclo[2.2.1]heptane-5-carboxylate (3) (7.20 g, 0.0338 mol) and sodium azide (4.39 g, 0.0675 mol) in 50 mL of absolute EtOH was stirred at 40° C. under N₂ overnight. TLC in 50% EtOAc/hexane after this period of time shows no starting material. The EtOH was evaporated and the remaining solid was partitioned between 100 mL of water and 100 mL of EtOAc. The water layer was extracted with 2×50 mL portions of EtOAc, and then the combined organic layers were extracted with 2×50 mL portions of water and dried over Na₂SO₄. Evaporation afforded 7.60 g (87% yield) of (4); ¹H NMR (400 MHz, CDCl₃) δ ppm 1.29 (br. s., 5H), 1.45 (br. s., 7H), 2.10 (br. s., 1H), 2.33 (br. s., 1H), 3.61 (br. s., 3H), 4.29 (br. s., 4H).

### 1-tert-Butyl-2-ethyl-(2S,4S)-4-{[(4-methylphenyl)sulfonyl]oxy}pyrrolidine-1,2-dicarboxylate (5)

p-Toluenesulfonyl chloride (11.2 g, 0.0586 mol) was added to a mixture of 1-tert-butyl-2-ethyl-(2S,4S)-4-hydroxypyrrolidine-1,2-dicarboxylate (4) (7.60 g, 0.0293 mol) in 50 mL of pyridine which was cooled in an ice bath under N₂. The mixture was stirred overnight and the ice bath was allowed to expire slowly. TLC in 50% EtOAc/hexane after this period of time shows no starting material. The pyridine was evaporated and the residue was partitioned between 100 mL of CH₂Cl₂ and 100 mL of water. The layers are separated and the water was extracted with 3-100 mL portions of CH₂Cl₂, and then the combined organic layers were washed with water (2×100 mL) and 1.0 N NaOH (2×100 mL) and then dried over Na₂SO₄. Evaporation afforded 15 g of a light yellow oil. Chromatography on 300 g of silica gel in 25% EtOAc/hexane afforded 8.3 g (68% yield) of (5); ¹H NMR (400 MHz, CDCl₃) [symbol] ppm 1.16-1.33 (m, 4 H), 1.37-1.54 (m, 7H), 2.28-2.58 (m, 4H), 3.51-3.80 (m, 2H), 4.16 (dd, J=13.68, 7.05 Hz, 2H), 4.28-4.48 (m, 1H), 4.88-5.21 (m, 1H), 7.37 (dd, J=7.88, 3.52 Hz, 2 H), 7.67-7.86 (m, 2H).

### 1-tert-Butyl-2-ethyl (2S,4R)-4-cyanopyrrolidine-1,2-dicarboxylate (6)

A mixture of 1-tert-butyl-2-ethyl-(2S,4S)-4-{[(4-methylphenyl)sulfonyl]oxy}pyrrolidine-1,2-dicarboxylate (5) (8.30 g, 0.0201 mol) and sodium cyanide (4.92 g, 0.100 mol) (finely ground) in 15 mL of DMSO was warmed in a 55° C. oil bath under N₂ overnight. After this period of time, TLC in 25% EtOAc/hexane showed no starting material. The mixture was cooled to room temperature and then diluted with 150 mL of 1:1 brine/water. The resulting aqueous mixture was extracted with 3-100 mL portions of EtOAc, and then the combined organic layers were washed with (2×100 mL) water, dried over Na₂SO₄ and concentrated in vacuo to afford yellow oil (5.3 g, crude). Chromatography on 150 g of silica gel in 5% EtOAc/CH₂Cl₂ gave 3.60 g (67% yield) of (6); ¹H NMR (400 MHz, CDCl₃) [symbol] ppm 1.30 (t, 3 H), 1.39-1.55 (m, 9H), 2.32-2.43 (m, 1H), 2.44-2.63 (m, 1H), 3.20-3.35 (m, 1H), 3.58-3.77 (m, 1H), 3.92 (br. s., 1H), 4.11-4.31 (m, 2H), 4.34-4.54 (m, 1H).

### 1-tert-Butyl-2-ethyl-(2S,4S)-4-{[(tert-butoxycarbonyl)amino]methyl}pyrrolidine-1,2-dicarboxylate (7)

A mixture of 1-tert-butyl-2-ethyl-(2S,4R)-4-cyanopyrrolidine-1,2-dicarboxylate (6) (3.50 g, 0.0130 mol), di-tert-butyldicarbonate (5.69 g, 0.0261 mol) and nickel (1.53 g, 0.0261 mol) in 50 mL THF was stirred at room temperature under 50 psi of H₂ on a par shaker overnight. TLC in 30% EtOAc/hexanes after this period of time shows no remaining starting material. The Ni(s) was removed by filtration through Celite 545 and the filtrate was evaporated. Chromatography of the crude product in 20% EtOAc/hexane (3 L) followed by 40% EtOAc/hexane (1 L) on 150 g of silica gel afforded 3.4 g of (7) (which eluted in the 40% EtOAc/hexane) as a clear oil. ¹H NMR (400 MHz, CDCl₃) [symbol] ppm 1.28 (q, J=7.05 Hz, 3H), 1.37-1.51 (m, 16 H), 1.86-2.15 (m, 2H), 2.39-2.61 (m, 1H), 2.96-3.27 (m, 3H), 3.59-3.77 (m, 1H), 4.07-4.24 (m, 2H), 4.24-4.41 (m, 1H), 4.67 (br. s., 1H).

### tert-Butyl-(2S,4S)-4-[(tert-butoxycarbonyl)amino]methyl-2-(hydroxymethyl)pyrrolidine-1-carboxylate (8)

Lithium tetrahydroborate (0.512 g, 0.0235 mol) was added to a mixture of 1-tert-butyl-2-ethyl-(2S,4S)-4-{[(tert-butoxycarbonyl)amino]methyl}pyrrolidine-1,2-dicarboxylate (7) (3.50 g, 0.00940 mol) in 50 mL of THF which was cooled in an ice bath under N₂. The mixture was stirred overnight while the ice bath was allowed to expire slowly. TLC in 50% EtOAc/hexane after this period of time showed no starting material. The reaction was quenched by the addition of 50 mL of water with ice bath cooling. The THF was evaporated and the mixture was extracted with 3-50 mL portions of CH₂Cl₂, and then dried over Na₂SO₄ and concentrated in vacuo to afford 2.6 g (84% yield) of (8) as a clear oil; ¹H NMR (400 MHz, CDCl₃) [symbol] ppm 1.38-1.56 (m, 18H), 1.64-1.92 (m, 3H), 2.25-2.59 (m, 1H), 3.04-3.22 (m, 3H), 3.42-3.53 (m, 1H), 3.63 (br. s., 2H), 4.02-4.13 (m, 1H), 4.39 (br. s., 1H), 4.68 (br. s., 1H).

### tert-Butyl-(2S,4S)-4-{[(tert-butoxycarbonyl)amino]methyl}-2-formylpyrrolidine-1-carboxylate (9)

Dimethyl sulfoxide (2.23 mL, 0.0315 mol) was added to a solution of oxalyl chloride (1.33 mL, 0.0157 mol) in 100 mL of CH₂Cl₂ which was cooled to −78° C. under N₂. The mixture was stirred at −78° C. for 10 minutes, then tert-butyl (2S,4S)-4-{[(tert-butoxycarbonyl)amino]methyl}-2-(hydroxymethyl)pyrrolidine-1-carboxylate (8) (2.60 g, 0.00787 mol) was added as a solution in 50 mL of CH₂Cl₂. The addition was done at a rate to keep the reaction temperature <−70° C. When the addition was complete the mixture was stirred for 10 minutes and then triethylamine (4.39 mL, 0.0315 mol) was added. The mixture was stirred at −78° C. for 1 hour, at which time TLC in 50% EtOAc/hexane showed no starting material. The reaction was quenched by the addition of 50 mL of water and the mixture was warmed to room temperature. The layers are separated and the aqueous phase was extracted with 50 mL of CH₂Cl₂, and then the combined organic layers were dried over Na₂SO₄ and concentrated in vacuo to afford 2.5 g (97% yield) of (9) as a clear oil; ¹H NMR (400 MHz, CDCl₃) δ ppm 1.35-1.59 (m, 18H), 1.73-1.87 (m, 1H), 2.03-2.23 (m, 1H), 2.24-2.52 (m, 1 H), 2.65 (s, 1H), 3.00-3.35 (m, 2H), 3.54-3.75 (m, 1H), 4.33 (br. s., 1H), 4.56-4.79 (m, 1H), 9.43-9.75 (m, 1H).

### tert-Butyl-(4S)-4-{[(tert-butoxycarbonyl)amino]methyl}-2-[(E)-2-methoxyvinyl]pyrrolidine-1-carboxylate (10)

Potassium tert-butoxide (1.81 g, 0.0161 mol) was added to a suspension of (methoxymethyl)triphenylphosphonium chloride (5.79 g, 0.0169 mol) in 50 mL of THF which was cooled in an ice bath. When the addition was complete, the ice bath was removed and the mixture was stirred at room temperature for 1 hour. tert-Butyl-(4S)-4-{[(tert-butoxycarbonyl)amino]methyl}-2-formylpyrrolidine-1-carboxylate (9) (2.50 g, 0.00761 mol) was added slowly as a solution in 50 mL of THF, and then the mixture was allowed to stir overnight at room temperature. TLC in 10% EtOAc/CH₂Cl₂ after this period of time shows no remaining starting material. The reaction was quenched by the addition of 50 mL of saturated NaHCO₃ and then the THF was evaporated. The resulting aqueous mixture was extracted with 3-50 mL portions of EtOAc. The combined EtOAc layers were washed with 50 mL of brine and dried over Na₂SO₄. Evaporation afforded 3 g of a dark solid. Chromatography in 10% EtOAc/CH₂Cl₂ on 150 g of silica gel gave 2.2 g (81% yield) of (10) as a clear oil; ¹H NMR (400 MHz, CDCl₃) [symbol] ppm 1.46 (s, 18H), 1.81 (br. s., 2H), 2.49 (br. s., 1H), 3.11 (br. s., 3 H), 3.46-3.58 (m, 3H), 3.57-3.64 (m, 1H), 4.56-4.80 (m, 1H), 5.86 (br. s., 1H), 6.30-6.64 (m, 1H).

### tert-Butyl-(4S)-4-{[(tert-butoxycarbonyl)amino]methyl}-2-(2-oxoethyl)pyrrolidine-1-carboxylate (11)

TFA in water (15 mL, 0.4%) was added to a solution of tert-butyl-(4S)-4-{[(tert-butoxycarbonyl)amino]methyl}-2-[(E)-2-methoxyvinyl]pyrrolidine-1-carboxylate (10) (2.20 g, 6.17 mmol) in 50 mL of CH₃CN at room temperature and the mixture was stirred for 2 hours. TLC in 10% EtOAc/CH₂Cl₂ shows no starting material. The reaction was quenched by addition of 50 mL of saturated NaHCO₃ and the CH₃CN was evaporated. The resulting aqueous mixture was extracted with 3-50 mL portions of CH₂Cl₂. The organic extracts were dried over Na₂SO₄. Evaporation afforded 1.7 g (80% yield) of (11) as a yellow oil; ¹H NMR (400 MHz, CDCl₃) [symbol] ppm 1.31 (s, 18H), 1.58-1.94 (m, 1H), 2.18-2.64 (m, 2H), 2.77-3.19 (m, 3H), 3.32-3.49 (m, 1H), 3.99-4.40 (m, 1H), 4.56-4.80 (m, 1H), 9.60-9.77 (m, 1H).

### tert-Butyl-(4S)-4-{[(tert-butoxycarbonyl)amino]methyl}-2-prop-2-yn-1-ylpyrrolidine-1-carboxylate (12)

A solution of dimethyl 2-oxopropylphosphonate (1.03 mL, 0.00745 mol) and potassium carbonate (2.06 g, 0.0149 mol) in 50 mL of CH₃CN was cooled in an ice bath for 10 min under N₂. A solution of tosyl azide (1.47 g, 7.45 mmol) in 10 mL of CH₃CN was added over 10 minutes. When addition was complete the suspension was stirred for 2 hr at room temperature. TLC (in 5% methanol in dichloromethane) revealed the complete formation of the diazophosphonate. Then tert-butyl (4S)-4-{[tert-butoxycarbonyl)amino]methyl}-2-(2-oxoethyl)pyrrolidine-1-carboxylate (11) (1.70 g, 4.96 mmol) in CH₃OH (40 mL) was added via addition funnel, and the mixture was stirred overnight. TLC in 40% ethyl acetate/hexanes after this period of time showed no starting material. The reaction was quenched with 50 mL of saturated NaHCO₃ and the CH₃CN/MeOH was removed in vacuo. The resulting aqueous layer was extracted with ethyl acetate (3×50 mL), and the combined organics were washed with water (2×25 mL) and brine (25 mL). The organic layer was then dried with sodium sulfate, filtered, and concentrated. Chromatography eluting with 10% EtOAc/CH₂Cl₂ on 150 g of silica gel gave 0.98 g (58% yield) of (12) as an oil; MS ES⁺ m/z 361 (M+Na) [C₁₈H₃₀N₂O₄+Na]⁺.

### Ethyl-8-{3-[(4S)-1-(tert-butoxycarbonyl)-4-{[(tert-butoxycarbonyl)amino]methyl}-pyrrolidin-2-yl]prop-1-yn-1-yl}-1-cyclopropyl-6,7difluoro-4-oxo-1,4-dihydroquinoline-3-carboxylate (14)

tert-Butyl-(4S)-4-{[(tert-butoxycarbonyl)amino]methyl}-2-prop-2-yn-1-ylpyrrolidine-1-carboxylate (12) (5.90 mg, 1.74 mmol) and ethyl-1-cyclopropyl-6,7-difluoro-4-oxo-8-{[(trifluoromethyl)sulfonyl]oxy}-1,4-dihydroquinoline-3-carboxylate (13) (769 mg, 1.74 mmol) were combined in a 40 mL scintillation vial and placed under N₂ via vacuum evacuation and backfill with nitrogen. Triphenylphosphine (110 mg, 0.44 mmol) and tetrahydrofuran (20 mL, 200 mmol) were added and the mixture was sparged with nitrogen for 3-4 minutes. Then, N,N-diisopropylethylamine (0.607 mL, 3.49 mmol) and tetrakis(triphenylphosphine)palladium(0) (201 mg, 0.174 mmol) were added with continued sparging (˜3 min) followed by copper(I) iodide (120 mg, 0.633 mmol). The resultant clear-colored yellow reaction mixture was heated at 60° C. overnight. TLC in 15% EtOAc/CH₂Cl₂ after this period of time revealed no starting material. The mixture was diluted with ethanol (˜5 mL) and then stirred for 5 minutes, finally concentrated in vacuo to give a dark oil. Chromatography on 150 g of silica gel eluting first with 40% EtOAc/CH₂Cl₂ (2 L) and then with 100% EtOAc (1 L) gave 0.52 g (47% yield) of (14) as a light yellow foam; MS ES⁺ m/z 630 (M+1) for [C₃₃H₄₁F₂N₃O₇]⁺.

### Ethyl-8-{(1Z)-3-[(4S)-1-(tert-butoxycarbonyl)-4-{[(tert-butoxycarbonyl)amino]methyl}-pyrrolidin-2-yl]prop-1-en-1-yl}-1-cyclopropyl-6,7-difluoro-4-oxo-1,4-dihydroquinoline-3-carboxylate (15)

Ethyl-8-{3-[(4S)-1-(tert-butoxycarbonyl)-4-{[(tert-butoxycarbonyl)amino]methyl}pyrrolidin-2-yl]prop-1-yn-1-yl}-1-cyclopropyl-6,7-difluoro-4-oxo-1,4-dihydroquinoline-3-carboxylate (14) (0.520 g, 0.826 mmol) was dissolved in ethanol (40 mL, 800 mmol). The mixture was sparged with N₂ and then 0.352 g of 5% palladium on barium sulfate, was added in one portion. Finally, triethylamine (0.022 mL, 0.16 mmol) was added to ensure that the reaction was basic. The mixture was sparged with nitrogen then hydrogen and then maintained under an atmosphere of hydrogen with a hydrogen balloon at room temperature. After 3 hrs, HPLC shows no starting materials. The catalyst was removed by filtration and the filtrate was evaporated to give 0.46 g of (15) as an oil; MS ES⁺ m/z 632 (M+1) for [C₃₃H₄₃F₂N₃O₇]⁺.

### Ethyl-(7a,9S)-9-(aminomethyl)-4-cyclopropyl-12-fluoro-1-oxo-4,7,7a,8,9,10-hexahydro-1H-pyrrolo[1′,2′:1,7]azepino[2,3-h]quinoline-2-carboxylate (16)

Ethyl-8-{(1Z)-3-[(4S)-1-(tert-butoxycarbonyl)-4-{[(tert-butoxycarbonyl)amino]methyl}pyrrolidin-2-yl]prop-1-en-1-yl}-1-cyclopropyl-6,7-difluoro-4-oxo-1,4-dihydroquinoline-3-carboxylate (15) (0.46 g, 0.73 mmol) was dissolved in CH₂Cl₂ (20 mL) and then trifluoroacetic acid (0.8 mL, 0.01 mol) was added. The mixture was stirred at room temperature for 4 hr after which, TLC in 5% MeOH/CH₂Cl₂ showed no starting material. The reaction was quenched by they addition of 10% aqueous ammonium hydroxide solution (50 mL), the layers were separated, and then the aqueous layer was extracted with 2-50 mL portions of CH₂Cl₂. The combined organic layers were washed with brine (50 mL), dried over sodium sulfate, filtered, and concentrated in vacuo. ¹H NMR shows no BOC moieties remaining. Acetonitrile (20 mL, 0.3 mol) was added followed by N,N-diisopropylethylamine (0.507 mL, 0.00291 mol) and the reaction mixture was stirred at room temperature for 16 hr. LCMS after this period of time shows incomplete reaction; consequently, N,N-diisopropylethylamine (0.5 mL) was added and the mixture was warmed at 40° C. for 3 hours. LCMS after this period of heating revealed no remaining starting material. The mixture was cooled to room temperature and the solvent was evaporated. The remaining oil was subject to chromatography on 15 g of silica gel eluting first with 10% MeOH/CH₂Cl₂ (1 L) and then with 10% MeOH containing 1% TEA/CH₂Cl₂ (1 L) to afford 90 mg of (16) as a light yellow solid; MS ES⁺ m/z 412 (M+1) for [C₂₃H₂₆FN₃O₃]⁺.

### (9S)-9-(Aminomethyl)-4-cyclopropyl-12-fluoro-1-oxo-4,7,7a,8,9,10-hexahydro-1H-pyrrolo-[1′,2′:1,7]azepino[2,3-h]quinoline-2-carboxylic acid trifluoroacetate (17)

Ethyl-(7a, 9S)-9-(aminomethyl)-4-cyclopropyl-12-fluoro-1-oxo-4,7,7a,8,9,10-hexahydro-1H-pyrrolo[1′,2′:1,7]azepino[2,3h]-quinoline-2-carboxylate (16) (0.0450 g, 0.000109 mol) in 5 mL of CH₃CN was treated with 5 mL of 0.25 N NaOH in a 50° C. shaker block. After 2 hours, LCMS shows no starting material. The mixture was cooled to room temperature and the reaction was quenched by the dropwise addition of acetic acid to pH ˜5. The solvent was evaporated under a stream of N₂, and the remaining oil was purified by preparative HPLC on a Phenomenex Luna C-18 250-30 mm column using the following method: 0.1% TFA in water, 0.07% TFA in CH₃CN. Gradient 95% water to 5% water over 20 minutes, 5 minutes hold, and recycle. The resulting oil was lyophilized to give 22 mg of (17) as a yellow solid. HPLC (Method C) shows >95area %; ¹H NMR (400 MHz, DMSO-d₆) [symbol] ppm 0.73-1.16 (m, 4H), 1.19-1.38 (m, 1H), 1.45-1.61 (m, 1H), 1.84-2.04 (m, 1H), 2.54-2.78 (m, 2H), 2.86-3.15 (m, 2H), 3.83-4.11 (m, 2H), 4.15-4.43 (m, 2H), 5.88-6.13 (m, 1H), 6.57-6.87 (m, 1H), 7.49-7.76 (m, 1H), 7.92 (br. s., 1H), 8.68-8.82 (m, 1H); MS ES⁺ m/z 384 (M+1) for [C₂₁H₂₂FN₃O₃]⁺.

### Example 23

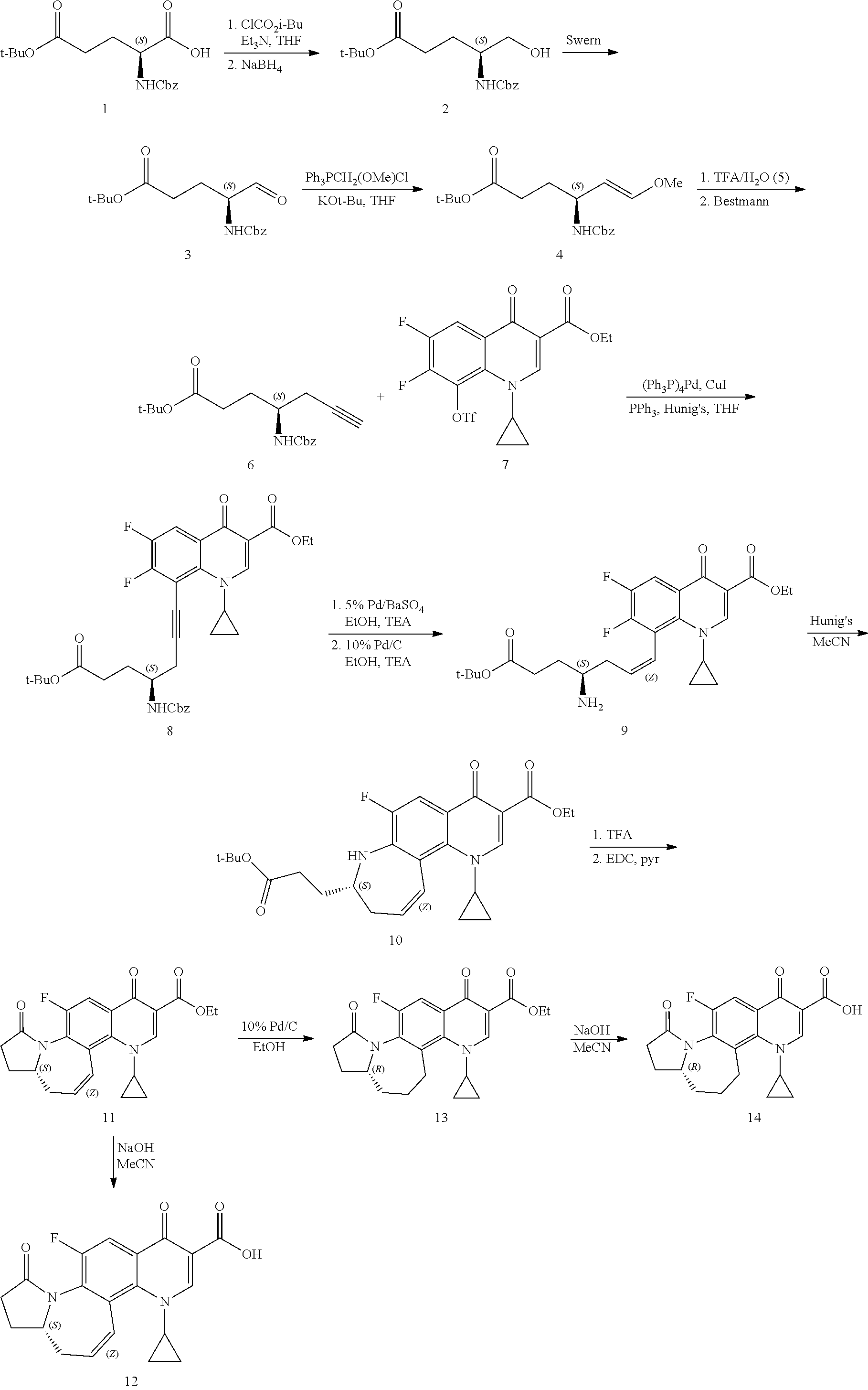

All reactions were performed under an atmosphere of nitrogen. Unless otherwise indicated, the reaction flask was evacuated with vacuum and then back-filled with nitrogen via a balloon (×3) and the reaction kept under nitrogen via balloon for the duration of the reaction.

Analytical HPLC was performed using an Agilent 1100 HPLC with one of the following methods:

Method A: Agilent Scalar C18 150×4.6 mm 5 micron column; 1.5 mL/min; solvent A—water (0.1% TFA); solvent B—acetonitrile (0.07% TFA, gradient: 10 min 95% A to 95% B; 5 min hold; then recycle; UV detection @ 214, 250 and 280 nm.

Method B: Agilent XDB C18 50×4.6 mm/1.8 micron column; 1.5 mL/min; solvent A—water (0.1% TFA), solvent B—acetonitrile (0.07% TFA); gradient: 5 min 95% A to 95% B then 1 min hold, 1 min 95% B to 95% A then 30 sec hold; UV detection @ 210, 254, and 280 nm.

Method C: Agilent Eclipse XBD C8 column; solvent A—water (0.1% TFA); solvent B—acetonitrile (0.07% TFA, gradient: 10 min 95% A to 95% B; 5 min hold; then recycle; UV detection @ 214, 250 and 280 nm.

Preparative HPLC condition—Method D: Phenomenex Luna 250×21.20 mm, 10 micron; solvent A is 0.07% TFA in acetonitrile; solvent B is 0.10% TFA in water; 26 minute run; gradient: 5% to 80% A over 10 minutes; from 80% to 100% A over 5 minutes; hold 100% A for 5 minutes; 100% to 5% A over 5 minutes; hold 1 minute then recycle; detection at 285 nm.

Prep HPLC conditions—Method E: Phenomenex Luna 250×30.00 mm, 10 micron; solvent A is 0.07% TFA in acetonitrile; solvent B is 0.10% TFA in water; rate is 20 mL/min; 30 minute run; 5% to 70% A over 14 minute ramp; 3 minute ramp from 80% to 100% A; hold 100% A for 3 minutes; ramp down from 100% to 5% A over 5 minutes; hold 10 minutes then recycle.

Thin layer chromatography (TLC) was performed using Analtech TLC plates GHLF, 250 microns, order #21521.

### tert-Butyl-(4S)-4-{[benzyloxy)carbonyl]amino}-5-hydroxypentanoate (2)

Triethylamine (2.27 mL, 0.0163 mol) and isobutyl chloroformate (2.11 mL, 0.0163 mol) were successively added to a −10° C. solution (ice/salt bath) of (2S)-2-{[(benzyloxy)carbonyl]amino}-5-tert-butoxy-5-oxopentanoic acid (1) (5.05 g, 0.0150 mol) in tetrahydrofuran (100 mL). A white precipitate (triethylammonium chloride) formed upon addition of isobutyl chloroformate. After 1 hour, the mixture was filtered through Celite® and the filter cake was washed with 50 mL of tetrahydrofuran. This filtrate was added drop-wise over 1 hour to a 0° C. mixture of sodium borohydride (1.68 g, 0.0445 mol) and water (20 mL). The reaction temperature was maintained at 0° C. for 3 hours and then allowed to warm to room temperature overnight. After this period of time, the solvent was removed by rotary evaporation and the reaction was quenched with water and 1N HCl (50 mL of each). The mixture was extracted with ethyl acetate (4×100 mL) and the combined organic layers were washed with 5% citric acid solution (100 mL), saturated sodium bicarbonate solution (100 mL), water (100 mL) and brine (100 mL). The organic layer was then dried with sodium sulfate, filtered and concentrated in vacuo. TLC (50% ethyl acetate in hexanes) shows some starting material is still present. The oil was dissolved in diethyl ether (200 mL) and washed with 1N sodium hydroxide (2×50 mL) and water (1×100 mL). The organic layer was then dried with sodium sulfate, filtered and concentrated in vacuo to give 4.84 g (91%) of the desired product (2) as a colorless oil. The material is clean enough to use as is; ¹H NMR (400 MHz, CDCl₃) δ ppm 1.44 (s, 9H), 1.72-1.95 (m, 7H), 2.26-2.55 (m, 3 H), 3.52-3.78 (m, 3H), 5.01-5.23 (m, 3H), 7.30-7.41 (m, 5H).

### tert-Butyl-(4S)-4-{[(benzyloxy)carbonyl]amino}-5-oxopentanoate (3)

Dimethyl sulfoxide (2.14 mL, 0.0302 mol) was added to a solution of oxalyl chloride (1.28 mL, 0.0151 mol) in CH₂Cl₂ (200 mL) which was cooled to −78° C. under a nitrogen atmosphere. The mixture was stirred at −78° C. for 15 minutes, and then tert-butyl-(4S)-4-{[(benzyloxy)carbonyl]amino}-5-hydroxypentanoate (2) (2.44, 0.00754 mol) was added as a solution in CH₂Cl₂ (50 mL). The substrate addition was done at a rate to keep the reaction temp lower than −75° C. When the addition was complete the mixture was stirred for 30 minutes and then triethylamine (4.21 mL, 0.0302 mol) was added. The mixture was stirred at −78° C. for 30 minutes and then allowed to warm to −35° C. over a 30 minute period of time. TLC (40% ethyl acetate in hexanes) at this time indicated complete consumption of the starting material. The reaction was quenched by addition of water (100 mL) and then mixture was warmed to room temperature. The layers were separated and the aqueous phase was extracted with dichloromethane (100 mL). The combined organic layers were washed with water (100 mL) and brine (50 mL), dried over sodium sulfate, filtered and evaporated to give 2.40 g (99%) of (3) as a viscous yellow oil. The crude product was carried on immediately to the next step without further purification.

### tert-Butyl-(4S,5E)-4-{[(benzyloxy)carbonyl]amino}-6-methoxyhex-5-enoate (4)

(Methoxymethyl)triphenyl-phosphonium chloride (8.3 g, 0.024 mol) and tetrahydrofuran (75 mL) were added to a 250-mL flask with stirring. Then, potassium tert-butoxide (2.6 g, 0.023 mol) was added and the suspension turned a deep red color. The reaction was stirred for 1 hr, and then tert-butyl-(4S)-4-{[(benzyloxy)carbonyl]amino}-5-oxopentanoate (3) (2.42 g, 7.53 mmol) was added as a solution in tetrahydrofuran (25 mL). The resulting solution was allowed to stir overnight. TLC (25% ethyl acetate in hexanes) after this period of time showed the reaction to be complete. The reaction was quenched with saturated sodium bicarbonate solution (50 mL) and concentrated to remove the tetrahydrofuran. The mixture was extracted with ethyl acetate (2×100 mL) and the combined organics were washed with water (50 mL) and brine (50 mL). The organic layer was then dried with sodium sulfate, filtered and concentrated in vacuo. The resulting dark orange oil was purified by silica gel chromatography on a Biotage 40L (120 g) using 20% ethyl acetate in hexanes as the elutant. The fractions containing the product were combined and concentrated to give 1.77 g (67%) of the product (4) as an orange oil; ¹H NMR (400 MHz, CDCl₃) δ ppm 1.44 (s, 9H), 1.69-1.96 (m, 2H), 2.18-2.35 (m, 2H), 3.44-3.66 (m, 3H), 3.89-4.91 (m, 3H), 5.09 (br. s., 2H), 5.87-6.76 (m, 1H), 7.26-7.40 (m, 5H); MS ES⁺372.2 m/z for [C₁₉H₂₇NO₅+Na]⁺.

### tert-Butyl (4S)-4-{[(benzyloxy)carbonyl]amino}hept-6-ynoate (6)

tert-Butyl-(4S,5E)-4-{[(benzyloxy)-carbonyl]amino}-6-methoxyhex-5-enoate (4) (1.77 g, 0.00506 mol) was dissolved in acetonitrile (60 mL), and then 3 mL of a 0.4 M solution of trifluoroacetic acid in water was added and the reaction was allowed to stir overnight. TLC (25% ethyl acetate in hexanes), after this period of time, indicated ˜70% conversion. Consequently, an additional 5 mL of 0.4 M aqueous trifluoroacetic acid was added to the mixture. After 4 hr the reaction was ˜90% complete. The reaction was quenched with a saturated solution of sodium bicarbonate (50 mL) and allowed to stir for 48 hr. The reaction mixture was then concentrated to remove the acetonitrile and the aqueous slurry was extracted with ethyl acetate (3×50 mL). The combined organic layers were washed with brine (50 mL), and then dried over sodium sulfate, filtered and concentrated in vacuo. The crude material was purified by silica gel chromatography on a Biotage 40L (120 g) using 20-30% ethyl acetate in hexanes as the elutant. The fractions containing the products were combined and concentrated to give 1.19 g (70%) of tert-butyl-(4S)-4-{[(benzyloxy)carbonyl]amino}-6-oxohexanoate (5) as a colorless oil; ¹H NMR (400 MHz, CDCl₃) δ ppm 1.44 (s, 9H), 1.86 (q, J=7.12 Hz, 2H), 2.33 (t, J=7.15 Hz, 2H), 2.70 (d, J=5.60 Hz, 2H), 3.99-4.20 (m, 1H), 5.04-5.23 (m, 3H), 7.29-7.42 (m, 5H), 9.77 (s, 1H). MS ES⁺358.2 m/z for [C₁₈H₂₅NO₅+Na]⁺.

Acetonitrile (15 mL), tosyl azide (0.988 g, 5.01 mmol) and potassium carbonate (1.38 g, 10.0 mmol) were added to a 100 mL flask. Then, dimethyl 2-oxopropylphosphonate (0.692 mL, 5.01 mmol) was added to the stirred suspension. The reaction was stirred for 2 hr before tert-butyl-(4S)-4-{[(benzyloxy)carbonyl]amino}-6-oxohexanoate (5) (1.12 g, 3.34 mmol) was added in methanol (30 mL) via cannula and the resultant mixture was allowed to stir overnight. TLC (25% ethyl acetate in hexanes) after this period of time indicated that the reaction was complete. The reaction was concentrated to remove the solvent and then partitioned between ethyl acetate and water (100 mL each). The layers were separated and the aqueous layer was extracted with ethyl acetate (2×75 mL). The combined organic layers were washed with water and brine (50 mL each) and then dried over sodium sulfate, filtered and concentrated in vacuo. The crude material was purified by silica gel chromatography on a Biotage 40L (120 g) using 15% ethyl acetate in hexanes as the elutant. The fractions containing the product were combined and concentrated to give 897 mg (81%) of the desired product (4) as a colorless oil; ¹H NMR (400 MHz, CDCl₃) δ ppm 1.44 (s, 9H), 1.89 (q, J=7.12 Hz, 2H), 2.02 (t, J=2.59 Hz, 1H), 2.25-2.56 (m, 4H), 3.83 (br. s., 1H), 4.99 (d, J=8.71 Hz, 1H), 5.10 (s, 2H), 7.29-7.42 (m, 5 H).

### Ethyl-8-[(4S)-4-{[(benzyloxy)carbonyl]amino}-7-tert-butoxy-7-oxohept-1-yn-1-yl]-1-cyclopropyl-6,7-difluoro-4-oxo-1,4-dihydroquinoline-3-carboxylate (8)

tert-Butyl-(4S)-4-{[(benzyloxy)carbonyl]amino}hept-6-ynoate (6) (450 mg, 1.4 mmol) and ethyl-1-cyclopropyl-6,7-difluoro-4-oxo-8-{[(trifluoromethyl)sulfonyl]oxy}-1,4-dihydroquinoline-3-carboxylate (7) (599 mg, 1.36 mmol) were combined in a 40 mL scintillation vial and then placed under nitrogen via vacuum evacuation and backfill with nitrogen. Then, triphenylphosphine (89 mg, 0.34 mmol) and tetrahydrofuran (10 mL) were added and the reaction was sparged with nitrogen for 3-4 minutes before adding N,N-diisopropylethylamine (0.473 mL, 2.72 mmol) and tetrakis(triphenylphosphine)palladium(0) (160 mg, 0.14 mmol) successively with continued sparging. Finally, copper(I) iodide (90 mg, 0.48 mmol) was added and the resultant clear-colored yellow reaction mixture was heated at 60° C. overnight. After heating for 16 hr, the reaction (a precipitated had formed) was checked by HPLC/LCMS and TLC (15% ethyl acetate in CH₂Cl₂) analysis which showed complete consumption of the starting triflate and alkyne. The crude reaction was diluted with ethanol (˜5 mL) and then stirred for 5 minutes before concentrating in vacuo. The residue was dissolved in CH₂Cl₂ and filtered to remove the precipitate. The crude reaction was then subjected to silica gel chromatography on a 120 g cartridge, eluting with 15-25% ethyl acetate in CH₂Cl₂ with 1% ethanol to afford 677 mg (80%) of the purified product (8) as a slightly yellow solid; ¹H NMR (400 MHz, CDCl₃) δ ppm 1.05-1.12 (m, 2H), 1.21-1.30 (m, 2H), 1.38-1.47 (m, 12H), 1.89-2.02 (m, 2H), 2.29-2.44 (m, 2H), 2.69-2.91 (m, 2H), 3.85-3.97 (m, 1H), 4.08-4.20 (m, 1H), 4.40 (q, J=7.19 Hz, 2H), 5.01-5.18 (m, 3H), 7.29-7.38 (m, 5H), 8.25 (dd, J=9.95, 8.91 Hz, 1H), 8.59 (s, 1H); MS ES⁺623.1 m/z for [C₃₄H₃₆F₂N₂O₇+H]⁺; MS ES″ 621.1 m/z for [C₃₄H₃₆F₂N₂O₇−H]⁻.

### Ethyl-8-[(1Z,4S)-4-amino-7-tert-butoxy-7-oxohept-1-en-1-yl]-1-cyclopropyl-6,7-difluoro-4-oxo-1,4-dihydroquinoline-3-carboxylate (9)

Ethyl-8-[(4S)-4-{[(benzyloxy)carbonyl]amino}-7-tert-butoxy-7-oxohept-1-yn-1-yl]-1-cyclopropyl-6,7-difluoro-4-oxo-1,4-dihydroquinoline-3-carboxylate (8) (0.673 g, 1.08 mmol) was dissolved in ethanol (50 mL), and then triethylamine (3 drops) was added to insure the reaction was basic to wet pH paper. The reaction was degassed by bubbling with nitrogen for 5 min before adding 0.350 g of 5% palladium on barium sulfate. The nitrogen line was then switched to a hydrogen balloon with bubbling for 3 min before removing the vent needle; then the reaction was allowed to stir under an atmosphere of hydrogen overnight. LC/MS after this period of time shows very little reaction. The reaction was rinsed into a Parr pressure vessel and an additional amount of 5% palladium on barium sulfate (0.474 g) was added. The reaction was placed on the Parr shaker under 20 psi of hydrogen for 6 hours. After this period of time, the reaction only contained a small amount of unreacted starting material, some desired alkene, and a larger amount of alkane (over-reduced) whereas the Cbz group remained intact. The reaction was filtered through a short plug of Celite 545 to remove the palladium catalyst and then the filtrate was concentrated in vacuo. The residue was redissolved in ethanol (50 mL) and cooled to 0° C. in an ice bath before adding triethylamine (3 drops) to insure the reaction was basic to wet pH paper. The reaction was sparged for 5 minutes with nitrogen, and then 10% Palladium on carbon (50 mg) was added and the reaction was sparged for an additional 5 minutes before the nitrogen line was switched to a hydrogen balloon with bubbling for 3 min. The vent needle was removed and the reaction was allowed to stir under and atmosphere of hydrogen at reduced temperature for 4 hr. LC/MS shows the reaction to be 95% complete. The reaction was sparged with nitrogen, filtered through Celite and concentrated in vacuo. The material was carried on as is to the cyclization reaction.

### Ethyl-(8S)-8-(3-tert-butoxy-3-oxopropyl)-1-cyclopropyl-6-fluoro-4-oxo-4,7,8,9-tetrahydro-1H-azepino[2,3-h]quinoline-3-carboxylate (10)

Acetonitrile (50 mL) was added to a flask containing ethyl-8-[(1Z,4S)-4-amino-7-tert-butoxy-7-oxohept-1-en-1-yl]-1-cyclopropyl-6,7-difluoro-4-oxo-1,4-dihydroquinoline-3-carboxylate (9) (0.600 g, 1.22 mmol). N,N-diisopropylethylamine (1.5 mL, 0.0086 mol) was added and the reaction was allowed to stir at room temperature for several hours. The reaction was progressing slowly (LCMS), so the reaction was heated at 50° C. overnight. LC/MS after this period of time indicated complete conversion to the product. The reaction was concentrated to remove the acetonitrile, and the crude material was purified by silica gel chromatography on a Biotage 40M (90 g) column using 25% ethyl acetate in CH₂Cl₂ with 1% ethanol as the elutant. The fractions containing the product were combined and concentrated to give 0.217 g (38%) of the desired product (10) as a white solid; ¹H NMR (400 MHz, CDCl₃) δ ppm 0.73-0.98 (m, 2H), 0.98-1.13 (m, 1H), 1.15-1.27 (m, 1H), 1.34-1.50 (m, 12H), 1.93-2.14 (m, 2H), 2.34-2.50 (m, 2H), 2.50-2.67 (m, 2H), 3.49-3.64 (m, 1 H), 3.79-3.93 (m, 1H), 4.29-4.50 (m, 2H), 5.18 (br. s., 1H), 5.99 (dt, J=12.02, 4.56 Hz, 1H), 6.69 (dd, J=12.13, 1.35 Hz, 1H), 7.88 (d, J=11.20 Hz, 1H), 8.60 (s, 1H); MS ES⁺471.1 m/z for [C₂₆H₃₁FN₂O₅+H]⁺; MS ES⁻ 469.2 m/z for [C₂₆H₃₁FN₂O₅−H]⁻.

### Ethyl-(7aS)-4-cyclopropyl-12-fluoro-1,10-dioxo-4,7,7a,8,9,10-hexahydro-1H-pyrrolo[1′,2′:1,7]azepino[2,3-h]quinoline-2-carboxylate (11)

Trifluoroacetic acid (0.20 mL, 2.6 mmol) was added to a solution of ethyl-(8S)-8-(3-tert-butoxy-3-oxopropyl)-1-cyclopropyl-6-fluoro-4-oxo-4,7,8,9-tetrahydro-1H-azepino[2,3-h]quinoline-3-carboxylate (10) (0.210 g, 0.446 mmol) in CH₂Cl₂ (10 mL). The reaction was allowed to stir at room temperature overnight. LC/MS after this period of time shows complete conversion to the desired product. The reaction was concentrated under a stream of nitrogen and then redissolved in pyridine (20 mL). N-(3-Dimethylaminopropyl)-N′-ethylcarbodiimide hydrochloride (0.278 g, 1.45 mmol) was added and the reaction was allowed to stir at room temperature overnight. After this time period, the LC/MS indicated that the reaction was 86% complete. An additional 140 mg of N-(3-dimethylaminopropyl)-N′-ethylcarbodiimide hydrochloride was added, and after an additional 2 hr, the reaction was complete based LC/MS analysis. The reaction was concentrated to remove the pyridine, and the residue was suspended between water and ethyl acetate (50 mL each). The aqueous layer was separated and extracted with ethyl acetate (3×50 mL), and the combined organics were washed with ice cold 0.5N HCl (3×25 mL) to remove the excess pyridine. The organic layer was washed with water (50 mL) and brine (50 mL), dried over sodium sulfate, filtered, and concentrated in vacuo to give 60 mg (31%) of the product (11) as a white solid; ¹H NMR (400 MHz, CDCl₃) δ ppm 0.91 (br. s., 1H), 1.12 (br. s., 1H), 1.26 (s, 2 H), 1.42 (t, J=7.15 Hz, 3H), 2.11 (s, 1H), 2.29 (s, 2H), 2.53 (dd, J=8.91, 2.07 Hz, 1 H), 2.64 (d, J=2.70 Hz, 1H), 2.62 (s, 1H), 3.82 (s, 1H), 4.41 (qd, J=7.08, 3.01 Hz, 2 H), 4.67 (br. s., 1H), 6.39 (t, J=6.95 Hz, 1H), 7.05 (dd, 1H), 8.22 (d, J=9.54 Hz, 1 H), 8.66 (s, 1H); MS ES⁺397.0 m/z for [C₂₂H₂₁FN₂O₄+H]⁺; MS ES⁻ 395.2 m/z for [C₂₂H₂₁FN₂O₄−H]⁻.

### (7aS)-4-Cyclopropyl-12-fluoro-1,10-dioxo-4,7,7a,8,9,10-hexahydro-1H-pyrrolo[1′,2′:1,7]azepino[2,3-h]quino-line-2-carboxylic acid (12)

Acetonitrile (5 mL) was added to a 2 dram vial containing ethyl-(7aS)-4-cyclopropyl-12-fluoro-1,10-dioxo-4,7,7a,8,9,10-hexahydro-1H-pyrrolo[1′,2′:1,7]azepino[2,3-h]quinoline-2-carboxylate (11) (0.030 g, 0.076 mmol). The, water (0.70 mL) and 0.23 mL of a 1.0 M aqueous solution of sodium hydroxide was then added. The reaction was heated at 50° C. in a reaction block. After 1 hr, the reaction was complete. The reaction was concentrated to ½ volume under a nitrogen stream, then subjected to prep HPLC (Method E). The fractions were checked by HPLC and the pure fractions were combined and lyophilized to give 8.1 mg (29%) of (12) as a white solid; ¹H NMR (400 MHz, CDCl₃) δ ppm 8.93 (s, 1H), 8.20 (m, 1H), 7.09 (d, J=10.57 Hz, 1H), 6.47 (dt, J=10.47, 7.05, 6.95 Hz, 5H), 4.74 (m, 1H), 3.98 (m, 1H), 2.72 (m, 2H), 2.56 (m, 1H), 2.33 (m, 2H), 2.16 (m, 1H), 1.44 (m, 1H), 1.18 (m, 1H), 0.97 (m, 2H); MS ES⁺369.1 m/z for [C₂₀H₁₇FN₂O₄+H]⁺; MS ES″ 367.1 m/z for [C₂₀H₁₇FN₂O₄−H]⁻.

### Ethyl-(7aR)-4-cyclopropyl-12-fluoro-1,10-dioxo-4,5,6,7,7a,8,9,10-octahydro-1H-pyrrolo[1′,2′:1,7]azepino[2,3-h]quinoline-2-carboxylate (13)

Ethyl-(7aS)-4-cyclopropyl-12-fluoro-1,10-dioxo-4,7,7a,8,9,10-hexahydro-1H-pyrrolo[1′,2′:1,7]azepino[2,3-h]quinoline-2-carboxylate (11) (0.030 g, 0.076 mmol) was dissolved in ethanol (10 mL) and then triethylamine (0.010 mL) added to make the reaction basic to wet pH paper. The reaction was sparged with nitrogen for 10 min before adding 10% palladium on carbon (0.016 g) was added with continued sparge for 5 min. Then, a balloon of hydrogen was added and was bubbled through the reaction mixture for 5 min before the vent needle was removed and the reaction was allowed to stir overnight under an atmosphere of hydrogen. LCMS after this period of time indicates complete reaction. The reaction was sparged with nitrogen and filtered through a Celite 545 pad. The solvent was removed by rotary evaporation to yield 30 mg (99%) of the product (13) that was carried on to the hydrolysis step; MS ES⁺399.1 m/z for [C₂₂H₂₃FN₂O₄+H]⁺.

### (7aR)-4-Cyclopropyl-12-fluoro-1,10-dioxo-4,5,6,7,7a,8,9,10-octahydro-1H-pyrrolo[1′,2′:1,7]azepino[2,3-h]quinoline-2-carboxylic acid (14)

Acetonitrile (5 mL) was added to a 2 dram vial containing ethyl-(7aR)-4-cyclopropyl-12-fluoro-1,10-dioxo-4,5,6,7,7a,8,9,10-octahydro-1H-pyrrolo[1′,2′:1,7]azepino[2,3-h]quinoline-2-carboxylate (13) (0.030 g, 0.076 mmol), and then water (0.70 mL) and 0.23 mL of an 1.0 M aqueous solution of sodium hydroxide was then added. The reaction was heated at 50° C. in a reaction block, and after 1 hr, the reaction was complete. The reaction was concentrated to remove the acetonitrile under a nitrogen stream, and quenched with a minimal amount of 10% acetic acid at which time a yellow precipitate crashed out. The solid was filtered, washed with water, and dried under high vacuum to give 19 mg of the yellow solid. This was ˜75% pure by HPLC. The product was dissolved in 1N sodium hydroxide (˜0.5 mL) and water (20 mL) and washed with ethyl acetate (3×10 mL) during which all of the yellow color was removed. The reaction was then acidified with 10% acetic acid and a white precipitate formed. The solid was filtered, washed with water, and dried under high vacuum to give 9.5 mg (34%) of (14) as a white solid; ¹H NMR (400 MHz, CDCl₃) δ ppm 14.41 (s, 1H), 8.96 (s, 1H), 8.17 (d, J=9.12 Hz, 1H), 4.13 (m, 1H), 3.97 (dd, J=15.45, 7.36 Hz, 1H), 3.72 (m, 1H), 2.67 (m, 4H), 2.25 (br. s., 1H), 2.00 (m, 3H), 1.67 (m, 1H), 1.30 (m, 2H), 1.05 (m, 1H), 0.93 (m, 1H); MS ES⁺371.0 m/z for [C₂₀H₁₉FN₂O₄+H]⁺; MS ES⁻ 369.1 m/z for [C₂₀H₁₉FN₂O₄−H]⁻.

### Example 24

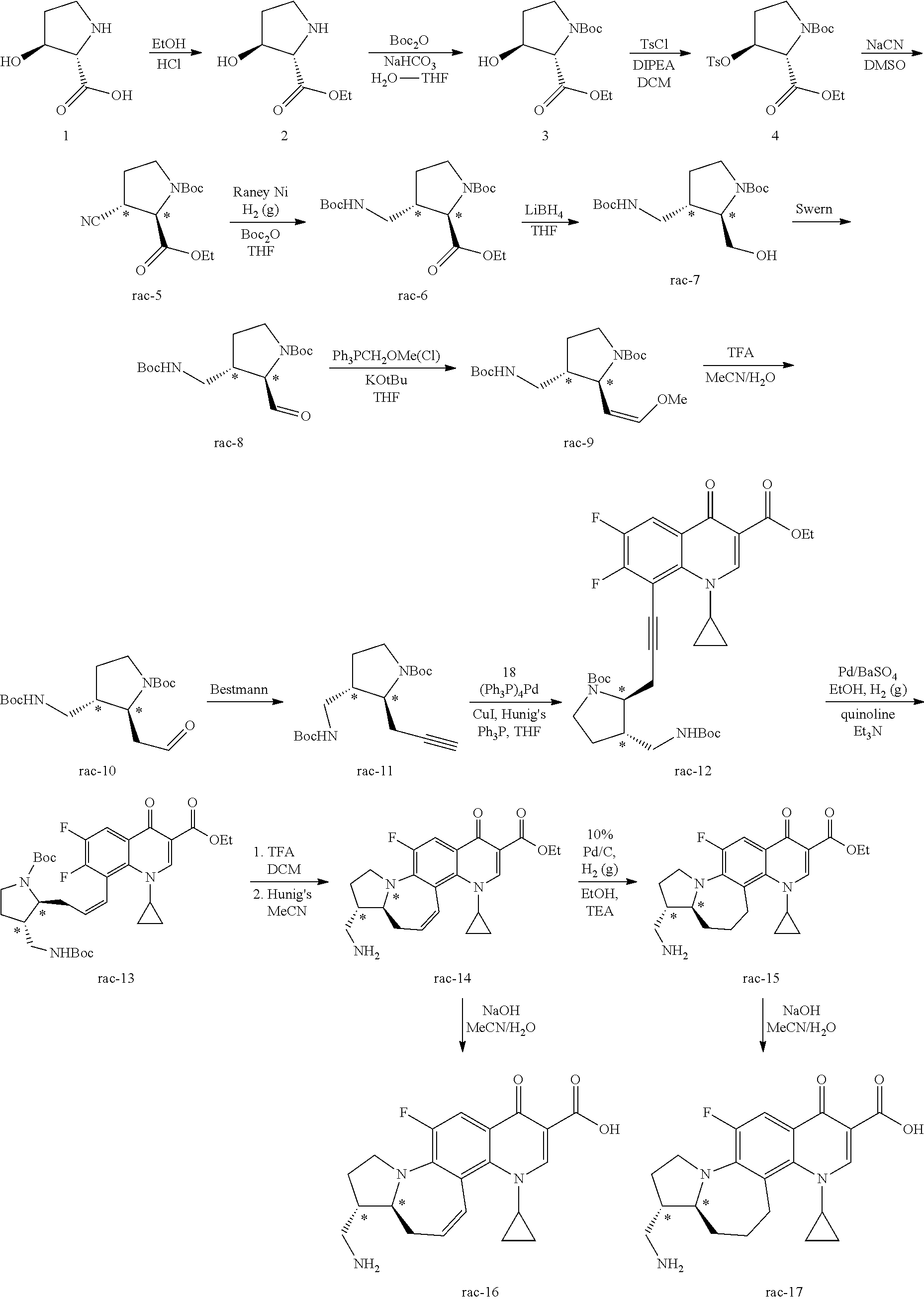

All reactions were performed under an atmosphere of nitrogen. Unless otherwise indicated, the reaction flask was evacuated with vacuum and then back-filled with nitrogen via a balloon (×3) and the reaction kept under nitrogen via balloon for the duration of the reaction.

Analytical HPLC was performed using an Agilent 1100 HPLC with one of the following methods:

Method A: Agilent Scalar C18 150×4.6 mm 5 micron column; 1.5 mL/min; solvent A—water (0.1% TFA); solvent B—acetonitrile (0.07% TFA, gradient: 10 min 95% A to 95% B; 5 min hold; then recycle; UV detection @ 214, 250 and 280 nm.

Method B: Agilent XDB C18 50×4.6 mm/1.8 micron column; 1.5 mL/min; solvent A—water (0.1% TFA), solvent B—acetonitrile (0.07% TFA); gradient: 5 min 95% A to 95% B then 1 min hold, 1 min 95% B to 95% A then 30 sec hold; UV detection @ 210, 254, and 280 nm.

Method C: Agilent Eclipse XBD C8 column; solvent A—water (0.1% TFA); solvent B—acetonitrile (0.07% TFA, gradient: 10 min 95% A to 95% B; 5 min hold; then recycle; UV detection @ 214, 250 and 280 nm.

Preparative HPLC conditions: Phenomenex Luna 250×21.20 mm, 10 micron; solvent A is 0.07% TFA in acetonitrile; solvent B is 0.10% TFA in water; 26 minute run; gradient: 5% to 80% A over 10 minutes; from 80% to 100% A over 5 minutes; hold 100% A for 5 minutes; 100% to 5% A over 5 minutes; hold 1 minute then recycle; detection at 285 nm.

Thin layer chromatography (TLC) was performed using Analtech TLC plates GHLF, 250 microns, order #21521.

### Ethyl-(3S)-3-hydroxy-L-prolinate (2) and 1-tert-butyl 2-ethyl (2S,3S)-3-hydroxypyrrolidine-1,2-dicarboxylate (3)

Thionyl chloride (1.60 mL, 22.0 mmol) was added to a solution of (3S)-3-hydroxy-L-proline (1) (2.62 g, 20.0 mmol) in ethanol (20.0 mL) that was cooled at 0° C. in an ice-water bath. After the addition was complete, the reaction was warmed to ambient temperature and then heated at reflux for 16 hr. When the reaction was complete (based on TLC analysis, 30% methanol/CHCl₃), the reaction was concentrated in vacuo and excess thionyl chloride was removed by dissolving the thick oil several times in absolute ethanol and concentrating to dryness. The crude ester (2) was then dissolved in tetrahydrofuran (100 mL) and water (20 mL) and sodium bicarbonate (8.40 g, 100 mmol) was added. The reaction was cooled at 0° C. and then di-tert-butyldicarbonate (6.5 g, 30 mmol) in tetrahydrofuran (20 mL) was added dropwise via an addition funnel, the water bath was removed, and then the reaction was stirred for 4-6 hr at ambient temperature. After this period of time, TLC (40% ethyl acetate in hexanes) shows complete consumption of the starting material and formation of a higher R_{f} product. The reaction was concentrated to remove the THF and then partitioned between ethyl acetate (200 mL) and water (50 mL). The water layer was extracted twice more with 50 mL ethyl acetate, the combined organic layers were washed with water (50 mL) and brine (50 mL), dried over MgSO₄, filtered and concentrated to afford the crude product. ¹H NMR of the crude material is clean except for residual t-BuOH. The crude product was purified by silica gel chromatography (40 g), eluting with 0, 5, 10, 15, 20, 25, and 30% ethyl acetate in hexanes to afford the desired product (3), 4.73 g in 91% yield for the two steps. ¹H NMR confirms; ¹H NMR (400 MHz, CDCl₃) δ ppm 4.46 (d, J=1.66 Hz, 1H), 4.21 (m, 3H), 3.64 (m, 2H), 2.13 (m, 2 H), 1.93 (m, 1H), 1.49 (s, 4H, t-Bu rotamer), 1.44 (s, 5H, t-Bu rotamer), 1.29 (m, 3 H).

### 1-tert-Butyl-2-ethyl (2R*,3R*)-3-cyanopyrrolidine-1,2-dicarboxylate (rac-5)

1-tert-Butyl-2-ethyl-(2S,3S)-3-hydroxypyrrolidine-1,2-dicarboxylate (3) (4.73 g, 18.2 mmol) was dissolved in CH₂Cl₂ (100 mL) and was cooled at 0° C. in an ice-water bath before adding triethylamine (7.6 mL, 55.0 mmol) and p-toluenesulfonyl chloride (3.8 g, 20.0 mmol) successively and then finally a catalytic amount of 4-dimethylaminopyridine (0.11 g, 0.91 mmol). The reaction was warmed to ambient temperature (23° C.) and stirred for 7 hr. TLC analysis after this period of time shows partial consumption of the starting material. The reaction was charged again with triethylamine (7.6 mL, 54.0 mmol) and p-toluenesulfonyl chloride (3.8 g, 20.0 mmol) with continued stirring overnight at the same temperature. After stirring overnight, the reaction was complete based on TLC analysis (30% ethyl acetate/hexanes). The reaction was quenched by the addition of ice/0.1M HCl (˜100 mL 0.1M HCl/100 mL ice) and the organic product extracted with CH₂Cl₂ (2×100 mL). The combined organic layers were washed with 0.1M HCl (2×50 mL), water (50 mL), saturated sodium bicarbonate (2×50 mL) and brine (50 mL). The organic layer was dried over MgSO₄, filtered and concentrated in vacuo to afford a dark oil that was subjected to silica gel chromatography, eluting with 0, 5, 10 and 15% ethyl acetate in hexanes to afford the desired product, 4.7 g in 62% yield, as a thick-clear oil. ¹H NMR confirms; (tosylate, 4)¹H NMR (400 MHz, CDCl₃) δ ppm 7.83 (d, J=8.29 Hz, 2H), 7.39 (d, J=7.88 Hz, 2H), 5.03 (d, J=13.27 Hz, 1H), 4.30 (d, J=7.26 Hz, 1H), 4.15 (m, 2H), 3.65 (m, 1H), 3.51 (m, 1H), 2.48 (s, 3H), 2.18 (m, 1H), 2.07 (m, 1H), 1.47 (m, 5H), 1.39 (s, 4H), 1.25 (m, 3H).

The tosylate (4, 4.7 g), was dissolved in dimethyl sulfoxide (50 mL) and was placed under an atmosphere of nitrogen. Then, sodium cyanide (2.7 g, 55.0 mmol) was added and the reaction was heated at 55° C. overnight. TLC after this period of time shows the complete consumption of the starting material and formation of a new product. The reaction was cooled to room temperature and then the reaction quenched by the addition of 200 mL of water and the organic product was extracted with diethyl ether (3×75 mL) and the combined organic layers were washed with water (50 mL) and brine (50 mL), dried over MgSO₄, filtered and concentrated in vacuo to afford the crude product which was purified on a 40 g silica gel cartridge, eluting with 0 to 15% ethyl acetate in hexanes to afford purified product, 2.58 g, in 53% yield, for the two steps; ¹H NMR confirms, but is complex (nitrile, rac-5); x-ray crystal structure determination of GG (rac-GG) shows that the structure is trans- and racemic—this racemization most likely occurred in this step; ¹H NMR (400 MHz, CDCl₃) δ ppm 4.53 (m, 1H), 4.28 (m, 2H), 3.74 (m, 2H), 3.34 (m, 1H), 2.32 (m, 2H), 1.46 (m, 9H), 1.35 (m, 3H).

### 1-tert-Butyl-2-ethyl-(2R*,3S*)-3-{[(tert-butoxycarbonyl)amino]methyl}pyrrolidine-1,2-dicarboxylate (rac-6)

1-tert-Butyl-2-ethyl-(2R*,3R*)-3-cyanopyrrolidine-1,2-dicarboxylate (rac-5) (8.13 g, 30.3 mmol), under an atmosphere of nitrogen (partial evacuation and backfill with nitrogen three times) was dissolved in tetrahydrofuran (100 mL) and then di-tert-butyldicarbonate (13.2 g, 60.6 mmol) was added followed by nickel (3.56 g, 60.6 mmol). The resulting reaction was partially evacuated and backfilled with hydrogen three times and then maintained under an atmosphere of hydrogen with a hydrogen-filled balloon. The reaction was checked after hr (the balloon had emptied by this time) and found to be ˜50% complete. The reaction was charged again with hydrogen gas and then maintained under hydrogen with a hydrogen-filled balloon and stirred overnight 12 hr. After this period of time, the TLC shows complete consumption of the starting nitrile and formation of a slightly lower R_{f} product—the Boc amine. The reaction was filtered through magnesol to remove the nickel and the filter cake was rinsed 3-4 times with ethyl acetate and then concentrated in vacuo. The resulting film was taken up in ethyl acetate and transferred to a 250 mL separatory funnel and washed once with brine (˜30 mL), dried over MgSO₄, filtered and concentrated in vacuo to afford the crude product (rac-6) (11.3 g) in quantitative yield; ¹H NMR confirms: ¹H NMR (400 MHz, CDCl₃) δ ppm 4.87 (br. s., 1H), 4.21 (m, 2H), 4.04 (d, J=4.98 Hz, 0.5H), 3.93 (d, J=5.39 Hz, 0.5H), 3.62 (m, 0.5H), 3.50 (m, 1.5H), 3.32 (m, 1H), 3.15 (m, 1H), 2.44 (m, 1H), 2.05 (m, 1H), 1.67 (m, 1H), 1.46 (s, 9H), 1.43 (s, 9H), 1.29 (m, 3H) (somewhat complex from rotamers).

### tert-Butyl-(2R*,3S*)-3-{[(tert-butoxycarbonyl)amino]methyl}-2-(hydroxymethyl)pyrrolidine-1-carboxylate (rac-7)

1-tert-Butyl-2-ethyl-(2R*,3S*)-3-{[(tert-butoxycarbonyl)amino]methyl}pyrrolidine-1,2-dicarboxylate (rac-6) (5.77 g, 15.5 mmol) and tetrahydrofuran (100 mL) were added to a 250 mL flask. The reaction was cooled at 0° C. with an ice bath. Lithium borohydride (0.506 g, 23.2 mmol) was added in one portion. The reaction was stirred at 0° C. for 1 hour then allowed to warm to room temperature and stir overnight. TLC (30% ethyl acetate in hexanes) after this time period showed the reaction to be complete. The reaction was cooled to 0° C. and water (50 mL) was added to quench. The reaction was concentrated to remove the THF and extracted with dichloromethane (3×100 mL). The combined organic layers were then dried with sodium sulfate, filtered and concentrated in vacuo to give 5.09 g (99%) of (rac-7) as a white foam. ¹H NMR (400 MHz, CDCl₃) δ ppm 1.35-1.51 (m, 18H), 1.51-1.66 (m, 1H), 1.77-2.17 (m, 3H), 3.05-3.33 (m, 3H), 3.44-3.83 (m, 5H), 4.60 (br. s., 1H), 4.86 (br. s., 1H).

### tert-Butyl-(2R*,3S*)-3-{[(tert-butoxycarbonyl)amino]methyl}-2-formylpyrrolidine-1-carboxylate (rac-8)

Dimethyl sulfoxide (4.36 mL, 61.4 mmol) was added to a solution of oxalyl chloride (2.60 mL, 30.7 mmol) in CH₂Cl₂ (400 mL) which was cooled to −78° C. under a nitrogen atmosphere. The mixture was stirred at −78° C. for 15 minutes, then tert-butyl-(2R*,3S*)-3-{[(tert-butoxycarbonyl)amino]methyl}-2-(hydroxymethyl)pyrrolidine-1-carboxylate (rac-7) (5.07 g, 15.3 mmol) was added as a solution in CH₂Cl₂ (50 mL). The addition was done at a rate to keep the reaction temp lower than −75° C. When the addition was complete the mixture was stirred for 30 minutes and triethylamine (8.55 mL, 61.4 mmol) was added. The mixture was stirred at −78° C. for 30 minutes and then allowed to warm to −35° C. over a 30 minute period of time. TLC (40% ethyl acetate in hexanes) at this time indicated complete consumption of the starting material. The reaction was quenched by addition of water (100 mL) and the mixture was warmed to room temperature. The layers were separated and the aqueous phase was extracted with dichloromethane (100 mL). The combined organic layers were washed with water (100 mL) and brine (50 mL), dried over sodium sulfate, filtered and evaporated to give 5.0 g (99%) of rac-8 as a viscous yellow oil. The crude product (rac-8) was carried on immediately to the next step. ¹H NMR (400 MHz, CDCl₃) [symbol] ppm 1.33-1.52 (m, 18H), 1.60-1.76 (m, 1H), 1.94-2.09 (m, 1H), 2.42 (br. s., 1H), 3.09-3.32 (m, 2H), 3.36-3.48 (m, 1H), 3.49-3.71 (m, 1H), 3.72-3.96 (m, 1H), 4.85 (d, J=6.63 Hz, 1H), 9.37-9.58 (m, 1H).

### tert-Butyl-(2R*,3S*)-3-{[(tert-butoxycarbonyl)amino]methyl}-2-[(E)-2-methoxyvinyl]pyrrolidine-1-carboxylate (rac-9)

Potassium tert-butoxide (5.30 g, 47.2 mmol) was added to a suspension of (methoxymethyl)triphenylphosphonium chloride (17.2 g, 50.2 mmol) in THF (150 mL) and then the reaction was allowed stir for 1.5 hr. After this period of time, tert-butyl-(2R*,3S*)-3-{[(tert-butoxycarbonyl)amino]methyl}-2-formylpyrrolidine-1-carboxylate (rac-8) (5.04 g, 15.3 mol) in THF (50 mL) was added drop-wise via cannula and the reaction was stirred overnight. TLC (50% ethyl acetate in hexanes) after 16 hours shows a new, higher R_{f} product and consumption of the starting material. The reaction was quenched by the addition of saturated sodium bicarbonate solution (100 mL) and concentrated in vacuo. The organic product was extracted with ethyl acetate (3×100 mL) and the combined organic layers were washed with brine, dried over sodium sulfate, filtered and concentrated in vacuo to afford the crude product. The crude product was purified by silica gel chromatography using a 450 g silica gel cartridge (65M) and eluting with 25% ethyl acetate in hexanes to afford 4.23 g (77%) of (rac-9) as a colorless oil; ¹H NMR (400 MHz, CDCl₃) δ ppm 1.45 (s, 18H), 1.60 (s, 1 H), 1.84-2.18 (m, 2H), 2.99-3.41 (m, 3H), 3.41-3.94 (m, 4H), 4.23-5.23 (m, 2H), 5.76-6.57 (m, 1H); MS ES⁺379.2 m/z for [C₁₈H₃₂N₂O₅+Na]⁺.

### tert-Butyl-(2S*,3S*)-3-{[(tert-butoxycarbonyl)amino]methyl}-2-oxoethyl)pyrrolidine-1-carboxylate (rac-10)

Acetonitrile (15 mL) was added to a flask containing tert-butyl (2R*,3S*)-3-{[(tert-butoxycarbonyl)amino]methyl}-2-[(E)-2-methoxyvinyl]pyrrolidine-1-carboxylate (rac-9) (4.23 g, 11.9 mmol) and then 0.4 M aqueous trifluoroacetic acid (2.0 mL) was added to the reaction mixture. The reaction was allowed to stir overnight and then analyzed by TLC (40% ethyl acetate in hexanes). After this period of time, the reaction was determined to be complete, and a saturated solution of sodium bicarbonate (10 mL) was added to quench. The reaction was concentrated to remove the acetonitrile and the organic product was extracted with ethyl acetate (3×50 mL). The combined organic layers were washed with brine (1×25 mL), dried over sodium sulfate, filtered and concentrated in vacuo to give 4.0 g (98%) of (rac-10) as a viscous light yellow oil. The product was carried on crude to the next step without further purification.

### tert-Butyl-(2S*,3S*)-3-{[(tert-butoxycarbonyl)amino]methyl}-2-prop-2-yn-1-ylpyrrolidine-1-carboxylate (rac-11)

Tosyl azide (3.51 g, 17.8 mmol), acetonitrile (50 mL) and dimethyl 2-oxopropylphosphonate (2.46 mL, 17.8 mmol) were added to a 500 mL flask and then potassium carbonate (4.92 g, 35.6 mmol) was added to the stirred suspension with continued stirring for 2 hours. TLC (5% methanol in dichloromethane) after this period of time showed complete formation of the diazophosphonate. Then, tert-butyl (2S*,3S*)-3-{[(tert-butoxycarbonyl)amino]methyl}-2-(2-oxoethyl)pyrrolidine-1-carboxylate (rac-10) (4.06 g, 11.8 mol) in methanol (100 mL) was then added via an addition funnel. The reaction was allowed to stir overnight and TLC analysis (ethyl acetate in hexanes) after this period of time indicated the reaction was complete. The reaction was quenched with saturated sodium bicarbonate solution (50 mL) and concentrated to remove the THF. The mixture was extracted with ethyl acetate (3×50 mL). The combined organic layers were washed with water (2×25 mL), brine (25 mL), dried over sodium sulfate, filtered and concentrated in vacuo to afford the crude product as an oily solid. The mixture was suspended in a minimal amount of CH₂Cl₂ and filtered to remove the white solid (tosyl amide), and the solid was washed with a minimal amount of CH₂Cl₂. The mixture was concentrated and purified by silica gel chromatography on a Biotage 40L (90 g) eluting 20% ethyl acetate in hexanes. The fractions containing the pure product were combined and concentrated to afford 1.34 g (33%) of (rac-11) as a colorless oil. ¹H NMR (400 MHz, CDCl₃) δ ppm 1.36-1.55 (m, 18H), 1.62 (br. s., 1H), 1.94-2.17 (m, 2H), 2.36-2.77 (m, 3H), 3.00-3.76 (m, 5H), 4.71 (br. s., 1H); MS ES⁺361.2 m/z for [C₁₈H₃₀N₂O₄+Na]⁺.

### Ethyl-8-{3-[(2S*,3S*)-1-(tert-butoxycarbonyl)-3-{[(tert-butoxycarbonyl)amino]methyl}pyrrolidin-2-yl]prop-1-yn-1-yl}-1-cyclopropyl-6,7-difluoro-4-oxo-1,4-dihydroquinoline-3-carboxylate (rac-12)

tert-Butyl-(2S*,3S*)-3-{[(tert-butoxycarbonyl)amino]methyl}-2-prop-2-yn-1-ylpyrrolidine-1-carboxylate (rac-11) (744 mg, 2.20 mmol) and ethyl-1-cyclopropyl-6,7-difluoro-4-oxo-8-{[(trifluoromethyl)sulfonyl]oxy}-1,4-dihydroquinoline-3-carboxylate (18) (970 mg, 2.20 mmol) were combined in a 40 mL scintillation vial and then placed under nitrogen via vacuum evacuation and backfill with nitrogen. Then, triphenylphosphine (140 mg, 0.52 mmol) and tetrahydrofuran (20 mL) were added and the reaction was sparged with nitrogen for 3-4 minutes. Then, N,N-diisopropylethylamine (0.766 mL, 4.40 mmol) and tetrakis(triphenylphosphine)palladium(0) (254 mg, 0.262 mmol) were added with continued sparging (˜3 minutes) and copper(I) iodide (150 mg, 0.77 mmol) was added and the reaction was stirred overnight at 60° C. in the sealed vessel. After this period of time, the reaction was checked by HPLC/LCMS and TLC (15% ethyl acetate in CH₂Cl₂), which showed complete consumption of the starting triflate and alkyne. The crude reaction was diluted with ethanol (5 mL) and then stirred for 5 minutes, filtered to remove the precipitated solids, and the filtrate was concentrated in vacuo and then purified by silica gel chromatography on a 120 g cartridge, eluting with 15% ethyl acetate in CH₂Cl₂ with 1% ethanol to afford the purified product (rac-12) as a yellow foam, 1.10 g, 79% yield, which was 95% pure by HPLC; HPLC: 6.939 min; ¹H NMR (400 MHz, CDCl₃) δ ppm 1.12 (br. s., 2H), 1.19-1.34 (m, 3H), 1.33-1.55 (m, 18H), 1.66 (s, 2H), 2.02-2.23 (m, 1H), 2.49 (br. s., 1H), 2.76-3.07 (m, 2H), 3.19 (d, J=5.18 Hz, 2H), 3.29-3.46 (m, 1H), 3.45-3.81 (m, 2H), 4.06-4.30 (m, 1H), 4.39 (q, J=7.19 Hz, 2H), 4.72 (br. s., 1H), 8.23 (t, J=9.43 Hz, 1H), 8.62 (s, 1H); MS ES⁺630.3 m/z (M+1) for [C₃₃H₄₁F₂N₃O₇+H]⁺.

### Ethyl-8-{(1Z)-3-[(2S*,3S*)-1-(tert-butoxycarbonyl)-3-[(tert-butoxycarbonyl)amino]methyl}pyrrolidin-2-yl]-prop-1-en-1-yl)-1-cyclopropyl-6,7-difluoro-4-oxo-1,4-dihydroquinoline-3-carboxylate (rac-13)

Ethyl-8-{3-[(2S*,3S*)-1-(tert-butoxycarbonyl)-3-{[(tert-butoxycarbonyl)amino]methyl}pyrrolidin-2-yl]prop-1-yn-1-yl}-1-cyclopropyl-6,7-difluoro-4-oxo-1,4-dihydroquinoline-3-carboxylate (rac-12) (1.10 g, 1.75 mmol) was dissolved in ethanol (100 mL). The reaction was sparged with nitrogen before adding 5% palladium on barium sulfate (0.744 g). Then triethylamine (0.050 mL, 0.36 mmol) and quinoline (0.050 mL, 0.42 mmol) were added to ensure that the reaction was basic. The reaction was sparged with nitrogen and hydrogen and then maintained under an atmosphere of hydrogen. The reaction was checked by LC/MS after 3 hr and the found to be 97% complete. The reaction was sparged with nitrogen for 5 minutes and then vacuum filtered through a short plug of Solka Floc and the filtrate concentrated in vacuo. The crude foam was placed under high vacuum overnight to afford 1.08 g (98%) of (rac-13) as a yellow foam; ¹H NMR (400 MHz, CDCl₃) δ ppm 0.76-1.28 (m, 5H), 1.31-1.52 (m, 19H), 1.52-2.34 (m, 6H), 2.82-3.93 (m, 6H), 4.40 (q, J=7.05 Hz, 2 H), 4.65 (br. s., 1H), 5.96 (br. s., 1H), 6.81 (d, J=10.99 Hz, 1H), 8.25 (t, J=9.23 Hz, 1H), 8.63 (s, 1H); MS ES⁺632.3 m/z (M+1) for [C₃₃N₄₃F₂N₃O₇+H]⁺.

### Ethyl-(7aS*,8S*)-8-(aminomethyl)-4-cyclopropyl-12-fluoro-1-oxo-4,7,7a,8,9,10-hexahydro-1H-pyrrolo[1′,2′:1,7]-azepino[2,3-h]quinoline-2-carboxylate (rac-14)

Ethyl-8-(1Z)-3-[(2S*,3S*)-1-(tert-butoxycarbonyl)-3-{[(tert-butoxycarbonyl)amino]methylpyrrolidin-2-yl]prop-1-en-1-yl}-1-cyclopropyl-6,7-difluoro-4-oxo-1,4-dihydroquino-line-3-carboxylate (rac-13) (1.08 g, 1.71 mmol) was dissolved in CH₂Cl₂ (50 mL) in a 500 mL round bottom flask and then trifluoroacetic acid (2 mL, 0.02 mol) was added. The reaction was stirred at room temperature for 4 hr and then checked for completion by HPLC (100%, 3.568 min) and found to be complete. The solvents were removed under a stream of nitrogen, 100 mL of CH₂Cl₂ was added, and then 50 mL of 10% aqueous ammonium hydroxide solution was added to neutralize the residual TFA. The aqueous layer was extracted with CH₂Cl₂ (2×50 mL) and the combined organic layers were washed with brine (50 mL), dried over sodium sulfate, filtered and concentrated in vacuo to afford the crude diamine. Next, acetonitrile (40 mL) was added followed by N,N-diisopropylethylamine (1.19 mL, 6.84 mmol) and the reaction mixture was stirred at room temperature for 16 hr. HPLC after this period of time reveals the formation of a new product (100% conversion, 4.205 min). The reaction was concentrated to remove the volatiles and then purified by silica gel chromatography using a 120 g silica gel cartridge, eluting with 10% ethanol in CH₂Cl₂ with 1% triethylamine followed by a trituration with CH₂Cl₂ to afford 570 mg (81%) of (rac-14) as a yellow solid over the two steps. ¹H NMR (400 MHz, DMSO-d₆) δ ppm 0.76-1.00 (m, 3H), 1.15-1.23 (m, 1H), 1.97 (dd, J=12.44, 6.22 Hz, 1H), 2.14-2.26 (m, 1H), 2.33 (q, J=7.05 Hz, 1H), 2.58 (br. s., 2H), 2.76-2.97 (m, 2H), 3.54-3.66 (m, 1H), 3.70-3.82 (m, 1H), 4.00-4.31 (m, 4H), 5.88-6.00 (m, 1H), 6.67 (d, J=12.44 Hz, 1H), 7.47 (d, J=14.93 Hz, 1H), 8.17 (br. s., 3H), 8.51 (s, 1H); MS ES⁺412.2 m/z (M+1) for [C₂₃H₂₆FN₃O₃+H]⁺.

### (7aS*,8S*)-8-(Aminomethyl)-4-cyclopropyl-12-fluoro-1-oxo-4,7,7a,8,9,10-hexahydro-1H-pyrrolo[1′,2′:1,7]aze-pino-[2,3-h]quinoline-2-carboxylic acid trifluoroacetate (rac-16)

Ethyl-(7aS*,8S*)-8-(aminomethyl)-4-cyclopropyl-12-fluoro-1-oxo-4,7,7a,8,9,10-hexahydro-1H-pyrrolo-[1′,2′:1,7]azepino[2,3-h]quinoline-2-carboxylate (rac-14) (94 mg, 0.23 mmol) was added to a 40 mL vial followed by water (4 mL) and 1.0 M aqueous sodium hydroxide (0.714 mL; 0.71 mmol). A white precipitate formed immediately upon addition of the base and so acetonitrile (2 mL) was added dissolve all of the reactants. The vial was capped and heated at 50° C. for 2 hr. HPLC and LC/MS after this period of time show the reaction to be complete (>95% purity). The reaction was allowed to cool to room temperature, and the basic mixture was neutralized with acetic acid to pH 5 (˜10 drops). The crude material was purified by preparatory HPLC. Four very clean fractions were isolated, combined, and lyophilized overnight to afford 74 mg (65%) of (rac-16) trifluoroacetate as a yellow solid; ¹H NMR (400 MHz, DMSO-d₆) δ ppm 8.76 (s, 1H), 7.82 (br. s., 3H), 7.62 (d, J=14.51 Hz, 1H), 6.71 (d, J=12.23 Hz, 1H), 6.00 (dt, J=12.49, 3.71 Hz, 1H), 4.25 (m, 2H), 3.77 (m, 2H), 2.88 (m, 2H), 2.60 (m, 2H), 2.28 (m, 2H), 1.95 (m, 1H), 1.28 (m, 1H), 1.01 (m, 3H); MS ES⁺384.2 m/z (M+1) for [C₂₁H₂₂FN₃O₃+H]⁺ and ES⁻ 382.2 m/z (M−1) for [C₂₁H₂₂FN₃O₃−H]⁻.

### Ethyl-(7aS*,8S*)-8-(aminomethyl)-4-cyclopropyl-12-fluoro-1-oxo-4,5,6,7,7a,8,9,10-octahydro-1H-pyrrolo-[1′,2′:1,7]azepino[2,3-h]quinoline-2-carboxylate (rac-15)

Ethyl-(7aS*,8S*)-8-(aminomethyl)-4-cyclopropyl-12-fluoro-1-oxo-4,7,7a,8,9,10-hexahydro-1H-pyrrolo[1′,2′:1,7]azepino[2,3-h]quinoline-2-carboxylate (rac-14) (0.183 g, 0.445 mmol), ethanol (10 mL), and triethylamine (0.100 mL, 0.717 mmol) were added to a 100 mL flask. Nitrogen was bubbled through the solution and 10% palladium on carbon (47 mg, 0.044 mmol) was added with continued bubbling for 5 minutes. The nitrogen line was replaced with a hydrogen balloon and after 5 minutes, the vent needle was removed and the reaction was stirred overnight under an atmosphere of hydrogen. LC/MS after this period of time showed the reaction to complete. Nitrogen was bubbled through the reaction mixture for 5 minutes and the mixture was filtered through a short plug of Solka Floc. The solution was concentrated to remove ethanol and placed under high vacuum overnight to give 170 mg (92%) of (rac-15) as a bright yellow solid; MS ES⁺414.2 m/z (M+1) for [C₂₃H₂₈FN₃O₃+H]⁺.

### (7aS*,8S*)-8-(Aminomethyl)-4-cyclopropyl-12-fluoro-1-oxo-4,5,6,7,7a,8,9,10-octahydro-1H-pyrrolo[1′,2′:1,7]-azepino[2,3-h]quinoline-2-carboxylic acid trifluoroacetate (rac-17)

Ethyl-(7aS*,8S*)-8-(aminomethyl)-4-cyclopropyl-12-fluoro-1-oxo-4,7,7a,8,9,10-hexahydro-1H-pyrrolo[1′,2′:1,7]azepino[2,3-h]quinoline-2-carboxylate (rac-15) (170 mg, 0.41 mmol) was added to a 40 mL vial followed by acetonitrile (2 mL), water (2 mL) and 1.0 M aqueous sodium hydroxide (1.23 mL; 0.0012 mol). The vial was capped and heated at 50° C. for 2 hr. HPLC and LC/MS after this period of time showed the reaction to be complete. The reaction was allowed to cool to room temperature, and the basic mixture was neutralized with acetic acid until a pH 5 was reached (˜10 drops). The crude material was purified by preparatory HPLC to afford one clean fraction that was lyophilized overnight, triturated from isopropanol, and crystallized from acetonitrile to give 23 mg (11%) of (rac-17) trifluoroacetate as a yellow solid; ¹H NMR (400 MHz, DMSO-d₆) δ ppm 8.75 (s, 1H), 7.70 (d, J=14.10 Hz, 1H), 4.30 (m, 1H), 4.03 (m, 1H), 3.64 (dd, J=14.93, 9.33 Hz, 1H), 3.49 (m, 2H), 2.87 (m, 2H), 2.21 (m, 2H), 1.88 (m, 3H), 1.62 (m, 2H), 1.19 (m, 2H), 0.99 (m, 1H), 0.75 (m, 1H); MS ES⁺386.2 m/z (M+1) for [C₂₁H₂₄FN₃O₃+H]⁺ and ES″ 384.3 m/z (M−1) for [C₂₁H₂₄FN₃O₃−H]⁻.

### Example 25

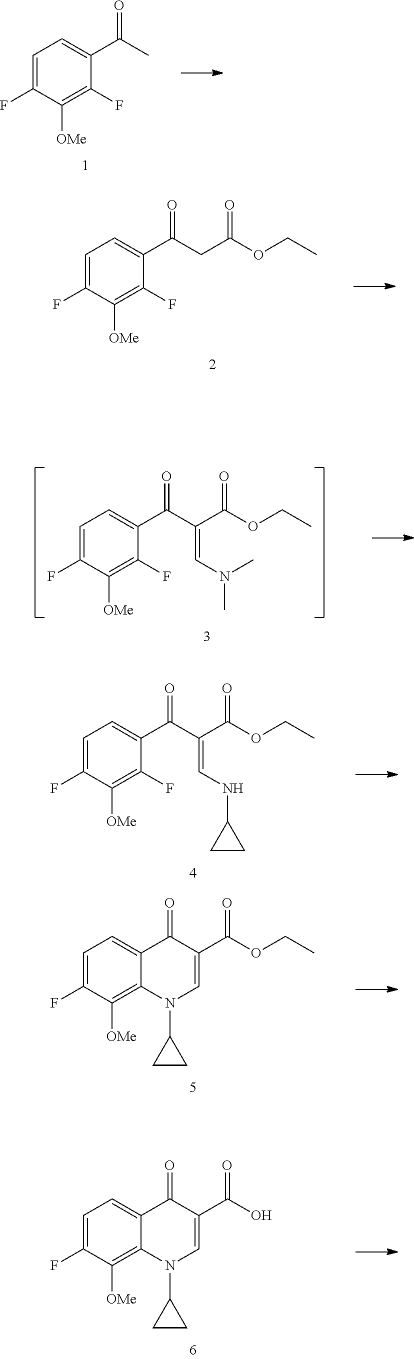

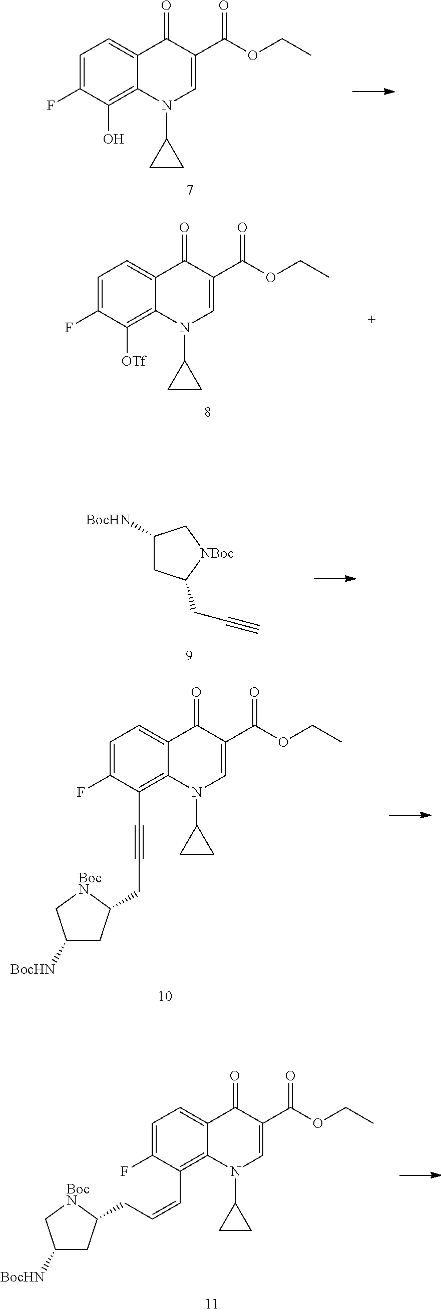

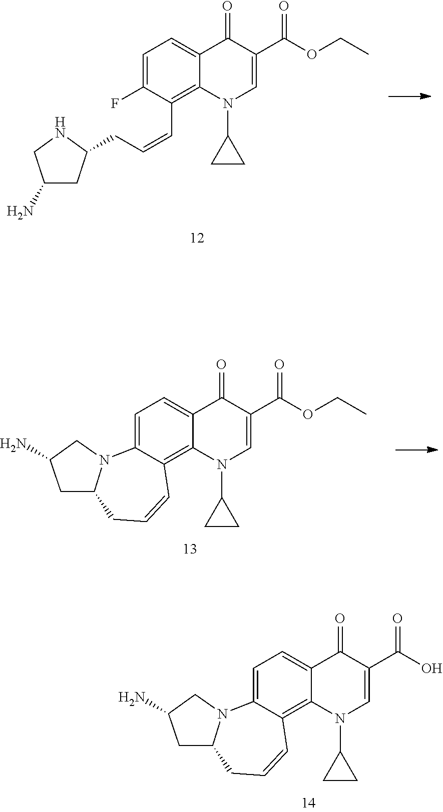

General Methods: All reactions were carried out under a nitrogen atmosphere unless otherwise noted. For compounds (1)-(13), analytical HPLC conditions: Agilent 1100 HPLC, Agilent Scalar C18 150×4.6 mm/5 micron column; 1.5 mL/min; solvent A: water (0.1% TFA), solvent B: acetonitrile (0.07% TFA); gradient: 10 min 95% A to 95% B then 5 min hold and recycle; detection @ 214 and 250 nm. For compound (14), analytical HPLC conditions: Agilent 1100 HPLC, Agilent XDB-C18 50×4.6 mm/1.8 micron column; 1.5 mL/min; solvent A: water (0.1% TFA), solvent B: acetonitrile (0.07% TFA); gradient: 5 min 95% A to 95% B then 1 min hold, 1 min 95% B to 95% A then 30 sec hold; detection @ 210, 254, and 280 nm. Preparative HPLC conditions: Phenomenex Luna 250×21.20 mm/10 micron column; solvent A: acetonitrile (0.07% TFA), solvent B: water (0.1% TFA); gradient: 1 min 10% A, 20 min 10% to 60%, hold 10 min, 3 min 60% to 95% A, hold 2 min, 5 min 5%.

### Ethyl-3-(2,4-difluoro-3-methoxyphenyl)-3-oxopropanoate (2)

A suspension of sodium hydride (2.3 g, 58 mmol, 60% in mineral oil) in toluene (25 mL, 230 mmol) was treated dropwise over 1 h with a solution of diethyl carbonate (7.8 mL, 64 mmol) in toluene (10 mL, 100 mmol) as the reaction mixture was heated to 90° C. A solution of 2,4-difluoro-3-methoxyacetophenone (1) (5.0 g, 27 mmol) in toluene (5 mL, 50 mmol) was added dropwise over 30 min to the white mixture at 90° C. to afford a clear deep yellow solution, which was stirred for 1 h and turned bright orange. HPLC indicated that the starting material had been consumed. The reaction mixture was allowed to cool to rt and was quenched with 1 M aqueous sulfuric acid (35 mL) with vigorous stirring. The separated aqueous layer was extracted with toluene (2×20 mL). The combined organics were concentrated in vacuo to a volume of ˜15 mL affording the title compound (2) as a pale yellow solution, used without further purification. HPLC purity 73% (ret. time of 2, 7.394 min).

### Ethyl-3-(cyclopropylamino)-2-(2,4-difluoro-3-methoxybenzoyl)acrylate (4)

To a solution of crude ethyl-3-(2,4-difluoro-3-methoxyphenyl)-3-oxopropanoate (2) (6.9 g, 27 mmol) in toluene (20 mL, 200 mmol) was added 1,1-dimethoxy-N,N-dimethylmethanamine (5.0 mL, 38 mmol) and the resulting bright orange mixture was heated at 95-103° C. for 1 h; HPLC analysis showed no starting material remaining. The reaction mixture was allowed to cool to rt and was treated with cyclopropylamine (2.2 mL, 32 mmol) and stirred for 1.5 h; HPLC analysis showed no starting material remaining. A 1 M aqueous solution of sulfuric acid (25 mL) was added with vigorous stirring, the mixture was diluted with toluene (25 mL), and the separated aqueous layer was extracted with toluene (2×10 mL). The combined organics were dried (Na₂SO₄) and concentrated in vacuo to afford a deep yellow oil. Extended time under reduced pressure gave the title compound (4) as a yellow solid, used without further purification. HPLC purity of 4, 63% (ret. time of 3, 6.489 min; ret. time of 4, 7.382 min).

### Ethyl-1-cyclopropyl-7-fluoro-8-methoxy-4-oxo-1,4-dihydroquinoline-3-carboxylate (5)

A solution of crude ethyl-3-(cyclopropylamino)-2-(2,4-difluoro-3-methoxybenzoyl)acrylate (4) (8.7 g, 0.027 mol) in toluene (100 mL, 0.9 mol) was treated with N,O-bis(trimethylsilyl)acetamide (6.6 mL, 0.027 mol), heated to reflux with stirring, and monitored by HPLC. At 2 h additional N,O-bis(trimethylsilyl)acetamide (3.3 mL, 0.014 mol) was added and the reaction mixture was heated at reflux for an additional 6 h, allowed to cool to rt and stirred for 15 h. The reaction mixture was heated at reflux with stirring for 1 h, treated with additional N,O-bis(trimethylsilyl)acetamide (3.3 mL, 0.014 mol), and heated at reflux for 3 h; HPLC analysis showed nearly complete conversion. The reaction mixture was allowed to cool to rt and was concentrated to half of the original volume under reduced pressure. The resulting precipitate (extremely fine needles) was collected by vacuum filtration, washed with toluene (15 mL) and water (3×10 mL), and allowed to air dry overnight to afford the title compound (5) as a slightly off-white, fine crystalline solid (1.96 g, 24%). Two additional crops of product afforded a total of 4.14 g, 51% over 3 steps. HPLC purity >95% (ret. time, 6.478 min); MS (ESI+) for C₁₆H₁₆FNO₄ m/z 306.2 (M+H)⁺.

### 1-Cyclopropyl-7-fluoro-8-hydroxy-4-oxo-1,4-dihydroquinoline-3-carboxylic Acid (6)

A vigorously stirred pale yellow solution of ethyl-1-cyclopropyl-7-fluoro-8-methoxy-4-oxo-1,4-dihydroquinoline-3-carboxylate (5) (1.91 g, 6.26 mmol) in 48% aqueous hydrogen bromide (14.1 mL, 125 mmol) was heated at 100° C. for 18.5 h. The resulting off-white mixture was allowed to cool to rt and the precipitate was collected by filtration, washed with water (5 mL) and dried in vacuo to afford a mixture of (6) and the decarboxylation product 1-cyclopropyl-7-fluoro-8-hydroxyquinolin-4(1H)-one as a chalky white solid (1.56 g, 97%) used without further purification. HPLC purity 89% (ret. time, 5.241 min); MS (ESI+) for C₁₃H₁₀FNO₄ m/z 264.2 (M+H)⁺.

### Ethyl-1-Cyclopropyl-7-fluoro-8-hydroxy-4-oxo-1,4-dihydroquinoline-3-carboxylate (7)

A suspension of 1-cyclopropyl-7-fluoro-8-hydroxy-4-oxo-1,4-dihydroquinoline-3-carboxylic acid (6) (1.07 g, 3.62 mmol) in ethanol (40 mL, 0.7 mol) was cooled to 0° C. (ice/NaCl) and treated dropwise in small portions with thionyl chloride (5 mL, 70 mmol) over 30 min to afford a fairly clear, pale yellow solution. The ice bath was removed and the reaction mixture was allowed to warm to rt and became more opaque. Additional thionyl chloride (1 mL) was added, and the reaction mixture was heated to reflux to afford a clear yellow solution after 15 min. Analytical HPLC showed complete consumption of the starting material after 3.5 h. The reaction mixture was allowed to cool to rt and was concentrated in vacuo to afford a yellow oil to which ice was added, forming a white precipitate with an orange/brown residue. The solid was isolated by filtration and washed with water (10 mL) to afford the title compound (7) as a chalky white solid (0.95 g, 75%). HPLC purity >95% (ret. time, 5.331 min); MS (ESI+) for C₁₅H₁₄FNO₄ m/z 292.2 (M+H)⁺; MS (ESI−) for C₁₅H₁₄FNO₄ m/z 290.3 (M−H)⁻.

### Ethyl-1-cyclopropyl-7-fluoro-4-oxo-8-[(trifluoromethyl)sulfonyl]oxy-1,4-dihydroquinoline-3-carboxylate (8)

To a suspension of ethyl-1-cyclopropyl-7-fluoro-8-hydroxy-4-oxo-1,4-dihydroquinoline-3-carboxylate (7) (2.0 g, 6.0 mmol) in tetrahydrofuran (40 mL, 500 mmol) was added N,N-diisopropylethylamine (2.1 mL, 12 mmol) to afford a bright yellow mixture. N-phenylbis(trifluoromethanesulphonimide) (2.27 g, 6.34 mmol) was added to the reaction mixture at rt to afford a clear, deep yellow solution, which was stirred overnight. At 17 h, LC MS showed complete consumption of the starting material. The reaction mixture was concentrated in vacuo to afford a light yellow solid, which was taken up in EtOAc (100 mL) and washed with aqueous 1 N citric acid (50 mL), saturated aqueous NaHCO₃ (50 mL), and brine (50 mL). The organic layer was dried (Na₂SO₄) and concentrated in vacuo to afford a white solid, which was recrystallized from i-PrOH to afford the title compound (8) as a needle-like white crystalline solid (1.91 g, 75%). MS (ESI+) for C₁₆H₁₃F₄NO₆S m/z 424.1 (M+H)⁺; LC purity >90%.

### Ethyl-8-(3-(2R,4S)-1-(tert-butoxycarbonyl)-4-[(tert-butoxycarbonyl)amino]pyrrolidin-2-ylprop-1-yn-1-yl)-1-cyclopropyl-7-fluoro-4-oxo-1,4-dihydroquinoline-3-carboxylate (10)

A solution of tert-butyl-(2R,4S)-4-[(tert-butoxycarbonyl)amino]-2-prop-2-yn-1-ylpyrrolidine-1-carboxylate (9) (0.261 g, 0.804 mmol) and ethyl-1-cyclopropyl-7-fluoro-4-oxo-8-[(trifluoromethyl)sulfonyl]oxy-1,4-dihydroquinoline-3-carboxylate (8) (0.34 g, 0.80 mmol) in tetrahydrofuran (6 mL, 70 mmol) was sparged with N₂ for 10 min. Triphenylphosphine (0.056 g, 0.21 mmol), tetrakis(triphenylphosphine)palladium(0) (0.14 g, 0.12 mmol), and N,N-diisopropylethylamine (0.28 mL, 1.6 mmol) were added with continued sparging. Copper(I) iodide (0.067 g, 0.35 mmol) was added to the bright yellow solution, followed by 3 min of additional sparging to give a darker yellow solution. The reaction mixture was heated to 60° C. and turned orange; heating at 60° C. was continued for 24 h to give a dark brown mixture. HPLC showed ˜90% conversion based on the amount of triflate present. The reaction mixture was allowed to cool to rt, was diluted with EtOH (5 mL), and concentrated in vacuo. The residue was taken up in CH₂Cl₂ (10 mL), filtered through Celite, and the filtrate concentrated in vacuo to afford a dark brown oil, which was submitted to column chromatography (2×25 cm silica; CH₂Cl₂, 15% EtOAc/CH₂Cl₂ with 1% EtOH, 25% EtOAc/CH₂Cl₂ with 1% EtOH) to afford the title compound (10) as a brown oil. Initial fractions (250 mg, ˜52%) isolated as a slightly lighter brown oil, LC MS purity 81% (19% OPPh₃ contaminate); later fractions (110 mg, 23%) isolated as a darker brown oil, LC MS purity >95%. HPLC ret. time, 8.113 min; MS (ESI+) for C₃₂H₄₀FN₃O₇ m/z 598.2 (M+H)⁺; R_{f} (30% EtOAc/CH₂Cl₂) 0.30.

### Ethyl-8-[(1Z)-3-(2R,4S)-1-(tert-butoxycarbonyl)-4-[(tert-butoxycarbonyl)amino]pyrrolidin-2-ylprop-1-en-1-yl]-1-cyclopropyl-7-fluoro-4-oxo-1,4-dihydroquinoline-3-carboxylate (11)

Ethyl-8-(3-(2R,4S)-1-(tert-butoxycarbonyl)-4-[(tert-butoxycarbonyl)amino]pyrrolidin-2-ylprop-1-yn-1-yl)-1-cyclopropyl-7-fluoro-4-oxo-1,4-dihydroquinoline-3-carboxylate (10) (204 mg, 0.341 mmol) was taken up in ethanol (10 mL, 200 mmol) and triethylamine (3 drops), sparged with nitrogen, and treated with 10% palladium on carbon (36 mg, 0.034 mmol). Hydrogen (˜1 L) was bubbled through the reaction mixture, which was heated at 40° C. and stirred for 23 h under hydrogen; HPLC analysis showed incomplete conversion. The reaction mixture was allowed to cool to rt and was filtered through a pad of Celite (10% MeOH/CHCl₃). The filtrate was concentrated in vacuo to afford a light brown oil. The above recovered mixture of 10 and 11 was taken up in ethanol (7.0 mL, 120 mmol) and the solution was sparged with nitrogen and treated with triethylamine (one drop) and 10% palladium on carbon (0.036 g, 0.034 mmol). Hydrogen (˜1 L) was bubbled through the reaction mixture, which was stirred for 8 h at 40° C. under hydrogen. Analytical HPLC showed complete consumption of the starting material. The reaction mixture was cooled to rt, filtered through Celite (10% MeOH/CHCl₃), and the filtrate was concentrated in vacuo to afford a yellow oil. Column chromatography (2×8 cm silica; CH₂Cl₂, 1% MeOH/CH₂Cl₂, 2% MeOH/CH₂Cl₂, 5% MeOH/CH₂Cl₂) afforded the title compound (11) as a pale yellow oil (0.15 g, 73%). HPLC ret. time, 7.810 min; MS (ESI+) for C₃₂H₄₂FN₃O₇ m/z 600.1 (M+H)⁺.

### Ethyl-8-(1Z)-3-[(2R,4S)-4-aminopyrrolidin-2-yl]prop-1-en-1-yl-1-cyclopropyl-7-fluoro-4-oxo-1,4-dihydroquinoline-3-carboxylate (12)

Ethyl-8-[(1Z)-3-(2R,4S)-1-(tert-butoxycarbonyl)-4-[(tert-butoxycarbonyl)amino]pyrrolidin-2-ylprop-1-en-1-yl]-1-cyclopropyl-7-fluoro-4-oxo-1,4-dihydroquinoline-3-carboxylate (11) (0.058 g, 0.097 mmol) was taken up in CH₂Cl₂ (4 mL, 70 mmol) and treated with trifluoroacetic acid (0.3 mL, 4 mmol). The reaction mixture was allowed to stir for 23 h at rt. Analytical HPLC showed complete consumption of the starting material. The reaction mixture was diluted with CHCl₃ (55 mL) and washed with 10% aqueous NH₄OH (1×15 mL). The separated aqueous layer was extracted with 10% MeOH/CHCl₃ (2×5 mL) and the combined organic layers were washed with brine (1×30 mL), dried (Na₂SO₄), and concentrated in vacuo to afford the title compound (12) as a dark yellow oil (43 mg, >100%), used without further purification. HPLC ret. time, 4.233 min; MS (ESI+) for C₂₂H₂₆FN₃O₃ m/z 400.1 (M+H)⁺.

### Ethyl-(7aR,9S)-9-amino-4-cyclopropyl-1-oxo-4,7,7a,8,9,10-hexahydro-1H-pyrrolo[1′,2′:1,7]azepino[2,3-h]quinoline-2-carboxylate (13)

Crude ethyl-8-(1Z)-3-[(2R,4S)-4-aminopyrrolidin-2-yl]prop-1-en-1-yl-1-cyclopropyl-7-fluoro-4-oxo-1,4-dihydroquinoline-3-carboxylate (12) (20 mg, 50 mmol) was taken up in N-methylpyrrolidinone (2 mL, 20 mmol) and treated with N,N-diisopropylethylamine (0.02 mL, 0.1 mmol) to afford a clear yellow solution. The flask was placed in a 100° C. oil bath and the reaction mixture was heated for 1 h with stirring. Analytical HPLC and LC MS indicated nearly complete consumption of the starting material. The reaction mixture was allowed to cool to rt and was diluted with water (5 mL) and EtOAc (5 mL). The separated aqueous layer was extracted with EtOAc (2×5 mL) and the combined organic layers were washed with water (3×1 mL) and brine (1×5 mL), dried (Na₂SO₄), and concentrated in vacuo to afford a yellow oil. Further extraction of the aqueous layer with CH₂Cl₂ (4×10 mL) afforded additional product. Preparative HPLC afforded the TFA salt of the title compound (13) as a bright yellow solid (10.8 mg, 43%). HPLC ret. time, 4.855 min; MS (ESI+) for C₂₂H₂₅N₃O₃ m/z 380.1 (M+H)⁺.

### (7aR,9S)-9-amino-4-cyclopropyl-1-oxo-4,7,7a,8,9,10-hexahydro-1H-pyrrolo[1′,2′:1,7]azepino[2,3-h]quinoline-2-carboxylic Acid (14)

A solution of ethyl-(7aR,9S)-9-amino-4-cyclopropyl-1-oxo-4,7,7a,8,9,10-hexahydro-1H-pyrrolo[1′,2′:1,7]azepino[2,3-h]quinoline-2-carboxylate (13) (6.2 mg, 0.016 mmol) in acetonitrile (0.8 mL, 0.02 mol) and water (0.06 mL, 0.003 mol) was treated with 0.5 M aqueous sodium hydroxide (0.042 mL, 0.020 mmol) and the reaction mixture was heated at 60° C. for 3 h; analytical HPLC showed complete consumption of the starting material. The reaction mixture was neutralized with acetic acid and purified by preparative HPLC to afford the TFA salt of the title compound (14) as a bright yellow solid (3.8 mg, 50%). HPLC purity >95% (ret. time, 2.236 min); MS (ESI+) for C₂₀H₂₁N₃O₃ m/z 352.1 (M+H)⁺.

### Example 26

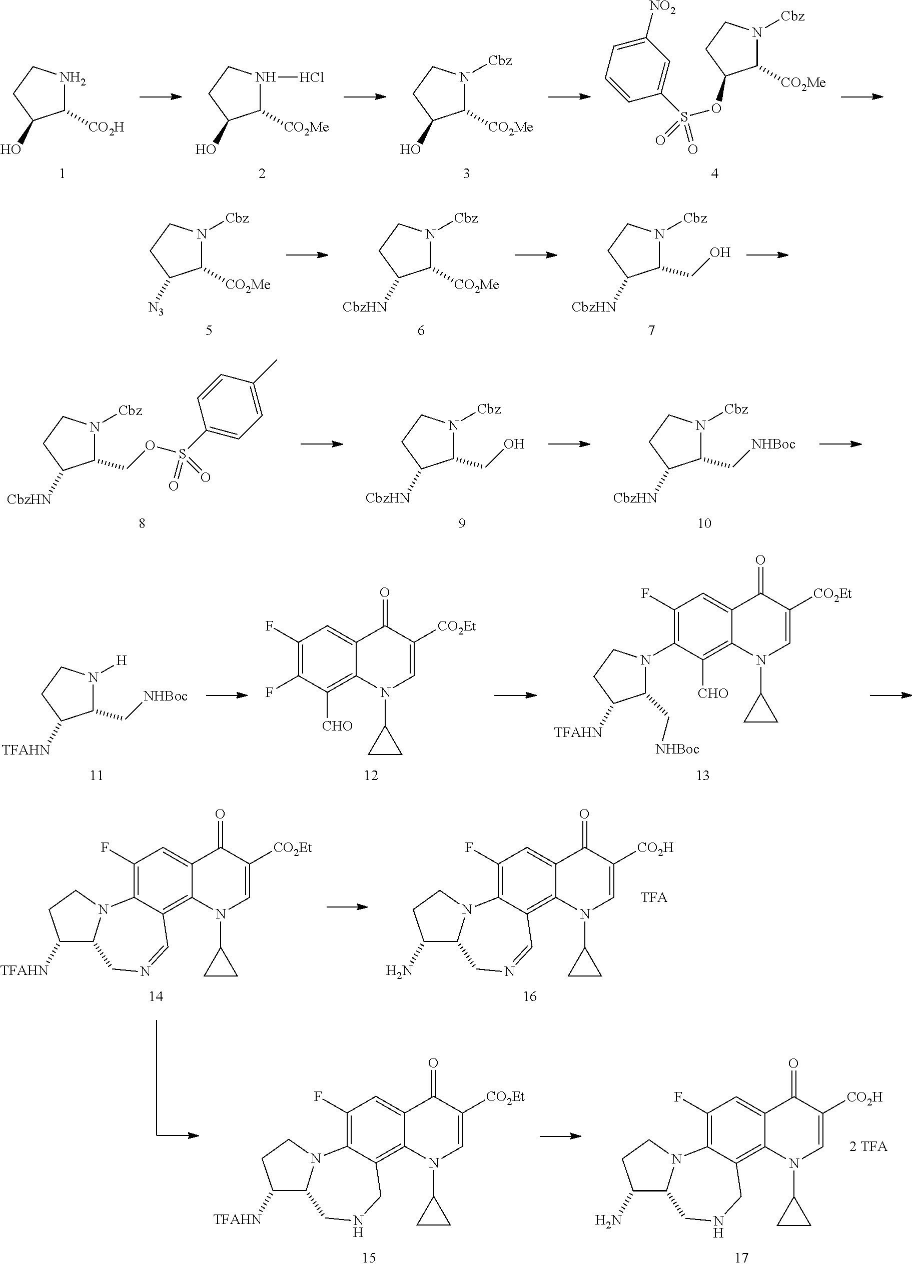

HPLC conditions: Agilent 1100 HPLC. Zorbax C8 150×4.6 mm column. Solvent A—Water (0.1% TFA); Solvent B—Acetonitrile (0.07% TFA). Flow rate, 1.50 mL/min. Gradient—10 min 95% A to 90% B; 2 min hold; then recycle. UV detection @ 214 and 254 nm or @ 214 and 290 nm. All reactions were conducted under an atmosphere of nitrogen.

### Methyl-(3S)-3-hydroxy-L-prolinate hydrochloride (2)

(3S)-3-hydroxy-L-proline (1) (10.0 g, 76.3 mmol) was added, in one portion, to an ice cooled stirred solution of acetyl chloride (7.6 mL, 110 mmol) in methanol (70 mL). After the addition, the ice-water bath was removed and the reaction warmed to ambient temperature followed by heating at 65° C. overnight. After 20 h, reaction was found to be complete by checking a reaction sample by ¹H NMR. The mixture was cooled to room temperature and about one half of the solvent was removed in vacuo to give a white slurry. The slurry was diluted with diethyl ether (150 mL) and filtered. The white solid was washed with two 25 mL portions of cold diethyl ether and placed in a vacuum oven at 40° C. overnight to yield the title compound (2) (13.17 g, 95%) as a white solid.

### 1-Benzyl-2-methyl (2S,3S)-3-hydroxypyrrolidine-1,2-dicarboxylate (3)

A suspension of methyl-(3S)-3-hydroxy-L-prolinate hydrochloride (2) (3.2 g, 18 mmol) in tetrahydrofuran (100 mL) and water (10 mL) was cooled at 0° C. and treated with sodium bicarbonate (3.7 g, 44 mmol) in one portion. The resulting mixture was treated dropwise with benzyl chloroformate (3.3 mL, 23 mmol) in 30 mL tetrahydrofuran. Upon completion of the addition the reaction was allowed to warm to room temperature and stir overnight After 23 h, the reaction appeared complete by TLC (30% EA/CH₂Cl₂). The solvent was removed in vacuo and the resultant aqueous phase was diluted with 30 mL H₂O and 30 mL EA. The mixture was extracted and the aqueous phase was extracted twice more with 30 mL portions of EA. The combined organic phase was washed with two 50 mL portions of H₂O and 50 mL brine, dried over Na₂SO₄, filtered and concentrated to yield a colorless, viscous oil. The material was placed under high vac, during which time a solid formed. The solid was treated with 10 mL hexanes and filtered, washed with additional cold hexanes (50 mL) and dried overnight to yield the title compound (3) (4.72 g; 96%) as a white solid: MS (ESI+) for C₁₄H₁₇NO₅ m/z 280.2 (M+H)⁺; HPLC purity 90% (ret. time, 6.16 min); TLC 30% EA/CH₂Cl₂, R_{f}=0.32.

### 1-Benzyl-2-methyl-(2S,3S)-3-[(3-nitrophenyl)sulfonyl]oxypyrrolidine-1,2-dicarboxylate (4)

A solution of 1-benzyl-2-methyl-(2S,3S)-3-hydroxypyrrolidine-1,2-dicarboxylate (3) (3.30 g, 11.8 mmol) in pyridine (20 mL) was cooled at 0° C. and treated with m-nitrobenzenesulfonyl chloride (3.93 g, 17.7 mmol) in four portions over 30 minutes. The reaction mixture was stirred and allowed to slowly warm to room temperature. After 64 h, the reaction was found to be complete by LCMS. The reaction mixture was concentrated to about one third the original volume and diluted with 100 mL 1N HCl solution and 70 mL EA. The mixture was extracted and the aqueous phase was washed with 50 mL of EA. The combined organic extracts were washed with 100 mL portions of H₂O and sat NaHCO₃, and dried over Na₂SO₄. The solution was filtered and concentrated to yield the title compound (4) (5.05 g; 91%) as a light tan viscous oil: MS (ESI+) for C₂₀H₂₀N₂O₉S m/z 465.0 (M+H)⁺; HPLC purity 89% (ret. time, 8.94 min).

### 1-Benzyl-2-methyl-(2S,3R)-3-azidopyrrolidine-1,2-dicarboxylate (5)

A solution of 1-benzyl-2-methyl-(2S,3S)-3-[(3-nitrophenyl)sulfonyl]oxypyrrolidine-1,2-dicarboxylate (4) (3.14 g, 6.76 mmol) in N,N-dimethylformamide (12 mL) was treated with sodium azide (1.76 g, 27.0 mmol) in one portion and the reaction mixture was heated at 50° C. After 16 h at 50° C., the starting material was consumed by HPLC. The mixture was diluted with 110 mL H₂O and extracted with two 60 mL portions of 1/1 EA/MTBE. The combined organic phase was washed with two 60 mL portions of H₂O and one 50 mL portion of brine, dried over Na₂SO₄, filtered and concentrated to yield the title compound (2.03 g; 99%) as a light tan oil which was about 75% title compound (5) along with 16% of an elimination side product: HPLC purity 74% (ret. time, 8.09 min).

### 1-Benzyl-2-methyl-(2S,3R)-3-[(benzyloxy)carbonyl]aminopyrrolidine-1,2-dicarboxylate (6)

A solution of crude 1-benzyl-2-methyl-(2S,3R)-3-azidopyrrolidine-1,2-dicarboxylate (5) (2.03 g, 5.34 mmol) in tetrahydrofuran (40 mL) was cooled at 0° C. and treated with triphenylphosphine (2.10 g, 8.00 mmol) in one portion. The mixture was stirred for one hour while slowly warming to room temperature and treated with water (5.4 mL, 300 mmol) dropwise. The mixture was stirred at 55° C. overnight. After 18 h at 55° C., LCMS indicated the reaction was complete. The reaction mixture was treated with sodium bicarbonate (1.4 g, 17 mmol) in one portion, followed by benzyl chloroformate (1.14 mL, 8.00 mmol) dropwise and the reaction mixture was stirred at room temperature. After 3 h, TLC (50% EA/hex) indicated the desired product had formed and reaction appeared to be complete. The reaction mixture was concentrated to remove most of the solvent and the resultant residue was taken up in 70 mL EA and 40 mL H₂O, extracted and the phases separated. The aqueous phase was washed once with 30 mL EA and the combined organic phase was washed with 40 mL portions of H₂O and brine and dried over Na₂SO₄. The organic phase was filtered and concentrated to a tan viscous oil. The product was isolated by flash chromatography (100 g flash silica gel, 20-60% EA/hex) to yield the title compound (6) (2.01 g, 91%) as a colorless viscous glass: MS (ESI+) for C₂₂H₂₄N₂O₆ m/z 435.1 (M+Na)⁺. HPLC purity 84% (ret. time, 8.41 min); TLC 50% EA/hex R_{f}=0.45.

### Benzyl-(2S,3R)-3-[(benzyloxy)carbonyl]amino-2-(hydroxymethyl)pyrrolidine-1-carboxylate (7)

A solution of 1-benzyl-2-methyl-(2S,3R)-3-[(benzyloxy)carbonyl]aminopyrrolidine-1,2-dicarboxylate (6) (2.77 g, 6.72 mmol) in tetrahydrofuran (30 mL, 400 mmol) was cooled at 0° C. and treated dropwise with a solution of lithium tetrahydroborate (325 mg, 13.4 mmol) in tetrahydrofuran (10 mL) and the mixture was allowed to stir and warm to room temperature. After 42 h at room temperature, the reaction was complete by HPLC. The reaction was carefully quenched by the addition of 30 mL sat NaHCO₃, and the mixture was diluted with 60 mL EA and 30 mL H₂O and extracted. The aqueous phase was washed with 60 mL EA and the combined organic phase was washed with two 50 mL portions of H₂O and 50 mL brine and dried over Na₂SO₄. The solution was filtered and concentrated to yield a colorless viscous liquid. The product was isolated by chromatography (100 g flash silica gel, 30-60% EA/hex) to yield the title compound (7) (2.20 g, 85%) as a colorless glass: MS (ESI+) for C₂₁H₂₄N₂O₅ m/z 385.2 (M+H)⁺; HPLC purity 100% (ret. time, 7.90 min); TLC 50% EA/hex R_{f}=0.19.

### Benzyl-(2S,3R)-3-[(benzyloxy)carbonyl]amino-2-([(4-methylphenyl)sulfonyl]oxymethyl)pyrrolidine-1-carboxylate (8)

A solution of benzyl-(2S,3R)-3-[(benzyloxy)carbonyl]amino-2-(hydroxymethyl)pyrrolidine-1-carboxylate (7) (1.66 g, 4.32 mmol) in pyridine (7.0 mL, 86 mmol) was cooled at 0° C. and treated with p-toluenesulfonyl chloride (1.23 g, 6.48 mmol) in four portions over a 30 minute period. The mixture was allowed to warm to room temperature and stir overnight. After 25 h at room temperature, reaction was complete by TLC (50% EA/hex). The reaction mixture was concentrated to remove most of the pyridine and the residue was taken up in 30 mL 1N HCl and 50 mL EA. The mixture was extracted and the aqueous phase was separated and washed with 30 mL EA. The combined organic phase was washed with 40 mL portions of H₂O and sat NaHCO₃ solution, dried over Na₂SO₄, filtered and concentrated to a viscous oil. The crude product was purified by chromatography (70 g flash silica gel, 25-45% EA/hex) to yield the title compound (8) (2.00 g, 86%) as a colorless glass: MS (ESI+) for C₂₈H₃₀N₂O₇S m/z 539.2 (M+H)⁺; HPLC purity 98% (ret. time, 9.648 min); TLC 50% EA/hex R_{f}=0.52.

### Benzyl-(2R,3R)-2-(azidomethyl)-3-[(benzyloxy)carbonyl]aminopyrrolidine-1-carboxylate (9)

A solution of benzyl-(2S,3R)-3-[(benzyloxy)carbonyl]amino-2-([(4-methylphenyl)sulfonyl]oxymethyl)pyrrolidine-1-carboxylate (8) (2.00 g, 3.71 mmol) in N,N-dimethylformamide (8.3 mL) was treated with sodium azide (0.483 g, 7.43 mmol) and heated at 50° C. overnight. After 108 h at 50° C., the reaction was found to be complete by HPLC. The reaction was allowed to cool to room temperature, diluted with 70 mL H₂O and extracted with two 60 mL portions of 1/1 MTBE/EA. The organic extracts were washed with three 60 mL portions of H₂O, dried over Na₂SO₄, filtered and concentrated to yield the title compound (9) (1.40 g, 92%) as a nearly colorless viscous oil: MS (ESI+) for C₂₁H₂₃N₅O₄ m/z 432.1 (M+Na)⁺. MS (ESI−) for C₂₁H₂₃N₅O₄ m/z 408.1 (M−H)⁻; HPLC purity 96% (ret. time, 9.09 min); TLC (50% EA/hex, R_{f}=0.70).

### Benzyl-(2R,3R)-3-[(benzyloxy)carbonyl]amino-2-[(tert-butoxycarbonyl)amino]methylpyrrolidine-1-carboxylate (10)

A solution of benzyl-(2R,3R)-2-(azidomethyl)-3-[(benzyloxy)carbonyl]aminopyrrolidine-1-carboxylate (9) (1.46 g, 3.36 mmol) in tetrahydrofuran (30 mL) was cooled at 0° C. and triphenylphosphine (1.46 g, 5.57 mmol) was added in one portion. The mixture was allowed to stir at 0° C. for about 30 min and the cooling bath was removed. After 1 h, water (3.7 mL, 210 mmol) was added dropwise and the solution was heated at 55° C. After 30 minutes di-tert-butyldicarbonate (1.22 g, 5.57 mmol) was added in one portion and the mixture was stirred at room temperature overnight. After 16 h at room temperature, HPLC indicated a new product had formed. The reaction mixture was concentrated and the residue taken up in 70 mL EA and 50 mL H₂O and extracted. The aqueous phase was washed with 40 mL EA and the combined organic phase was washed with 50 mL portions of H₂O and brine and dried over Na₂SO₄. The solution was filtered and concentrated to a colorless viscous oil. The material was purified by chromatography (100 g silica gel, 20-50% EA/hex) to yield the title compound (10) (0.46 g, 26%) as a colorless glass: MS (ESI+) for C₂₆H₃₃N₃O₆ m/z 484.1 (M+H)⁺; HPLC purity 100% (ret. time, 9.66 min).

### tert-Butyl-((2R,3R)-3-[(trifluoroacetyl)amino]pyrrolidin-2-ylmethyl)carbamate (11)

A solution of benzyl-(2R,3R)-3-[(benzyloxy)carbonyl]amino-2-[(tert-butoxycarbonyl)amino]methylpyrrolidine-1-carboxylate (10) (452 mg, 0.935 mmol) in methanol (20 mL) was carefully treated with 10% palladium on carbon (60 mg). The reaction vessel was evacuated and filled with hydrogen gas three times and the reaction mixture was allowed to stir under an atmosphere of hydrogen. After 2 h at room temperature, the reaction was found to be complete by LCMS. The reaction mixture was filtered through a pad of Celite and the pad was washed with 20 mL MeOH. The filtrate was concentrated to yield the diamine as a colorless glass. The crude diamine was taken up in tetrahydrofuran (5 mL), cooled at 0° C., and treated dropwise with ethyl trifluoroacetate (112 uL, 0.935 mmol). The mixture was stirred and allowed to slowly warm to room temperature. After 16 h, LCMS indicated some starting diamine remained. The reaction was treated with an additional 10 uL of ethyl trifluoroacetate and stirring was continued at 0° C. The reaction mixture was concentrated after 1 h, and the resultant oil was placed on high vac to yield the title compound (11) (310 mg, 106%) as a slightly yellow stiff foam which was found to be a mixture of mono and bis TFA amides: MS (ESI+) for C₁₂H₂₀F₃N₃O₃ m/z 312.2 (M+H)⁺; MS (ESI−) for C₁₂H₂₀F₃N₃O₃ m/z 310.2 (M−H)⁻

### Ethyl-7-(2R,3R)-2-[(tert-butoxycarbonyl)amino]methyl-3-[(trifluoroacetyl)amino]pyrrolidin-1-yl-1-cyclopropyl-6-fluoro-8-formyl-4-oxo-1,4-dihydroquinoline-3-carboxylate (13)

A mixture of tert-butyl-((2R,3R)-3-[(trifluoroacetyl)amino]pyrrolidin-2-ylmethyl)carbamate (11) (315 mg, 1.01 mmol) and ethyl-1-cyclopropyl-6,7-difluoro-8-formyl-4-oxo-1,4-dihydroquinoline-3-carboxylate (12) (260 mg, 0.810 mmol) in N-methylpyrrolidinone (4 mL) was treated with N,N-diisopropylethylamine (0.20 mL, 1.1 mmol) dropwise and the mixture was heated at 50° C. After 5 h, reaction was complete by LCMS. The reaction mixture was cooled to room temperature and diluted with 50 mL EA. The organic phase was washed with two 50 mL portions of H₂O, and 50 mL brine, dried over Na₂SO₄, filtered and concentrated to yield a brown, stiff foam. The material was purified by chromatography (30 g flash silica gel, 20-50% EA/CH₂Cl₂) to yield the title compound (13) (241 mg, 49%) as a yellow solid: MS (ESI+) for C₂₈H₃₂F₄N₄O₇ m/z 613.1 (M+H)⁺; MS (ESI−) for C₂₈H₃₂F₄N₄O₇ m/z 611.2 (M−H); HPLC purity 97% (ret. time, 8.21 min); TLC 5% MeOH/CH₂Cl₂ R_{f}=0.28.

### Ethyl-(3aR,4R)-13-cyclopropyl-8-fluoro-10-oxo-4-[(trifluoroacetyl)amino]-3a,4,5,6,10,13-hexahydro-3H-pyrrolo[2′,1′:3,4][1,4]diazepino[5,6-h]quinoline-11-carboxylate (14)

A solution of ethyl-7-(2R,3R)-2-[(tert-butoxycarbonyl)amino]methyl-3-[(trifluoroacetyl)amino]pyrrolidin-1-yl-1-cyclopropyl-6-fluoro-8-formyl-4-oxo-1,4-dihydroquinoline-3-carboxylate (13) (227 mg, 0.370 mmol) in methylene chloride (5 mL) was cooled at 0° C. and treated with trifluoroacetic acid (1.3 mL, 17 mmol) dropwise and the reaction was allowed to slowly warm to room temperature. After about 3 h, the reaction was complete by HPLC. The reaction mixture was concentrated, the yellow residue was dissolved in 20 mL CH₂Cl₂ and washed with 15 mL sat NaHCO₃. The aqueous phase was back extracted with 10 mL CH₂Cl₂, and the combined organic extracts were dried over Na₂SO₄, filtered and concentrated to a light yellow solid. The material was purified by prep TLC on two 20 cm×20 cm×1.0 mm prep TLC plates eluting with 7% MeOH/CH₂Cl₂ to yield the title compound (14) (150 mg, 87%) as a light yellow solid: MS (ESI+) for C₂₃H₂₂F₄N₄O₄ m/z 495.1 (M+H)⁺; MS (ESI−) for C₂₃H₂₂F₄N₄O₄ m/z 493.1 (M−H)⁻; HPLC purity 93% (ret. time, 5.12 min); TLC 5% MeOH/CH₂Cl₂, R_{f}=0.25

### (3aR,4R)-4-Amino-13-cyclopropyl-8-fluoro-10-oxo-3a,4,5,6,10,13-hexahydro-3H-pyrrolo[2′,1′:3,4][1,4]diazepino[5,6-h]quinoline-11-carboxylic acid trifluoroacetate (16)

A solution of ethyl-(3aR,4R)-13-cyclopropyl-8-fluoro-10-oxo-4-[(trifluoroacetyl)amino]-3a,4,5,6,10,13-hexahydro-3H-pyrrolo[2′,1′:3,4][1,4]diazepino[5,6-h]quinoline-11-carboxylate (14) (50.0 mg, 0.101 mmol) in methanol (2.8 mL) and water (1.1 mL) was treated with potassium carbonate (55.9 mg, 0.404 mmol) in one portion and the mixture was allowed to stir at room temperature. After 50 h, an additional equivalent of potassium carbonate (14 mg, 0.101 mmol) was added and stirring was continued. After 132 h, the reaction was complete by HPLC. The methanol was removed under reduced pressure and the aqueous phase was neutralized to pH˜7 with 10% aqueous HOAc. The solution was placed in the fridge for 1 h, whereupon a solid precipitated out. The solid was filtered and washed with a little water to yield 19 mg of a light yellow solid. The material was dissolved in 0.1N aq trifluoroacetic acid (1.3 mL) and the yellow solution was lyophilized to yield the title compound (16) (29 mg, 59%) as a yellow solid: MS (ESI+) for C₁₉H₁₉FN₄O₃ m/z 371.0 (M+H)⁺. HPLC purity 100% (ret time, 3.46 min).

### Ethyl-(3aR,4R)-13-cyclopropyl-8-fluoro-10-oxo-4-[(trifluoroacetyl)amino]-2,3,3a,4,5,6,10,13-octahydro-1H-pyrrolo[2′,1′:3,4][1,4]diazepino[5,6-h]quinoline-11-carboxylate (15)

A solution of ethyl-(3aR,4R)-13-cyclopropyl-8-fluoro-10-oxo-4-[(trifluoroacetyl)amino]-3a,4,5,6,10,13-hexahydro-3H-pyrrolo[2′,1′:3,4][1,4]diazepino[5,6-h]quinoline-11-carboxylate (14) (40.0 mg, 0.0809 mmol) in methylene chloride (3.3 mL) was treated with sodium triacetoxyborohydride (34.3 mg, 0.162 mmol) in one portion and the mixture was stirred at room temperature for 24 h upon which it was determined the reaction was complete by HPLC. The reaction mixture was diluted with 15 mL CH₂Cl₂ and washed with 10 mL sat NaHCO₃. The aqueous phase was washed with 10 mL CH₂Cl₂, and the combined organic phase was dried over Na₂SO₄, filtered and concentrated to a light yellow solid. The material was purified by prep TLC on a 20 cm×20 cm×0.5 mm prep TLC plate eluting with 10% MeOH/CH₂Cl₂ to yield the title compound (15) (31 mg, 77%) as a slightly yellow solid: MS (ESI+) for C₂₃H₂₄F₃N₄O₄ m/z 497.1 (M+H)⁺; MS (ESI−) for C₂₃H₂₄F₃N₄O₄ m/z 495.1 (M−H)⁻; HPLC purity 93% (ret. time, 5.26 min); TLC 10% MeOH/CH₂Cl₂ R_{f}=0.21.

### (3aR,4R)-4-Amino-13-cyclopropyl-8-fluoro-10-oxo-2,3,3a,4,5,6,10,13-octahydro-1H-pyrrolo[2′,1′:3,4][1,4]diazepino[5,6-h]quinoline-11-carboxylic acid bis(trifluoroacetate) (17)

A solution of ethyl-(3aR,4R)-13-cyclopropyl-8-fluoro-10-oxo-4-[(trifluoroacetyl)amino]-2,3,3a,4,5,6,10,13-octahydro-1H-pyrrolo[2′,1′:3,4][1,4]diazepino[5,6-h]quinoline-11-carboxylate (15) (31.0 mg, 0.0624 mmol) in methanol (1.73 mL) and water (0.658 mL) was treated with potassium carbonate (34.5 mg, 0.250 mmol) in one portion and the mixture was allowed to stir at room temperature for 30 min, then heated at 50° C. for 14 h, during which time the reaction was found to be complete by HPLC. The reaction mixture was cooled to room temperature and concentrated under reduced pressure to remove the methanol. The aqueous phase was neutralized to pH˜7 with 10% aqueous HOAc. A solid began to form and the solution was allowed to stand in the fridge for 1 h. The solid was filtered and washed with cold water and air dried to yield 18 mg of crude product. The solid was taken up in 2.2 mL of 0.1N aqueous TFA, the solution was filtered to remove a small amount of insoluble material and lyophilized to yield the title compound (17) (20 mg, 53%) as a light yellow solid: MS (ESI+) for C₁₉H₂₁FN₄O₃ m/z 373.0 (M+H)⁺; HPLC purity 100% (ret. time, 3.72 min).

### Example 27

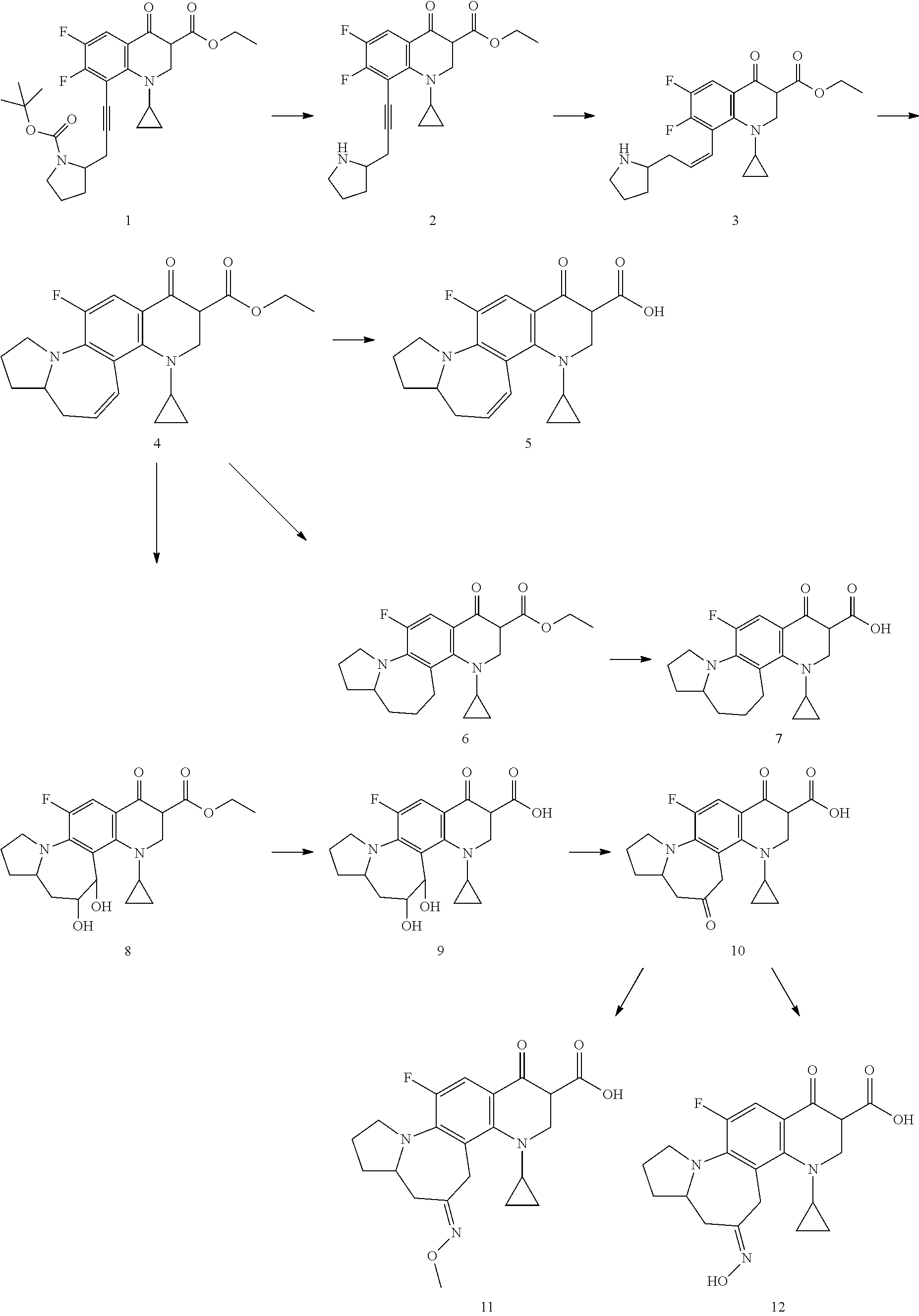

### Ethyl-8-{3-[1-(tert-butoxycarbonyl)pyrrolidin-2-yl]prop-1-yn-1-yl}-1-cyclopropyl-6,7-difluoro-4-oxo-1,4-dihydroquinoline-3-carboxylate (1)

tert-Butyl-2-prop-2-yn-1-ylpyrrolidine-1-carboxylate (100.0 mg, 0.478 mmol), N,N-diisopropylethylamine (0.13 mL, 0.76 mmol), ethyl-1-cyclopropyl-6,7-difluoro-4-oxo-8-{[(trifluoromethyl)sulfonyl]oxy}-1,4-dihydroquinoline-3-carboxylate (170 mg, 0.38 mmol), and tetrakis(triphenylphosphine)palladium(0) (40 mg, 0.04 mmol) were dissolved in dry THF (6 mL). The dark yellow solution was degassed with nitrogen for 5 minutes. Copper(I) iodide (7 mg, 0.04 mmol) was then added and the solution was heated to 60° C. for 1 hour. The dark reaction mixture was cooled to room temperature and filtered thru a pad of silica gel eluting with EtOAc. The solvent was concentrated in vacuo and the resulting residue applied to a 40 g silica gel column which had been preconditioned with 1:9 EtOAc/chloroform. Elution using a gradient of 1:9 to 3:7 EtOAc/chloroform affords 149 mg (78%) of the title compound (1) as a yellow solid. MS (ESI+) for C₂₇H₃₀F₂N₂O₅ m/z 501.3 (M+H)⁺.

### Ethyl-1-cyclopropyl-6,7-difluoro-4-oxo-8-(3-pyrrolidin-2-ylprop-1-yn-1-yl)-1,4-dihydroquinoline-3-carboxylate (2)

Ethyl-8-{3-[1-(tert-butoxycarbonyl)pyrrolidin-2-yl]prop-1-yn-1-yl}-1-cyclopropyl-6,7-difluoro-4-oxo-1,4-dihydroquinoline-3-carboxylate] (1) (149 mg, 0.298 mmol) was taken up in dichloromethane (5 mL). Trifluoroacetic acid (0.25 mL, 3.2 mmol) was added at ambient temperature and the reaction mixture stirred at room temperature for 3.5 hours. The solvent was removed in vacuo and the resulting residue was placed on the vacuum pump overnight affording the title compound (2) as a pale yellow solid (120 mg, 100%). MS (ESI+) for C₂₂H₂₄F₂N₂O₃ m/z 401.2 (M+H)⁺.

### Ethyl-1-cyclopropyl-6,7-difluoro-4-oxo-8-[(1Z)-3-pyrrolidin-2-ylprop-1-en-1-yl]-1,4-dihydroquinoline-3-carboxylate (3)

Ethyl-1-cyclopropyl-6,7-difluoro-4-oxo-8-(3-pyrrolidin-2-ylprop-1-yn-1-yl)-1,4-dihydroquinoline-3-carboxylate] (2) (120 mg, 0.30 mmol) was taken up in EtOH (6 mL) and EtOAc (1 mL). The pH of the solution was adjusted to pH 9 by the addition of a couple of drops of N,N-diisopropylethylamine. 5% Palladium on barium sulfate (3 mg, 0.03 mmol) was added, the flask was flushed with H₂ (one atmosphere—balloon) and the reaction mixture was stirred at room temperature overnight. The reaction mixture was filtered through a pad of Celite washing with EtOH. The solvent was removed in vacuo affording the title compound (3) as a yellow solid which was used immediately for the next reaction. MS (ESI+) for C₂₂H₂₄F₂N₂O₃ m/z 403.1 (M+H)⁺.

### Ethyl-4-cyclopropyl-12-fluoro-1-oxo-4,7,7a,8,9,10-hexahydro-1H-pyrrolo[1′,2′:1,7]azepino[2,3-h]quinoline-2-carboxylate (4)

Ethyl-1-cyclopropyl-6,7-difluoro-4-oxo-8-[(1Z)-3-pyrrolidin-2-ylprop-1-en-1-yl]-1,4-dihydroquinoline-3-carboxylate (3) was immediately taken up into acetonitrile (5 mL) and N,N-diisopropylethylamine (0.209 mL, 1.20 mmol) was added at room temperature. The reaction mixture was stirred at room temperature overnight. The solvent was removed in vacuo and the residue was filtered through a short pad of silica gel eluting with EtOAc. Removal of the solvent afforded the title compound (4) as a yellow solid (90 mg, 78% for the two steps). MS (ESI+) for C₂₂H₂₃FN₂O₃ m/z 383.2 (M+H)⁺.

### 4-Cyclopropyl-12-fluoro-1-oxo-4,7,7a,8,9,10-hexahydro-1H-pyrrolo[1′,2′:1,7]azepino[2,3-h]quinoline-2-carboxylic acid 5

Ethyl-4-cyclopropyl-12-fluoro-1-oxo-4,7,7a,8,9,10-hexahydro-1H-pyrrolo[1′,2′:1,7]azepino[2,3-h]quinoline-2-carboxylate (4) (21.4 mg, 0.056 mmol) was taken up in THF (2 mL) and flushed under nitrogen. Potassium trimethylsilanolate (9.57 mg, 0.067 mmol, 90% tech) was added to the yellow solution in one portion. An immediate change in color to a dark yellow-orange solution occurred and the reaction mixture was stirred at room temperature for 2 hours. The solvent was removed in vacuo affording a yellow powder which was dissolved in water (2 mL). The aqueous layer was washed with ether (3×10 mL) and the organic extracts discarded. The pH of the aqueous layer was adjusted to pH 3 using 1N HCl causing a precipitate to form. The aqueous layer was extracted with chloroform (3×10 mL) and the combined chloroform extracts were dried over sodium sulfate. Filtration and removal of solvent in vacuo affords a dark yellow solid. The solids were triturated twice with 5:1 ether:methanol and the resulting solids were placed on the vacuum pump overnight affording 7 mg (40%) of (5) as a cream colored solid. MS (ESI+) for C₂₀H₁₉FN₂O₃ m/z 355.2 (M+H)⁺. 1H NMR (400 MHz, CDCl₃) δ ppm 8.85 (s, 1H), 7.73 (d, J=14.93 Hz, 1H), 6.50 (d, J=12.02 Hz, 1H), 6.03 (td, J=7.88, 4.15 Hz, 1H), 4.21-4.07 (m, 1H), 4.04-3.84 (m, 3H), 2.77-2.63 (m, 1H), 2.61-2.50 (m, 1H), 2.28-2.01 (m, 3H), 1.85 (d, J=9.95 Hz, 1H), 1.42-1.29 (m, 1H), 1.15-0.96 (m, 2H), 0.93-0.82 (m, 1H).

### Ethyl-4-cyclopropyl-12-fluoro-1-oxo-4,5,6,7,7a,8,9,10-octahydro-1H-pyrrolo[1′,2′:1,7]azepino[2,3-h]quinoline-2-carboxylate (6)

Ethyl-4-cyclopropyl-12-fluoro-1-oxo-4,7,7a,8,9,10-hexahydro-1H-pyrrolo[1′,2′:1,7]azepino[2,3-h]quinoline-2-carboxylate (4) (75.0 mg, 0.196 mmol) was taken up in a 5:1 EtOH:EtOAc mixture (6 mL). 10% Palladium on carbon (2.1 mg, 0.020 mmol) was added and the reaction flask was flushed with hydrogen (one atmosphere—balloon). The reaction mixture was stirred at room temperature overnight and then filtered through a pad of Celite washing with EtOAc. The solvent was removed in vacuo affording a pale yellow solid. The material was applied to 4 10×20 cm 0.5 mm TLC plates and eluted with EtOAc (×2). The desired band was removed and extracted into EtOAc. Filtration and concentration of the solvent in vacuo afforded 46 mg (61%) of the title product (6) as a cream colored solid. MS (ESI+) for C₂₂H₂₅FN₂O₃ m/z 385.2 (M+H)⁺.

### 4-Cyclopropyl-12-fluoro-1-oxo-4,5,6,7,7a,8,9,10-octahydro-1H-pyrrolo[1′,2′:1,7]azepino[2,3-h]quinoline-2-carboxylic acid (7) Ethyl-4-cyclopropyl-12-fluoro-1-oxo-4,5,6,7,7a,8,9,10-octahydro-1H-pyrrolo[1′,2′:1,7]azepino[2,3-h]quinoline-2-carboxylate (6) was then taken up in THF (5 mL), the reaction mixture flushed under nitrogen and then potassium trimethylsilanolate (20 mg, 0.1 mmol) was added in one portion at room temperature and stirring was continued for 2 hours. The solvent was removed in vacuo and the residue was taken up in water (5 mL) and acidified to pH 3 with 1N HCl. The aqueous layer was extracted with chloroform (3×15 mL) and the combined organic layers were washed once with brine (5 mL). The organic layers were dried over sodium sulfate, filtered and the solvent removed in vacuo affording a yellow oil. The yellow oil was triturated with ether (solids formed immediately as the solvent is added, triturated 2×3 mL). The resulting solids were recrystallized from acetonitrile (2 mL). The crystals were collected by filtration, washed with hexanes and then dried under vacuum affording 6.2 mg (9%) of (7) as cream colored crystals. MS (ESI+) for C₂₀H₂₁FN₂O₃ m/z 357.3 (M+H)⁺. ¹H NMR (400 MHz, CDCl₃) δ ppm 8.83 (s, 1H), 7.83 (d, J=14.51 Hz, 1H), 4.02 (td, J=7.05, 3.32 Hz, 2H), 3.82-3.62 (m, 3H), 3.56 (dd, J=15.34, 9.95 Hz, 1H), 2.59 (dd, J=15.34, 9.12 Hz, 1H), 2.24-1.95 (m, 4H), 1.92-1.64 (m, 4H), 1.33 (ddd, J=10.16, 6.63, 6.43 Hz, 1H), 1.16 (ddd, J=16.38, 6.63, 6.43 Hz, 1H), 1.07-0.98 (m, J=7.05, 6.74, 6.74, 3.11 Hz, 1H), 0.83 (ddd, J=13.16, 10.88, 4.15 Hz, 1H).

### Ethyl-4-cyclopropyl-12-fluoro-5,6-dihydroxy-1-oxo-4,5,6,7,7a,8,9,10-octahydro-1H-pyrrolo[1′,2′:1,7]azepino[2,3-h]quinoline-2-carboxylate (8)

Ethyl-4-cyclopropyl-12-fluoro-1-oxo-4,7,7a,8,9,10-hexahydro-1H-pyrrolo[1′,2′:1,7]azepino[2,3-h]quinoline-2-carboxylate (4) (950.0 mg, 2.484 mmol) in 1,4-dioxane (42 mL, 530 mmol) and water (8 mL, 500 mmol) was treated with osmium tetroxide at room temperature, (2.5 wt % in 2-methyl-2-propanol, 0.62 mL) and the mixture was stirred for 5 minutes. N-Methylmorpholine N-oxide (306 mg, 2.61 mmol) was added, and the mixture stirred at room temperature overnight. The reaction mixture was diluted with water (30 mL) and extracted with EtOAc (2×75 mL). The combined organic layers were washed with brine (20 mL), dried over sodium sulfate and concentrated in vacuo to give a yellow solid. The solids were chromatographed on a 120 g silica gel flash column which had been pre-conditioned with 1:1 EtOAc:CHCl₃. Gradient elution from 1:1 EtOAc:CHCl₃ to 1:19 EtOH:EtOAc followed by removal of the solvent in vacuo affords 340 mg (34%) of the title compound (8) as a pale yellow solid. MS (ESI+) for C₂₂H₂₅FN₂O₅ m/z 417.3 (M+H)⁺.

### 4-Cyclopropyl-12-fluoro-5,6-dihydroxy-1-oxo-4,5,6,7,7a,8,9,10-octahydro-1H-pyrrolo[1′,2′:1,7]azepino[2,3-h]quinoline-2-carboxylic acid (9)

Ethyl-4-cyclopropyl-12-fluoro-5,6-dihydroxy-1-oxo-4,5,6,7,7a,8,9,10-octahydro-1H-pyrrolo[1′,2′:1,7]azepino[2,3-h]quinoline-2-carboxylate (8) (100.1 mg, 0.2404 mmol) was dissolved in acetonitrile (7 mL), tetrahydrofuran (4 mL) and water (0.9 mL). 0.5M Sodium hydroxide (0.961 mL) was added at room temperature. The reaction mixture was stirred at room temperature overnight. The reaction was quenched by the addition of acetic acid to pH 4, the solvent removed in vacuo and the resultant solid triturated thrice with ether to afford 100 mg of crude product as a dark brown solid. A portion of the crude material (50 mg) was dissolved in 5 mL of 2:1 water:acetonitrile and injected in 2 portions onto a preparative reverse phase HPLC (Column: Phenomenex Luna 250×30 mm, 10 micron; Solvent A: Acetonitrile with 0.07% TFA; Solvent B: Water with 0.10% TFA; 30 min run time, 0-10 min ramp from 5% A:95% B to 80% A:20% B; 10-15 min ramp from 80% A:20% B to 100% A; 15-20 min hold at 100% A; 20-25 min recycle back to 5% A:95% B; then hold at 5%:95% B from 25-30 min). The desired diol acid has a retention time of 10.71 minutes. The desired fractions were combined and placed on the lyophilizer overnight affording 21.5 mg (46%) of (9) as a yellow fluffy solid. MS (ESI+) for C₂₀H₂₁FN₂O₅ m/z 389.3 (M+H)⁺. 1H NMR (400 MHz, DMSO-d₆) δ ppm 8.78 (s, 1H), 7.65 (d, J=14.10 Hz, 1H), 5.38 (d, J=4.98 Hz, 1H), 4.80 (d, J=5.39 Hz, 2H), 4.34-4.15 (m, 2H), 3.93 (br. s., 1H), 3.67-3.42 (m, 2H), 2.17-1.92 (m, 5H), 1.77-1.55 (m, 2H), 1.28-1.08 (m, 2H), 1.04-0.93 (m, 1H), 0.80-0.69 (m, 1H).

### 4-Cyclopropyl-12-fluoro-1,6-dioxo-4,5,6,7,7a,8,9,10-octahydro-1H-pyrrolo[1′,2′:1,7]azepino[2,3-h]quinoline-2-carboxylic acid (10)

The remainder of the crude diol acid (9) (50 mg) was taken up in 50 mL of a 1:1 mix of the prep HPLC solvents (Solvent A: Acetonitrile with 0.07% TFA; Solvent B: Water with 0.10% TFA) and allowed to stand overnight. The solvent was removed by lyophilzation and the resultant solid material taken up in 4 mL of a 1:1 water:acetonitrile mix. The solution was injected in 2 portions onto a preparative reverse phase HPLC (Column: Phenomenex Luna 250×30 mm, 10 micron; Solvent A: Acetonitrile with 0.07% TFA; Solvent B: Water with 0.10% TFA; 30 min run time, 0-10 min ramp from 5% A:95% B to 80% A:20% B; 10-15 min ramp from 80% A:20% B to 100% A; 15-20 min hold at 100% A; 20-25 min recycle back to 5% A:95% B; then hold at 5%:95% B from 25-30 min). The desired keto acid has a retention time of 12.79 minutes. The desired fractions were combined and placed on the lyophilizer overnight affording 14.9 mg (33.5%) of (10) as an orange solid. MS (ESI⁺) for C₂₀H₁₉FN₂O₄ m/z 371.4 (M+H)⁺. ¹H NMR (400 MHz, CDCl₃) δ ppm 8.86 (s, 1H), 7.97 (d, J=13.27 Hz, 1H), 4.37 (d, J=15.34 Hz, 1H), 3.83-3.76 (m, 1H), 3.77 (d, J=15.34 Hz, 1H), 2.98-2.78 (m, 2H), 2.23-1.73 (m, 5H), 1.35-1.25 (m, 1H), 1.12 (dd, J=10.37, 5.80 Hz, 1H), 0.93 (ddd, J=13.06, 7.05, 3.52 Hz, 1H), 0.76 (dt, J=11.20, 6.43 Hz, 1H).

### 4-Cyclopropyl-12-fluoro-6-(methoxyimino)-1-oxo-4,5,6,7,7a,8,9,10-octahydro-1H-pyrrolo[1′,2′:1,7]azepino[2,3-h]quinoline-2-carboxylic acid (11)

4-Cyclopropyl-12-fluoro-1,6-dioxo-4,5,6,7,7a,8,9,10-octahydro-1H-pyrrolo[1′,2′:1,7]azepino[2,3-h]quinoline-2-carboxylic acid (10) (10.3 mg, 0.0278 mmol) was taken up in pyridine (0.5 mL, 6 mmol). Methoxylamine hydrochloride (10.2 mg, 0.122 mmol) was added in one portion at room temperature. The reaction mixture was stirred at room temperature for 30 minutes. Water (3 mL) was added followed by sodium bicarbonate (10.2 mg, 0.122 mmol). The reaction mixture was placed on the lyophilizer overnight affording 11.0 mg (99%) of (11) (mixture of E and Z isomers) as a yellow fluffy solid. MS (ESI+) for C₂₁H₂₂FN₃O₄ m/z 400.2 (M+H)⁺. 1H NMR (400 MHz, DMSO-d₆) δ ppm 8.62 (s, 0.5H), 8.58 (s, 0.5H), 7.71 (d, J=13.27 Hz, 0.5H), 7.56 (d, J=15.34 Hz, 0.5H), 4.58-4.34 (m, 1H), 4.18-3.79 (m, 2H), 3.73 (s, 1.5H), 3.69 (s, 1.5H), 3.60-3.39 (m, 1H), 3.15-2.38 (m, 5H), 2.07-1.53 (m, 4 H), 1.42-1.06 (m, 2H), 0.97-0.59 (m, 2H).

### 4-Cyclopropyl-12-fluoro-6-(hydroxyimino)-1-oxo-4,5,6,7,7a,8,9,10-octahydro-1H-pyrrolo[1′,2′:1,7]azepino[2,3-h]quinoline-2-carboxylic acid (12)

4-Cyclopropyl-12-fluoro-1,6-dioxo-4,5,6,7,7a,8,9,10-octahydro-1H-pyrrolo[1′,2′:1,7]azepino[2,3-h]quinoline-2-carboxylic acid (10) (10.1 mg, 0.0273 mmol) was taken up pyridine (0.5 mL). Hydroxylamine hydrochloride (10.3 mg, 0.148 mmol) was added in one portion at room temperature. The reaction mixture was stirred for 30 minutes. Water (3 mL) was added followed by sodium bicarbonate (12.5 mg, 0.148 mmol). The reaction mixture was placed on the lyophilizer overnight affording 10.1 mg (96%) of (12) as a cream colored fluffy solid. MS (ESI+) for C₂₀H₂₀FN₃O₄ m/z 386.2 (M+H)⁺. ¹H NMR (400 MHz, DMSO-d₆) δ ppm 10.58 (br. s., 1H), 8.63 (s, 0.5H), 8.57 (s, 0.5H), 7.70 (d, J=14.10 Hz, 0.5H), 7.54 (d, J=15.76 Hz, 0.5H), 4.59-4.40 (m, 1H), 4.39-3.91 (m, 2H), 3.85-3.42 (m, 2H), 3.20-2.61 (m, 4H), 2.09-1.49 (m, 4H), 1.40-1.01 (m, 2H), 0.98-0.51 (m, 2H)

### Example 28

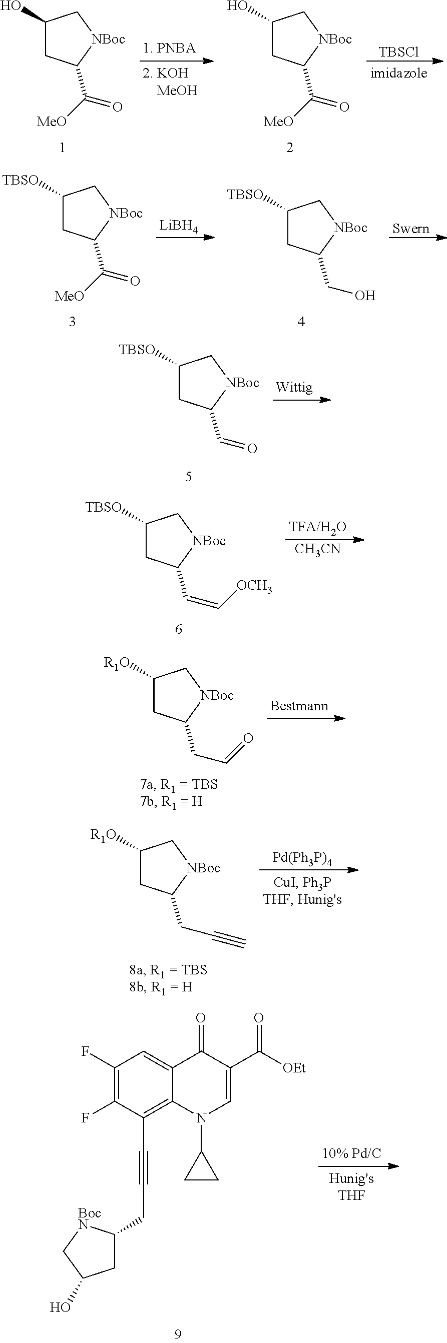

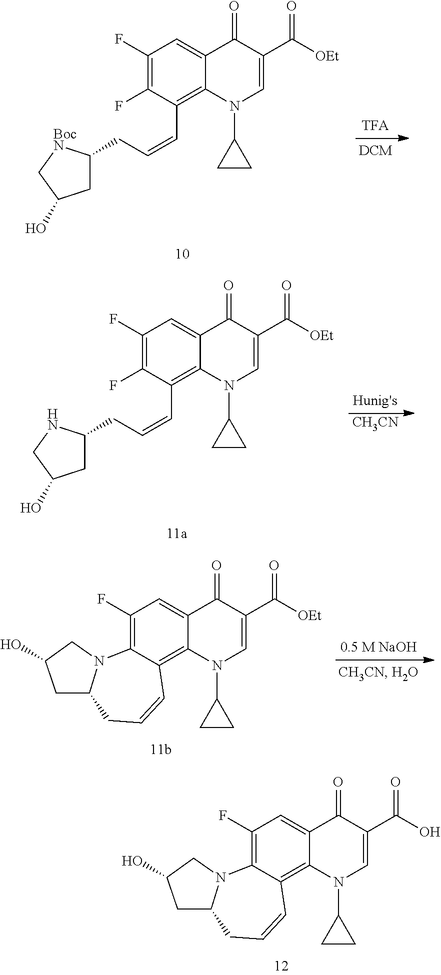

All reactions were performed under an atmosphere of nitrogen. Unless otherwise indicated, the reaction flask was evacuated with vacuum and then back-filled with nitrogen via a balloon (×3) and the reaction kept under nitrogen via balloon for the duration of the reaction. Analytical HPLC was performed using an Agilent 1100 HPLC with one of the following methods:

Method A: Agilent Scalar C18 150×4.6 mm 5 micron column; 1.5 mL/min; solvent A—water (0.1% TFA); solvent B—acetonitrile (0.07% TFA, gradient: 10 min 95% A to 95% B; 5 min hold; then recycle; UV detection @ 214, 250 and 280 nm.

Method B: Agilent XDB C18 50×4.6 mm/1.8 micron column; 1.5 mL/min; solvent A—water (0.1% TFA), solvent B—acetonitrile (0.07% TFA); gradient: 5 min 95% A to 95% B then 1 min hold, 1 min 95% B to 95% A then 30 sec hold; UV detection @ 210, 254, and 280 nm.

Method C: Agilent Eclipse XBD C8 column; solvent A—water (0.1% TFA); solvent B—acetonitrile (0.07% TFA, gradient: 10 min 95% A to 95% B; 5 min hold; then recycle; UV detection @ 214, 250 and 280 nm.

Preparative HPLC condition—Method D: Phenomenex Luna 250×21.20 mm, 10 micron; solvent A is 0.07% TFA in acetonitrile; solvent B is 0.10% TFA in water; 26 minute run; gradient: 5% to 80% A over 10 minutes; from 80% to 100% A over 5 minutes; hold 100% A for 5 minutes; 100% to 5% A over 5 minutes; hold 1 minute then recycle; detection at 285 nm.

Prep HPLC conditions—Method E: Phenomenex Luna 250×30.00 mm, 10 micron; solvent A is 0.07% TFA in acetonitrile; solvent B is 0.10% TFA in water; rate is 20 mL/min; 30 minute run; 5% to 70% A over 14 minute ramp; 3 minute ramp from 80% to 100% A; hold 100% A for 3 minutes; ramp down from 100% to 5% A over 5 minutes; hold 10 minutes then recycle.

Thin layer chromatography (TLC) was performed using Analtech TLC plates GHLF, 250 microns, order #21521.

### 1-tert-Butyl-2-methyl-(2S,4S)-4-[(4-nitrobenzoyl)oxy]pyrrolidine-1,2-dicarboxylate (2)

Methyl-(2S,4R)—N-tert-butoxycarbonyl-4-hydroxy-2-pyrrolidinecarboxylate (1) (20.0 g, 0.0815 mol; Synthetec) was dissolved in tetrahydrofuran (700 mL) and was cooled at 0° C. in an ice-water bath. Then, triphenylphosphine (25.7 g, 0.0978 mol) and 4-nitrobenzoic acid (16.4 g, 0.0978 mol) were added and finally, diisopropyl azodicarboxylate (19.3 mL, 0.0978 mol) was added as a solution in tetrahydrofuran (300 mL). The reaction was stirred at 0° C. for 20 minutes and then the ice bath was removed with continued stirring at ambient temperature overnight (˜12 hr). TLC analysis (30% ethyl acetate/DCM) after this period of time shows consumption of the starting alcohol and clean formation of a new spot. The reaction was diluted with 200 mL of saturated sodium bicarbonate and the THF was removed in vacuo. The resulting aqueous layer was partitioned between ethyl acetate (300 mL) and water (200 mL) and the aqueous layer was extracted once more with ethyl acetate (200 mL). The combined organic layers were washed with water, saturated sodium bicarbonate and brine (100 mL each), dried over MgSO₄, filtered, concentrated in vacuo. The crude product was filtered through a plug of silica gel in a 600 mL scintered glass funnel and 300 mL fractions were collected, with the product eluting in the first 600 mL. The combined fractions were concentrated in vacuo and then further purified by silica gel chromatography, eluting with 0 to 30% ethyl acetate in hexanes to afford two lots-Lot 1: (19.9 g that is 80.3% by mass pure by ¹H NMR analysis—other impurities are Ph₃PO, 5.3% and diisopropyl hydrazine 1,2-dicarboxylate, 14.4%, to afford a corrected mass of 16.0 g of the desired benzoate ester) and Lot 2: (33.6 g that is 38.6% pure by mass based on ¹H NMR analysis—other impurities are Ph₃PO, 15.6% and diisopropyl hydrazine 1,2-dicarboxylate, 45.8%, to afford a corrected mass of 12.96 g of the desired benzoate ester). Thus a total of 29.0 g of the benzoate ester was obtained in two lots of 16.0 g and 13.0 g in 90% combined yield; (benzoate ester) ¹H NMR (400 MHz, CDCl₃) δ ppm 8.31 (d, J=9.12 Hz, 2H), 8.17 (d, J=8.91 Hz, 2H), 5.59 (m, 1H), 4.63 (dd, J=8.91, 1.24 Hz, 0.5H), 4.50 (dd, J=9.33, 1.45 Hz, 0.5H), 3.85 (m, 2H), 3.72 (s, 1.33H, OMe rotamer), 3.71 (s, 1.67H, OMe rotamer), 2.59 (m, 1H), 2.48 (m, 1H), 1.50 (s, 4H, Ot-Bu rotamer), 1.47 (s, 5H, Ot-Bu rotamer); the two lots were subjected to hydrolysis conditions as outlined in the next step.

1-tert-Butyl-2-methyl-(2S,4S)-4-[(4-nitrobenzoyl)oxy]pyrrolidine-1,2-dicarboxylate (29.0 g, 0.0735 mol) was dissolved in methanol (400 mL) and then cooled at 0° C. in an ice-water bath. Then, potassium hydroxide (4.33 g, 0.0772 mol) as a solution in methanol (45 mL) was added dropwise over 30 minutes to the cooled solution. After the addition was complete, the reaction was monitored by TLC (40% ethyl acetate/hexanes) for completion. TLC after this period of time shows complete consumption of the starting benzoate ester and formation of a new, lower R_{f} product. The reaction was taken to neutral pH by the addition of 1M HCl (approximately 70 mL). The reaction mixture was concentrated in vacuo and then the organic product was extracted with ethyl acetate (3×150 mL) and the combined organic layers washed with water and brine, dried over MgSO₄, filtered and concentrated in vacuo to afford the crude product. The crude product was purified by filtration through a 600 mL plug of silica gel, eluting with 0-50% ethyl acetate in CH₂Cl₂ (in 10% increments, 300 mL each) to afford the desired alcohol (2), 25.3 g (˜70% pure in 98% yield, corrected for Ph₃PO impurity), which was carried on without further purification; ¹H NMR (400 MHz, CDCl₃) δ ppm 4.26 (m, 2H), 3.73 (s, 1H, OMe rotamer), 3.71 (s, 2H, OMe rotamer), 3.60 (m, 1H), 3.46 (m, 1.5H), 3.23 (d, J=10.16 Hz, 0.5H, OH), 2.26 (m, 1 H), 2.02 (m, 1H), 1.40 (s, 4H, Ot-Bu rotamer), 1.35 (s, 5H, Ot-Bu rotamer).

### 1-tert-Butyl-2-methyl-(2S,4S)-4-{[tert-butyl(dimethyl)silyl]oxy}pyrrolidine-1,2-dicarboxylate (3)

1-tert-Butyl-2-methyl-(2S,4S)-4-hydroxypyrrolidine-1,2-dicarboxylate (2) (25.30 g, 0.08046 mol) was dissolved in N,N-dimethylformamide (800 mL) and 1H-imidazole (10.5 g, 0.155 mol) was added followed by tert-butyldimethylsilyl chloride (17.1 g, 0.113 mol). The reaction was stirred at ambient temperature for 24 hr. After this period of time, TLC showed the reaction to be complete (20% ethyl acetate:hexanes). The reaction was quenched by the addition of 300 mL of water and the organic product was extracted with diethyl ether (3×100 mL). The combined organic layers were washed with water (3×50 mL), saturated sodium bicarbonate (1×50 mL) and brine (1×50 mL) before drying over MgSO₄. Filtration to remove the drying agent and concentration in vacuo afforded the crude product (3) (28.9 g, 99% yield) which was used without further purification; ¹H NMR (400 MHz, CDCl₃) δ ppm 4.31 (dd, J=8.81, 4.04 Hz, 0.4H, rotamer), 4.24 (m, 1H), 4.20 (dd, J=8.71, 4.35 Hz, 0.6H, rotamer), 3.61 (s, 3H), 3.54 (dd, J=11.20, 5.39 Hz, 0.6H, rotamer), 3.47 (dd, J=10.99, 5.18 Hz, 0.4H, rotamer), 3.23 (dd, J=10.88, 3.21 Hz, 0.6H, rotamer), 3.18 (dd, J=11.09, 3.01 Hz, 0.4H, rotamer), 2.19 (m, 1H), 2.00 (m, 1H), 1.37 (s, 4H, t-Bu rotamer), 1.32 (s, 5H, t-Bu rotamer), 0.76 (s, 4H, t-Bu rotamer), 0.75 (s, 5H, t-Bu rotamer), −0.06 (s, 3H), −0.07 (s, 3H).

### tert-Butyl-(2S,4S)-4-{[tert-butyl(dimethyl)silyl]oxy}-2-(hydroxymethyl)pyrrolidine-1-carboxylate (4)

1-tert-Butyl-2-methyl-(2S,4S)-4-{[tert-butyl(dimethyl)silyl]oxy}pyrrolidine-1,2-dicarboxylate (3) (28.9 g, 0.0804 mol) was dissolved in tetrahydrofuran (1.0 L) and was cooled at 0° C. in an ice-water bath. Then, lithium tetrahydroborate (2.6 g, 0.12 mol) was added in two portions. The ice bath was allowed to slowly expire overnight (˜14 hr) with continued stirring. TLC after this period of time showed complete consumption of the ester and formation of a single, lower R_{f} product. The reaction was quenched with ice water/1M HCl to acidic pH and then the product was extracted (3×100 mL) with chloroform and the combined organic layers were washed with water and brine, dried over MgSO₄, filtered and concentrated in vacuo to afford the crude product. Silica gel chromatography, eluting with 0 to 40% ethyl acetate in CH₂Cl₂ over a 1 hr gradient on 120 g of silica gel afforded the purified product (4), 25.2 g in 95% yield; ¹H NMR (400 MHz, CDCl₃) δ ppm 4.55 (d, J=7.05 Hz, 1H, OH), 4.29-4.23 (m, 1H), 4.00-3.85 (m, 1H), 3.81-3.57 (m, 1.5H), 3.54-3.43 (complex w/dd, J=11.5, 5.1 Hz, 1.5H), 3.31-3.28 (m, 0.5H), 3.15 (dd, J=11.40, 1.45 Hz, 0.5H), 2.24-2.09 (m, 0.8H), 1.77 (d, J=14.7 Hz, 0.2H), 1.54 (m, 1H), 1.39 (s, 9 H), 0.81 (s, 9H), −0.00 (s, 6H).

### tert-Butyl-(2S,4S)-4-{[tert-butyl(dimethyl)silyl]oxy}-2-formylpyrrolidine-1-carboxylate (5)

Dimethyl sulfoxide (11.3 mL, 0.160 mol) was added slowly dropwise to a stirred solution of oxalyl chloride (6.76 mL, 0.0799 mol) in CH₂Cl₂ (500 mL, 8 mol) that was cooled at −78° C. in a dry ice-acetone bath. After the addition was complete, the reaction was stirred for 5 minutes before adding tert-butyl (2S,4S)-4-{[tert-butyl(dimethyl)silyl]oxy}-2-(hydroxymethyl)pyrrolidine-1-carboxylate (4) (13.25 g, 0.03997 mol) as a solution in CH₂Cl₂ (75 mL). After the addition of the alcohol was complete, the reaction was stirred for 30 minutes at reduced temperature and then was checked for completion by TLC after a small reaction aliquot was quenched in triethylamine. The reaction was nearly complete (>95%) at this time and so was stirred an additional 15 minutes before adding triethylamine (22.3 mL, 0.160 mol) dropwise via a pressure equalizing dropping funnel and the reaction was stirred overnight at −78° C. and had warmed to approximately −20° C., at which time the reaction was quenched with ˜250 mL of saturated sodium bicarbonate. The reaction solution was transferred to a 1 L separatory funnel and then the layers separated. The aqueous layer was extracted twice more (150 mL each) with CH₂Cl₂ and the combined organic layers were washed twice with water (100 mL), once with 1M HCl (˜100 mL) and once with brine (˜100 mL) before drying over sodium sulfate. The drying agent was removed by filtration and the solvent was removed in vacuo before silica gel chromatography (90 g silica gel column), eluting with 0 to 30% ethyl acetate/hexanes over a 1.5 hr period of time to afford the desired aldehyde (5), 4.88 g in 37% isolated yield; ¹H NMR (400 MHz, CDCl₃) δ ppm 9.56 (d, J=1.45 Hz, 0.4H, aldehyde CH rotamer), 9.52 (d, J=2.07 Hz, 0.60H, aldehyde CH rotamer), 4.32 (br. s., 1H), 4.15 (d, J=8.91 Hz, 0.4H, rotamer), 4.04 (d, J=9.33 Hz, 0.6H, rotamer), 3.41 (m, 2H), 2.15 (m, 1H), 2.02 (m, 1H), 1.45 (s, 4H, rotamer, NBoc t-Bu), 1.41 (s, 5H, rotamer, NBoc t-Bu), 0.81 (s, 9H), 0.03 (s, 3 H), −0.00 (s, 3H).

### tert-Butyl-(2R,4S)-4-hydroxy-2-prop-2-yn-1-ylpyrrolidine-1-carboxylate (8b)

Step 1. Wittig Reaction. (Methoxymethyl)triphenylphosphonium chloride (11.4 g, 0.0333 mol) was added in three portions to a suspension of potassium tert-butoxide (3.49 g, 0.0311 mol) in tetrahydrofuran (200 mL) that was cooled at 0° C. in an ice-water bath. The ice bath was removed from the dark red reaction solution with continued stirring for 2 hr. After this period of time, the reaction was cooled once more at 0° C. and then tert-butyl-(2S,4S)-4-{([tert-butyl(dimethyl)silyl]oxy}-2-formylpyrrolidine-1-carboxylate (5) (4.88 g, 0.0148 mol) in tetrahydrofuran (50 mL, 0.6 mol) was added dropwise via a pressure equalizing dropping funnel and the resultant solution was stirred overnight and the ice-bath was allowed to expire. TLC after this period of time (20% ethyl acetate/hexanes) shows consumption of the starting aldehyde and formation of the desired enol ether. The reaction was quenched by the addition of saturated ammonium chloride and the organic product was extracted with ethyl acetate (3×125 mL). The combined organic layers were washed with brine, dried over MgSO₄, filtered and concentrated in vacuo to afford the crude enol ether (6). Silica gel chromatography, eluting with 0 to 30% ethyl acetate/hexanes (90 g silica gel) over a 1 hr gradient afforded the desired enol ether (6) with ¹H NMR showing the enol H's and no aldehyde present. The product was used directly in the next step; enol ether: ¹H NMR (400 MHz, CDCl₃) δ ppm 6.38 (m, 06H), 5.74 (m, 0.4H), 4.93 (dd, J=12.65, 9.33 Hz, 0.6H), 4.60 (m, 0.4H), 4.25 (m, 1H), 4.07 (m, 1H), 3.47 (m, 4H), 3.22 (m, 1 H), 2.11 (m, 1H), 1.62 (m, 1H), 1.37 (s, 9H), 0.82 (s, 5H), 0.80 (s, 4H), −0.00 (s, 3 H), −0.02 (s, 3H);

Step 2. Enol ether hydrolysis: The enol ether (6) was dissolved in acetonitrile (200 mL) and 5% aqueous TFA solution (24.0 mL) was added. The reaction was stirred for 2 hr at ambient temperature and then checked by TLC (20% ethyl acetate/hexanes) and the starting enol ether had been consumed. The reaction was quenched by the addition of 300 mL of saturated sodium bicarbonate to pH <7 and the organic product was extracted with ethyl acetate (3×100 mL). The combined organic layers were washed with brine, dried over sodium sulfate, filtered and concentrated in vacuo to afford the crude aldehyde (a mixture of (7a) and (7b), inconsequential to the next step) which was subjected directly to the Bestmann conditions described in the next step.

Step 3. Bestmann Reaction: Dimethyl 2-oxopropylphosphonate (3.1 mL, 0.022 mol) was dissolved in acetonitrile (20 mL) and then was cooled at 0° C. in an ice-water bath before adding potassium carbonate (6.1 g, 0.044 mol). The reaction was then stirred for 5 minutes at reduced temperature before adding 4-methylbenzenesulfonyl azide (4.4 g, 0.022 mol) dropwise via a pressure equalizing dropping funnel as a solution in acetonitrile (8 mL). The ice-bath was removed and the reaction stirred for 2 hr at ambient temperature. TLC after this period of time (30% ethyl acetate:CH₂Cl₂) showed consumption of the starting materials and formation of the diazo species. Next, the aldehyde (7a/7b) was added as a solution in methanol (200 mL) via a pressure equalizing dropping funnel with continued stirring overnight at ambient temperature. TLC of the crude reaction mixture after this period of time shows consumption of the aldehyde and formation of the desired product—both the TBS ether (8a) and the hydroxyl compound (8b) were observed. The reaction was quenched by the addition of saturated aqueous sodium bicarbonate (˜100 mL) and then the reaction mixture was concentrated in vacuo to remove the methanol and acetonitrile. The crude slurry was partitioned between ethyl acetate (˜300 mL) and water (˜100 mL) and then the aqueous layer was extracted twice more with 50 mL portions of ethyl acetate. The combined organic layers were washed with 1M KOH (2×50 mL) and brine (1×50 mL), dried over MgSO₄, filtered and concentrated in vacuo to afford the crude products. Silica gel chromatography with ethyl acetate in hexanes (0 to 20%) afforded the TBS ether (8a, 2.5 g) and alcohol (8b, 1.5 g). The TBS ether (8a) was treated with 1.0 M of tetra-n-butylammonium fluoride in tetrahydrofuran (7.5 mL) in tetrahydrofuran (100 mL) that was cooled at 0° C. in an ice-water bath. The TBS moiety was rapidly removed under these reaction conditions (<30 min). The reaction mixture was concentrated in vacuo and then taken up in water and the organic product extracted with ethyl acetate (3×100 mL) and the combined organic layers washed once with water (˜50 mL) and once with brine (˜50 mL) to afford the crude product (8b), a mixture of diastereomers, epimeric at the α-stereocenter. The desired product (cis-8b) is higher R_{f}. Silica gel chromatography, eluting with 0 to 30% ethyl acetate in CH₂Cl₂ afforded the purified products, lower R_{f} (trans, undesired, 1.01 g, 30%) and higher R_{f} (cis, 1.20 g, 36%) in a combined yield of 66% for the 4 steps; ¹H NMR (8b, cis, 400 MHz, CDCl₃) δ ppm 4.39 (br. s., 1H), 3.95 (m, 1H), 3.64 (m, 1H), 3.36 (d, J=12.02 Hz, 1H), 2.68 (m, 2H), 2.22 (m, 1H), 2.07 (m, 1.5H), 1.97 (t, J=2.18 Hz, 1H, alkyne CH), 1.90 (m, 0.5H), 1.43 (br. s., 9H).

### Ethyl-8-{3-[(2R,4S)-1-(tert-butoxycarbonyl)-4-hydroxypyrrolidin-2-yl]prop-1-yn-1-yl}-1-cyclopropyl-6,7-difluoro-4-oxo-1,4-dihydroquinoline-3-carboxylate (9)

tert-Butyl-(2R,4S)-4-hydroxy-2-prop-2-yn-1-ylpyrrolidine-1-carboxylate (8b) (1.20 g, 0.00533 mol), ethyl-1-cyclopropyl-6,7-difluoro-4-oxo-8-{[(trifluoromethyl)sulfonyl]oxy}-1,4-dihydroquinoline-3-carboxylate (2.35 g, 0.00533 mol) and triphenylphosphine (0.35 g, 0.0013 mol) were transferred to a 100-mL round bottom flask equipped with a reflux condensor and then the vessel was placed under an atmosphere of nitrogen by partial evacuation and back-filling with nitrogen. Then, tetrahydrofuran (34 mL) was added and the reaction mixture was sparged with nitrogen for 2-3 minutes before adding N,N-diisopropylethylamine (1.86 mL, 0.0106 mol) and tetrakis(triphenylphosphine)palladium(0) (0.62 g, 0.00053 mol). The vessel was sparged for an additional 2-3 minutes and then copper(I) iodide (0.25 g, 0.0013 mol) was added and the reaction vessel was allowed to stir overnight at 60° C. for ˜10 hr before checking. HPLC and LCMS after this period of time show consumption of the triflate and alkyne and formation of a major product that is the desired Songogashira coupled product (9). The reaction was cooled to ambient temperature and then ethanol (25 mL) was added to the reaction vessel with continued stirring for 10 minutes. After this period of time, the reaction was filtered to remove the precipitated salts and the filtrate concentrated in vacuo. Silica gel chromatography (120 g cartridge), eluting with 0 to 50% ethyl acetate in CH₂Cl₂ afforded the desired coupled product (9), 2.16 g in 75% yield; ¹H NMR (400 MHz, CDCl₃) δ ppm 8.60 (s, 1H), 8.17 (t, J=9.23 Hz, 1H), 4.53 (m, 1H), 4.39 (q, J=7.12 Hz, 2H), 4.21 (m, 1H), 4.14-4.03 (m, 1H), 3.68-3.61 (m, 1H), 3.44 (dd, J=11.82, 1.87 Hz, 1H), 3.19-3.05 (m, 2H), 2.27 (m, 2H), 1.49 (s, 9H), 1.41 (t, J=7.15 Hz, 3H), 1.30 (m, 2H), 1.11 (m, 2H). MS: 517.0 m/z (M+1) for [C₂₇H₃₀F₂N₂O₆+H]⁺; HPLC: 4.072 min; Method B.

### Ethyl-8-{(1Z)-3-[(2R,4S)-1-(tert-butoxycarbonyl)-4-hydroxypyrrolidin-2-yl]prop-1-en-1-yl}-1-cyclopropyl-6,7-difluoro-4-oxo-1,4-dihydroquinoline-3-carboxylate (10)

Ethyl-8-{3-[(2R,4S)-1-(tert-butoxycarbonyl)-4-hydroxypyrrolidin-2-yl]prop-1-yn-1-yl}-1-cyclopropyl-6,7-difluoro-4-oxo-1,4-dihydroquinoline-3-carboxylate (9) (1.75 g, 0.00339 mol) was placed under an atmosphere of N₂ and then dissolved in ethanol (100 mL). The resulting solution was sparged with a gentle stream of nitrogen for 5 minutes and then the reaction vessel was charged with 5% palladium on barium sulfate (0.17 g), triethylamine (0.10 mL, 0.00072 mol) and quinoline (0.10 mL, 0.00085 mol). The resultant mixture was sparged with nitrogen for 2 minutes and then placed under an atmosphere of hydrogen by partial evacuation and back-fill of the flask with hydrogen via balloon (×3). Then, 2-1 L hydrogen balloons were bubbled through the reaction mixture and the reaction was maintained under an atmosphere of hydrogen with a balloon. The reaction was stirred vigorously for 5 hr and then checked for completion by HPLC/LCMS. After this period of time, the reaction had not progressed very far. The reaction was charged once more with a hydrogen balloon and then stirred overnight. After this period of time, the reaction had not progressed any further, and so the reaction was filtered through a short plug of Celite 545 and the filter cake rinsed with 3-20 mL portions of ethanol. The filtrate was concentrated in vacuo and then resubjected to hydrogenation conditions as follows: The yellow foam was dissolved in ethanol (100 mL) and the solution was sparged with nitrogen for 5-7 minutes. Then, 10% palladium on carbon (0.36 g) and triethylamine (0.10 mL, 0.72 mol) was added with continued sparging for 2-3 minutes before placing the reaction vessel under an atmosphere of hydrogen by partial evacuation and back-fill with hydrogen (3×). Then 2-1 L balloons filled with hydrogen were bubbled through the reaction mixture and then the reaction maintained under an atmosphere of hydrogen with a balloon. The reaction was stirred for 10 hr under hydrogen and then checked by HPLC/LCMS at which time the reaction was determined to be complete. The reaction was filtered through a short plug of Celite 545 and the filter cake rinsed with 3-10 mL portions of ethanol and then the filtrate was concentrated in vacuo. Silica gel chromatography (40 g) eluting with 0 to 50% ethyl acetate in CH₂Cl₂ afforded, 1.50 g (85%) of the desired cis-olefin (10); ¹H NMR confirms; ¹H NMR (400 MHz, CDCl₃) d ppm 8.65 (s, 1H), 8.23 (t, J=9.54 Hz, 1H), 6.83 (d, J=11.20 Hz, 1H), 5.98-6.06 (m, 1H), 4.41 (q, J=7.05 Hz, 2H), 4.28-4.36 (m, 1H), 3.85-3.99 (m, 1H), 3.75-3.82 (m, 1H), 3.41-3.66 (m, 1H), 3.13 (d, J=12.02 Hz, 1H), 2.39-2.55 (m, 1H), 1.94-2.21 (m, 2H), 1.53-1.69 (m, 2H), 1.43 (br. s., 9H), 1.43 (t, J=7.05 Hz, 3H), 1.19-1.26 (m, 1H), 1.10-1.16 (m, 1H), 0.99-1.07 (m, 1H), 0.84-0.92 (m, 1H); MS: ES⁺519.0 m/z (M+1) for [C₂₇H₃₂F₂N₂O₆+1]⁺ and ES″ (M+formate) for [C₂₇H₃₂F₂N₂O₆+formate]⁻; HPLC: 3.894 min (Method B).

### Ethyl-(7aR,9S)-4-cyclopropyl-12-fluoro-9-hydroxy-1-oxo-4,7,7a,8,9,10-hexahydro-1H-pyrrolo-[1′,2′:1,7]azepino[2,3-h]quinoline-2-carboxylate (11b)

Ethyl-8-{(1Z)-3-[(2R,4S)-1-(tert-butoxycarbonyl)-4-hydroxypyrrolidin-2-yl]prop-1-en-1-yl}-1-cyclopropyl-6,7-difluoro-4-oxo-1,4-dihydroquinoline-3-carboxylate (10) (1.50 g, 0.00289 mol) was dissolved in CH₂Cl₂ (50 mL) and trifluoroacetic acid (TFA, 3.5 mL, 0.045 mol) was added. The reaction was stirred overnight (˜12 hr) at ambient temperature and then checked by HPLC which showed complete consumption of the starting material. The TFA was removed by a gentle stream of nitrogen and then the reaction was diluted with 5% methanol/CHCl₃ (˜200 mL) and then ˜75 mL of a 10% solution of ammonium hydroxide was added and the contents transferred to a 500 mL separatory funnel. The layers were separated and then the resultant aqueous layer was washed twice with 100 mL portions of 5% methanol in CHCl₃ and then the combined organic layers washed with brine (˜75 mL) and then concentrated in vacuo. LCMS of the crude product shows a small amount of the cyclized material (11b) along with the uncyclized, des-Boc intermediate (11a) and some TFA-amide (11a-TFA amide). The foam solid was dissolved in anhydrous methanol (˜200 mL) and then potassium carbonate (0.420 g, 0.00304 mol) was added. The reaction was stirred for 16 hr at ambient temperature at which time LCMS shows absence of the TFA amide and a 1:1 mixture of (11a):(11b)—although both are now a mixture of Me and Et ester. The reaction was neutralized to pH 7 with dropwise addition of acetic acid and then the reaction mixture was concentrated to remove the methanol. The crude slurry/oil was partitioned between 5% methanol/chloroform (˜150 mL) and water (˜75 mL). The water layer was found to contain mostly uncyclized amino-ester (49a) and the organic layer contained mostly the desired cyclized product (11b)—both as a mixture of methyl and ethyl esters (MS: ES+385.0, M+1 for methyl ester; 399.1 m/z, M+1 for ethyl ester). The organic layer was concentrated to dryness and subjected to silica gel chromatography (40 g) eluting with 0 to 10% methanol in chloroform to afford 450 mg of the desired product as a mixture of methyl and ethyl esters (˜62%:38%) by HPLC at 280 nm. The aqueous layer was lyophilized to remove the water and the resultant solid was taken up in acetonitrile (50 mL) and then N,N-diisopropylethylamine (3.0 mL, 0.017 mol) was added. The resultant reaction was stirred at ambient temperature for 6 hr before checking by HPLC. HPLC after this period of time shows complete formation of the fully cyclized product (11b). The reaction was concentrated in vacuo and then purified as above to afford 260 mg of mixture of methyl and ethyl ester for a combined yield of 710 mg, 62% yield; (ethyl ester): ¹H NMR (400 MHz, CDCl₃) δ ppm 8.57 (s, 1H), 7.71 (d, J=15.13 Hz, 1H), 6.26-6.33 (m, 1H), 5.79-5.87 (m, 1H), 4.60-4.64 (m, 1H), 4.38-4.45 (m, 2H), 4.20-4.27 (m, 1H), 3.95-4.01 (m, 1H), 3.75-3.86 (m, 2H), 3.13 3.21 (m, 1H), 2.97 (br. s., 1H), 2.43-2.51 (m, 1H), 2.24-2.32 (m, 1H), 1.90 (d, J=13.27 Hz, 1H), 1.44 (t, J=7.15 Hz, 3H), 1.21-1.29 (m, 1H), 0.88-0.99 (m, 2H), 0.68-0.75 (m, 1H); (methyl ester): ¹H NMR (400 MHz, CDCl₃) δ ppm 8.59 (s, 1H), 7.72 (d, J=15.34 Hz, 1H), 6.27-6.33 (m, 1H), 5.80-5.87 (m, 1H), 4.61-4.64 (m, 1H), 4.20-4.27 (m, 1 H), 3.95-4.00 (m, 1H), 3.94 (s, 3H), 3.80-3.86 (m, 1H), 3.75-3.80 (m, 1H), 3.11-3.21 (m, 1H), 2.90 (br. s., 1H), 2.43-2.51 (m, 1H), 2.24-2.33 (m, 1H), 1.90 (d, J=13.27 Hz, 1H), 1.21-1.29 (m, 1H), 0.87-0.99 (m, 2H), 0.67-0.75 (m, 1H).

### (7aR,9S)-4-Cyclopropyl-12-fluoro-9-hydroxy-1-oxo-4,7,7a,8,9,10-hexahydro-1H-pyrrolo[1′,2′:1,7]azepino[2,3-h]quinoline-2-carboxylic acid (12)

Ethyl-(7aR,9S)-4-cyclopropyl-12-fluoro-9-hydroxy-1-oxo-4,7,7a,8,9,10-hexahydro-1H-pyrrolo[1′,2′:1,7]azepino[2,3-h]quinoline-2-carboxylate (11b) (0.26 g, 0.00065 mol) was dissolved in water (1 mL) and acetonitrile (15 mL) and then treated with 0.500 M aqueous sodium hydroxide (2 mL). The reaction was heated at 60° C. for 6 hr and then checked by HPLC/LCMS. Analysis after this period of time shows complete consumption of the starting material and formation of a new product with MS consistent with the desired hydroxy acid. The reaction was cooled to rt and then neutralized by the dropwise addition of acetic acid. Then, the reaction mixture was concentrated in vacuo and then the resultant film was partitioned between chloroform (50 mL) and water (20 mL). The aqueous layer was extracted twice more with 5% methanolic chloroform (50 mL each) and then the combined organic layers were washed with brine, dried over sodium sulfate, filtered and concentrated in vacuo to afford a yellow solid. The solid was triturated with methyl tert-butyl ether and the resulting solid dried overnight on high vacuum to afford, 220 mg, 91% yield of the desired hydroxy acid (12) with ¹H NMR consistent with the proposed structure; ¹H NMR (400 MHz, DMSO-d₆) δ ppm 8.73 (s, 1H), 7.58 (d, J=15.13 Hz, 1H), 6.65 (d, J=12.23 Hz, 1H), 5.98 (td, J=7.77, 4.15 Hz, 1H), 5.15 (d, J=2.28 Hz, 1H), 4.43-4.46 (m, 1H), 4.22-4.28 (m, 1H), 4.12-4.19 (m, 1H), 3.91 (t, J=9.12 Hz, 1H), 3.72 (dd, J=11.51, 4.87 Hz, 1H), 3.16-3.27 (m, 1H), 2.53-2.57 (m, 1H), 2.24-2.33 (m, 1H), 1.78 (d, J=13.89 Hz, 1H), 1.22-1.31 (m, 1H), 0.92-1.06 (m, 2H), 0.79-0.87 (m, 1H); MS: ES⁺371.0 m/z (M+1) for [C₂₀H₂₉FN₂O₄+1]⁺; ES⁻ 369.0 m/z (M−1) for [C₂₀H₂₉FN₂O₄−1]⁺; HPLC: 3.159 min (Method B).

### Example 29

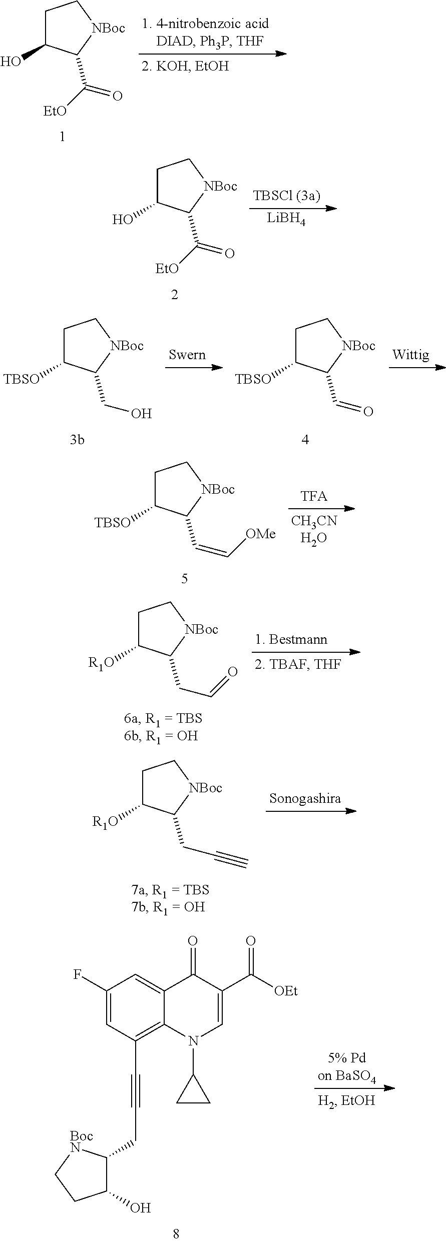

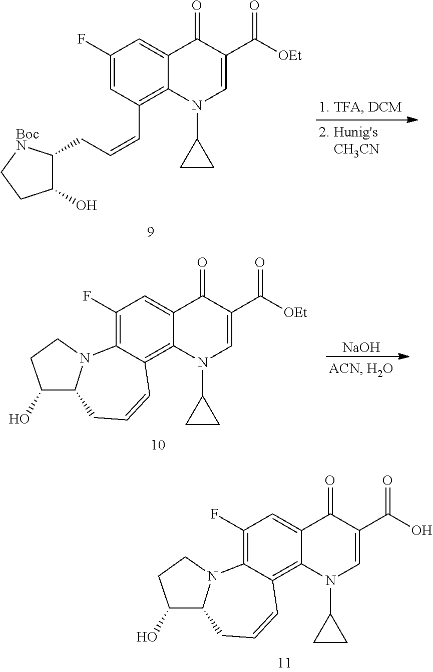

### 1-tert-Butyl 2-ethyl-(2S,3R)-3-hydroxypyrrolidine-1,2-dicarboxylate (2)

1-tert-Butyl-2-ethyl-(2S,3S)-3-hydroxypyrrolidine-1,2-dicarboxylate (1) (25.89 g, 99.85 mmol), 4-nitrobenzoic acid (20.0 g, 120 mmol), and triphenylphosphine (31.4 g, 120 mmol) were placed under an atmosphere of nitrogen in a 2 L round bottom flask. Then, tetrahydrofuran (900 mL) was added and the reaction vessel was cooled at 0° C. using an ice-bath. Then, diisopropyl azodicarboxylate (23.6 mL, 120 mmol) as a solution in tetrahydrofuran (310 mL) was added dropwise via a pressure equalizing dropping funnel and the reaction was stirred at reduced temperature for 1 hr before checking by TLC (30% EtOAc/hexanes). TLC after this period of time shows that the starting material is consumed. The reaction was diluted with 200 mL of CH₂Cl₂ and then transferred to a 2-L separatory funnel. The reaction was washed with saturated sodium bicarbonate (200 mL×1), water (200 mL×1) and brine (200 mL×1), dried over MgSO₄, filtered and concentrated in vacuo to afford the crude product. Silica gel chromatography (120 g silica gel ×2) eluting with 0 to 35% ethyl acetate in hexanes afforded purified product, 65.09 g (˜63% pure), 100% (corrected) yield of the p-nitrobenzoate ester, based on correction for DIAD by-product and Ph₃PO; (benzoate ester): ¹H NMR (400 MHz, CDCl₃) δ ppm 8.31 (d, J=8.71 Hz, 2H), 8.18 (d, J=8.71 Hz, 2H), 5.76 (q, J=6.22 Hz, 1H), 4.72 (d, J=6.84 Hz, 0.33H, CH rotamer), 4.65 (d, J=6.84 Hz, 0.67H, CH rotamer), 4.10 (m, 2H), 3.70 (m, 2H), 2.30 (m, 2H), 1.51 (s, 4H, Ot-Bu rotamer), 1.45 (s, 5H, Ot-Bu rotamer), 1.13 (m, 3H). The ester was hydrolyzed in the next step as follows:

The p-nitrobenzoate ester was dissolved in anhydrous methanol (480 mL) and then cooled at 0° C. in an ice-water bath. Then, potassium hydroxide (5.60 g, 99.8 mmol) in methanol (110 mL) was added via a pressure equalizing dropping funnel and the reaction stirred for 1 hr at reduced temperature before checking by TLC (35% ethyl acetate/hexanes). TLC after this period of time (35% ethyl acetate/CH₂Cl₂) shows consumption of the starting benzoate ester and formation of a lower R_{f} product. It appears, based on TLC, (and later determined by ¹H NMR) that some trans-esterification had taken place and a small amount of methyl ester is present. The ¹H NMR and TLC R_{f} of the product (2, lower R_{f}, cis-diastereomer) are different than the starting material (1, higher R_{f}, trans-diastereomer). The reaction was quenched by pouring into ice-water and neutralized by the careful addition of 1M HCl (cold) and then the organic product was extracted with ethyl acetate (3×200 mL). The combined organic layers were washed with water, dried over MgSO₄, filtered and concentrated in vacuo to afford the crude product. Silica gel chromatography (2×120 g) eluting with 0 to 40% ethyl acetate/hexanes afforded purified product, 25.7 g in 99% yield; (alcohol) ¹H NMR (400 MHz, CDCl₃) δ ppm 4.61 (m, 1H), 4.38 (m, 1H), 4.25 (m, 2H), 3.64 (m, 1H), 3.49 (m, 1H), 2.49 (m, 1H), 2.12 (m, 1H), 2.03 (m, 1H), 1.47 (s, 4H), 1.43 (s, 5H), 1.30 (m, 3H).

### 1-tert-Butyl-2-ethyl-(2S,3R)-3-{[tert-butyl(dimethyl)silyl]oxy}pyrrolidine-1,2-dicarboxylate (3a)

1-tert-Butyl-2-ethyl-(2S,3R)-3-hydroxypyrrolidine-1,2-dicarboxylate (2) (3.8 g, 0.015 mol) was dissolved in N,N-dimethylformamide (100 mL) and then 1H-imidazole (1.5 g, 0.022 mol) and tert-butyldimethylsilyl chloride (2.32 g, 0.0154 mol) were added successively. The reaction was stirred overnight at ambient temperature and determined to be complete (TLC, 30% ethyl acetate/hexanes) after this period of time. The reaction was diluted with 200 mL of water and then the product extracted with diethyl ether (3×100 mL) and the combined organic layers washed with water (3×50 mL), saturated sodium bicarbonate (2×50 mL), and brine (1×50 mL). The organic layer was dried over MgSO₄, filtered and concentrated in vacuo to afford the crude product (3a) (4.9 g, 90% yield) which was used in the next step without further purification; ¹H NMR (400 MHz, CDCl₃) δ ppm 4.46 (m, 1H), 4.28 (m, 0.5H), 4.16 (m, 1H), 4.14, (m, 0.5H), 4.00 (m, 1H), 3.59-3.53 (m, 1H), 3.31 (m, 1H), 1.92 (m, 2 H), 1.33 (s, 9H), 1.19 (m, 3H), 0.79 (s, 9H), −0.00 (s, 3H), −0.01 (s, 3H).

### tert-Butyl-(2R,3R)-3-{[tert-butyl(dimethyl)silyl]oxy}-2-(hydroxymethyl)pyrrolidine-1-carboxylate (3b)

1-tert-Butyl-2-ethyl-(2S,3R)-3-{[tert-butyl(dimethyl)silyl]oxy}pyrrolidine-1,2-dicarboxylate (3a) (4.5 g, 0.012 mol) was dissolved in tetrahydrofuran (150 mL) and was cooled at 0° C. in an ice-water bath. Then, lithium tetrahydroborate (1.84 g, 0.0847 mol) was added in one portion and then the ice-bath was removed. Subsequently, the reaction was heated at reflux for 24 hr before checking. TLC after this period of time shows a new, lower R_{f} product. The reaction was cooled to ambient temperature and quenched by the addition of water and the organic product was extracted with ethyl acetate (3×75 mL), the combined organic layers were washed with brine, dried over MgSO₄, filtered and concentrated in vacuo to afford the crude product. The crude product was purified to afford the desired alcohol (3b), 2.34 g in 58% yield; ¹H NMR (400 MHz, CDCl₃) δ ppm 4.44-4.30 (m, 1H, CH), 3.79-3.70 (m, 2H, CH+CH of CH₂), 3.61 (m, 1H, CH of CH₂), 3.39-3.25 (m, 2H, ring CH₂), 2.65 (m, 1H, OH shows cosy signal to methylene), 1.80 (m, 2H, ring CH₂), 1.38 (s, 9H, t-Bu), 0.80 (br. s., 9H, t-Bu), 0.01 (m, 6H, Me of TBS).

### tert-Butyl-(2S,3R)-3-{[tert-butyl(dimethyl)silyl]oxy}-2-formylpyrrolidine-1-carboxylate (4)

Dimethyl sulfoxide (2.00 mL, 0.0282 mol) was added (dropwise via syringe over ˜30 min) to a stirred solution of oxalyl chloride (1.19 mL, 0.0141 mol) in CH₂Cl₂ (100 mL) that was cooled at −78° C. in a dry-ice acetone bath. After this addition was complete, the reaction was stirred for 30 minute before adding the substrate, tert-butyl-(2R,3R)-3-{[tert-butyl(dimethyl)silyl]oxy}-2-(hydroxymethyl)pyrrolidine-1-carboxylate (3b) (2.34 g, 0.00706 mol) as a solution in CH₂Cl₂ (25 mL) over a 20 minute period of time, to maintain the reaction temperature at −65° C. or less. The reaction was stirred for 15 minutes at the same temperature and the cold bath was removed briefly for ˜15 minutes to allow the reaction to warm to −35° C. Then, the reaction was cooled once more at −78° C. and triethylamine (3.94 mL, 0.0282 mol) in CH₂Cl₂ (11 mL) was added dropwise and the reaction was stirred overnight and the cold bath was allowed to slowly expire (to −20° C.). After this period of time, the reaction was complete and was quenched by the addition of water and the organic product was extracted with CH₂Cl₂ (3×100 mL) and the combined organic layers were washed with water (3×100 mL) and brine (1×100 mL) and then dried over MgSO₄, filtered and concentrated in vacuo to afford the crude aldehyde. Silica gel chromatography using a 40 g silica gel column, eluting with 0 to 25% ethyl acetate in hexanes afforded the desired aldehyde (4), 2.03 g in 87% yield; ¹H NMR (400 MHz, CHCl₃) δ ppm 9.41 (d, J=2.49 Hz, 0.33H, rotamer CHO), 9.34 (d, J=3.11 Hz, 0.67H, rotamer CHO), 4.62 (m, 1H), 4.03 (dd, J=5.18, 2.70 Hz, 0.33H, rotamer α-CH), 3.90 (dd, J=5.08, 3.21 Hz, 0.67H, α-CH rotamer), 3.57 (m, 2H), 1.85 (m, 2H), 1.42 (s, 4H, t-Boc rotamer), 1.35 (s, 5H, t-Boc rotamer), 0.79 (s, 9H), −0.00 (s, 3H), −0.02 (s, 3H).

### tert-Butyl-(2R,3R)-3-hydroxy-2-prop-2-yn-1-ylpyrrolidine-1-carboxylate (7b)

Step 1. Wittig Reaction. (Methoxymethyl)triphenylphosphonium chloride (4.75 g, 0.0139 mol) was added in three equal portions over a 5-minute period of time to a stirred mixture of potassium tert-butoxide (1.45 g, 0.0129 mol) in tetrahydrofuran (100 mL) that was cooled at 0° C. in an ice-water bath. After this addition was complete, the ice bath was removed and the reaction was warmed to ambient temperature with continued stirring for 2 hr. After this period of time, the dark red reaction mixture was cooled at 0° C. in an ice-water bath and tert-butyl-(2S,3R)-3-{[tert-butyl(dimethyl)silyl]oxy}-2-formylpyrrolidine-1-carboxylate (4) (2.03 g, 0.00616 mol) was added dropwise via an addition funnel as a solution in tetrahydrofuran (50 mL, 0.6 mol) with continued stirring overnight (˜12 hr) during which time the ice-bath was allowed to expire. After this period of time, the reaction was complete based on TLC. The reaction was quenched by the addition of saturated ammonium chloride (˜100 mL) and the organic product was extracted with ethyl acetate (3×75 mL) and the combined organic layers washed with water (˜75 mL), brine (˜75 mL), dried over sodium sulfate, filtered and concentrated in vacuo to afford the crude enol ether. Rapid silica gel chromatography using a 40 g silica gel column afforded the desired enol ether (5, very complex ¹H NMR) that was used in the next step without further purification.

Step 2. Enol ether hydrolysis. The crude enol ether was dissolved in acetonitrile (60 mL) and then a 5% aqueous TFA solution (9.97 mL) was added and the reaction was stirred for 2 hr at ambient temperature. After this period of time, TLC compared to the starting enol ether shows consumption of one of the spots, and the reaction was quenched by the addition of 200 mL of saturated aqueous sodium bicarbonate to pH <7. Then, the acetonitrile was removed in vacuo and the organic product was extracted (3×100 mL) with ethyl acetate and the combined organic layers were washed with brine, dried over sodium sulfate, filtered and concentrated in vacuo to afford the crude aldehyde (a mixture of (6a) and (6b)) which was subjected directly to the Bestmann conditions as described in the next step.

Step 3. Bestmann reaction. Dimethyl 2-oxopropylphosphonate (1.3 mL, 0.0092 mol) was dissolved in acetonitrile (6 mL) and then the reaction vessel was cooled at 0° C. in an ice-water bath. Then potassium carbonate (2.6 g, 0.018 mol) was added in one portion and the reaction was stirred for 5 minutes before adding 4-methylbenzenesulfonyl azide (1.8 g, 0.0092 mol) as a solution in acetonitrile (3 mL) via a pressure equalizing dropping funnel. After the addition was complete, the reaction was warmed to ambient temperature and stirred for 2 hr before checking for the desired diazo intermediate (TLC 30% ethyl acetate/DCM). TLC analysis shows complete formation of the desired intermediate. Next, the crude aldehyde (6a/6b) was added dropwise via a pressure equalizing dropping funnel as a solution in methanol (100 mL). The resultant mixture was stirred at ambient temperature for 18 hr before checking. TLC after this period of time shows that the reaction is complete. The reaction was quenched by the addition of 20 mL of saturated aqueous sodium bicarbonate and then concentrated in vacuo to remove the methanol and acetonitrile. The crude film was partitioned between water (100 mL) and ethyl acetate (250 mL) and the aqueous layer was extracted an additional two times with 100 mL of ethyl acetate. The combined organic layers were washed with 1M KOH (2×50 mL), brine, dried over MgSO₄, filtered and concentrated in vacuo to afford the crude alkyne product as a mixture of TBS ether (7a) (320 mg) and alcohol (7b) (490 mg) obtained after silica gel chromatography (40 g) eluting with 0 to 20% ethyl acetate in hexanes. The two products were separated by silica gel chromatography and then the TBS ether was treated with 1.0 M of tetra-n-butylammonium fluoride in tetrahydrofuran (1.0 mL) in tetrahydrofuran (50 mL) that was cooled at 0° C. in an ice-water bath to afford the desired alcohol (7b) after an aqueous workup. The combined lots of alcohol were purified by silica gel chromatography, eluting with 0 to 30% ethyl acetate in CH₂Cl₂ to afford the desired product after careful chromatography. The desired product (7b, 134 mg, 10% yield) was the higher R_{f} (non-UV active) of the three products isolated. ¹H NMR and MS confirm; ¹H NMR (400 MHz, CDCl₃) δ ppm 4.55 (m, 1H), 3.89 (m, 1 H), 3.50 (m, 2H), 2.96 (m, 0.5H), 2.77 (m, 0.5H), 2.55 (dd, 0.5H, J=2.1, 8.4 Hz), 2.51 (dd, 0.5H, J=2.2, 8.9 Hz), 2.19 (m, 1H), 2.04 (m, 3H), 1.47 (s, 9H); MS: ES⁺248.1 m/z (M+Na) for [C₁₂H₁₉NO₃+Na]⁺.

### Ethyl-8-{3-[(2R,3R)-1-(tert-butoxycarbonyl)-3-hydroxypyrrolidin-2-yl]prop-1-yn-1-yl}-1-cyclopropyl-6,7-difluoro-4-oxo-1,4-dihydroquinoline-3-carboxylate (8)

tert-Butyl-(2R,3R)-3-hydroxy-2-prop-2-yn-1-ylpyrrol-idine-1-carboxylate (7b) (0.134 g, 0.595 mmol), ethyl-1-cyclopropyl-6,7-difluoro-4-oxo-8-{[(trifluoromethyl)sulfon-yl]oxy}-1,4-dihydroquinoline-3-carboxylate (0.262 g, 0.000595 mol) and triphenylphosphine (0.039 g, 0.15 mmol) were transferred to a 100-mL round bottom flask equipped with a reflux condensor and then the vessel was placed under an atmosphere of nitrogen by partial evacuation and back-filling with nitrogen. Then, tetrahydrofuran (4 mL) was added and the reaction mixture was sparged with nitrogen for 2-3 minutes before adding N,N-diisopropylethylamine (0.207 mL, 1.19 mmol) and tetrakis(triphenylphosphine)palladium(0) (0.069 g, 0.059 mmol). Then the reaction was sparged with nitrogen for 2-3 additional minutes and finally copper(I) iodide (0.028 g, 0.15 mmol) was added and the reaction vessel was allowed to stir overnight at 60° C. for ˜10 hr before checking. HPLC and LCMS after this period of time show consumption of the triflate and alkyne and formation of a major product that is the desired Songogashira coupled product (8). Ethanol (25 mL) was added to the reaction vessel with continued stirring for 10 minutes. After this period of time, the reaction was filtered to remove the precipitated salts and the filtrate concentrated in vacuo. Silica gel chromatography (120 g cartridge), eluting with 0 to 30% ethyl acetate in CH₂Cl₂ afforded the desired coupled product (8), 2.16 g in 75% yield; ¹H NMR (400 MHz, CDCl₃) δ ppm 8.48 (br. s., 1H), 8.09 (t, J=8.81 Hz, 1H), 4.57 (d, J=4.35 Hz, 1 H), 4.31 (q, J=7.19 Hz, 2H), 4.11-4.18 (m, 1H), 3.92-3.98 (m, 1H), 3.39-3.45 (m, 2H), 3.12-3.21 (m, 1H), 2.78-2.86 (m, 1H), 1.93-2.05 (m, 3H), 1.40 (br. s., 9H), 1.33 (t, J=7.15 Hz, 3H), 1.24 (d, J=6.84 Hz, 2H), 1.02 (d, J=3.32 Hz, 2H); HPLC: 4.088 min (Method B); MS: 517.0 m/z (M+1) for [C₂₇H₃₀F₂N₂O₆+1]⁺.

### Ethyl-8-{(1Z)-3-[(2R,3R)-1-(tert-butoxycarbonyl)-3-hydroxypyrrolidin-2-yl]prop-1-en-1-yl}-1-cyclopropyl-6,7-difluoro-4-oxo-1,4-dihydroquinoline-3-carboxylate (9)

Ethyl-8-{3-[(2R,3R)-1-(tert-butoxycarbonyl)-3-hydroxypyrrolidin-2-yl]prop-1-yn-1-yl}-1-cyclopropyl-6,7-difluoro-4-oxo-1,4-dihydroquinoline-3-carboxylate (8) (0.25 g, 0.48 mmol) was placed under an atmosphere of nitrogen and then ethanol (25 mL) was added and the reaction was sparged with a gentle stream of nitrogen for 5-7 minutes before adding palladium on barium sulfate (0.26 g). The reaction was sparged for 2 minutes longer and then 2-1 L balloons filled with hydrogen were bubbled through the reaction mixture. Then, the outlet needle was removed and the reaction was maintained under an atmosphere of hydrogen with a balloon for 4 hr before checking by HPLC/LCMS. HPLC after this period of time shows that the reaction is nearly complete (˜80%). The reaction was charged once more with another hydrogen balloon and then stirred overnight at ambient temperature before filtering to remove the palladium catalyst. The filtrate was concentrated in vacuo and the crude product was purified on 40 g of silica gel, eluting with 0 to 50% ethyl acetate/CH₂Cl₂ to afford the desired product (9), 207 mg, in 82% yield; ¹H NMR (400 MHz, CDCl₃) δ ppm 8.58 (s, 1H), 8.13 (t, J=9.54 Hz, 1H), 6.70 (d, J=10.78 Hz, 1H), 6.03 (dt, J=11.20, 7.15 Hz, 1H), 4.30 (q, J=7.05 Hz, 2H), 4.18-4.25 (m, 1H), 3.75-3.84 (m, 2 H), 3.10-3.40 (m, 2H), 1.85-2.13 (m, 2H), 1.59 (dd, J=12.65, 7.67 Hz, 1H), 1.53 (br. s., 2H), 1.34 (s, 9H), 1.30-1.33 (m, 3H), 1.07-1.12 (m, 2H), 0.87-0.93 (m, 2 H) MS: 519.0 m/z (M+1) for [C₂₇H₃₂F₂N₂O₆+1]⁺; HPLC: 3.905 min (Method B).

### Ethyl-(7aR,8R)-4-cyclopropyl-12-fluoro-8-hydroxy-1-oxo-4,7,7a,8,9,10-hexahydro-1H-pyrrolo[1′,2′:1,7]azepino-[2,3-h]quinoline-2-carboxylate (10)

Ethyl-8-{(1Z)-3-[(2R,3R)-1-(tert-butoxycarbonyl)-3-hydroxypyrrolidin-2-yl]prop-1-en-1-yl}-1-cyclopropyl-6,7-difluoro-4-oxo-1,4-dihydroquinoline-3-carboxylate (9) (0.207 g, 0.399 mmol) was dissolved in CH₂Cl₂ (15 mL) and then trifluoroacetic acid (1.0 mL, 0.013 mol) was added. The reaction was stirred overnight at ambient temperature and then checked by HPLC/MS. HPLC analysis after this period of time shows disappearance of the starting N-Boc compound and formation of a much more polar product; MS confirms product as well (MS: ES⁺399.0 m/z [M+1-F]⁺ for [C₂₂H₂₄F₂N₂O₄+1−F]⁺ and 419.0 m/z [M+1]⁺ for [C₂₂H₂₄F₂N₂O₄+1]⁺). The reaction was concentrated by a gentle stream of nitrogen and then the oil was taken up in chloroform (50 mL) and washed once with 10% aqueous ammonium hydroxide solution. The aqueous layer was extracted twice more with 5% methanolic chloroform and then the combined organic layers were washed with brine, dried over sodium sulfate, filtered and concentrated in vacuo to afford a mixture of the deprotected intermediate and also some cyclized product. The crude foam was dissolved in acetonitrile (20 mL) and N,N-diisopropylethylamine (2.0 mL, 0.011 mol) was added and the reaction was stirred overnight at ambient temperature. HPLC after this period of time shows conversion of the remaining uncyclized intermediate (HPLC: 2.491 min, Method B) to the desired cyclized product (10), HPLC: 3.123 min, Method B); MS also confirms (MS: ES⁺399.1 m/z, [M+1]⁺ for [C₂₂H₂₃FN₂O₄+1]⁺. The reaction was diluted with 100 mL of chloroform and then washed once with water (10 mL), once with 0.1 M HCl (25 mL), once with brine (25 mL), dried over sodium sulfate, filtered and concentrated in vacuo to afford a crude foam which was purified by silica gel chromatography, 40 g silica gel, eluting with 0 to 10% methanol in chloroform to afford, 92 mg, 57% yield of the desired hydroxy ester (10). The product was used directly in the next step without further characterization.

### (7aR,8R)-4-Cyclopropyl-12-fluoro-8-hydroxy-1-oxo-4,7,7a,8,9,10-hexahydro-1H-pyrrolo[1′,2′:1,7]aze-pino[2,3-h]quinoline-2-carboxylic acid (11)

Ethyl-(7aR,8R)-4-cyclopropyl-12-fluoro-8-hydroxy-1-oxo-4,7,7a,8,9,10-hexahydro-1H-pyrrolo[1′,2′:1,7]azepino[2,3-h]quinoline-2-carboxylate (10) (0.0902 g, 0.226 mmol) was dissolved in water (1 mL) and acetonitrile (5 mL) before adding 0.500 M aqueous sodium hydroxide (0.679 mL). Then the reaction was heated at 60° C. for 6 hours before checking by HPLC. HPLC after this period of time shows complete consumption of the starting ester and formation of the carboxylic acid The reaction was neutralized to pH ˜5 with by the dropwise addition of glacial acetic acid and then the solvent removed in vacuo. The resultant solid was suspended in water and then the water layer was extracted ×3 with chloroform (30 mL each) and then the combined organic layers were washed with brine, dried over sodium sulfate, and concentrated in vacuo. During the extraction, a fine yellow precipitate remained that did not go into either the aqueous or organic layers. This solid was isolated by filtration and HPLC/LCMS analysis showed that this was pure product. Only a small amount of impure carboxylic acid was present in the product isolated from the organic layer and no product was detected in the aqueous layer after the workup was complete. The isolated solid was dried on high vacuum to afford 44 mg in 52% yield of (11); ¹H NMR (400 MHz, DMSO-d₆) δ ppm 8.73 (s, 1H), 7.56 (d, J=14.93 Hz, 1H), 6.65 (d, J=12.23 Hz, 1H), 6.11 (td, J=7.98, 3.94 Hz, 1H), 5.32 (d, J=4.56 Hz, 1H), 4.38-4.47 (m, 1H), 4.21-4.27 (m, 1H), 3.97-4.10 (m, 1H), 3.79-3.87 (m, 1H), 3.71-3.78 (m, 1H), 2.56-2.67 (m, 1H), 2.43-2.50 (m, 1H), 2.07-2.15 (m, 1H), 1.87-1.99 (m, 1H), 1.22-1.31 (m, 1H), 0.97-1.05 (m, 1H), 0.90-0.97 (m, 1H), 0.82-0.88 (m, 1H); (MS: ES⁺370.9 m/z, M+1 for [C₂₀H₁₉FN₂O₄+H]⁺ and 392.92 m/z (M+Na) for [C₂₀H₁₉FN₂O₄+Na]⁺; HPLC (the sample was prepared by dissolving in CH₃CN with 1-2 drops of 0.50 M aqueous NaOH) 3.249 min (Method B).

### Example 30

### Example 30A

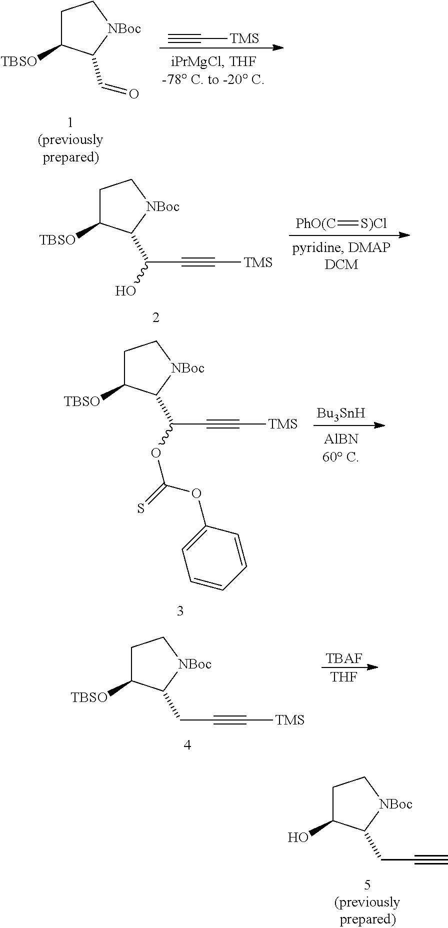

### tert-Butyl-(2R,3S)-3-{[tert-butyl(dimethyl)silyl]oxy}-2-[1-hydroxy-3-(trimethylsilyl)prop-2-yn-1-yl]pyrrolidine-1-carboxylate (2)

(Trimethylsilyl)acetylene (4.84 mL, 34.3 mmol) was dissolved in tetrahydrofuran (200 mL) and the solution was cooled at 0° C. in an ice-water bath before adding 1.8 M isopropylmagnesium chloride in tetrahydrofuran (16.5 mL) dropwise via syringe. The light yellow reaction mixture was stirred for 2 hr with continued cooling. After this period of time, the reaction was cooled in a dry-ice/acetone bath (−78° C.) and tert-butyl-(2S,3S)-3-{[tert-butyl(dimethyl)silyl]oxy}-2-formylpyrrolidine-1-carboxylate (1) (7.53 g, 22.8 mmol) in tetrahydrofuran (100 mL) was added dropwise over a 20 minute period of time via a pressure equalizing dropping funnel. The reaction was stirred for 4 hr and then checked by LCMS and TLC. TLC analysis after this period of time shows two diastereomeric alcohols, but the major diastereomer nearly co-spots with the starting aldehyde (20% ethyl acetate/hexanes). The spots were resolved by using-25-30% diethyl ether in hexanes as the eluting solvent, which showed a small amount of the starting aldehyde (MS: ES⁺230.2 m/z for [C₁₁H₂₄NO₂Si]⁺ for [M+H-t-Boc]⁺). The reaction was transferred to a freezer that was cooled at −20° C. and left overnight (˜9 hr). TLC analysis after this period of time showed complete consumption of the starting material. The reaction was quenched with saturated sodium bicarbonate and then subjected to an aqueous workup as follows. About 300 mL of ethyl acetate was added to the reaction mixture along with ˜100 mL of brine and the two layers separated. The aqueous layer was extracted once more with 100 mL of ethyl acetate and then the combined organic layers washed with brine, dried over MgSO₄, filtered and concentrated in vacuo to afford the crude product (2). ¹H NMR confirms. The crude product was used without further purification in the next step; (˜2:1 mixture of diastereomers): ¹H NMR (400 MHz, CDCl₃) δ ppm 5.66 (d, J=8.91 Hz, 0.65H), 4.75 (d, J=4.77 Hz, 0.35H), 4.36 (dd, J=8.71, 1.24 Hz, 0.65H), 4.23 (d, J=1.87 Hz, 0.35H), 3.96-4.01 (m, 0.65H), 3.89 (dd, J=10.26, 4.25 Hz, 0.35H), 3.71 (d, J=9.74 Hz, 0.33H), 3.63 (d, J=2.49 Hz, 0.33H), 3.54-3.59 (m, 0.35H), 3.29-3.44 (m, 1H), 3.13-3.27 (m, 1H), 1.89-2.00 (m, 0.50H), 1.50-1.69 (m, 1.50H), 1.30 (s, 9H), 0.70 (s, 9H), −0.00 (s, 4.5H), −0.02 (s, 4.5H), −0.08, −0.09, −0.10 (s, s, s, total to 6H) MS: ES⁺450.0 m/z (M+Na) for [C₂₁H₄₁NO₄Si₂+Na]⁺; 372.0 m/z (M-t-Bu) for [C₁₇H₃₄NO₄Si₂]⁺; 328.1 m/z (M-t-Boc) for [C₁₆H₃₄NO₂Si₂]⁺.

### tert-Butyl-(2R,3S)-3-{[tert-butyl(dimethyl)silyl]oxy}-2-{1-[(phenoxycarbonothioyl)oxy]-3-(trimethyl-silyl)prop-2-yn-1-yl}pyrrolidine-1-carboxylate (3)

tert-Butyl-(2R,3S)-3-{[tert-butyl(dimethyl)silyl]-oxy}-2-[1-hydroxy-3-(trimethylsilyl)prop-2-yn-1-yl]pyrrolidine-1-carboxylate (2) (10.53 g, 0.02462 mol) was dissolved in CH₂Cl₂ (500 mL) and then pyridine (15 mL, 0.19 mol) and 4-dimethylaminopyridine (0.60 g, 0.0049 mol) were added successively before adding phenyl chlorothionocarbonate (5.11 mL, 0.0369 mol) rapidly via syringe. After the addition of the chlorothionocarbonate, the reaction was bright orange. The reaction was stirred overnight at ambient temperature and then checked for completion by TLC. TLC analysis after this period of time reveals the complete consumption of the starting material and formation of a much higher R_{f} product that is the desired thionocarbonate (3). The reaction was quenched by the addition of 250 mL of saturated aqueous sodium bicarbonate and transferred to a 1 L separatory funnel and the layers separated. The organic layer was washed once more with 250 mL of saturated aqueous sodium bicarbonate, twice with 1M aqueous hydrochloric acid (200 mL each), once with brine (˜250 mL) and finally the organic layer was dried over sodium sulfate, filtered, and concentrated in vacuo to afford the crude product. Silica gel chromatography, using 90 g of silica gel, eluting with 0 to 20% ethyl acetate in hexanes afforded the desired thionocarbonate (3), 12.7 g in 91.5% yield, as a mixture of diastereomeric products; ¹H NMR confirms; ¹H NMR (400 MHz, CDCl₃) δ ppm 7.17-7.28 (m, 2H), 6.99-7.14 (m, 2H), 6.86-6.91 (m, 1H), 6.23 (d, J=2.49 Hz, 0.4H), 5.99-6.04 (m, 0.6H), 4.45 (dd, J=12.23, 4.15 Hz, 0.6H), 4.34-4.38 (m, 0.4H), 3.75-3.83 (m, 0.6H), 3.63 (d, J=2.28 Hz, 0.4H), 3.18-3.46 (m, 2H), 2.10-2.20 (m, 0.4H), 1.84-1.97 (m, 0.6H), 1.61-1.67 (m, 0.6H), 1.53 (dd, J=12.96, 6.32 Hz, 0.4H), 1.28 (s, 9H), 0.68 (s, 9H), −0.04 to 0.01 (m, 9H), −0.10 to −0.06 (m, 6H).

### tert-Butyl-(2R,3S)-3-hydroxy-2-prop-2-yn-1-ylpyrrolidine-1-carboxylate (5)

tert-Butyl-(2R,3S)-3-{[tert-butyl(dimethyl)silyl]oxy}-2-{1-[(phenoxycarbonothioyl)oxy]-3-(trimethylsilyl)prop-2-yn-1-yl}pyrrol-idine-1-carboxylate (3) (6.44 g, 0.0114 mol) was dissolved in toluene (60 mL) and then tri-n-butyltin hydride (4.61 mL, 0.0171 mol) was added and the reaction was heated at 80° C. After the reaction had reached this temperature, a solution of 2,2′-azo-bis-isobutyronitrile (0.94 g, 0.0057 mol) in toluene (26 mL) was added dropwise (over ˜15-20 min) via a pressure equalizing dropping funnel. The reaction was stirred for 2 hr at the same temperature and then checked by TLC for completion (15% diethyl ether in hexanes, ninhydrin stain), which shows complete consumption of the starting thionocarbonate and formation of a higher R_{f} product. The reaction was cooled to ambient temperature and then concentrated to ½ volume and then diluted with 100 mL of diethyl ether. The resulting solution was washed ×2 with 1M NaOH (50 mL each) and then brine (50 mL), dried over MgSO₄, filtered and concentrated in vacuo to afford the intermediate deoxygenated compound (4). The intermediate was subjected to silica gel chromatography on 90 g of silica gel, eluting with 0 to 20% ethyl acetate in hexanes over a 1.5 hr gradient, to afford the desired intermediate, 3.55 g in 76% yield (¹H NMR confirms). The intermediate was then dissolved in tetrahydrofuran (150 mL) and 1.00 M of tetra-n-butylammonium fluoride in tetrahydrofuran (28 mL) was added dropwise via a pressure equalizing dropping funnel and the reaction was stirred at ambient temperature for 1 hr. TLC of the product formed from this route co-spots with previously prepared (5) obtained from a different route (compare Example 16 (previous route) [α]²²_{D} is −215.9° (average over 5 readings) and this route [α]²²_{D} is −215.9° (average over 5 readings)). ¹H NMR is also identical to that previously prepared in the synthesis of compound (16) of Example 16 and compound (18) of Example 16; (intermediate (4)) ¹H NMR (400 MHz, CDCl₃) δ ppm 4.23-4.30 (m, 1H), 3.65 (dd, J=9.95, 3.32 Hz, 0.5H, α-H rotamer), 3.51 (dd, J=9.54, 3.32 Hz, 0.5H, α-H rotamer), 3.23-3.48 (m, 2H), 2.64 (dd, J=16.90, 3.42 Hz, 0.5H, rotamer), 2.52 (dd, J=16.90, 3.63 Hz, 0.5H, rotamer), 2.02-2.10 (dd, 1H, J=16.8, 9.6 Hz), 1.85-1.96 (m, 1H), 1.62-1.70 (m, 1H), 1.38 (s, 4.5H, t-Boc rotamer), 1.36 (s, 4.5H, t-Boc rotamer), 0.78 (s, 9H, TBS t-Bu), 0.05 (s, 5H, TMS rotamer), 0.04 (s, 4H, TMS rotamer), 0.01 (s, 3 H), −0.00 (s, 3H); (final product, (5)): ¹H NMR (400 MHz, CDCl₃) δ ppm 4.41-4.45 (m, 1H), 3.71-3.84 (m, 1H), 3.51-3.66 (m, 1H), 3.40-3.51 (m, 1H), 2.63-2.83 (m, 1H), 2.22-2.33 (m, 1H), 2.14-2.22 (m, 1H), 2.01-2.06 (m, 1H, alkyne CH), 1.88-1.94 (m, 1H), 1.86 (d, J=3.73 Hz, 1H, OH), 1.49 (br. s., 9H).

### Example 30B

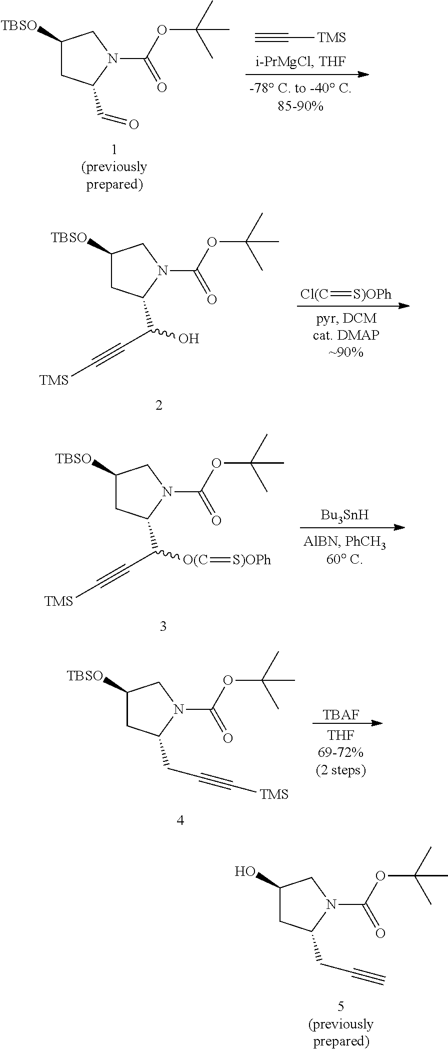

### tert-Butyl-(2S,4R)-4-{[tert-butyl(dimethyl)silyl]oxy}-2-[1-hydroxy-3-(trimethylsilyl)prop-2-yn-1-yl]pyrrolidine-1-carboxylate (2)

(Trimethylsilyl)acetylene (0.69 mL, 0.0048 mol) was dissolved in tetrahydrofuran (20 mL) and then the solution was cooled at 0° C. using an ice-water bath. After the solution was cooled, a solution of 2.00 M isopropylmagnesium chloride in tetrahydrofuran (3 mL) was added dropwise via syringe over a 2-3 minute period of time. After the addition was complete, the reaction was stirred at reduced temperature for 30 minutes and then cooled at −40° C. using a dry ice acetone bath. When the reaction was sufficiently cooled, the aldehyde tert-butyl-(2S,4R)-4-{[tert-butyl(dimethyl)silyl]oxy}-2-formylpyrrolidine-1-carboxylate (1) (1.25 g, 0.00379 mol) was added dropwise via syringe as a solution in tetrahydrofuran (10 mL). The reaction was stirred for 45 minutes, maintained at the reduced temperature, and then checked by TLC. TLC after this period of time shows that the reaction is essentially complete. The reaction was placed in a refrigerator at −20° C. for 24 hr and then quenched by the addition of 100 mL of saturated sodium bicarbonate. The organic product was extracted with ethyl acetate (3×50 mL) and the combined organic layers were washed with water and brine, dried over MgSO₄, filtered and concentrated in vacuo to afford the crude product. Silica gel chromatography using a 40 g silica gel cartridge afforded purified product as a mixture of diastereomers (eluted with 0 to 30% ethyl acetate in hexanes), (2) 1.47 g in 91% yield; ¹H NMR (400 MHz, CDCl₃) δ ppm 5.96 (d, J=9.12 Hz, 0.65H, OH diastereomer), 5.00 (d, J=2.70 Hz, 0.35H, OH diastereomer), 4.20-3.97 (m, 3 H), 3.29 (m, 1H), 3.19 (m, 1H), 1.67-1.93 (m, 2H), 1.31 (s, 6H, t-Bu of Boc), 1.29 (s, 3H, t-Bu of Boc), 0.70 (s, 9H, t-Bu of TBS), −0.00 (s, 4H, SiMe₃ on alkyne), −0.02 (s, H, SiMe₃ on alkyne), −0.10 (br. s., 2H, SiMe₂ on TBS), −0.10 (s, 4H, SiMe₂ on TBS).

### tert-Butyl-(2S,4R)-4-{[tert-butyl(dimethyl)silyl]oxy}-2-{1-[(phenoxycarbonothioyl)oxy]-3-(trimethylsilyl)prop-2-yn-1-yl}pyrrolidine-1-carboxylate (3)

The alcohol, tert-butyl-(2S,4R)-4-{[tert-butyl(dimethyl)silyl]oxy}-2-[1-hydroxy-3-(trimethylsilyl)prop-2-yn-1-yl]pyrrolidine-1-carboxylate (2) (1.55 g, 3.62 mmol), was dissolved in CH₂Cl₂ (20 mL) at ambient temperature. Then, pyridine (0.99 mL, 12 mmol) and 4-dimethylaminopyridine (0.04 g, 0.3 mmol) were added followed by phenyl chlorothionocarbonate (0.55 mL, 4.0 mmol). The reaction was stirred for 8 hr at room temperature and then checked for completion by TLC. TLC after this period of time shows some starting material remaining but significant progress toward product has occurred. The reaction was charged with more pyridine (1.5 mL, 18 mmol) and phenyl chlorothionocarbonate (0.25 mL, 1.8 mmol) with continued stirring another 14 hr. TLC after this period of time shows complete consumption of the starting material. The reaction was quenched by the addition of 100 mL of water and then the organic product was extracted with 3-60 mL portions of CH₂Cl₂. The combined organic layers were washed with 0.1 M HCl (2×50 mL), water (50 mL), saturated aqueous sodium bicarbonate (50 mL) and finally with a brine solution (50 mL). The organic layer was then dried over magnesium sulfate, vacuum filtered to remove the drying agent and then concentrated in vacuo to afford the crude product. Silica gel chromatography (40 g) eluting with 0 to 20% ethyl acetate in hexanes afforded purified product, (3) 2.09 g, as a mixture of diastereomers in quantitative yield; ¹H NMR (400 MHz, CDCl₃) δ ppm 7.01-7.30 (m, 4H), 6.89-6.96 (m, 1H), 6.09-6.24 (m, 1H), 4.20-4.37 (m, 1H), 3.99-4.18 (m, 1H), 3.12-3.41 (m, 2H), 2.12-2.23 (m, 1H), 1.82-1.95 (m, 1H), 1.25-1.31 (m, 9H), 0.69 (s, 9H), 0.02 (s, 4.5H), −0.00 (s, 4.5H), −0.11 (m, 6H).

### tert-Butyl-(2R,4R)-4-hydroxy-2-prop-2-yn-1-ylpyrrolidine-1-carboxylate (5)

tert-Butyl-(2S,4R)-4-{[tert-butyl(dimethyl)silyl]oxy}-2-{1-[(phenoxycarbonothioyl)oxy]-3-(trimethylsilyl)prop-2-yn-1-yl}pyrrolidine-1-carboxylate (3) (2.52 g, 4.47 mmol) was dissolved in toluene (50 mL) and tri-n-butyltin hydride (2.70 mL, 10.0 mmol) was added. The reaction was heated at 65° C. and then 2,2′-azo-bis-isobutyronitrile (0.37 g, 2.2 mmol) in toluene (50 mL) was added dropwise, via syringe, over a 10 minute period of time. After 2 hr at reflux, the reaction was checked by TLC (15% Et₂O/Hexanes) for completion. TLC after this period of time shows a slightly lower R_{f} product that is not UV active, whereas the thionocarbonate is UV active. The reaction was cooled to room temperature and then a solution of 1.0 M of tetra-n-butylammonium fluoride in tetrahydrofuran (20 mL) was added with continued stirring for 2 hr. TLC after ˜30 minutes shows that the initial starting material is consumed, but the final product is present only in a small amount. After the two hour period of time, the reaction mixture was concentrated in vacuo and then taken up in acetonitrile (˜50 mL). The acetonitrile layer was washed with hexanes (3×30 mL) and then the acetonitrile layer was concentrated in vacuo to afford a thick yellow oil that was purified by silica gel chromatography (40 g silica gel cartridge, eluting with 0 to 30% ethyl acetate in CH₂Cl₂) to afford the final product (5), 751 mg in 75% yield for the two steps (yield corrected for allene). ¹H NMR of the final product revealed a small amount (˜7% by ¹H NMR integration) of allene product formation (signal at 4.83 ppm (dd, J=6, 2 Hz, 2H))—yield is corrected for this; ¹H NMR, are identical to product previously prepared via another route (see Example 7) in the synthesis of compounds 17 and 18 of Example 7B; MS: ES⁺248.1 m/z (M+Na)⁺.

### Example 31

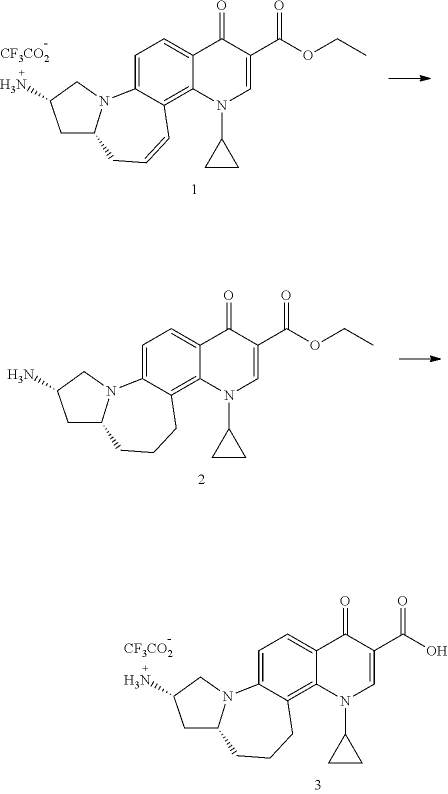

General Methods: All reactions were carried out under a nitrogen atmosphere unless otherwise noted. Analytical HPLC conditions: Agilent 1100 HPLC, Agilent XDB-C18 50×4.6 mm/1.8 micron column; 1.5 mL/min; solvent A: water (0.1% TFA), solvent B: acetonitrile (0.07% TFA); gradient: 5 min 95% A to 95% B then 1 min hold, 1 min 95% B to 95% A then 30 sec hold; detection @ 210, 254, and 280 nm. Preparative HPLC conditions: Phenomenex Luna 250×21.20 mm/10 micron column; solvent A: acetonitrile (0.07% TFA), solvent B: water (0.1% TFA); gradient: 1 min 10% A, 20 min 10% to 60%, hold 10 min, 3 min 60% to 95% A, hold 2 min, 5 min 5%.

### (7aR,9S)-2-Carboxy-4-cyclopropyl-1-oxo-4,5,6,7,7a,8,9,10-octahydro-1H-pyrrolo[1′,2′:1,7]azepino[2,3-h]quinolin-9-aminium trifluoroacetate (3)

To a sample of (7aR,9S)-4-cyclopropyl-2-(ethoxycarbonyl)-1-oxo-4,7,7a,8,9,10-hexahydro-1H-pyrrolo[1′,2′:1,7]azepino[2,3-h]quinolin-9-aminium trifluoroacetate (1) (29 mg, 0.059 mmol) was added tetrahydrofuran (3 mL, 40 mmol), Et₃N (3 drops) until basic by wet pH paper, and water (0.6 mL, 30 mmol) to dissolve the salts. The clear, bright yellow solution was sparged with nitrogen (10 min) and treated with 10% palladium on carbon (6.2 mg, 0.0059 mmol). Hydrogen (˜1 L) was bubbled through the reaction mixture, which was allowed to stir for 20 h at rt under hydrogen; HPLC showed remaining starting material. Additional THF (1 mL) was added and hydrogen (˜1 L) was bubbled through the reaction mixture, which was allowed to stir for an additional 45 h; HPLC showed complete conversion. The reaction mixture was filtered (0.45 μm PTFE/MeOH) and the filtrate was concentrated in vacuo to afford crude ethyl-(7aR,9S)-9-amino-4-cyclopropyl-1-oxo-4,5,6,7,7a,8,9,10-octahydro-1H-pyrrolo[1′,2′:1,7]azepino[2,3-h]quinoline-2-carboxylate (2) as a bright yellow residue. HPLC purity >95% at 280 nm (ret. time, 2.373 min); MS (ESI+) for C₂₂H₂₇N₃O₃ m/z 382.0 (M+H)⁺.

A solution of the above crude (2) in acetonitrile (3 mL, 60 mmol) and water (0.2 mL, 10 mmol) was treated with 0.5 M aqueous sodium hydroxide (0.20 mL, 0.1 mmol). The reaction mixture was heated at 60° C. for 2 h; HPLC showed no remaining starting material. The reaction mixture was allowed to cool to rt and was neutralized with acetic acid (2 drops), followed by concentration to ˜2 mL. Purification by preparative HPLC gave the title compound (3) as an orange solid (8.9 mg, 31% over two steps). HPLC purity >95% at 280 nm (ret. time, 2.305 min); MS (ESI+) for C₂₀H₂₃N₃O₃ m/z 354.0. (M+H)⁺.

### Example 32

The following compounds were prepared according to the synthetic routes described in the examples above.

TABLE 1

| Com- | | |
|---|---|---|
| pound # | Structure | MH⁺ |
| 1 | 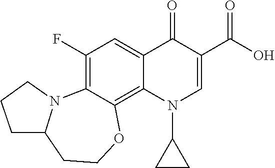 | 359.1 |
| 2 | 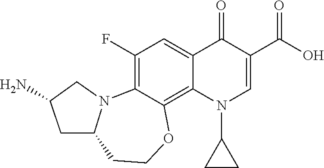 | 374.1 |
| 3 | 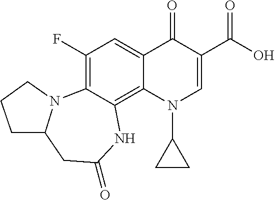 | 372.1 |
| 4 | 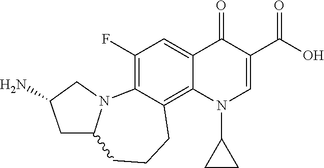 | 371.41 |
| 5 | 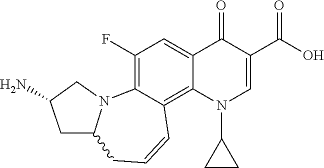 | 369.39 |

### Example 33

Minimum inhibitory concentrations (MIC) were determined by reference Clinical and Laboratory Standards Institute (CLSI) broth microdilution methods per M7-A7 [2006]. Quality control ranges utilizing E. coli ATCC 25922, P. aeruginosa ATCC 27853 and S. aureus ATCC 29213, and interpretive criteria for comparator agents were as published in CLSI M100-S17 [2007]. Briefly, serial two-fold dilutions of the test compounds were prepared at 2× concentration in Mueller Hinton Broth. The compound dilutions were mixed in 96-well assay plates in a 1:1 ratio with bacterial inoculum. The inoculum was prepared by suspension of a colony from an agar plate that was prepared the previous day. Bacteria were suspended in sterile saline and added to each assay plate to obtain a final concentration of 5×10⁵ CFU/mL. The plates were incubated at 35° C. for 20 hours in ambient air. The MIC was determined to be the lowest concentration of the test compound that resulted in no visible bacterial growth as compared to untreated control. Data for certain representative compounds is shown in Tables 2 and 3 below.

TABLE 2

| | AECO | AECO | AABA | AABA | APAE | APAE | ASAU | ASAU | ASPN | ASPN |
|---|---|---|---|---|---|---|---|---|---|---|
| Ex #/Cpd # | 001 | 066 | 1065 | 1009 | 001 | 054 | 001 | 1031 | 001 | 1027 |
| Ciprofloxacin | A | C | A | C | A | C | A | C | B | C |
| Moxifloxacin | A | C | A | C | B | C | A | B | A | B |
| Gemifloxacin | A | C | A | C | A | C | A | C | A | B |
| Levofloxacin | A | C | A | C | B | C | A | C | B | C |
| Comparative | C | C | C | — | C | — | B | — | — | — |
| Compound 1 | | | | | | | | | | |
| 1/10 | A | C | B | C | C | C | A | B | A | C |
| 3/15 | A | B | A | C | B | C | A | B | A | B |
| 5/10 | A | C | B | C | B | C | B | C | B | C |
| 5/11 | A | C | B | C | B | C | B | C | B | C |
| 6/7 | A | B | A | B | A | C | A | B | A | A |
| 7B/17 | A | B | A | C | B | C | A | B | A | B |
| 7B/18 | A | B | A | C | A | C | A | B | A | B |
| 7B/20 | A | C | A | C | B | C | A | B | A | C |
| 7C/29 | B | C | B | C | C | C | B | C | C | C |
| 7C/30 | B | C | B | C | C | C | B | C | B | B |
| 7D/39 | A | C | A | C | B | C | A | C | B | C |
| 7D/40 | A | C | A | C | B | C | A | C | B | C |
| 7E/49 | C | C | C | C | C | C | B | C | C | C |
| 7E/50 | B | C | C | C | C | C | C | C | B | B |
| 8/10 | B | C | B | C | C | C | A | C | C | C |
| 8/12 | C | C | C | C | C | C | B | C | C | C |
| 8/14 | B | C | B | C | C | C | B | C | C | C |
| 10/6 | A | C | B | C | B | C | A | B | A | B |
| 11/8 | A | C | A | C | C | C | A | B | A | C |
| 12/4 | A | C | C | C | B | C | A | C | B | C |
| 13/5 | A | C | A | C | B | C | A | C | B | C |
| 14/4 | B | C | C | C | C | C | C | C | C | C |
| 15/3 | A | C | A | C | C | C | A | C | B | C |
| 16/16 | A | B | A | B | A | C | A | A | A | B |
| 16/18 | A | B | A | B | A | C | A | B | A | B |
| 17/8 | A | B | A | C | A | C | A | B | A | B |
| 17/10 | A | C | A | C | B | C | A | B | A | B |
| 18/6 | A | C | B | C | C | C | A | B | A | C |
| 18/8 | A | C | B | C | C | C | A | B | A | C |
| 18/10 | B | C | C | C | C | C | A | C | B | C |
| 18/11 | A | C | A | C | B | C | A | A | A | A |
| 19/5 | B | C | B | C | C | C | A | C | B | C |
| 20/14 | A | C | A | C | B | C | A | C | A | C |
| 20/16 | A | C | A | C | B | C | A | C | A | C |
| 21/9 | B | C | C | C | C | C | B | C | C | C |
| 22/17 | B | C | B | C | B | C | A | C | B | C |
| 23/12 | C | C | C | C | C | C | B | C | C | C |
| 23/14 | C | C | C | C | C | C | C | C | C | C |
| 24/rac-17 | A | C | A | C | B | C | A | B | A | B |
| 24/rac-16 | A | C | A | C | B | C | A | B | A | B |
| 25/14 | A | C | A | C | B | C | A | C | A | B |
| 26/16 | A | C | B | C | B | C | B | C | B | C |
| 26/17 | A | C | B | C | B | C | B | C | B | C |
| 27/5 | A | C | B | C | C | C | A | B | A | C |
| 27/7 | B | C | C | C | C | C | A | B | B | C |
| 27/9 | B | C | B | C | C | C | B | C | C | C |
| 27/10 | B | C | B | C | C | C | B | C | C | C |
| 27/11 | B | C | B | C | C | C | A | C | B | C |
| 27/12 | B | C | B | C | C | C | A | B | B | C |
| 32/1 | A | C | B | C | C | C | A | C | B | C |
| 32/2 | A | C | A | C | A | C | A | C | A | C |
| 32/3 | C | C | C | — | C | — | C | — | — | — |
| 32/4 | A | C | C | C | B | C | A | C | A | B |
| 32 /5 | A | C | A | C | A | C | A | B | A | B |

| * Key: | | |
|---|---|---|
| Strain | ACH Code | Phenotype |
| E. coli | AECO001 | Fluoroquinolone susceptible, ATCC25922 |
| | AECO066 | Fluoroquinolone-resistant |
| A. baumannii | AABA1065 | Fluoroquinolone-susceptible |
| | AABA1009 | Fluoroquinolone-resistant |
| P. aeruginosa | APAE001 | Fluoroquinolone-susceptible, ATCC27853 |
| | APAE054 | Fluoroquinolone-resistant |
| S. aureus | ASAU001 | Fluoroquinolone-susceptible, ATCC29213 |
| | ASAU1031 | Fluoroquinolone-resistant |
| S. pneumoniae | ASPN001 | Fluoroquinolone-susceptible, ATCC49619 |
| | ASPN1027 | Fluoroquinolone-resistant |

** MIC Key:
MIC's of 1.0 μg/mL or less = A
MIC's of greater than 1.0 μg/mL to 8.0 μg/mL = B
MIC's of greater than 8.0 μg/mL = C
*** Comparative Compound 1:

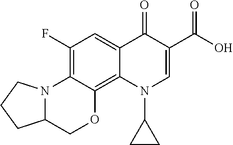

TABLE 3

| | Ex #/Cpd # | | | | | | | | | | | | | | |
|---|---|---|---|---|---|---|---|---|---|---|---|---|---|---|---|
| | | | | | | | | | | | | | Comp | Comp | Comp |
| | 3/15 | 7D/39 | 7C/29 | 7E/49 | 7B/18 | 7D/40 | 7C/30 | 7E/50 | 16/18 | 17/10 | 16/16 | 17/8 | Cpd 1 | Cpd 2 | Cpd 3 |
| AECO001 | A | A | C | D | A | A | B | C | A | A | A | A | A | A | A |
| AECO002 | A | A | C | D | A | A | B | D | A | A | A | A | A | A | A |
| AECO066 | C | D | D | D | C | D | D | D | C | D | C | C | D | D | D |
| AECO086 | C | D | D | D | C | D | D | D | C | D | B | B | D | D | D |
| AABA1065 | A | A | C | D | A | A | C | D | A | A | A | A | A | A | A |
| AABA1020 | D | D | D | D | C | D | D | D | D | D | C | D | D | D | D |
| APAE001 | A | C | D | D | A | B | D | D | A | B | A | A | B | B | A |
| APAE002 | A | B | D | D | A | B | D | D | A | A | A | A | A | B | A |
| APAE054 | D | D | D | D | D | D | D | D | D | D | D | D | D | D | D |
| ASAU001 | A | A | C | C | A | A | B | D | A | A | A | A | A | A | A |
| ASAU002 | A | A | C | C | A | A | B | C | A | A | A | A | A | A | A |
| ASAU1033 | D | D | D | D | D | D | D | D | C | D | B | C | D | D | D |
| ASAU1031 | C | D | D | D | C | D | D | D | A | B | A | A | D | C | D |
| ASPN001 | A | B | D | D | A | B | C | C | A | A | A | A | B | A | B |
| ASPN002 | A | B | D | D | A | B | C | C | A | A | A | A | B | A | B |
| ASPN1018 | B | D | D | C | B | D | C | C | B | C | A | B | D | C | D |
| ASPN1027 | C | D | D | D | C | D | C | C | A | B | A | A | D | C | D |

| | | |
|---|---|---|
| * Key: | | |
| Strain | ACH Code | Phenotype |
| E. coli | AECO001 | Fluoroquinolone susceptible, ATCC25922 |
| | AECO002 | Fluoroquinolone susceptible |
| | AECO066 | Fluoroquinolone-resistant |
| | AECO086 | Fluoroquinolone-resistant |
| A. baumannii | AABA1065 | Fluoroquinolone-susceptible |
| | AABA1020 | Fluoroquinolone-resistant |
| P. aeruginosa | APAE001 | Fluoroquinolone-susceptible, ATCC27853 |
| | APAE002 | Fluoroquinolone susceptible |
| | APAE0054 | Fluoroquinolone-resistant |
| S. aureus | ASAU001 | Fluoroquinolone-susceptible, ATCC29213 |
| | ASAU002 | Fluoroquinolone-susceptible, ATCC33591 (MRSA) |
| | ASAU1033 | Fluoroquinolone-resistant |
| | ASAU1031 | Fluoroquinolone-resistant |
| S. pneumoniae | ASPN001 | Fluoroquinolone-susceptible, ATCC49619 |
| | ASPN002 | Fluoroquinolone-susceptible, ATCC10813 |
| | ASPN1018 | Fluoroquinolone-resistant |
| | ASPN1027 | Fluoroquinolone-resistant |

** MIC Key:
MIC's of less than 1.0 = A
MIC's of 1.0 μg/mL to 2.0 μg/mL = B
MIC's of 2.0 μg/mL to 8.0 μg/mL = C
MIC's of greater than 8.0 μg/mL = D
*** Comparative Compounds:
Comp Cpd 1 is Levofloxacin
Comp Cpd 2 is Moxifloxacin
Comp Cpd 3 is Ciprofloxacin

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound having the following structure (I): 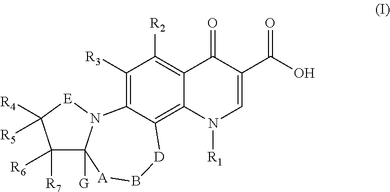
or a stereoisomer, pharmaceutically acceptable salt or prodrug thereof,
wherein:
A, B and D are as follows:
A is —C(R_{8b})₂—, —C(═O)—, —C(R_{8b})(OR₈ₐ)—, —C(R_{8b})(N(R₈ₐ)₂)—, —C(═NOR₈ₐ)—, —S(═O)— or —SO₂—;
B is —C(R_{8b})₂—, —C(═O)—, —C(R_{8b})(OR₈ₐ)—, —C(R_{8b})(N(R₈ₐ)₂)—, —C(═NOR₈ₐ)—, —O—, —S—, —S(═O)—, —SO₂— or —N(R₈ₐ)—; and
D is —C(R_{8b})₂—, —C(═O)—, —O—, —S—, —S(═O)—, —SO₂—, —N(R₈ₐ)—, —C(R_{8b})(OR₈ₐ)— or —C(R_{8b})(N(R₈ₐ)₂)—;
or taken together, are —C(R_{8b})═C(R_{8b})—, —C(R_{8b})═N— or 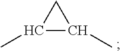
or
or B-D, taken together, are —C(R_{8b})═C(R_{8b})—, —N═C(R_{8b})— or 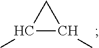
E is —C(R_{8c})₂— or —C(═O)—;
G is hydrogen or methyl;
R₁ is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl, optionally substituted heteroaryl or optionally substituted heteroarylalkyl;
R₂ is hydrogen, methyl or amino;
R₃ is hydrogen, fluorine or chlorine;
R₄, R₅, R₆, R₇ are, independently, hydrogen, halogen, amino, hydroxyl, optionally substituted alkyl, optionally substituted cycloalkyl, optionally substituted alkoxy, optionally substituted alkylamino or —N(R₈ₐ)₂, or R₄ and R₅, taken together, are ═CHR_{8b}, ═NOR₈ₐ, ═NNR₈ₐ or ═O or, together with the atom to which they are attached, form an optionally substituted heterocyclic ring having from 3 to 6 ring atoms, or R₆ and R₇, taken together, are ═CHR_{8b}, ═NOR₈ₐ, ═NNR₈ₐ or ═O, or, together with the atom to which they are attached, form an optionally substituted heterocyclic ring having from 3 to 6 ring atoms, or R₅ and R₆, R₅ and R₇, R₄ and R₆, or R₄ and R₇, taken together with the atoms to which they are attached, form a heterocyclic ring having from 3 to 6 ring atoms;
each R₈ₐ is, independently, hydrogen, C₁-C₆ alkyl, C₁-C₆ cycloalkyl, C₁-C₆ cycloalkylalkyl or —C(═O)R_{8c};
each R_{8b} is, independently, hydrogen, halogen, C₁-C₆ alkyl, C₁-C₆ cycloalkyl, C₁-C₆ haloalkyl or C₁-C₆ cycloalkylalkyl; and
each R_{8c} is, independently, hydrogen or C₁-C₆ alkyl.

2. A compound of claim 1 wherein A-B-D, taken together, are —CH₂CH₂CH₂—, —CH(CH₃)CH₂CH₂—, —C(CH₃)₂CH₂CH₂—, —CH₂CH═CH—, —CH═CHCH₂—, CH(OR₈ₐ)CH₂CH₂—, —C(CH₃)(OR₈ₐ)CH₂CH₂—, —CH(N(R₈ₐ)₂)CH₂CH₂—, —C(CH₃)(N(R₈ₐ)₂)CH₂CH₂—, —C(═NOR₈ₐ)CH₂CH₂—, —C(═O)N(R₈ₐ)CH₂—, —CH₂CH(OR₈ₐ)CH₂—, —CH₂C(CH₃)(OR₈ₐ)CH₂—, —CH₂CH(N(R₈ₐ)₂)CH₂—, —CH₂C(═NOR₈ₐ)CH₂—, —CH₂C(═O)CH₂—, —CH₂C(CH₃)₂CH₂—, —CH₂OCH₂—, —CH₂SCH₂—, —CH₂S(═O)CH₂—, —CH₂SO₂CH₂—, —CH₂N(R₈ₐ)CH₂—, —CH₂CH₂CH(CH₃)—, —CH₂CH₂C(CH₃)₂—, —CH₂C(═O)N(R₈ₐ)—, —CH₂N(R₈ₐ)C(═O)—, —CH₂SO₂N(R₈ₐ)—, —CH₂N(R₈ₐ)SO₂—, —CH₂CH₂O—, —CH₂CH₂N(R₈ₐ)—, —CH₂CH₂S(═O)— or —CH₂CH₂SO₂—.

3. A compound of claim 1 wherein A-B-D, taken together, are —C(R_{8b})₂C(R_{8b})₂C(R_{8b})₂—, —C(R_{8b})₂C(R_{8b})═C(R_{8b})— or —C(R_{8b})═C(R_{8b})C(R_{8b})₂—.

4-6. (canceled)

7. A compound of claim 3 wherein each R_{8b} is hydrogen.

8. A compound of claim 1 wherein A is —CH₂—.

9. A compound of claim 8 wherein B-D, taken together, are —C(R_{8b})₂O—, —OC(R_{8b})₂—, —C(R_{8b})₂S—, —SC(R_{8b})₂—, —C(R_{8b})₂N(R₈ₐ)—, —N(R₈ₐ)C(R_{8b})₂—, —C(R_{8b})₂C(R_{8b})₂—, —C(R_{8b})═C(R_{8b})— or —N═C(R_{8b})—.

10-13. (canceled)

14. A compound of claim 1 wherein B is —O—.

15-32. (canceled)

33. A compound of claim 1 wherein R₄, R₅, R₅ and R₇ are, independently, hydrogen, amino, optionally substituted alkyl, optionally substituted cycloalkyl, optionally substituted alkylamino or —N(R₈ₐ)₂.

34. (canceled)

35. A compound of claim 33 wherein R₄, R₅ and R₆ are each hydrogen and R₇ is amino, substituted alkyl, substituted cycloalkyl, alkylamino, or —N(R₈ₐ)₂, wherein substituted alkyl is —(C₁-C₆ alkyl)N(R₈ₐ)₂ and substituted cycloalkyl is —(C₃-C₆ cycloalkyl)N(R₈ₐ)₂.

36. (canceled)

37. A compound of claim 35 wherein R₇ is —NH₂, —CH₂NH₂, —CH(CH₃)NH₂, —C(CH₃)₂NH₂, or 1-amino-cycloprop-1-yl.

38-48. (canceled)

49. A compound of claim 1 wherein R₁ is optionally substituted cycloalkyl.

50. A compound of claim 49 wherein R₁ is cyclopropyl.

51. A compound of claim 1 wherein R₂ is hydrogen.

52. A compound of claim 1 wherein R₃ is fluorine.

53. (canceled)

54. A compound of claim 1 wherein E is —CH₂—.

55-56. (canceled)

57. A compound of claim 1 wherein G is hydrogen.

58. A compound of claim 1 wherein the compound has the following structure:

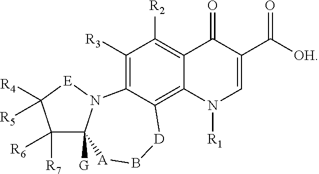

59. A compound of claim 1 wherein the compound is one of the following:
Example #/Compound #
```
  1/10
  2/16
  3/15
  4/4
  5/10
  5/11
  6/7
7B/17
7B/18
7B/20
7C/29
7C/30
7D/39
7D/40
7E/49
7E/50
  8/10
  8/12
  8/14
  9/8
10/6
11/8
12/4
13/5
14/4
15/3
16/16
16/18
17/8
17/10
18/6
18/8
18/10
18/11
19/5
20/14
20/16
21/9
22/17
23/12
23/14
24/rac-17
24/rac-16
25/14
26/16
26/17
27/5
27/7
27/9
27/10
27/11
27/12
32/1
32/2
32/3
32/4
32/5
```

60. A pharmaceutical composition comprising a compound of claim 1, or a stereoisomer, pharmaceutically acceptable salt or prodrug thereof, and a pharmaceutically acceptable carrier, diluent or excipient.

61. A method of treating a bacterial infection in a mammal in need thereof, comprising administering to the mammal an effective amount of a compound of claim 1.

62-112. (canceled)
